(19) United States
(12) Patent Application Publication
(10) Pub. No.: US 2024/0336541 A1
(43) Pub. Date: Oct. 10, 2024

(54) AMINE COMPOUND AND ORGANIC LIGHT-EMITTING DEVICE COMPRISING SAME

(71) Applicant: LT MATERIALS CO., LTD., Yongin-si, Gyeonggi-do (KR)

(72) Inventors: Na-Yeong KIM, Yongin-si (KR); Nam-Jin LEE, Yongin-si (KR); Won-Jang JEONG, Yongin-si (KR); Dong-Jun KIM, Yongin-si (KR)

(73) Assignee: LT MATERIALS CO., LTD., Yongin-si, Gyeonggi-do (KR)

(21) Appl. No.: 18/573,332

(22) PCT Filed: Sep. 8, 2022
(86) PCT No.: PCT/KR2022/013544

§ 371 Date: Dec. 21, 2023

(30) Foreign Application Priority Data
Oct. 28, 2021 (KR) 10-2021-0145354

(51) Int. Cl. C07B 59/00; C07C 211/61; C07D 307/91; C07D 333/76; C09K 11/06; H10K 50/11; H10K 50/15; H10K 85/60

(52) U.S. Cl. CPC C07B 59/001; C07B 59/002; C07C 211/61; C07D 307/91; C07D 333/76; C09K 11/06; H10K 50/11; H10K 50/15; H10K 85/633; H10K 85/6574; H10K 85/6576; C07B 2200/05

(57) ABSTRACT

The present specification relates to an amine compound and an organic light emitting device including the same.

### TECHNICAL FIELD

The present specification relates to an amine compound and an organic light emitting device including the same.

This application claims priority to and the benefits of Korean Patent Application No. 10-2021-0145354, filed with the Korean Intellectual Property Office on Oct. 28, 2021, the entire contents of which are incorporated herein by reference.

### BACKGROUND ART

An electroluminescence device is a kind of self-emitting type display device, and has an advantage in that the viewing angle is wide, the contrast is excellent, and the response speed is fast.

An organic light emitting device has a structure in which an organic thin film is disposed between two electrodes. When a voltage is applied to an organic light emitting device having the structure, electrons and holes injected from the two electrodes combine with each other in an organic thin film to make a pair, and then, emit light while being extinguished. The organic thin film may be composed of a single layer or multi layers, if necessary.

A material for the organic thin film may have a light emitting function, if necessary. For example, as the material for the organic thin film, it is also possible to use a compound, which may itself constitute a light emitting layer alone, or it is also possible to use a compound, which may serve as a host or a dopant of a host-dopant-based light emitting layer. In addition, as a material for the organic thin film, it is also possible to use a compound, which may play a role such as a hole injection, hole transport, electron blocking, hole blocking, electron transport or electron injection.

In order to improve the performance, service life, or efficiency of the organic light emitting device, there is a continuous need for developing a material for an organic thin film.

### RELATED ART DOCUMENT

### Patent Document

(Patent Document 1) U.S. Pat. No. 4,356,429

### DISCLOSURE

### Technical Problem

The present specification has been made in an effort to provide an amine compound and an organic light emitting device including the same.

### Technical Solution

In an exemplary embodiment of the present specification, provided is an amine compound represented by the following Chemical Formula 1 and having a deuterium content of more than 0% and 100% or less.

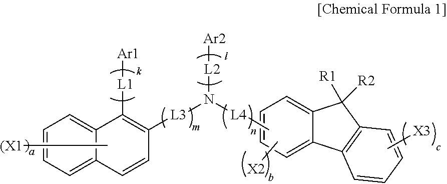

In Chemical Formula 1,
L1 to L4 are the same as or different from each other, and are each independently a direct bond; a substituted or unsubstituted arylene group having 6 to 60 carbon atoms; or a substituted or unsubstituted heteroarylene group having 2 to 60 carbon atoms,
Ar1 is a phenyl group substituted with deuterium; a substituted or unsubstituted aryl group having 8 to 60 carbon atoms; or a substituted or unsubstituted heteroaryl group having 2 to 60 carbon atoms,
Ar2 is a substituted or unsubstituted aryl group having 6 to 60 carbon atoms; or a substituted or unsubstituted heteroaryl group having 2 to 60 carbon atoms,
R1 and R2 are the same as or different from each other, and are each independently a substituted or unsubstituted alkyl group having 1 to 60 carbon atoms; a substituted or unsubstituted aryl group having 6 to 60 carbon atoms; or a substituted or unsubstituted heteroaryl group having 2 to 60 carbon atoms, or may be bonded to each other to form a substituted or unsubstituted hydrocarbon ring having 3 to 60 carbon atoms; or a substituted or unsubstituted hetero ring having 2 to 60 carbon atoms,
X1 is hydrogen; or deuterium, X2 and X3 are the same as or different from each other, and are each independently hydrogen; or deuterium; a substituted or unsubstituted aryl group having 6 to 60 carbon atoms; or a substituted or unsubstituted heteroaryl group having 2 to 60 carbon atoms,
k, l, m and n are each an integer from 1 to 3,
a is an integer from 0 to 6,
b is an integer from 0 to 3,
c is an integer from 0 to 4, and
when k, l, m, n, a, b, and c are each 2 or higher, substituents in the parenthesis are the same as or different from each other.

In another exemplary embodiment of the present specification, provided is an organic light emitting device including: a first electrode; a second electrode; and an organic material layer having one or more layers provided between the first electrode and the second electrode, in which one or more layers of the organic material layer include one or more types of amine compound.

### Advantageous Effects

When used in an organic light emitting device, the amine compound described in the present specification can lower the driving voltage of the device, improve the light efficiency, and improve the service life characteristics of the device. Specifically, in the amine compound of the present invention, a substituent is bonded to carbon 1 of naphthalene, an amine group including a fluorene structure is substituted at the ortho position of the substituent, and at least one deuterium is included to have an effect of improving the service life of the device.

### DESCRIPTION OF DRAWINGS

FIGS. 1 to 4 are views each exemplarily illustrating a stacking structure of an organic light emitting device according to an exemplary embodiment of the present specification.

### MODE FOR INVENTION

Hereinafter, the present specification will be described in more detail.

When one part “includes” one constituent element in the present specification, unless otherwise specifically described, this does not mean that another constituent element is excluded, but means that another constituent element may be further included.

In the present specification,

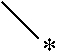 [SMILES: *C]

of a chemical formula means a position to which a constituent element is bonded.

The term “substitution” means that a hydrogen atom bonded to a carbon atom of a compound is changed into another substituent, and a position to be substituted is not limited as long as the position is a position at which the hydrogen atom is substituted, that is, a position at which the substituent may be substituted, and when two or more are substituted, the two or more substituents may be the same as or different from each other.

In the present specification, “substituted or unsubstituted” means being unsubstituted or substituted with one or more substituents selected from the group consisting of deuterium; a halogen group; —CN; a C1 to C60 alkyl group; a C2 to C60 alkenyl group; a C2 to C60 alkynyl group; a C3 to C60 cycloalkyl group; a C2 to C60 heterocycloalkyl group; a C6 to C60 aryl group; a C2 to C60 heteroaryl group; —SiRR′R″; —P(═O)RR′; and —NRR′, or with a substituent to which two or more substituents selected among the exemplified substituents are linked, and R, R′ and R″ are each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group.

In the present specification, “when a substituent is not indicated in the structure of a chemical formula or compound” means that a hydrogen atom is bonded to a carbon atom. However, since deuterium (²H) is an isotope of hydrogen, some hydrogen atoms may be deuterium.

In an exemplary embodiment of the present application, “when a substituent is not indicated in the structure of a chemical formula or compound” may mean that all the positions that may be reached by the substituent are hydrogen or deuterium. That is, deuterium is an isotope of hydrogen, and some hydrogen atoms may be deuterium which is an isotope, and in this case, the content of deuterium may be 0% to 100%.

In an exemplary embodiment of the present application, in “the case where a substituent is not indicated in the structure of a chemical formula or compound”, when the content of deuterium is 0%, the content of hydrogen is 100%, and all the substituents do not explicitly exclude deuterium such as hydrogen, hydrogen and deuterium may be mixed and used in the compound.

In an exemplary embodiment of the present application, deuterium is one of the isotopes of hydrogen, is an element that has a deuteron composed of one proton and one neutron as a nucleus, and may be represented by hydrogen-2, and the element symbol may also be expressed as D or ²H.

In an exemplary embodiment of the present application, the isotope means an atom with the same atomic number (Z), but different mass numbers (A), and the isotope may be interpreted as an element which has the same number of protons, but different number of neutrons.

In an exemplary embodiment of the present application, when the total number of substituents of a basic compound is defined as T1 and the number of specific substituents among the substituents is defined as T2, the content T % of the specific substituent may be defined as

$$T 2 / T 1 \times 100 = T \% .$$

That is, in an example, the deuterium content of 20% in a phenyl group represented by

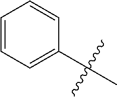 [SMILES: *c1ccccc1]

may be represented by 20% when the total number of substituents that the phenyl group can have is 5 (T1 in the formula) and the number of deuteriums among the substituents is 1 (T2 in the formula). That is, a deuterium content of 20% in the phenyl group may be represented by the following structural formula.

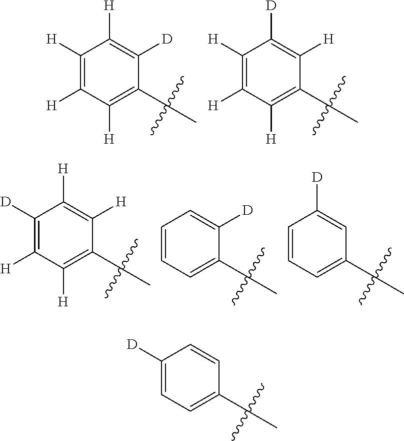

Further, in an exemplary embodiment of the present application, “a phenyl group having a deuterium content of 0%” may mean a phenyl group that does not include a deuterium atom, that is, has five hydrogen atoms.

In the present specification, the halogen may be fluorine, chlorine, bromine or iodine.

In the present specification, an alkyl group includes a straight-chain or branched-chain having 1 to 60 carbon atoms, and may be additionally substituted with another substituent. The number of carbon atoms of the alkyl group may be 1 to 60, specifically 1 to 40, and more specifically 1 to 20. Specific examples thereof include a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group, and the like, but are not limited thereto.

In the present specification, an alkenyl group includes a straight-chain or branched-chain having 2 to 60 carbon atoms, and may be additionally substituted with another substituent. The number of carbon atoms of the alkenyl group may be 2 to 60, specifically 2 to 40, and more specifically 2 to 20. Specific examples thereof include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl)vinyl-1-yl group, a 2,2-bis(diphenyl-1-yl)vinyl-1-yl group, a stilbenyl group, a styrenyl group, and the like, but are not limited thereto. In the present specification, the alkynyl group includes a straight-chain or branched-chain having 2 to 60 carbon atoms, and may be additionally substituted with another substituent. The number of carbon atoms of the alkynyl group may be 2 to 60, specifically 2 to 40, and more specifically 2 to 20.

In the present specification, the cycloalkyl group includes a monocycle or polycycle having 3 to 60 carbon atoms, and may be additionally substituted with another substituent. Here, the polycycle means a group in which a cycloalkyl group is directly linked to or fused with another cyclic group. Here, another cyclic group may also be a cycloalkyl group, but may also be another kind of cyclic group, for example, a heterocycloalkyl group, an aryl group, a heteroaryl group, and the like. The number of carbon atoms of the cycloalkyl group may be 3 to 60, specifically 3 to 40, and more specifically 5 to 20. Specific examples thereof include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group, and the like, but are not limited thereto.

In the present specification, the heterocycloalkyl group includes O, S, Se, N, or Si as a heteroatom, includes a monocycle or polycycle having 2 to 60 carbon atoms, and may be additionally substituted with another substituent. Here, the polycycle means a group in which a heterocycloalkyl group is directly linked to or fused with another cyclic group. Here, another cyclic group may also be a heterocycloalkyl group, but may also be another kind of cyclic group, for example, a cycloalkyl group, an aryl group, a heteroaryl group, and the like. The number of carbon atoms of the heterocycloalkyl group may be 2 to 60, specifically 2 to 40, and more specifically 3 to 20.

In the present specification, the aryl group includes a monocycle or polycycle having 6 to 60 carbon atoms, and may be additionally substituted with another substituent. Here, the polycycle means a group in which an aryl group is directly linked to or fused with another cyclic group. Here, another cyclic group may also be an aryl group, but may also be another kind of cyclic group, for example, a cycloalkyl group, a heterocycloalkyl group, a heteroaryl group, and the like. The aryl group includes a spiro group. The number of carbon atoms of the aryl group may be 6 to 60, specifically 6 to 40, and more specifically 6 to 25. Specific examples of the aryl group include a phenyl group, a biphenyl group, a terphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused cyclic group thereof, and the like, but are not limited thereto.

In the present specification, the terphenyl group may be selected from the following structures.

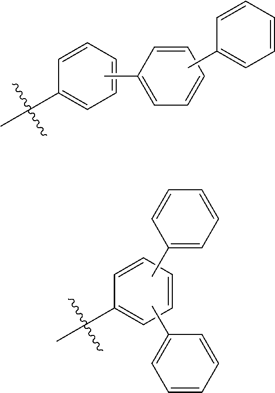

In the present specification, the fluorenyl group may be substituted, and adjacent substituents may be bonded to each other to form a ring.

When the fluorenyl group is substituted, the substituted fluorenyl group may be

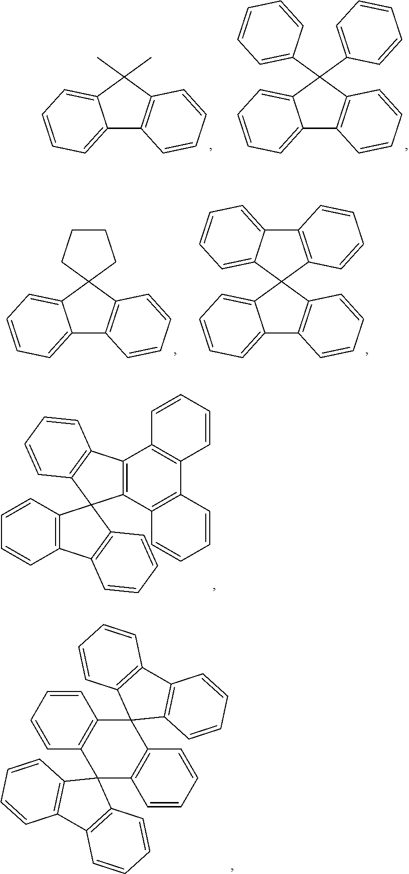

and the like, but is not limited thereto.

In the present specification, the heteroaryl group includes S, O, Se, N, or Si as a heteroatom, includes a monocycle or polycycle having 2 to 60 carbon atoms, and may be additionally substituted with another substituent. Here, the polycycle means a group in which a heteroaryl group is directly linked to or fused with another cyclic group. Here, another cyclic group may also be a heteroaryl group, but may also be another kind of cyclic group, for example, a cycloalkyl group, a heterocycloalkyl group, an aryl group, and the like. The number of carbon atoms of the heteroaryl group may be 2 to 60, specifically 2 to 40, and more specifically 3 to 25. Specific examples of the heteroaryl group include a pyridyl group, a pyrrolyl group, a pyrimidyl group, a pyridazinyl group, a furanyl group, a thiophene group, an imidazolyl group, a pyrazolyl group, an oxazolyl group, an isoxazolyl group, a thiazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a thiazinyl group, a dioxynyl group, a triazinyl group, a tetrazinyl group, a quinolyl group, an isoquinolyl group, a quinazolinyl group, an isoquinazolinyl group, a quinozolilyl group, a naphthyridyl group, an acridinyl group, a phenanthridinyl group, an imidazopyridinyl group, a diaza naphthalenyl group, a triazaindene group, an indolyl group, an indolizinyl group, a benzothiazolyl group, a benzoxazolyl group, a benzimidazolyl group, a benzothiophene group, a benzofuran group, a dibenzothiophene group, a dibenzofuran group, a carbazolyl group, a benzocarbazolyl group, a dibenzocarbazolyl group, a phenazinyl group, a dibenzosilole group, spirobi (dibenzosilole) group, a dihydrophenazinyl group, a phenoxazinyl group, a phenanthridyl group, an imidazopyridinyl group, a thienyl group, an indolo[2,3-a]carbazolyl group, an indolo[2,3-b]carbazolyl group, an indolinyl group, a 10,11-dihydro-dibenzo[b,f]azepin group, a 9,10-dihydroacridinyl group, a phenanthrazinyl group, a phenothiathiazinyl group, a phthalazinyl group, a naphthylidinyl group, a phenanthrolinyl group, a benzo[c][1,2,5]thiadiazolyl group, 2,3-dihydrobenzo[b]thiophene, 2,3-dihydrobenzofuran, a 5,10-dihydrodibenzo[b,e][1,4]azasilinyl, a pyrazolo[1,5-c]quinazolinyl group, a pyrido[1,2-b]indazolyl group, a pyrido[1,2-a]imidazo[1,2-e]indolinyl group, a 5,11-dihydroindeno[1,2-b]carbazolyl group, and the like, but are not limited thereto.

In the present specification, a silyl group includes Si and is a substituent to which the Si atom is directly linked as a radical, and is represented by —Si(R101) (R102) (R103), and R101 to R103 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. Specific examples of the silyl group include

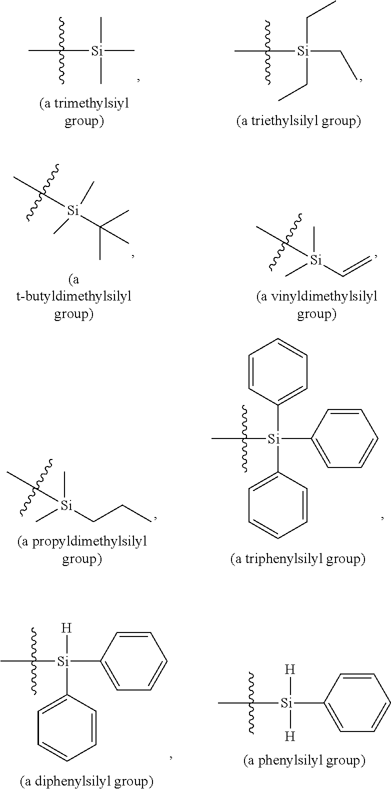

and the like, but are not limited thereto.

In the present specification, the phosphine oxide group is represented by —P(═O) (R104) (R105), and R104 and R105 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. Specifically, the phosphine oxide group may be substituted with an alkyl group or an aryl group, and the above-described example may be applied to the alkyl group and the aryl group. Examples of the phosphine oxide group include a dimethylphosphine oxide group, a diphenylphosphine oxide group, dinaphthylphosphine oxide, and the like, but are not limited thereto.

In the present specification, the amine group is represented by —N(R106) (R107), and R106 and R107 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. The amine group may be selected from the group consisting of —NH₂; a monoalkylamine group; a monoarylamine group; a monoheteroarylamine group; a dialkylamine group; a diarylamine group; a diheteroarylamine group; an alkylarylamine group; an alkylheteroarylamine group; and an arylheteroarylamine group, and the number of carbon atoms thereof is not particularly limited, but is preferably 1 to 30. Specific examples of the amine group include a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group, and the like, but are not limited thereto.

In the present specification, the above-described examples of the aryl group may be applied to an arylene group except for a divalent arylene group.

In the present specification, the above-described examples of the heteroaryl group may be applied to a heteroarylene group except for a divalent heteroarylene group.

An exemplary embodiment of the present specification provides the amine compound represented by Chemical Formula 1.

In an exemplary embodiment of the present specification, the deuterium content of Chemical Formula 1 may be more than 0% and 100% or less.

The amine compound according to one embodiment of the present specification needs to include deuterium to have an effect of improving the service life of an organic light emitting device when used as a material for the device.

Specifically, the photochemical characteristics of a compound which includes deuterium and a compound which does not include deuterium are almost similar, but when deposited on a thin film, the deuterium-containing material tends to be packed with a narrower intermolecular distance. Accordingly, when an electron only device (EOD) and a hole only device (HOD) are manufactured and the current density thereof according to voltage is confirmed, it can be confirmed that the compound of Chemical Formula 1 according to the present application, which includes deuterium, exhibits a much more balanced charge transport characteristics than the compound which does not include deuterium.

Further, when the surface of a thin film is observed using an atomic force microscope (AFM), it can be confirmed that the thin film made of a compound including deuterium is deposited with a more uniform surface without any aggregated portion.

Additionally, since the single bond dissociation energy of carbon and deuterium is higher than the single bond dissociation energy of carbon and hydrogen, the stability of the entire molecule is increased, so that there is an effect of improving the service life of the device.

In an exemplary embodiment of the present specification, Chemical Formula 1 may be dividedly represented by the following Structures A and B.

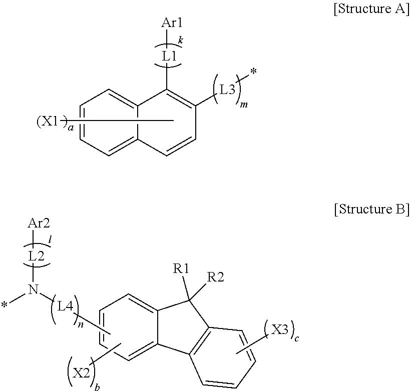

In Structures A and B, the definition of each substituent is the same as that in Chemical Formula 1, and

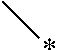 [SMILES: *C]

is a position where Structures A and B are bonded to each other.

In an exemplary embodiment of the present specification, Chemical Formula 1 may be represented by a bond of Structure A and Structure B.

In an exemplary embodiment of the present specification, at least one of Structures A and B includes deuterium.

In an exemplary embodiment of the present specification, Structure A includes deuterium, and Structure B may not include deuterium.

In an exemplary embodiment of the present specification, Structure B includes deuterium, and Structure A may not include deuterium.

In an exemplary embodiment of the present specification, Structures A and B may include deuterium.

In an exemplary embodiment of the present specification, at least one of Structures A and B may have a deuterium content of 50% to 100%.

In an exemplary embodiment of the present specification, at least one of Structures A and B may have a deuterium content of 70% to 100%.

In an exemplary embodiment of the present specification, Structures A and B may each have a deuterium content of 50% to 100%.

In an exemplary embodiment of the present specification, Structures A and B may each have a deuterium content of 70% to 100%.

In an exemplary embodiment of the present specification, L1 to L4 are each independently a direct bond; a substituted or unsubstituted arylene group having 6 to 30 carbon atoms; or a substituted or unsubstituted heteroarylene group having 2 to 30 carbon atoms.

In an exemplary embodiment of the present specification, L1 to L4 are each independently a direct bond; or a substituted or unsubstituted arylene group having 6 to 30 carbon atoms.

In an exemplary embodiment of the present specification, L1 to L4 are each independently a direct bond; or a substituted arylene group having 6 to 20 carbon atoms.

In an exemplary embodiment of the present specification, L1 to L4 are each independently a direct bond; or an arylene group having 6 to 20 carbon atoms, which is unsubstituted or substituted with deuterium.

In an exemplary embodiment of the present specification, L1 to L4 are each independently a direct bond; or a phenylene group which is unsubstituted or substituted with deuterium.

In an exemplary embodiment of the present specification, X1 is hydrogen; or deuterium.

In an exemplary embodiment of the present specification, X2 is hydrogen; or deuterium.

In an exemplary embodiment of the present specification, X3 is hydrogen; or deuterium.

In an exemplary embodiment of the present specification, Ar1 may be a phenyl group substituted with deuterium; a substituted or unsubstituted aryl group having 8 to 30 carbon atoms; or a substituted or unsubstituted heteroaryl group having 2 to 30 carbon atoms.

In an exemplary embodiment of the present specification, Ar1 may be a phenyl group substituted with deuterium; a substituted or unsubstituted aryl group having 8 to 30 carbon atoms; or a substituted or unsubstituted heteroaryl group having 2 to 30 carbon atoms.

In an exemplary embodiment of the present specification, Ar1 may be a phenyl group substituted with deuterium; a biphenyl group which is unsubstituted or substituted with deuterium; a terphenyl group which is unsubstituted or substituted with deuterium; a naphthyl group which is unsubstituted or substituted with deuterium; a phenanthrenyl group which is unsubstituted or substituted with deuterium; a dimethylfluorenyl group which is unsubstituted or substituted with deuterium; a diphenylfluorenyl group which is unsubstituted or substituted with deuterium; a spirobifluorenyl group which is unsubstituted or substituted with deuterium; a 9-phenyl-9H-carbazole group which is unsubstituted or substituted with deuterium; a dibenzofuran group which is unsubstituted or substituted with deuterium; or a dibenzothiophene group which is unsubstituted or substituted with deuterium.

In an exemplary embodiment of the present specification, Ar1 may be a biphenyl group which is unsubstituted or substituted with deuterium; a terphenyl group which is unsubstituted or substituted with deuterium; a naphthyl group which is unsubstituted or substituted with deuterium; a dimethylfluorenyl group which is unsubstituted or substituted with deuterium; a diphenylfluorenyl group which is unsubstituted or substituted with deuterium; a spirobifluorenyl group which is unsubstituted or substituted with deuterium; a dibenzofuran group which is unsubstituted or substituted with deuterium; or a dibenzothiophene group which is unsubstituted or substituted with deuterium.

In an exemplary embodiment of the present specification, Ar2 may be a substituted or unsubstituted aryl group having 6 to 30 carbon atoms; or a substituted or unsubstituted heteroaryl group having 2 to 30 carbon atoms.

In an exemplary embodiment of the present specification, Ar2 may be a phenyl group which is unsubstituted or substituted with deuterium; a biphenyl group which is unsubstituted or substituted with deuterium; a terphenyl group which is unsubstituted or substituted with deuterium; a naphthyl group which is unsubstituted or substituted with deuterium; a phenanthrenyl group which is unsubstituted or substituted with deuterium; a dimethylfluorenyl group which is unsubstituted or substituted with deuterium; a diphenylfluorenyl group which is unsubstituted or substituted with deuterium; a spirobifluorenyl group which is unsubstituted or substituted with deuterium; a 9-phenyl-9H-carbazole group which is unsubstituted or substituted with deuterium; a dibenzofuran group which is unsubstituted or substituted with deuterium; or a dibenzothiophene group which is unsubstituted or substituted with deuterium.

In an exemplary embodiment of the present specification, at least one of Ar1 and Ar2 may include deuterium.

In an exemplary embodiment of the present specification, at least one of Ar1 and Ar2 may be a phenyl group substituted with deuterium; a biphenyl group substituted with deuterium; a terphenyl group substituted with deuterium; a naphthyl group substituted with deuterium; a phenanthrenyl group substituted with deuterium; a dimethylfluorenyl group substituted with deuterium; a diphenylfluorenyl group substituted with deuterium; a spirobifluorenyl group substituted with deuterium; a 9-phenyl-9H-carbazole group substituted with deuterium; a dibenzofuran group substituted with deuterium; or a dibenzothiophene group substituted with deuterium.

The substituents substituted with deuterium mean those substituted with one or more deuteriums. That is, the substituents have a deuterium content of more than 0% and 100% or less.

In an exemplary embodiment of the present specification, R1 and R2 are the same as or different from each other, and are each independently a substituted or unsubstituted alkyl group having 1 to 20 carbon atoms; or a substituted or unsubstituted aryl group having 6 to 30 carbon atoms, or may be bonded to each other to form a substituted or unsubstituted hydrocarbon ring having 3 to 30 carbon atoms; or a substituted or unsubstituted hetero ring having 2 to 30 carbon atoms.

In an exemplary embodiment of the present specification, R1 and R2 are the same as or different from each other, and are each independently a substituted or unsubstituted alkyl group having 1 to 10 carbon atoms; or a substituted or unsubstituted aryl group having 6 to 20 carbon atoms, or may be bonded to each other to form a substituted or unsubstituted hydrocarbon ring having 3 to 30 carbon atoms.

In an exemplary embodiment of the present specification, Chemical Formula 1 may be represented by the following Chemical Formula 2 or 3.

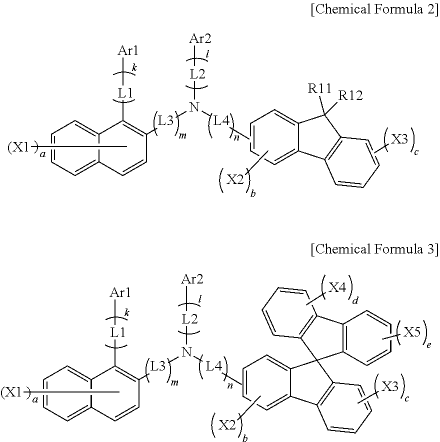

In Chemical Formulae 2 and 3,
R11 and R12 are each independently a substituted or unsubstituted alkyl group having 1 to 10 carbon atoms; or a substituted or unsubstituted aryl group having 6 to 20 carbon atoms,
X4 and X5 are each independently hydrogen; or deuterium,
d and e are each an integer from 0 to 4, and when d and e are 2 or higher, substituents in the parenthesis are the same as or different from each other, and
the definitions of the other substituents are the same as those in Chemical Formula 1.

In an exemplary embodiment of the present specification, R11 and R12 are each independently a substituted or unsubstituted alkyl group having 1 to 10 carbon atoms; or a substituted or unsubstituted aryl group having 6 to 20 carbon atoms.

In an exemplary embodiment of the present specification, R11 and R12 are each independently an alkyl group having 1 to 10 carbon atoms, which is unsubstituted or substituted with deuterium; or an aryl group having 6 to 20 carbon atoms, which is unsubstituted or substituted with deuterium.

In an exemplary embodiment of the present specification, R11 and R12 are each independently a methyl group which is unsubstituted or substituted with deuterium; or a phenyl group which is unsubstituted or substituted with deuterium.

In an exemplary embodiment of the present specification, X4 is hydrogen; or deuterium.

In an exemplary embodiment of the present specification, X5 is hydrogen; or deuterium.

In an exemplary embodiment of the present specification, Chemical Formula 1 may be represented by any one of the following Chemical Formulae 1-1 to 1-4.

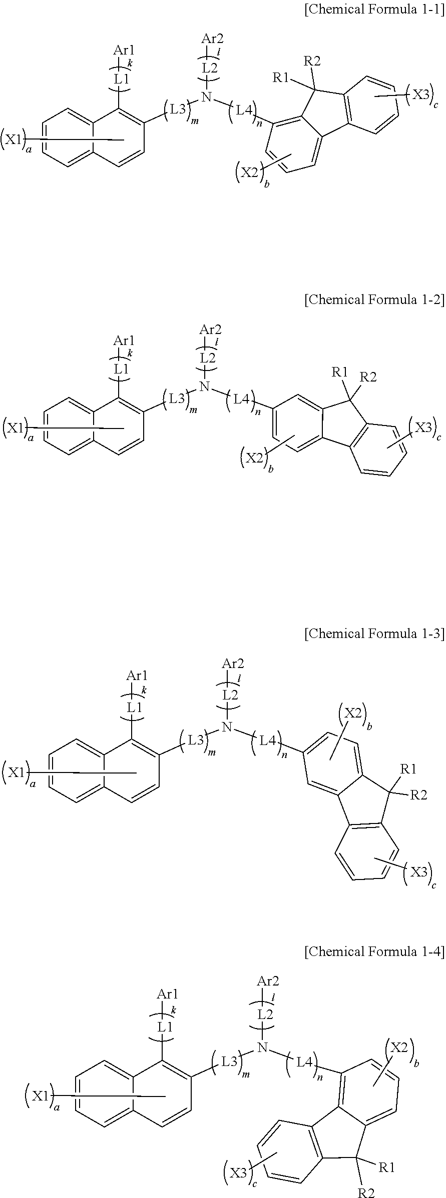

In Chemical Formulae 1-1 to 1-4, the definition of each substituent is the same as that in Chemical Formula 1.

In an exemplary embodiment of the present specification, the deuterium content of Chemical Formula 1 is more than 0% and 100% or less.

In an exemplary embodiment of the present specification, the deuterium content of Chemical Formula 1 may be 5% to 100%.

In an exemplary embodiment of the present specification, the deuterium content of Chemical Formula 1 may be 10% to 100%.

In an exemplary embodiment of the present specification, the deuterium content of Chemical Formula 1 may be 20% to 100%.

In an exemplary embodiment of the present specification, the deuterium content of Chemical Formula 1 may be 30% to 100%.

In an exemplary embodiment of the present specification, the deuterium content of Chemical Formula 1 may be 40% to 100%.

In an exemplary embodiment of the present specification, the deuterium content of Chemical Formula 1 may be 50% to 100%.

In an exemplary embodiment of the present specification, the deuterium content of Chemical Formula 1 may be 70% to 100%.

In an exemplary embodiment of the present specification, Chemical Formula 1 may be represented by any one of the following compounds.

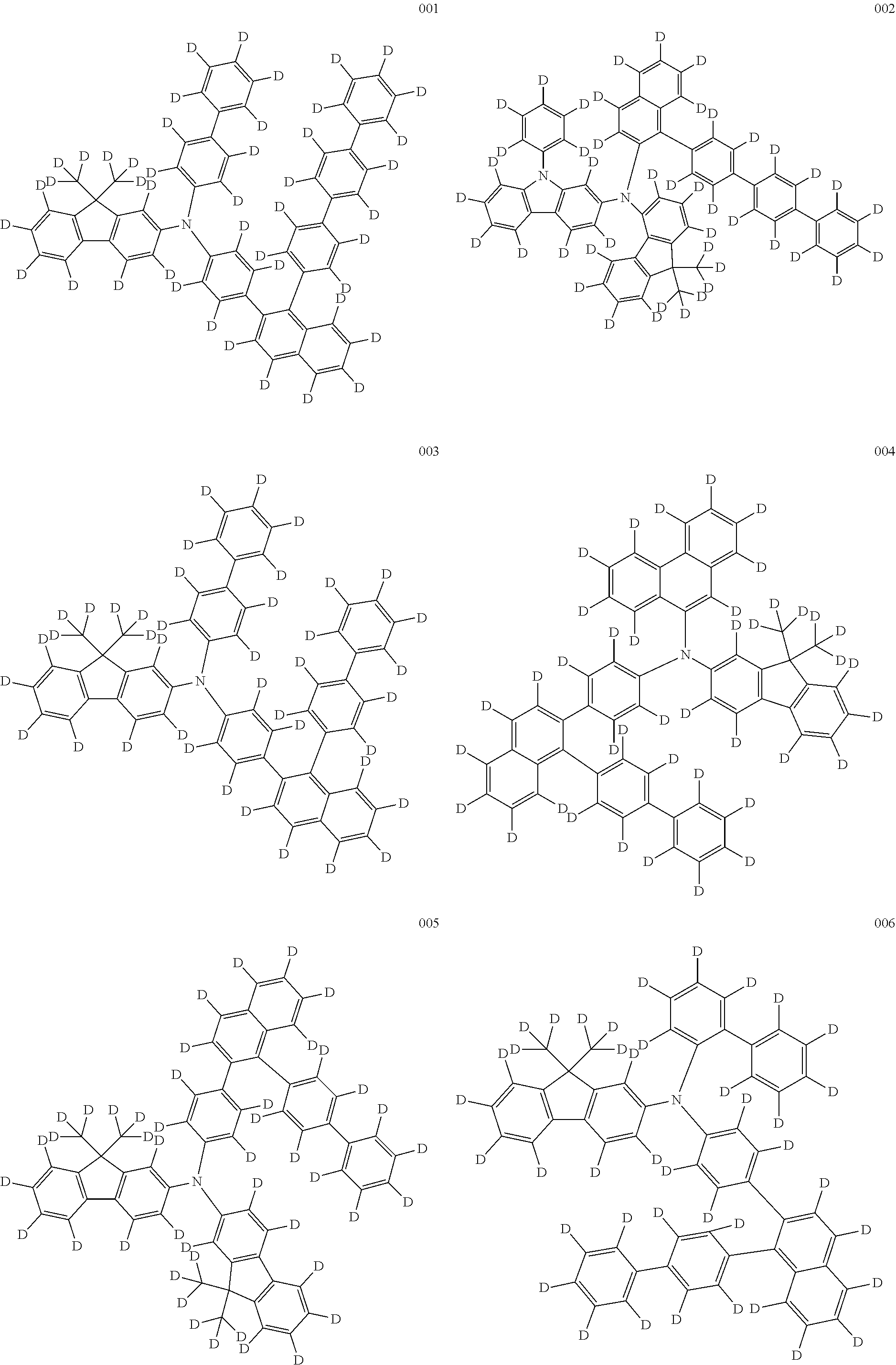

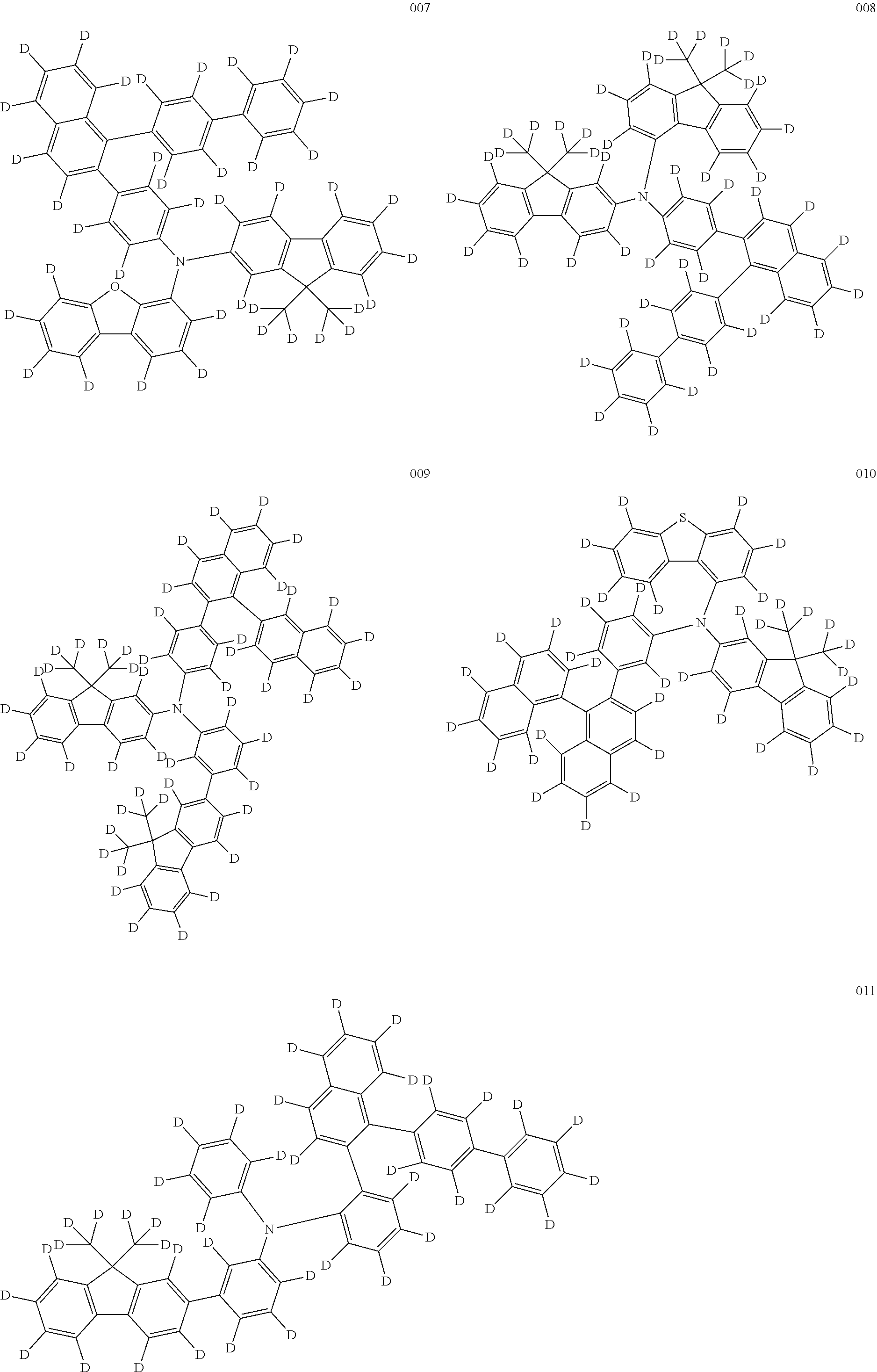

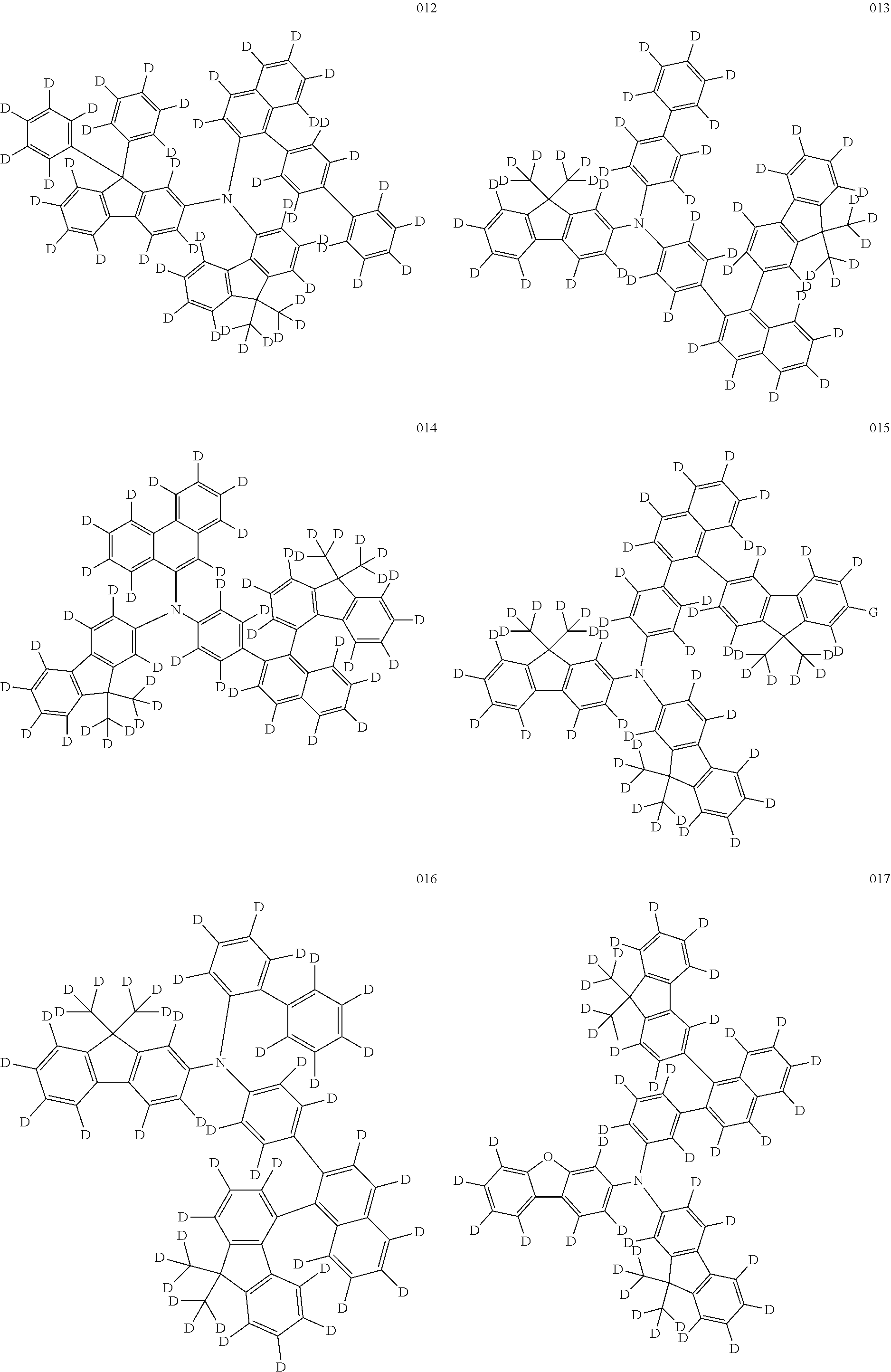

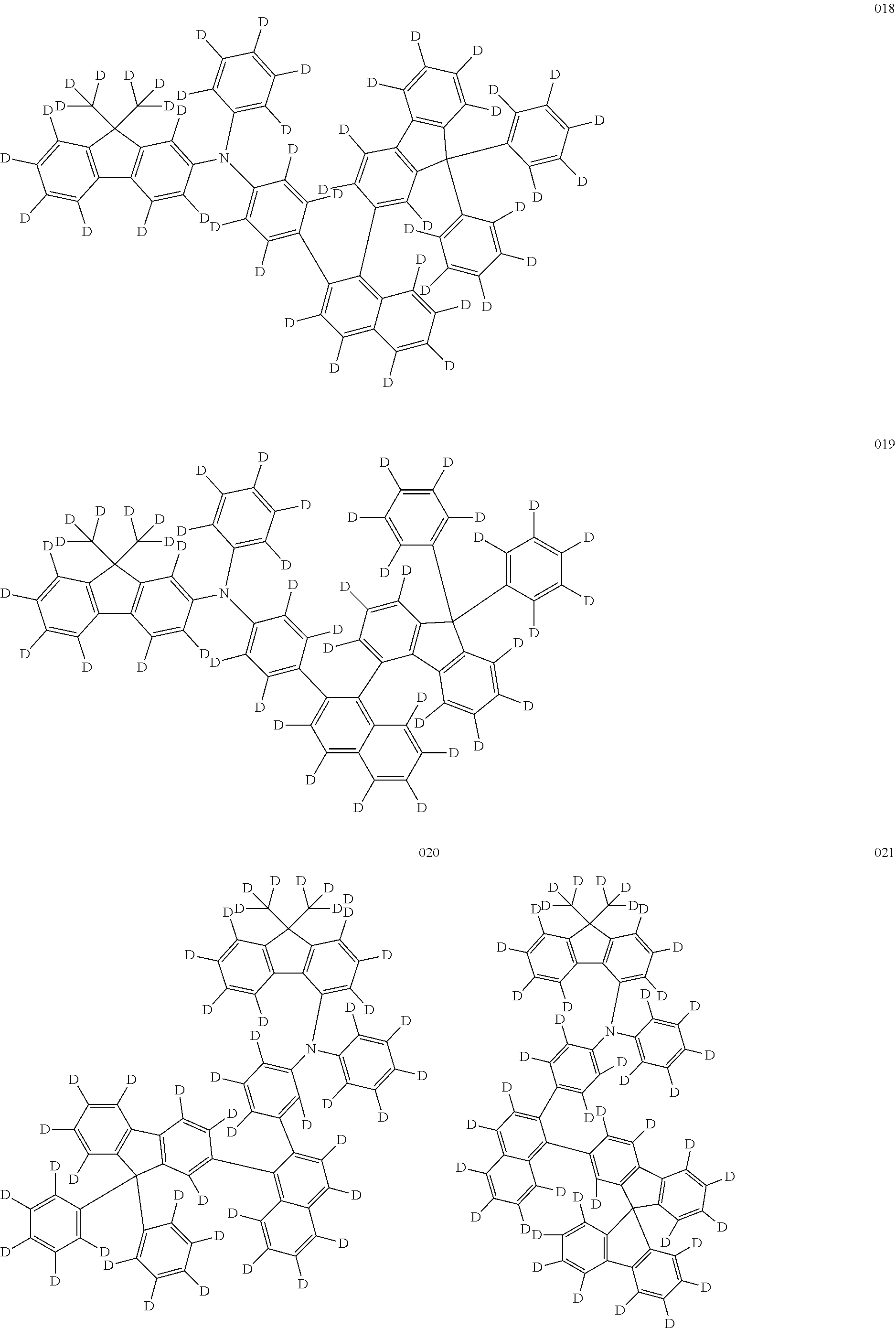

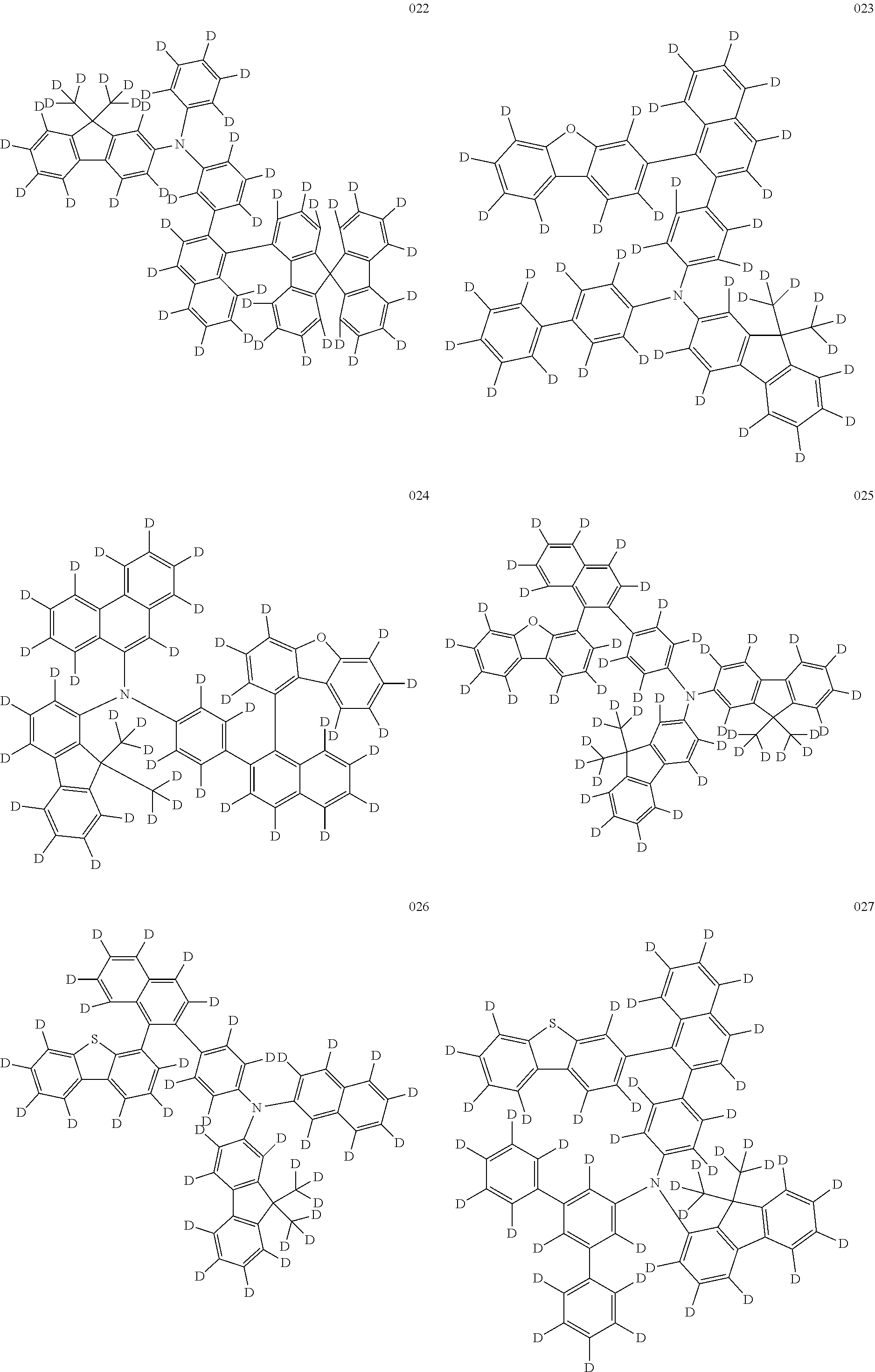

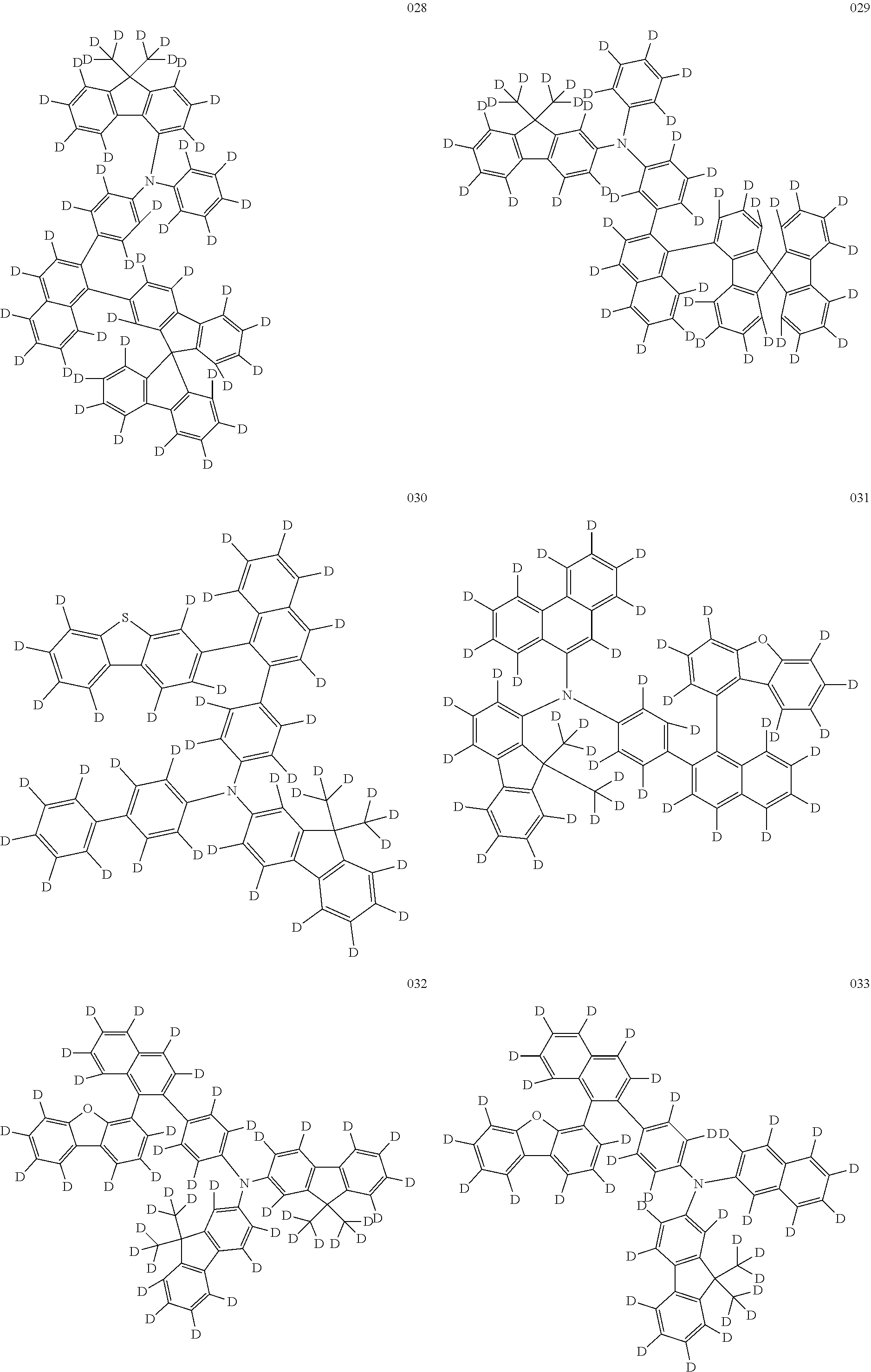

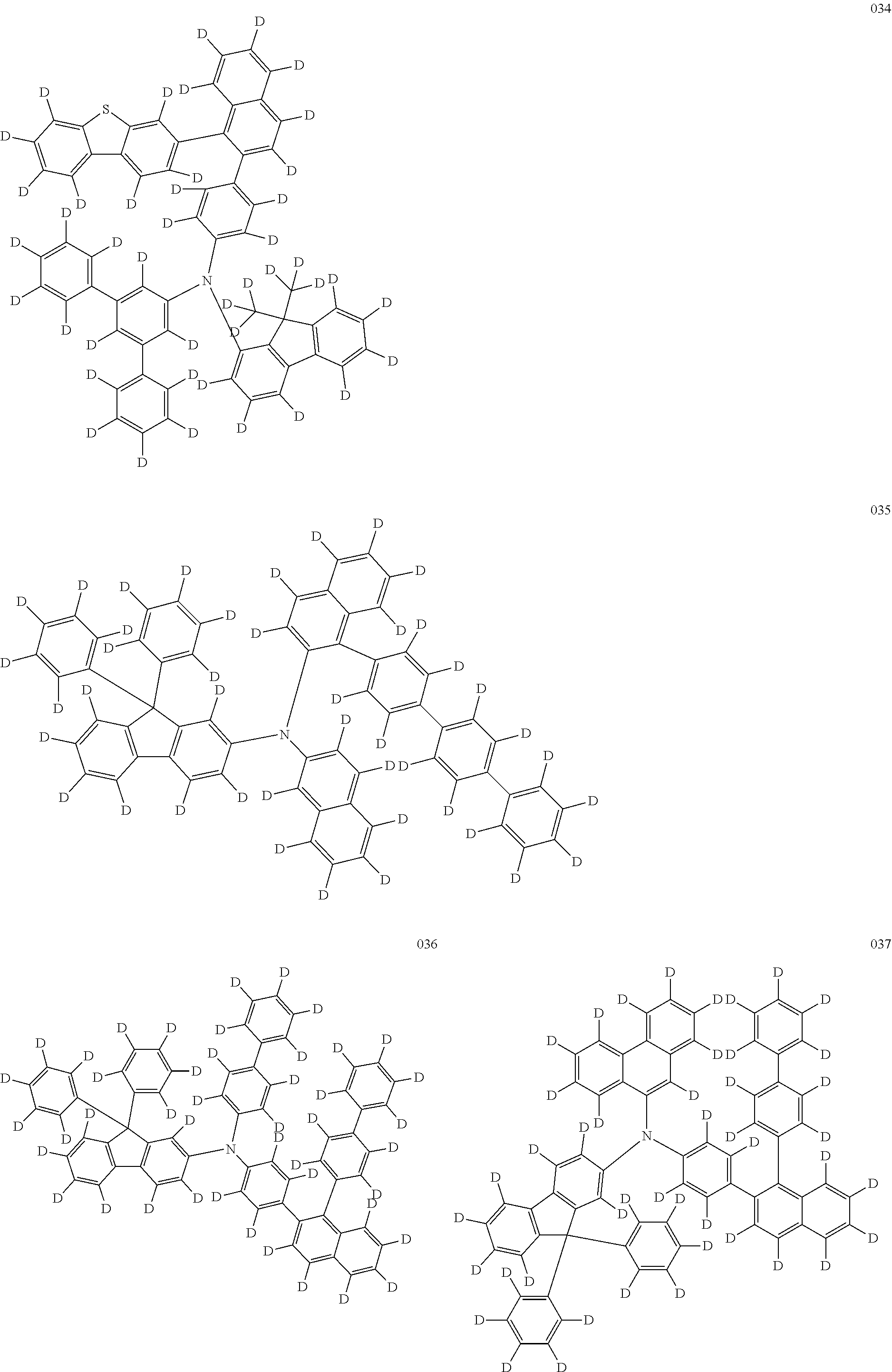

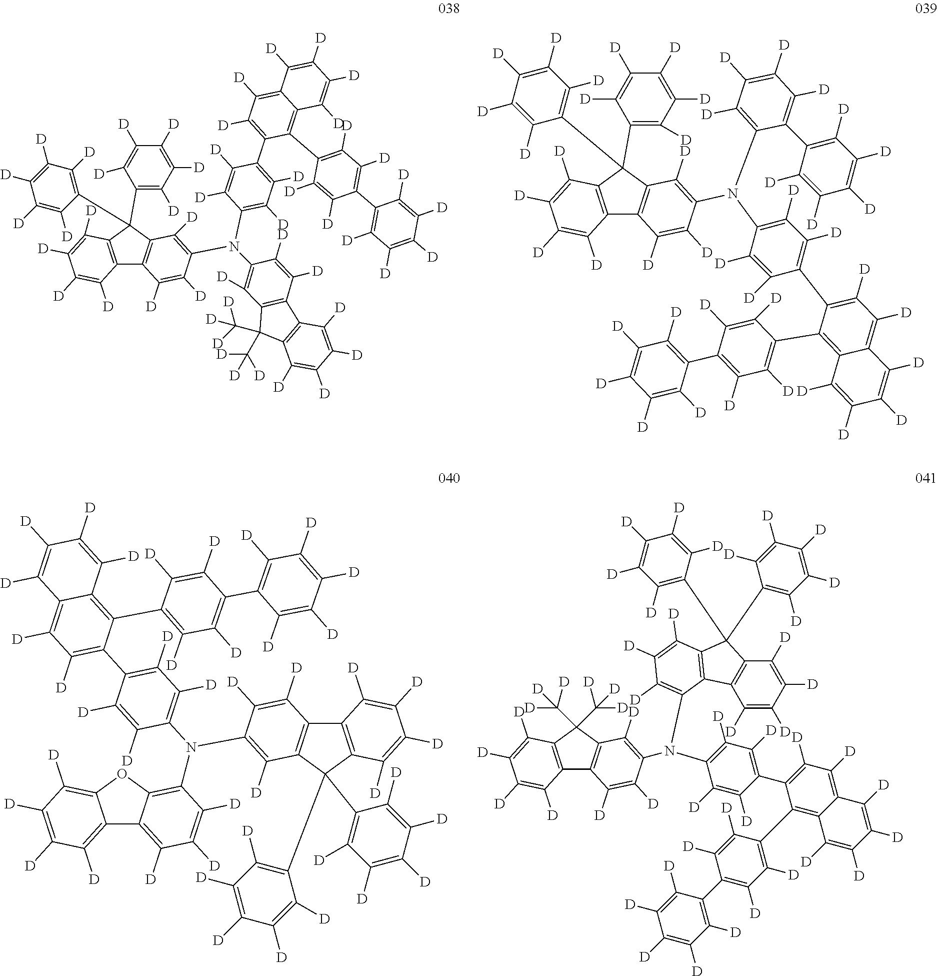

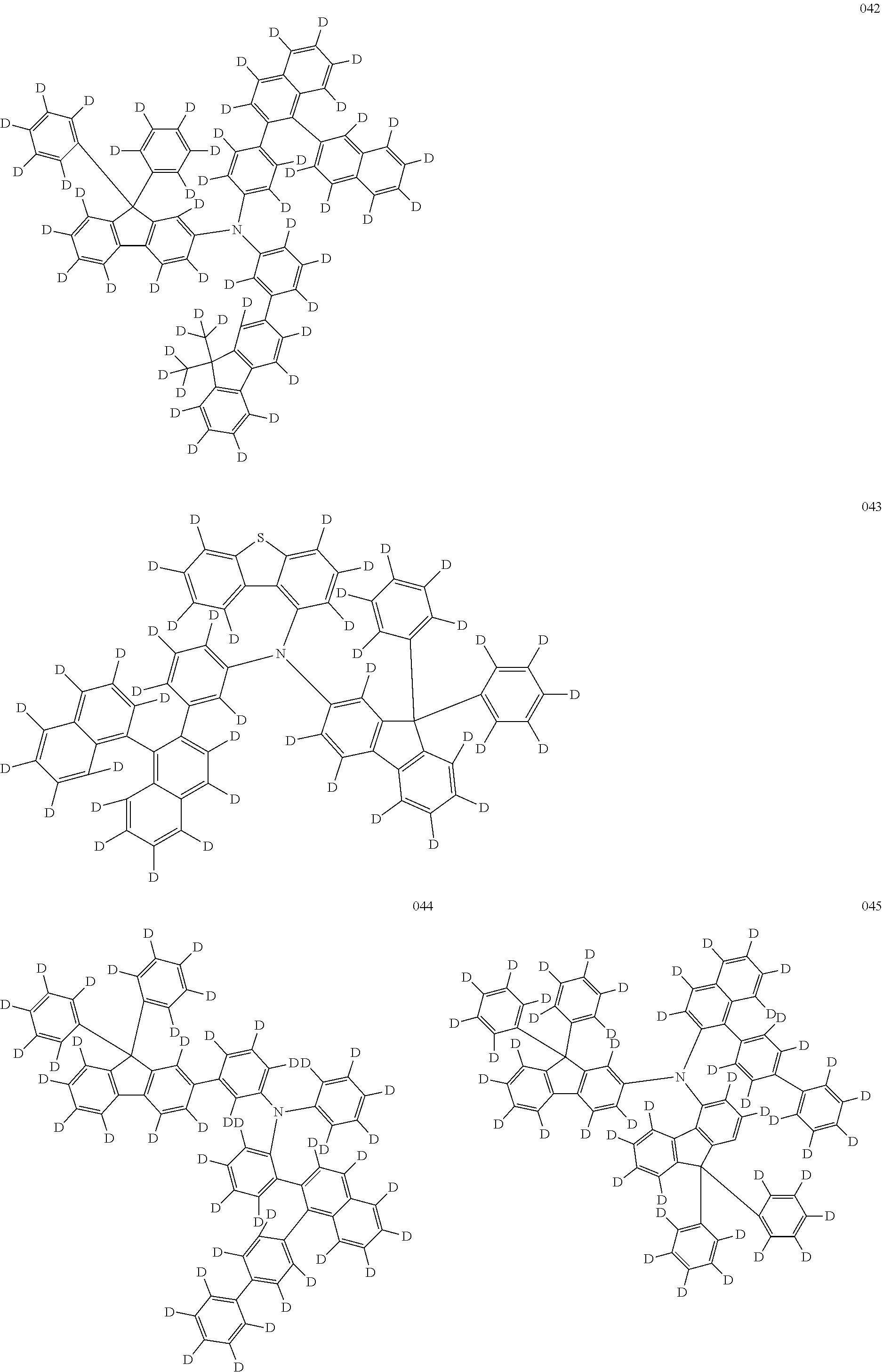

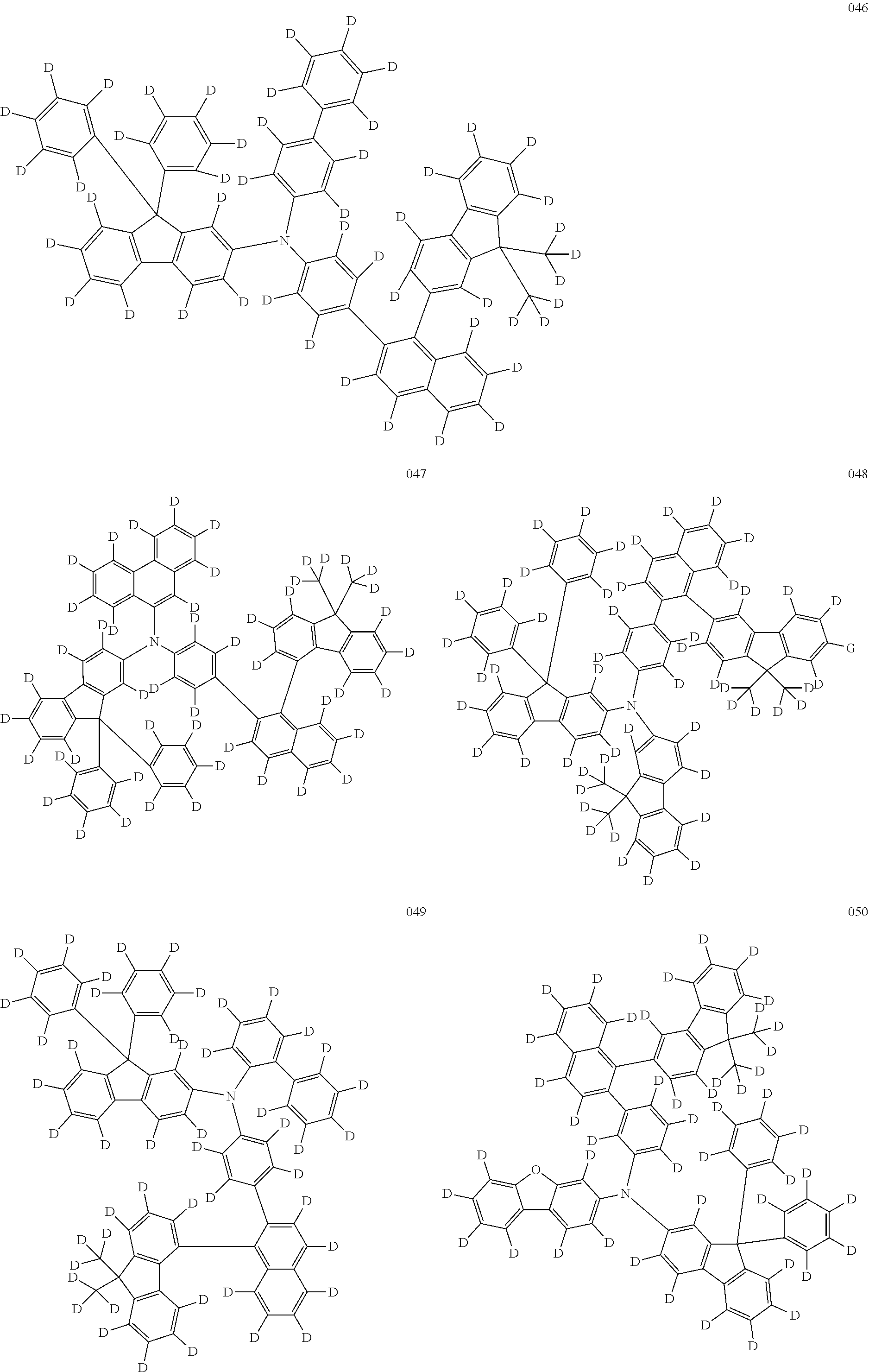

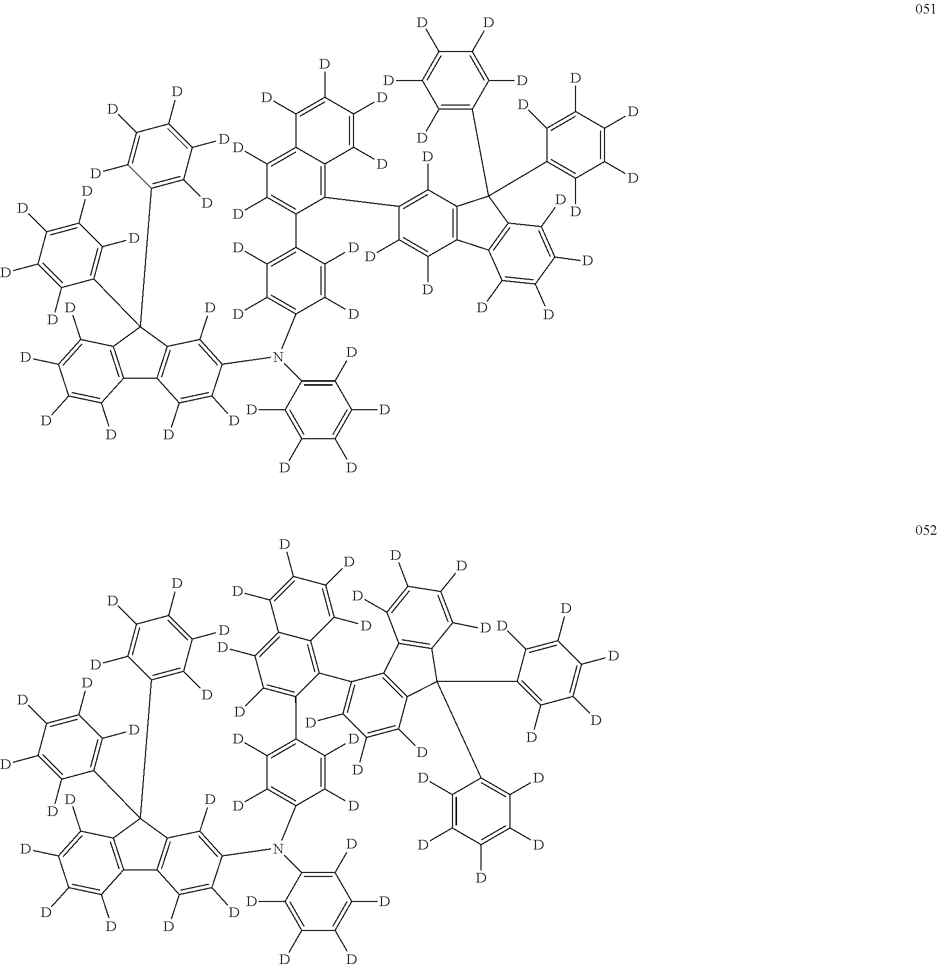

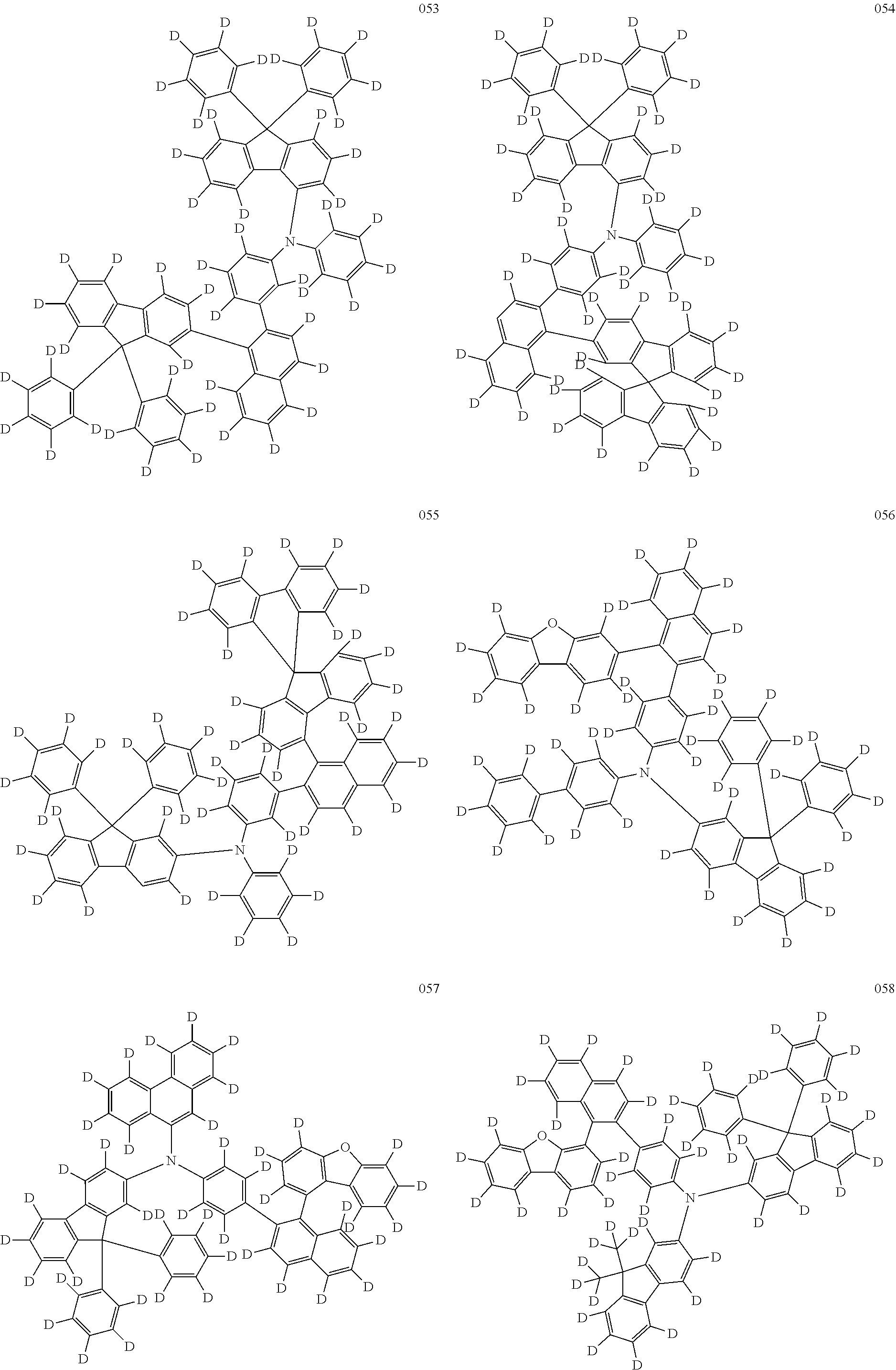

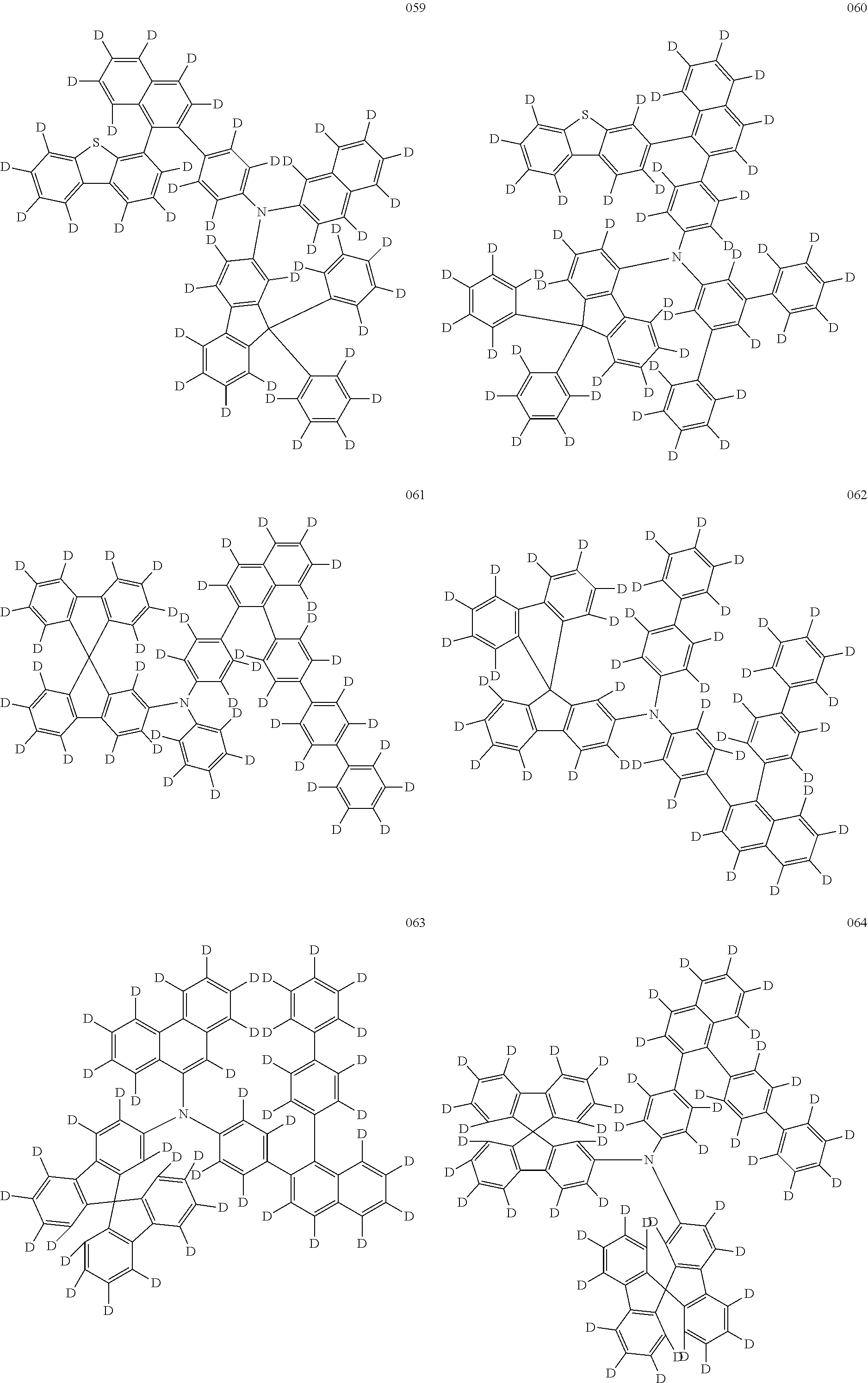

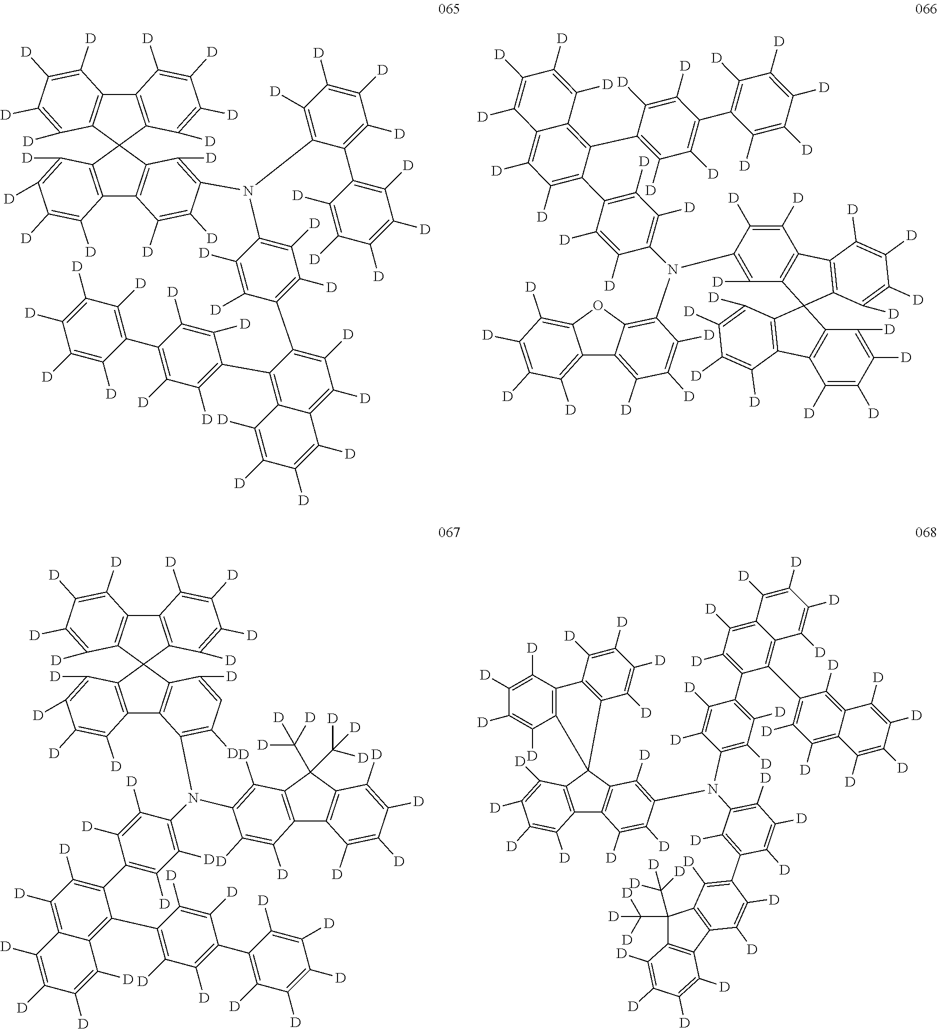

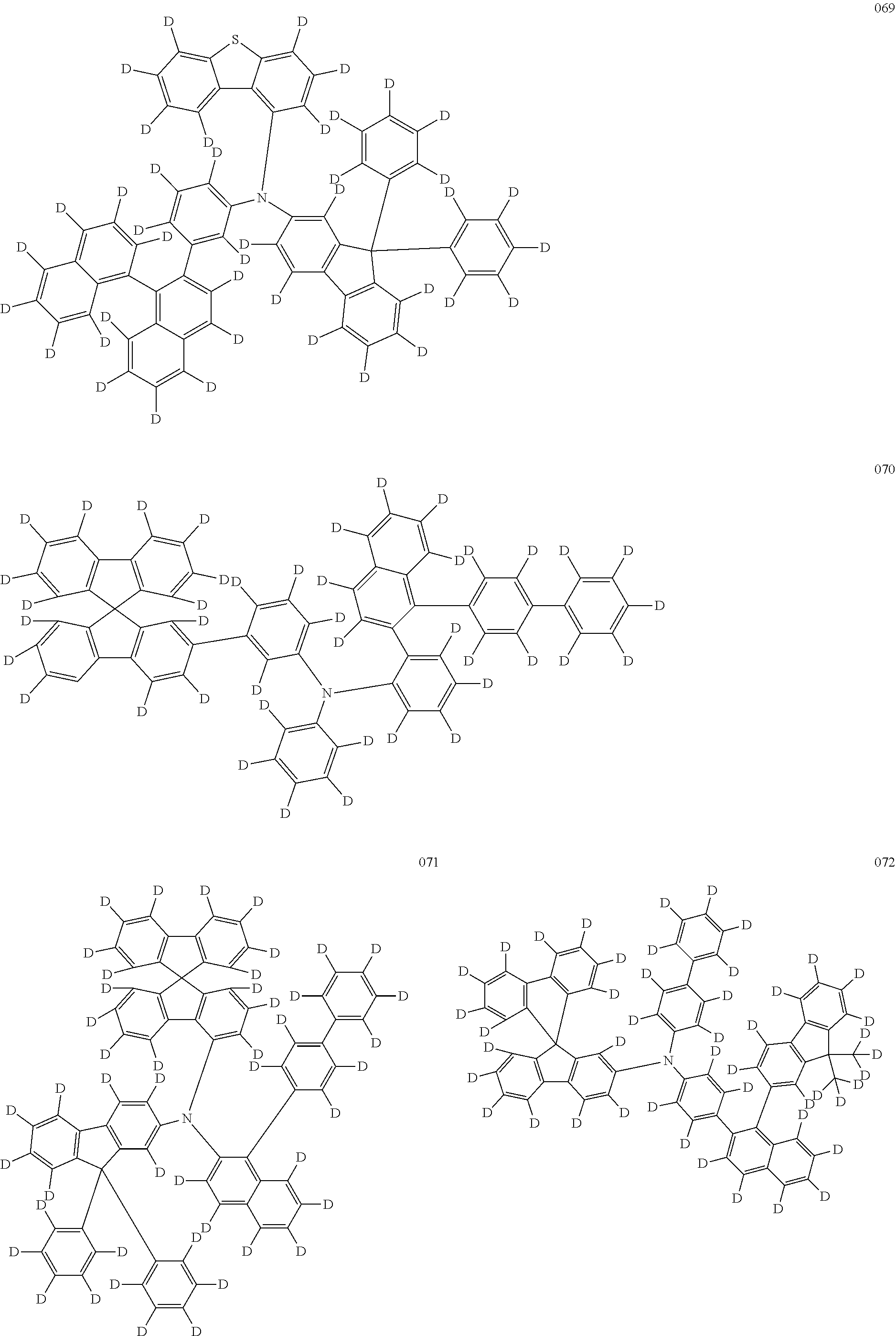

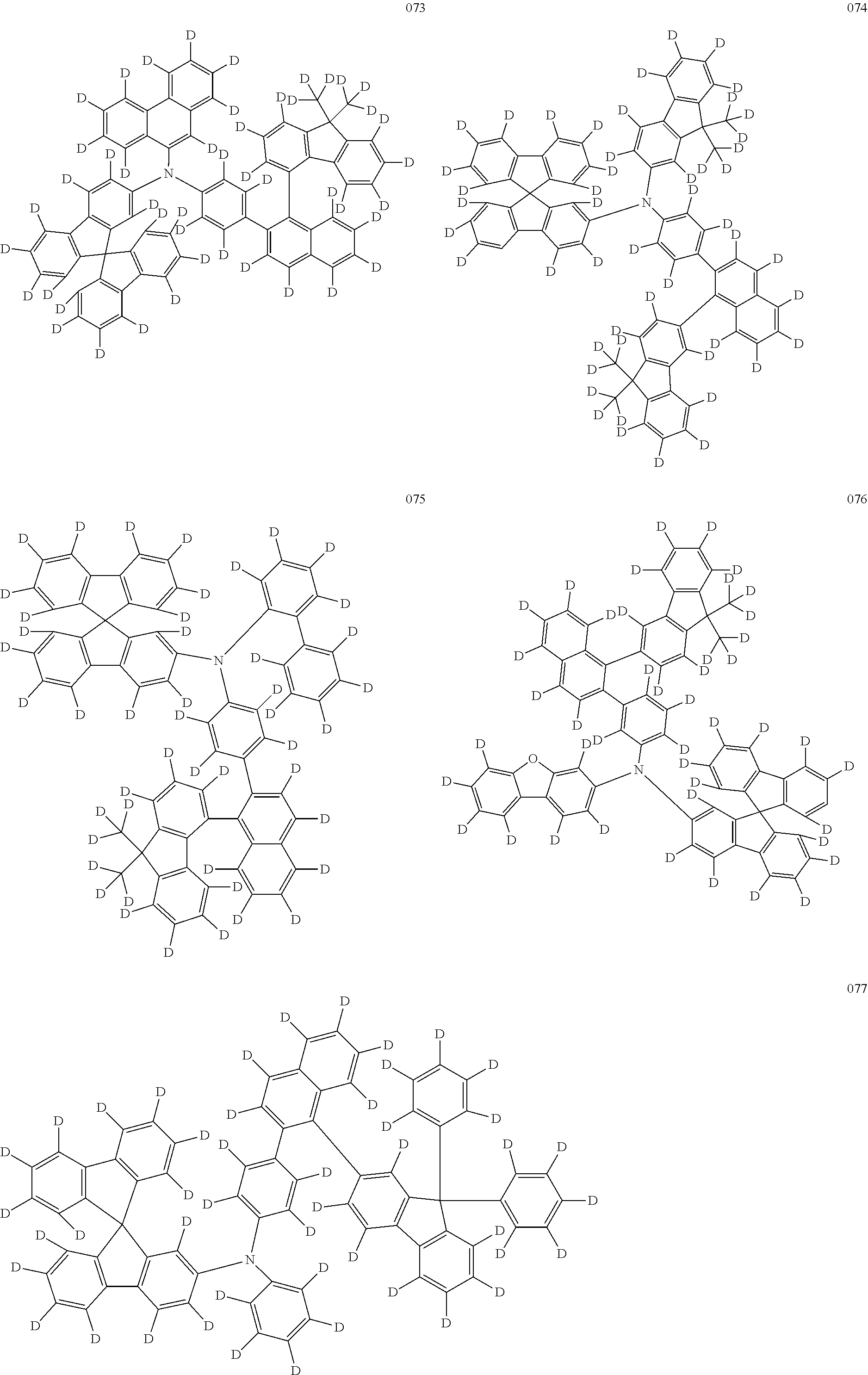

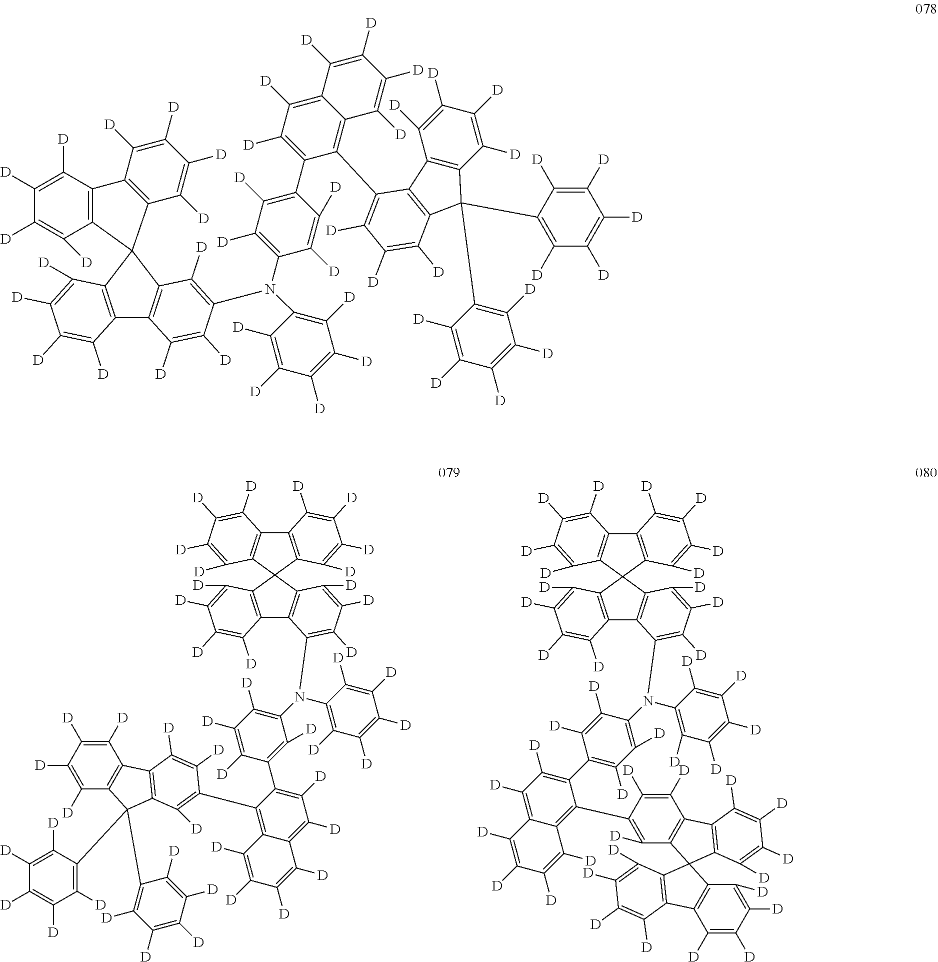

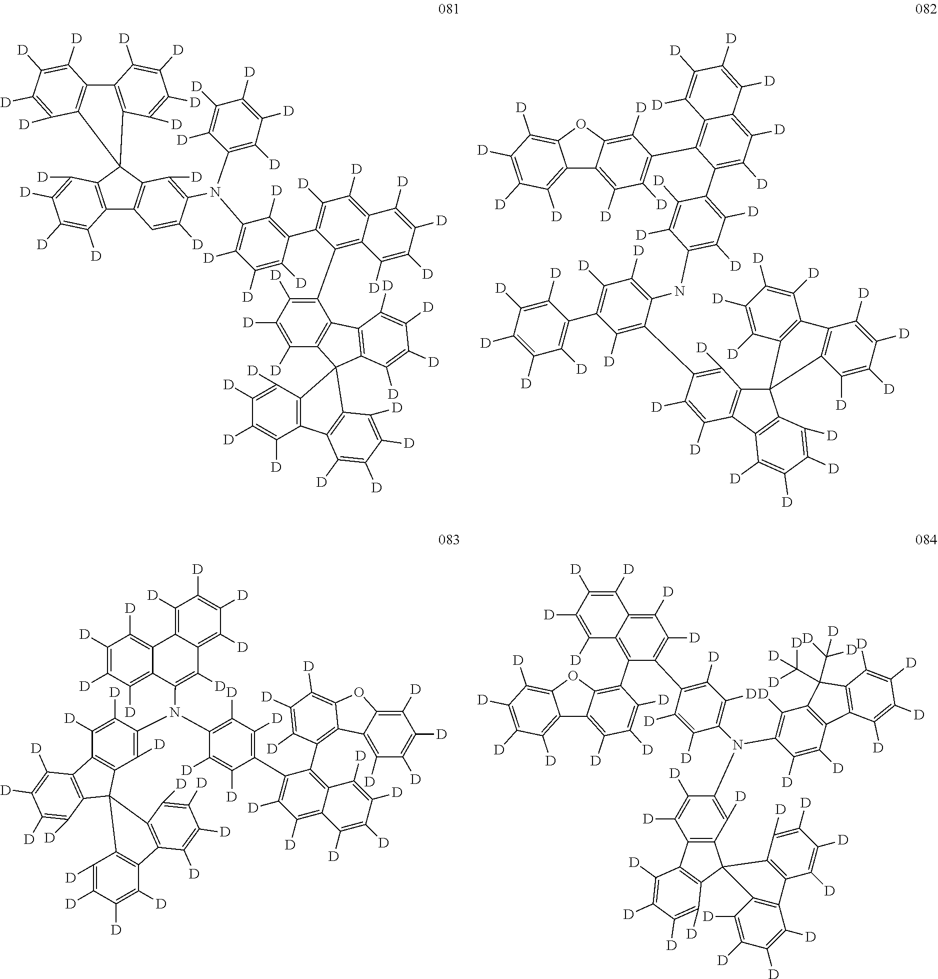

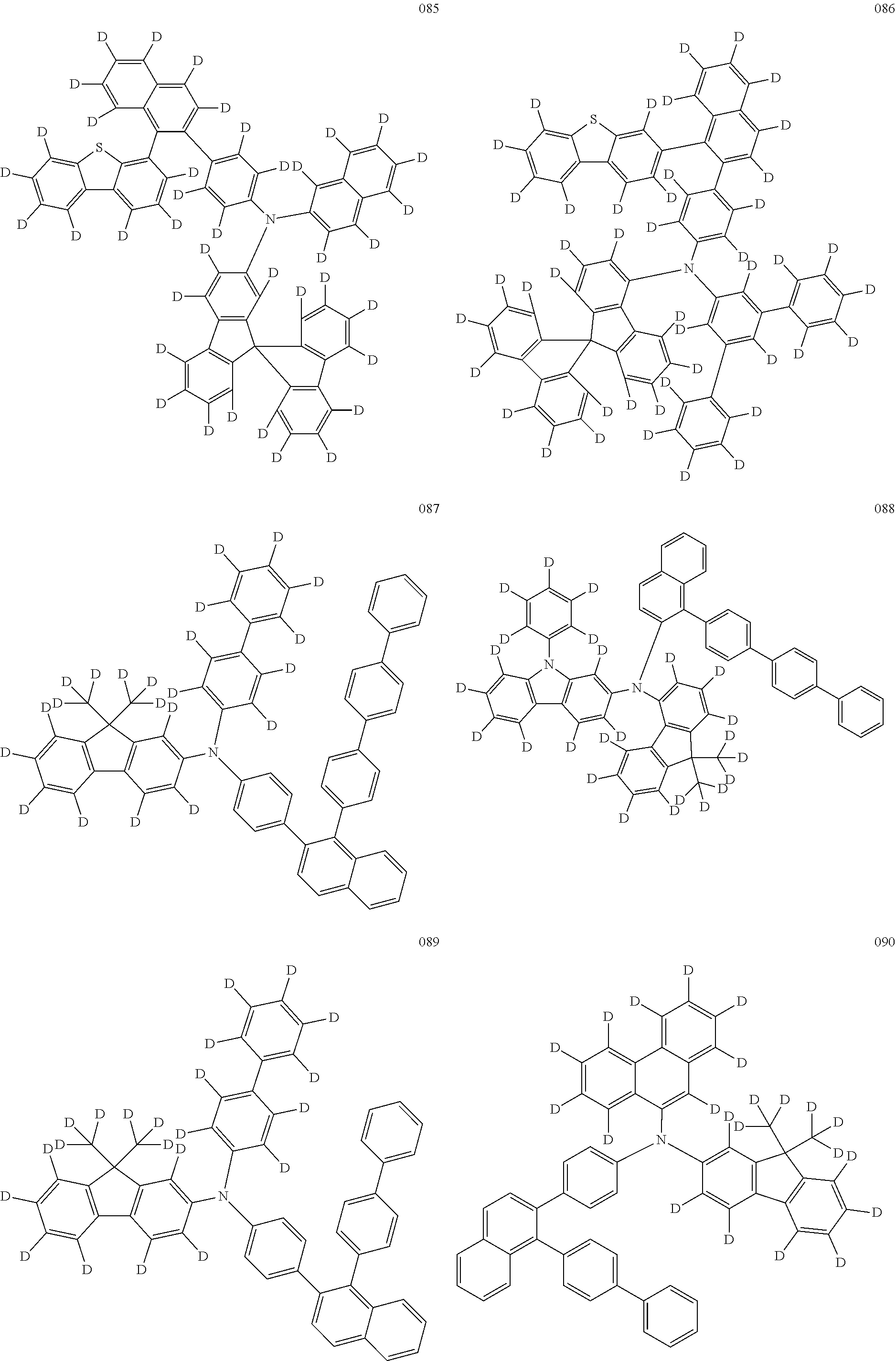

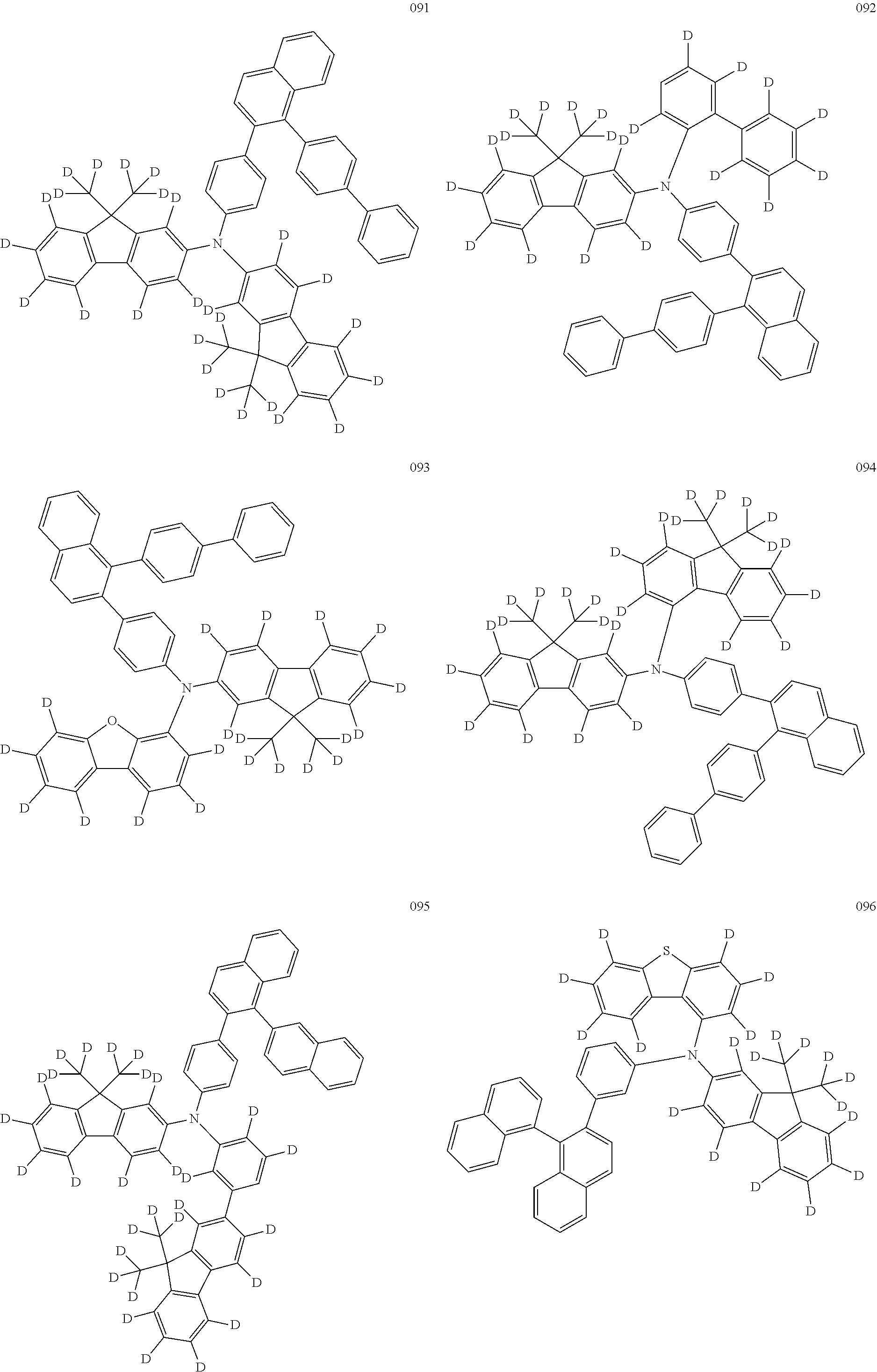

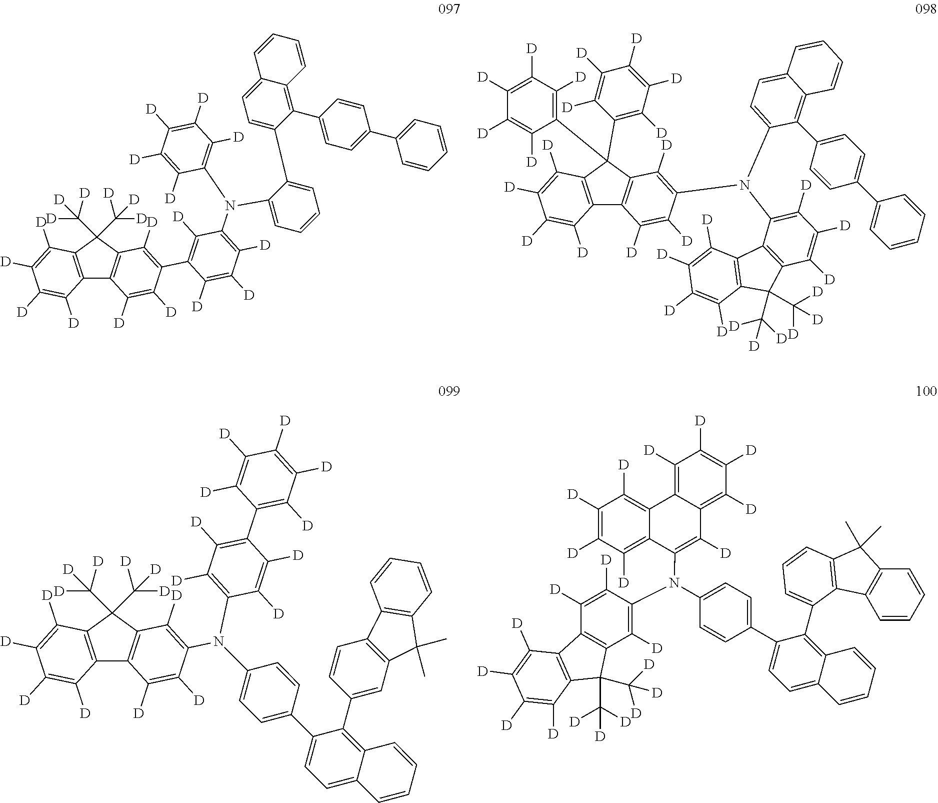

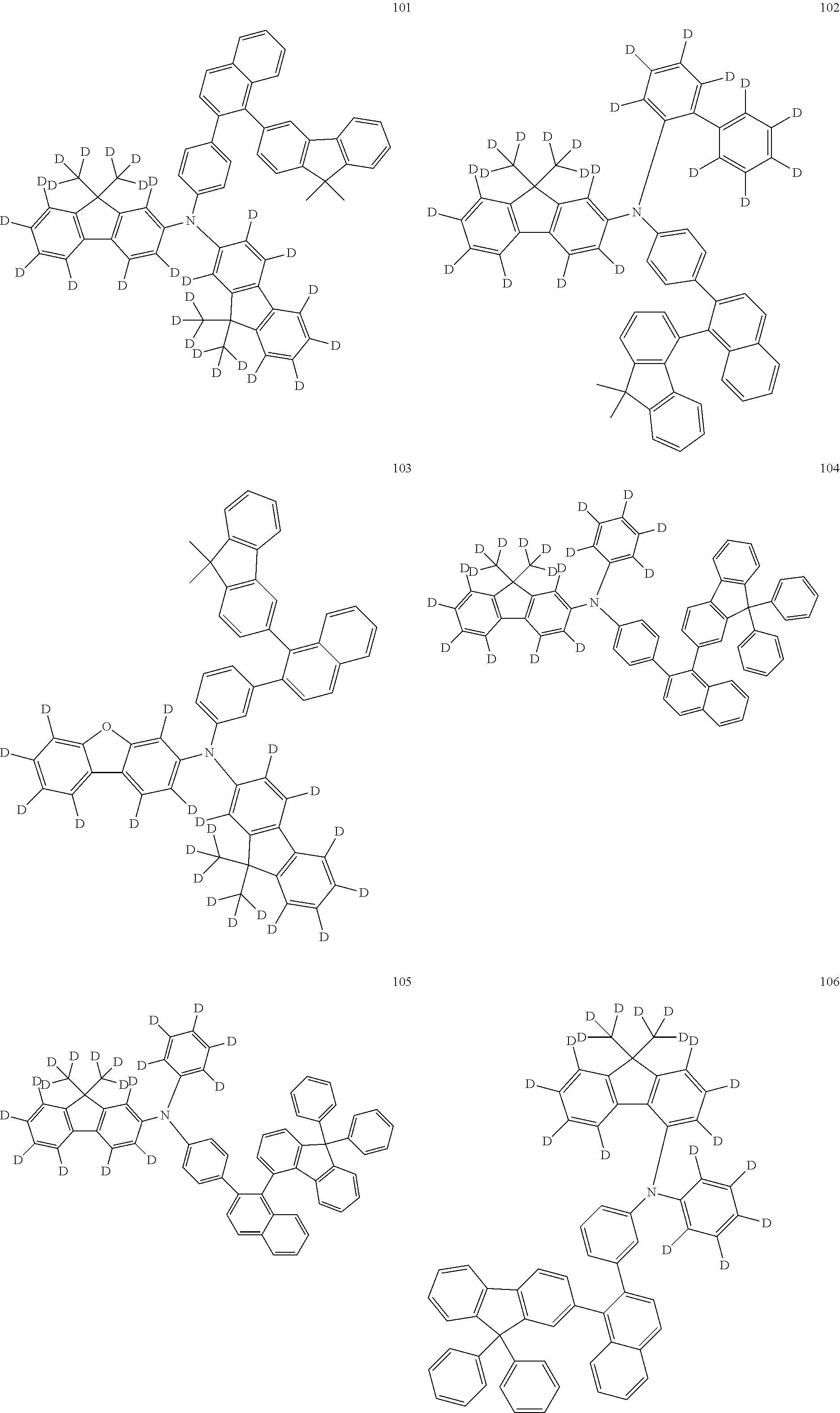

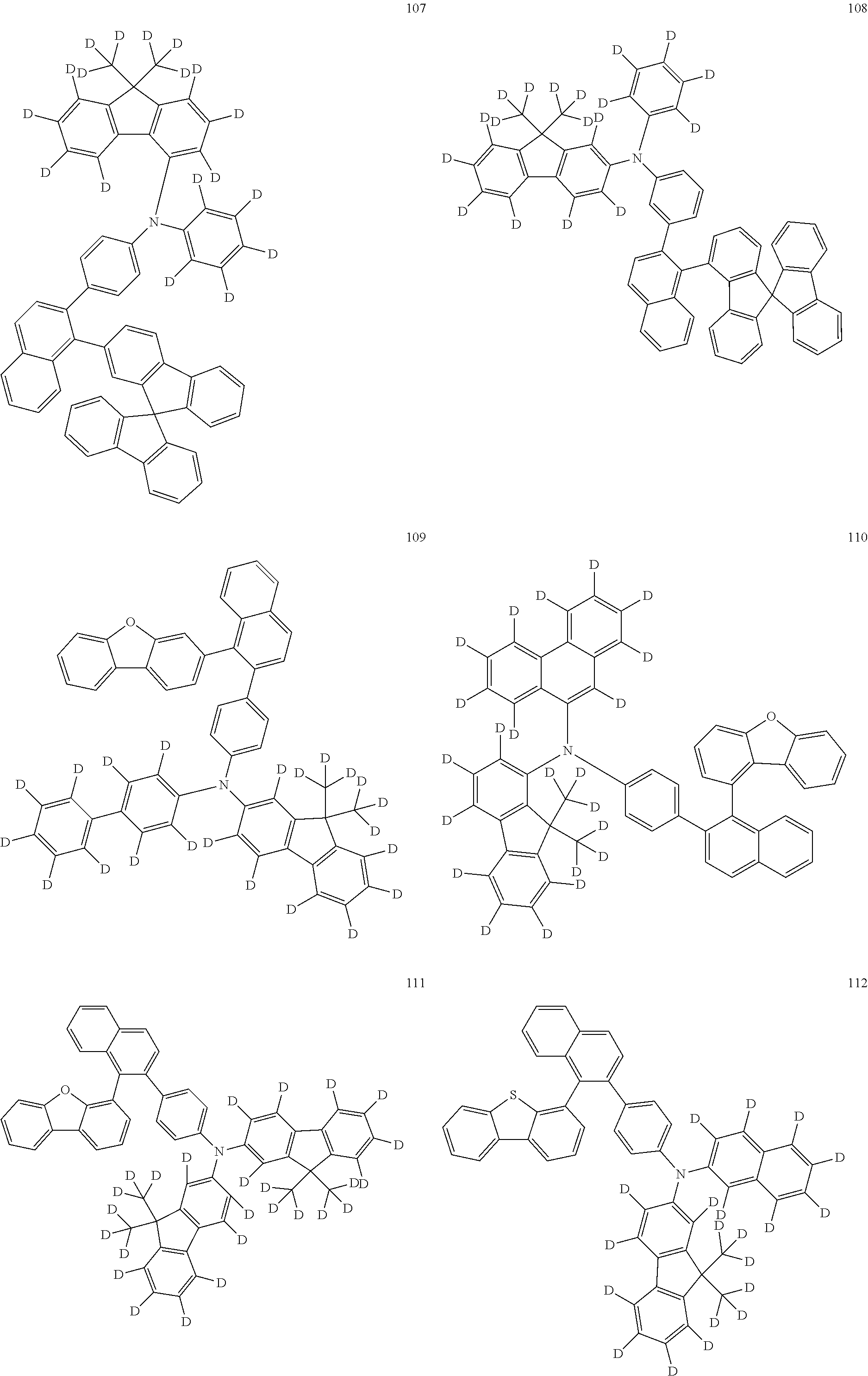

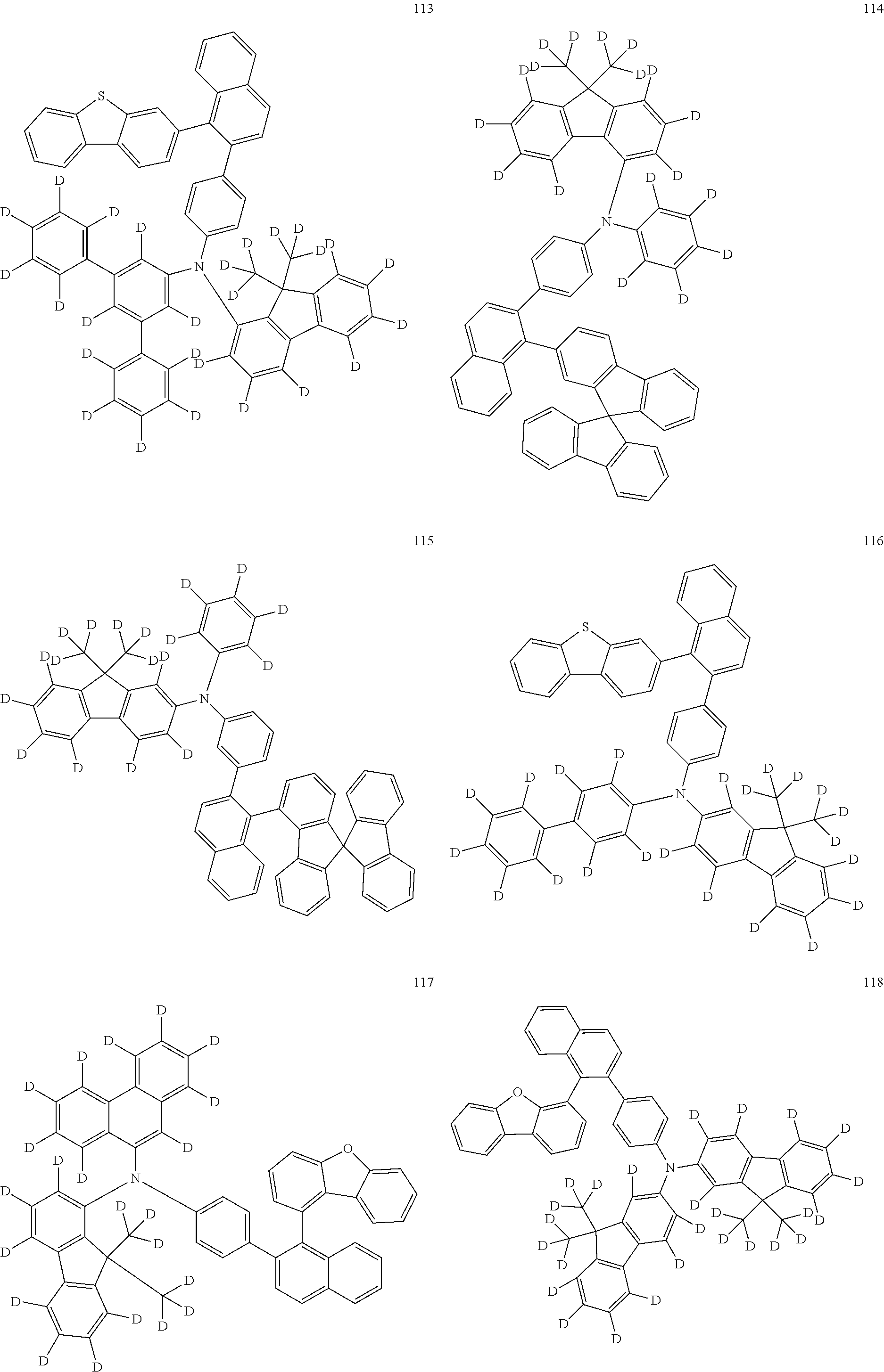

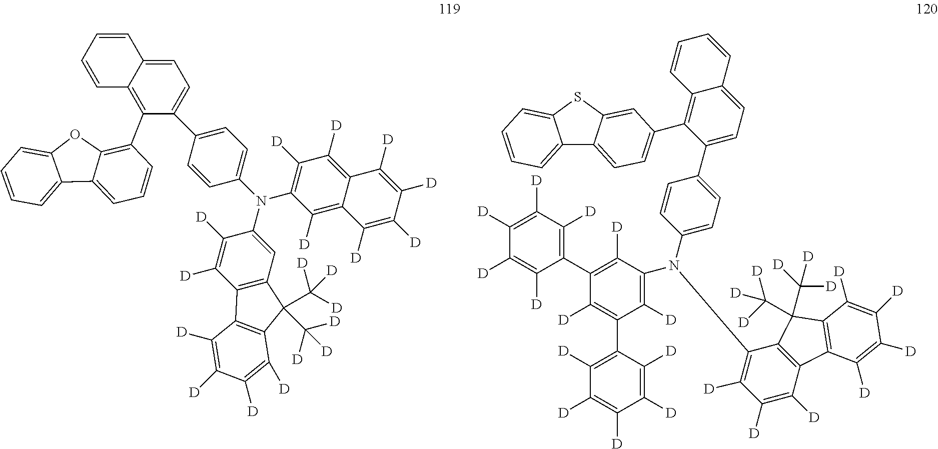 [SMILES: [2H]c1c(N(c2ccc(-c3ccc4ccccc4c3-c3cccc4c3oc3ccccc34)cc2)c2c([2H])c([2H])c3c([2H])c([2H])c([2H])c([2H])c3c2[2H])cc2c(c1[2H])-c1c([2H])c([2H])c([2H])c([2H])c1C2(C([2H])([2H])[2H])C([2H])([2H])[2H] ; [2H]c1c([2H])c([2H])c(-c2c([2H])c(-c3c([2H])c([2H])c([2H])c([2H])c3[2H])c([2H])c(N(c3ccc(-c4ccc5ccccc5c4-c4ccc5c(c4)sc4ccccc45)cc3)c3c([2H])c([2H])c([2H])c4c3C(C([2H])([2H])[2H])(C([2H])([2H])[2H])c3c([2H])c([2H])c([2H])c([2H])c3-4)c2[2H])c([2H])c1[2H]]

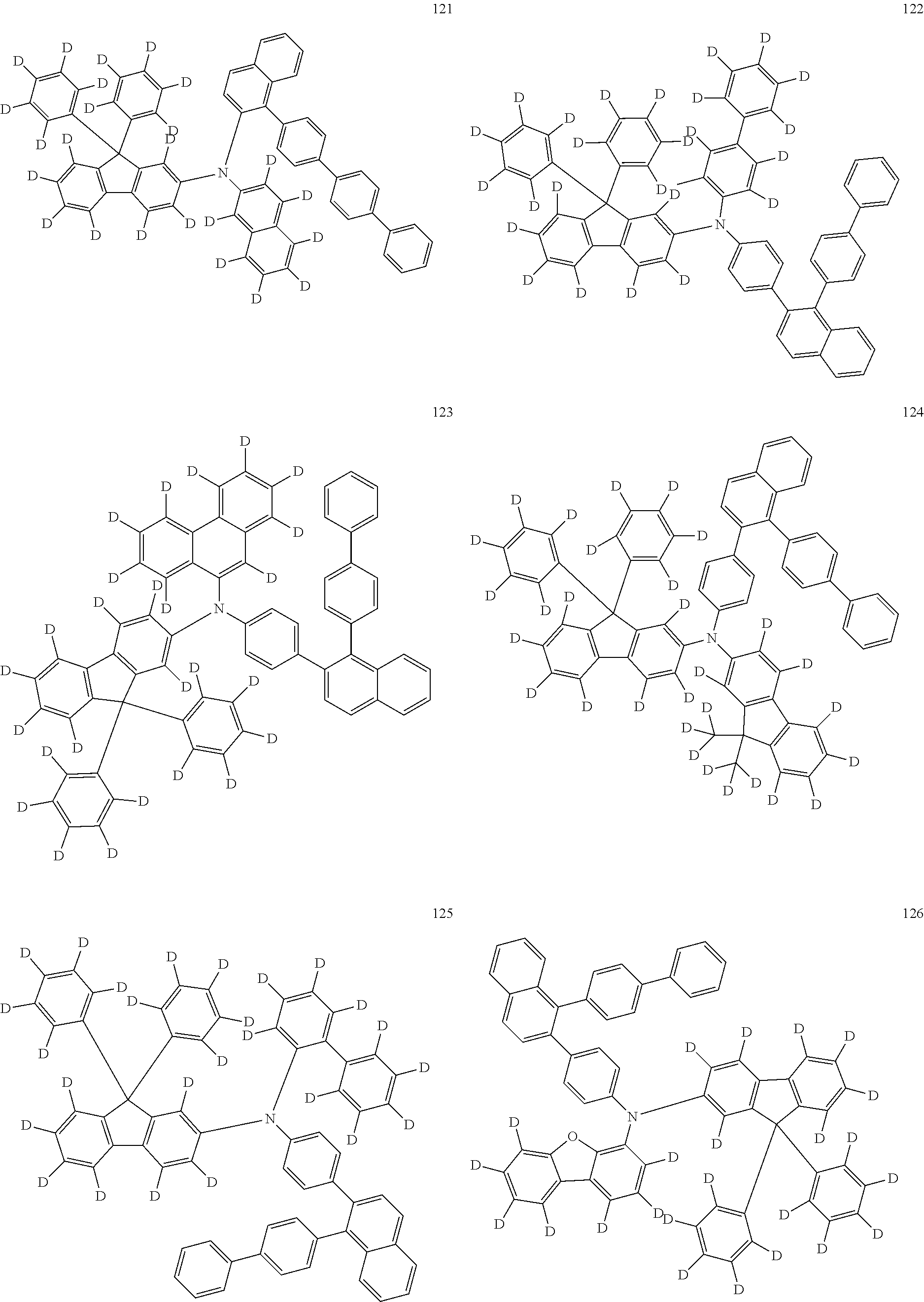

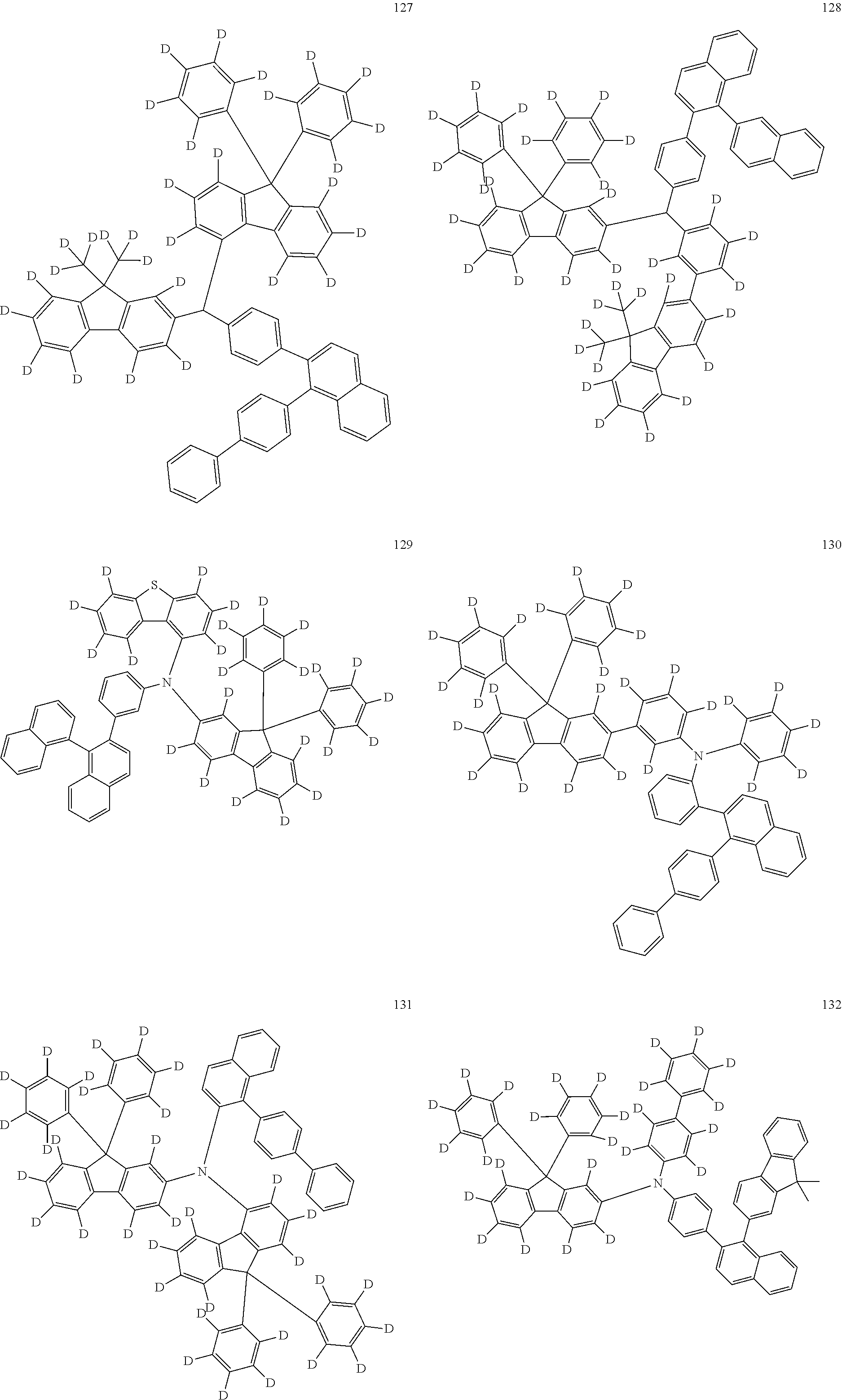

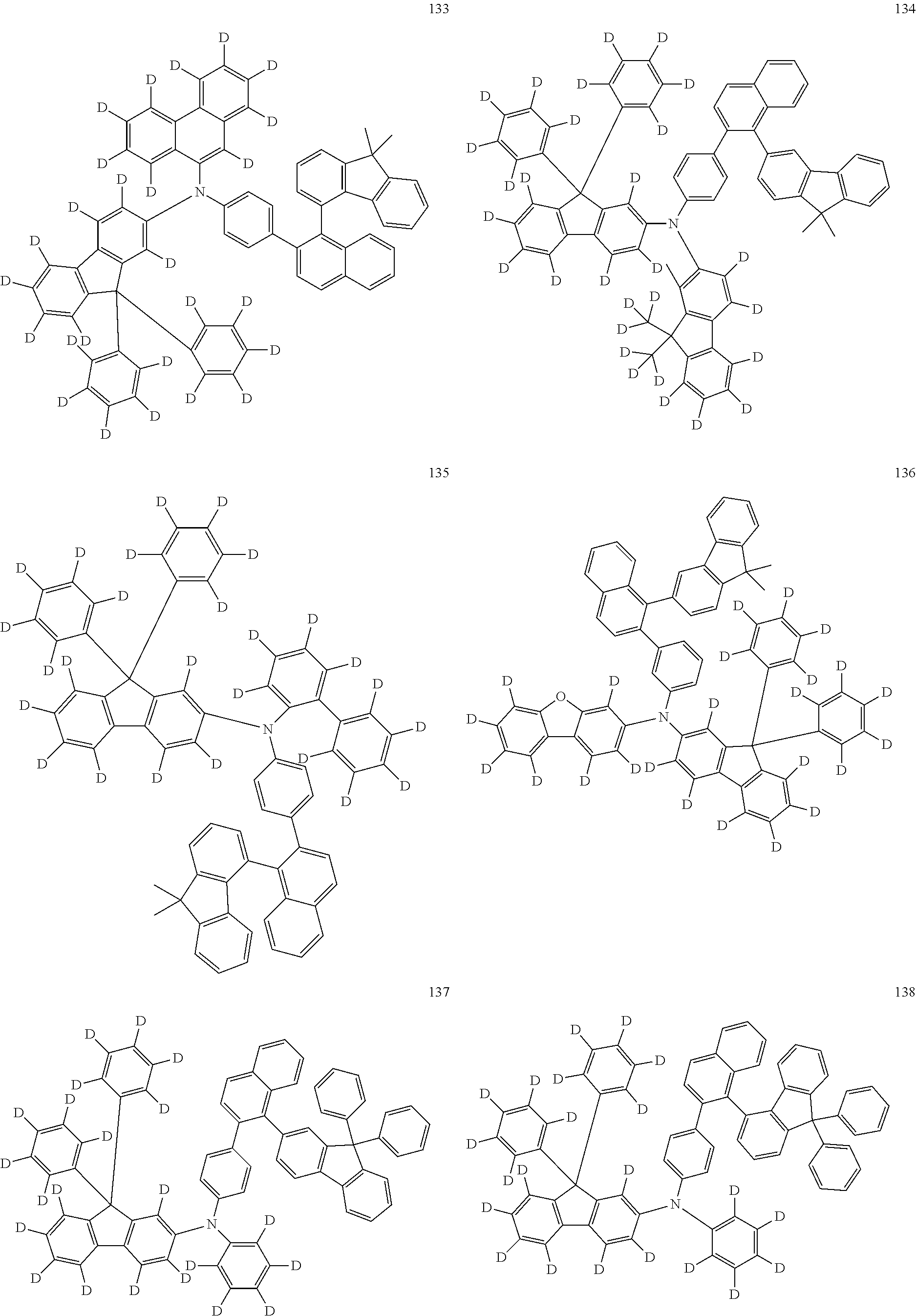

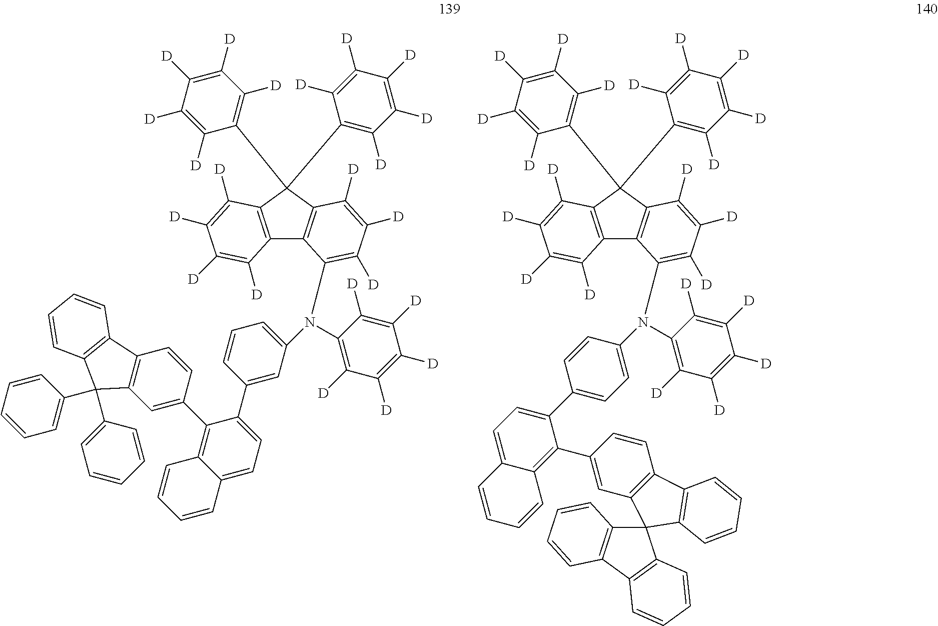 [SMILES: [2H]c1c([2H])c([2H])c(N(c2cccc(-c3ccc4ccccc4c3-c3ccc4c(c3)C(c3ccccc3)(c3ccccc3)c3ccccc3-4)c2)c2c([2H])c([2H])c([2H])c3c2-c2c([2H])c([2H])c([2H])c([2H])c2C3(c2c([2H])c([2H])c([2H])c([2H])c2[2H])c2c([2H])c([2H])c([2H])c([2H])c2[2H])c([2H])c1[2H] ; [2H]c1c([2H])c([2H])c(N(c2ccc(-c3ccc4ccccc4c3-c3ccc4c(c3)C3(c5ccccc5-c5ccccc53)c3ccccc3-4)cc2)c2c([2H])c([2H])c([2H])c3c2-c2c([2H])c([2H])c([2H])c([2H])c2C3(c2c([2H])c([2H])c([2H])c([2H])c2[2H])c2c([2H])c([2H])c([2H])c([2H])c2[2H])c([2H])c1[2H]]

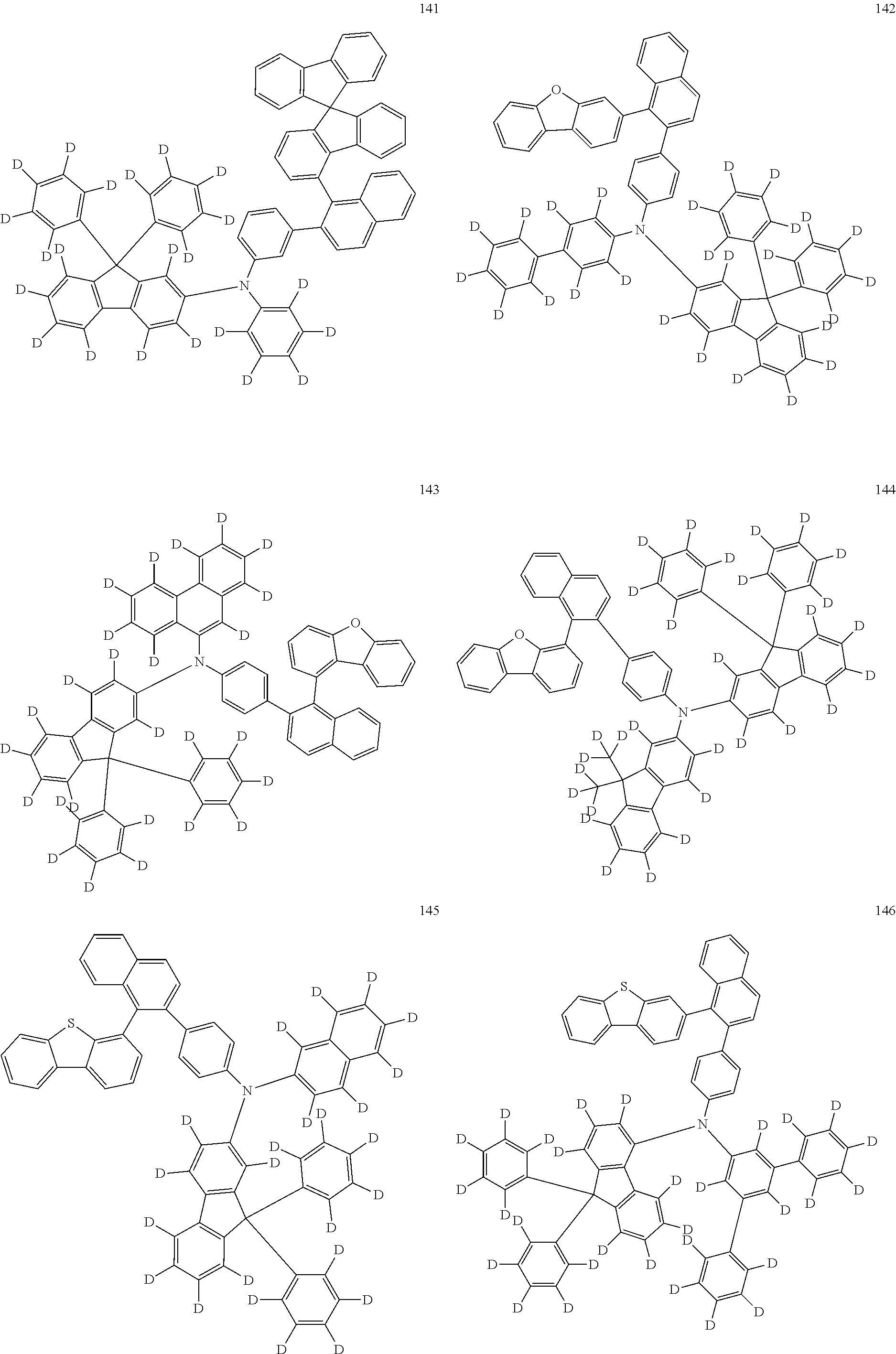

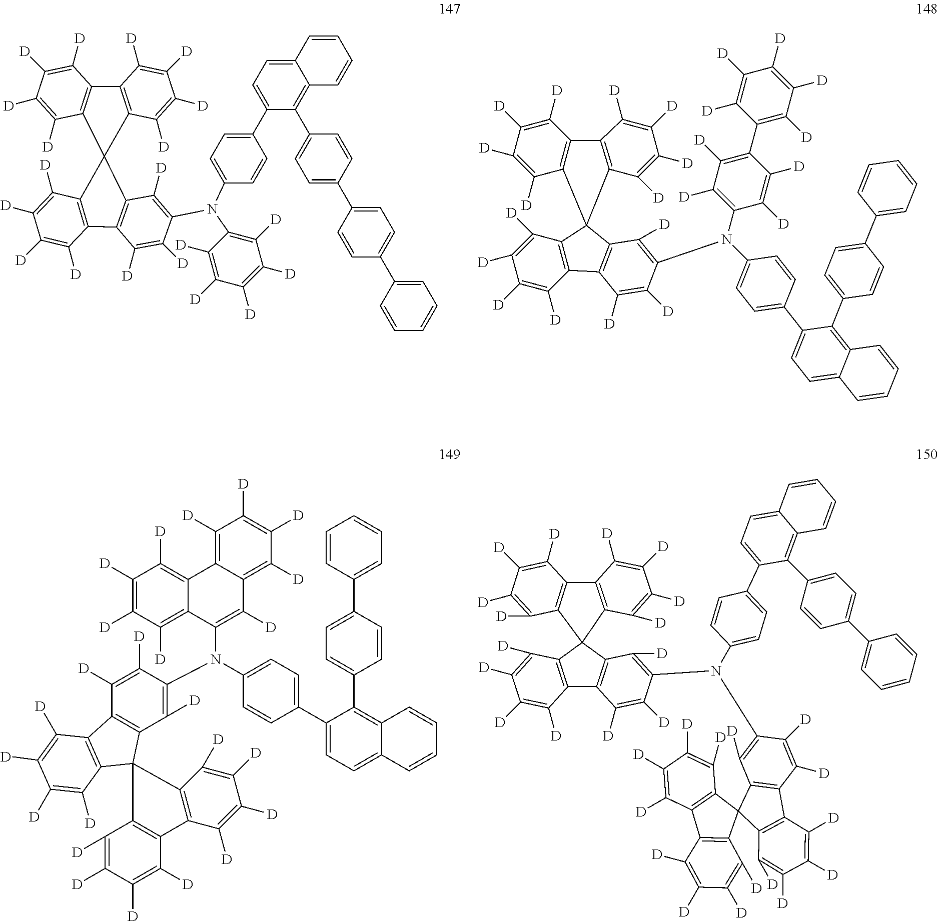

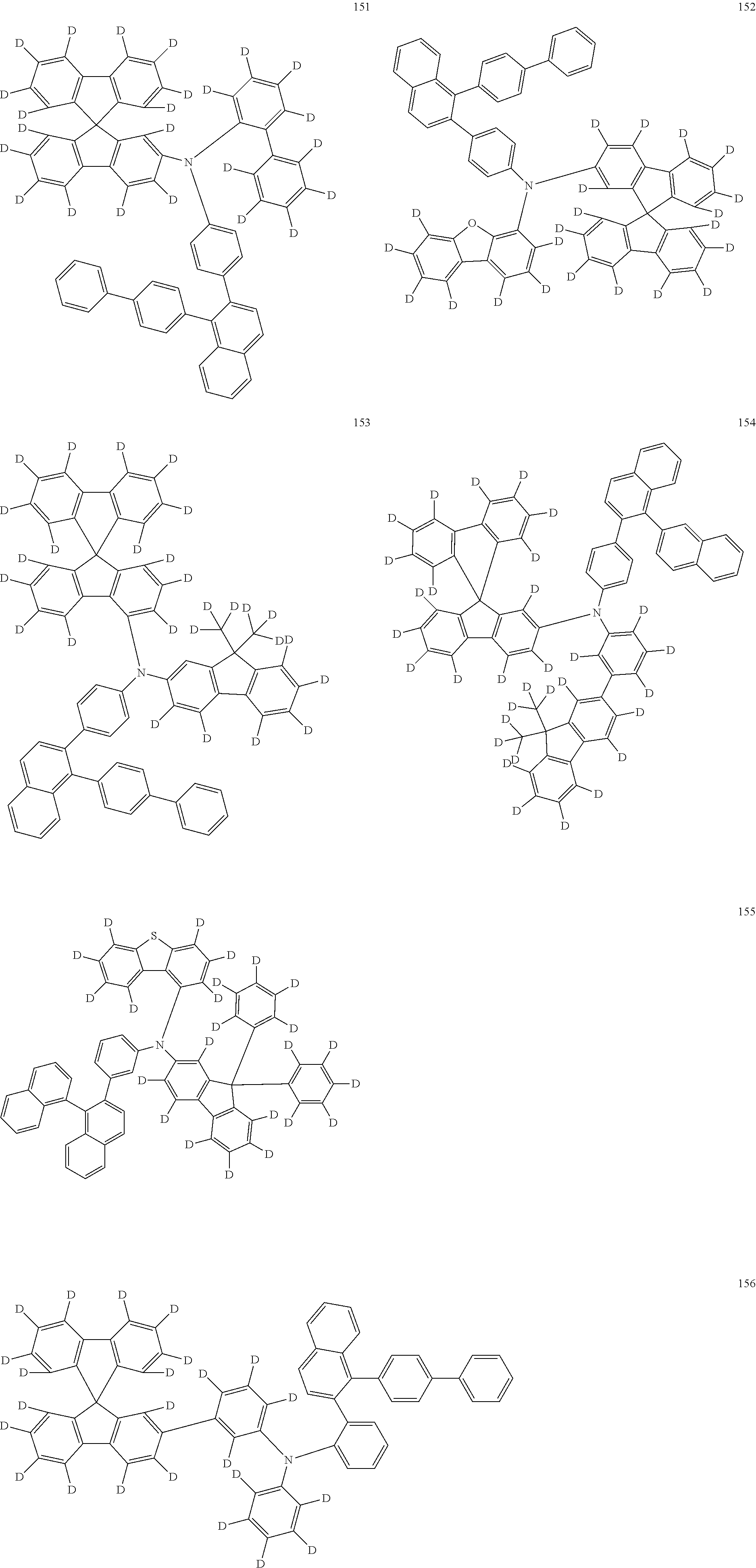

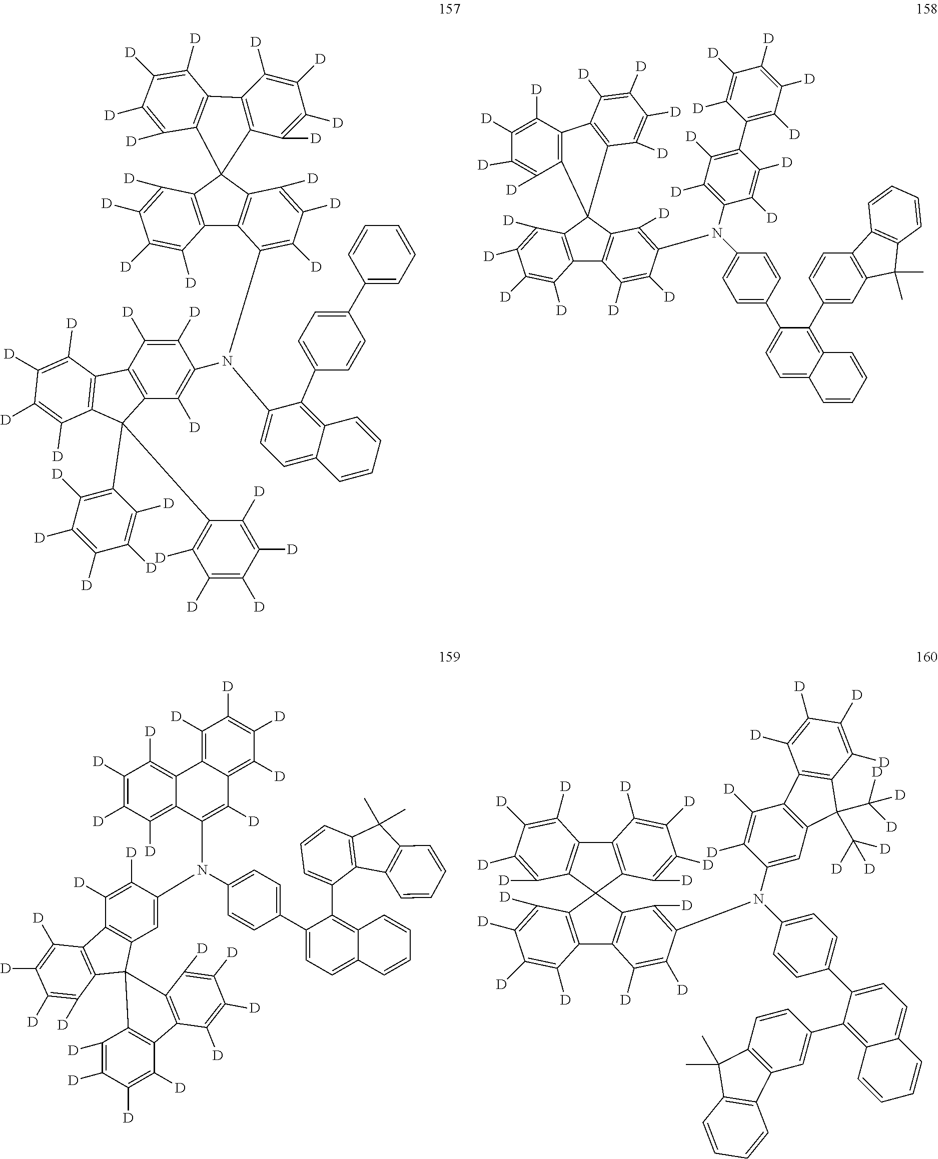

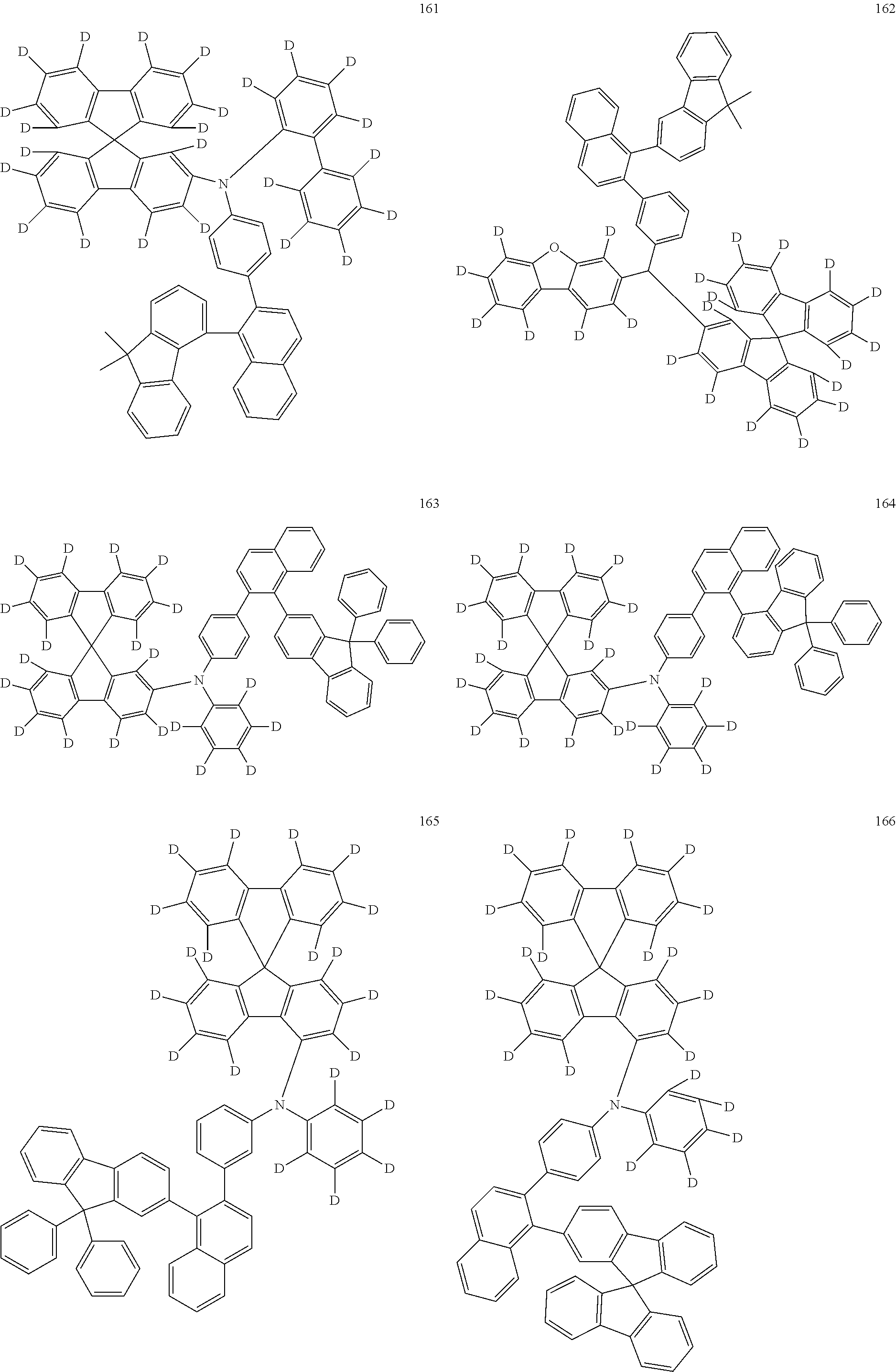

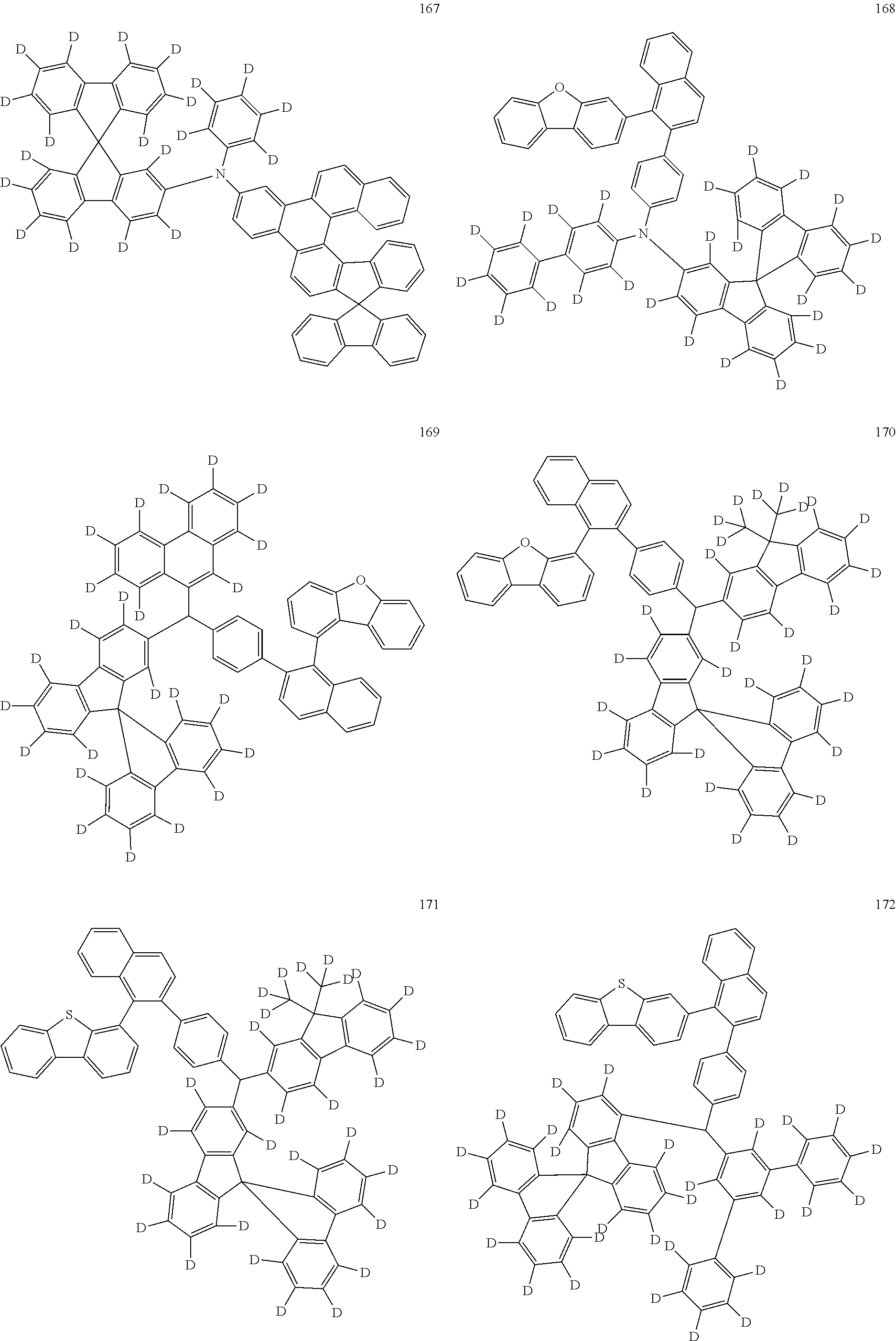

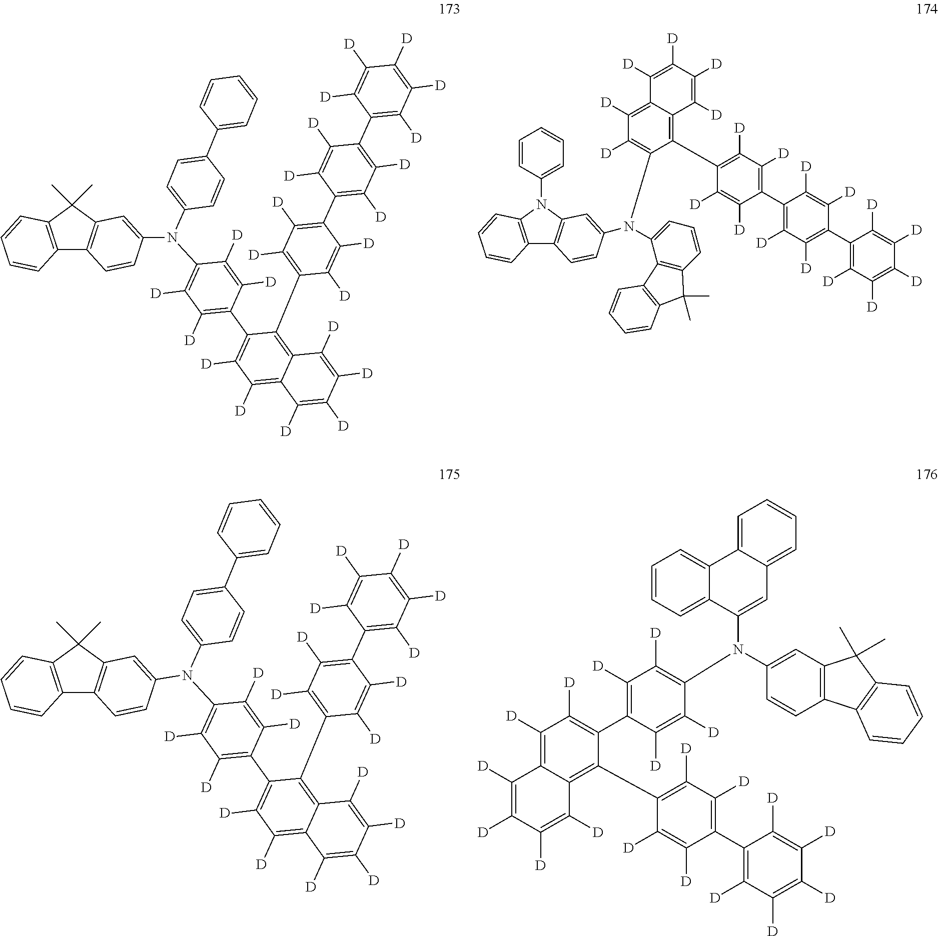

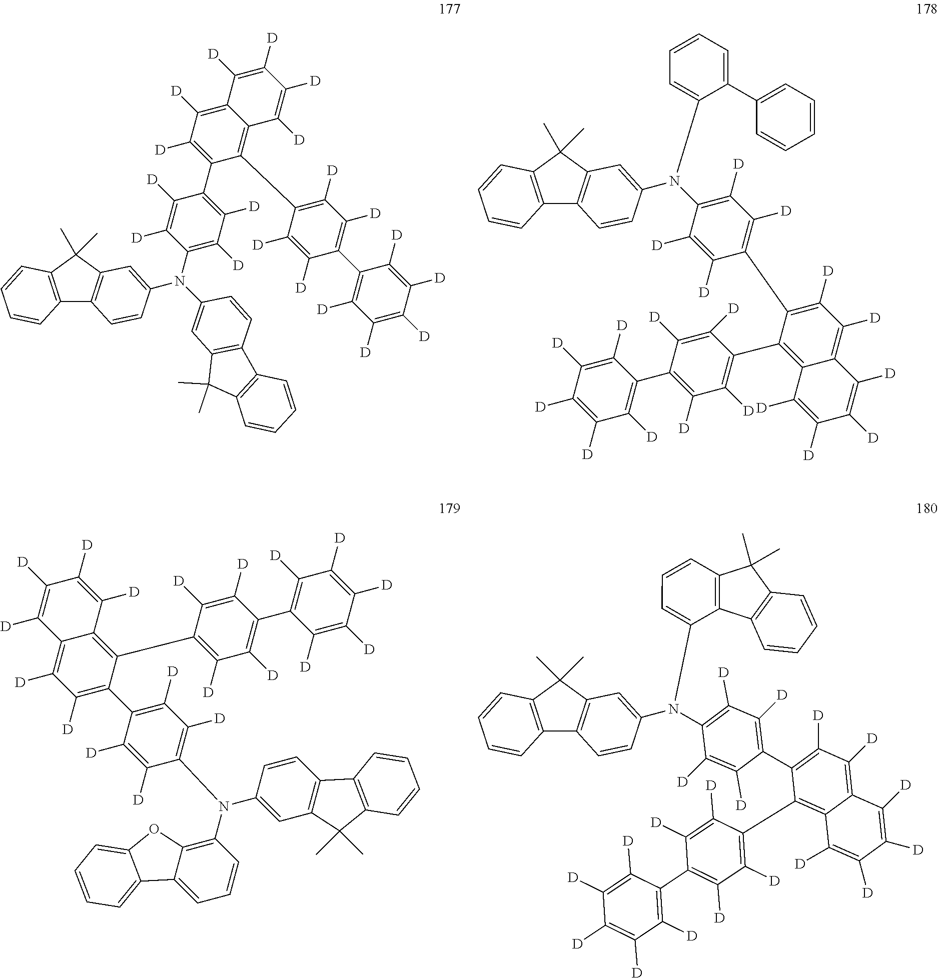

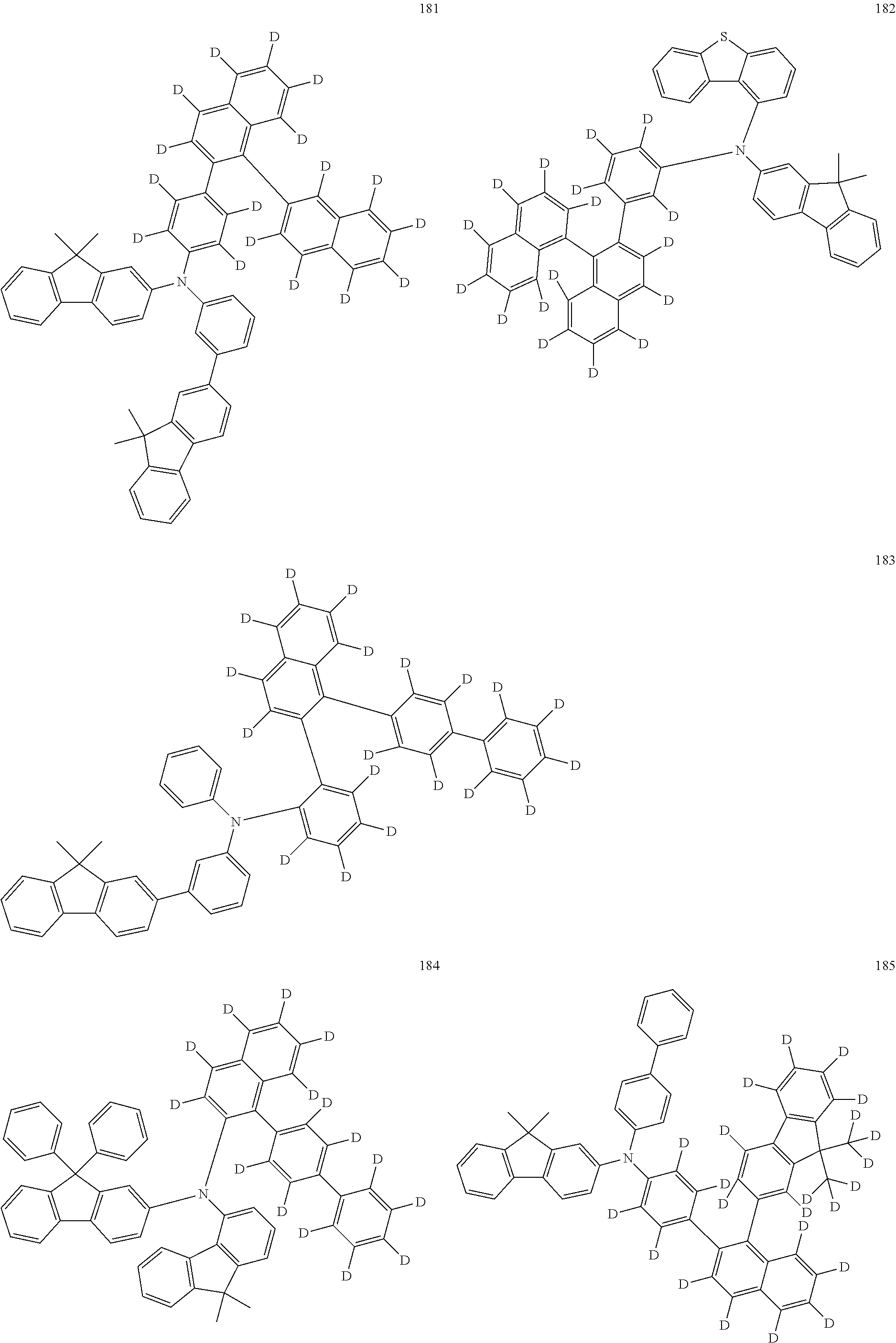

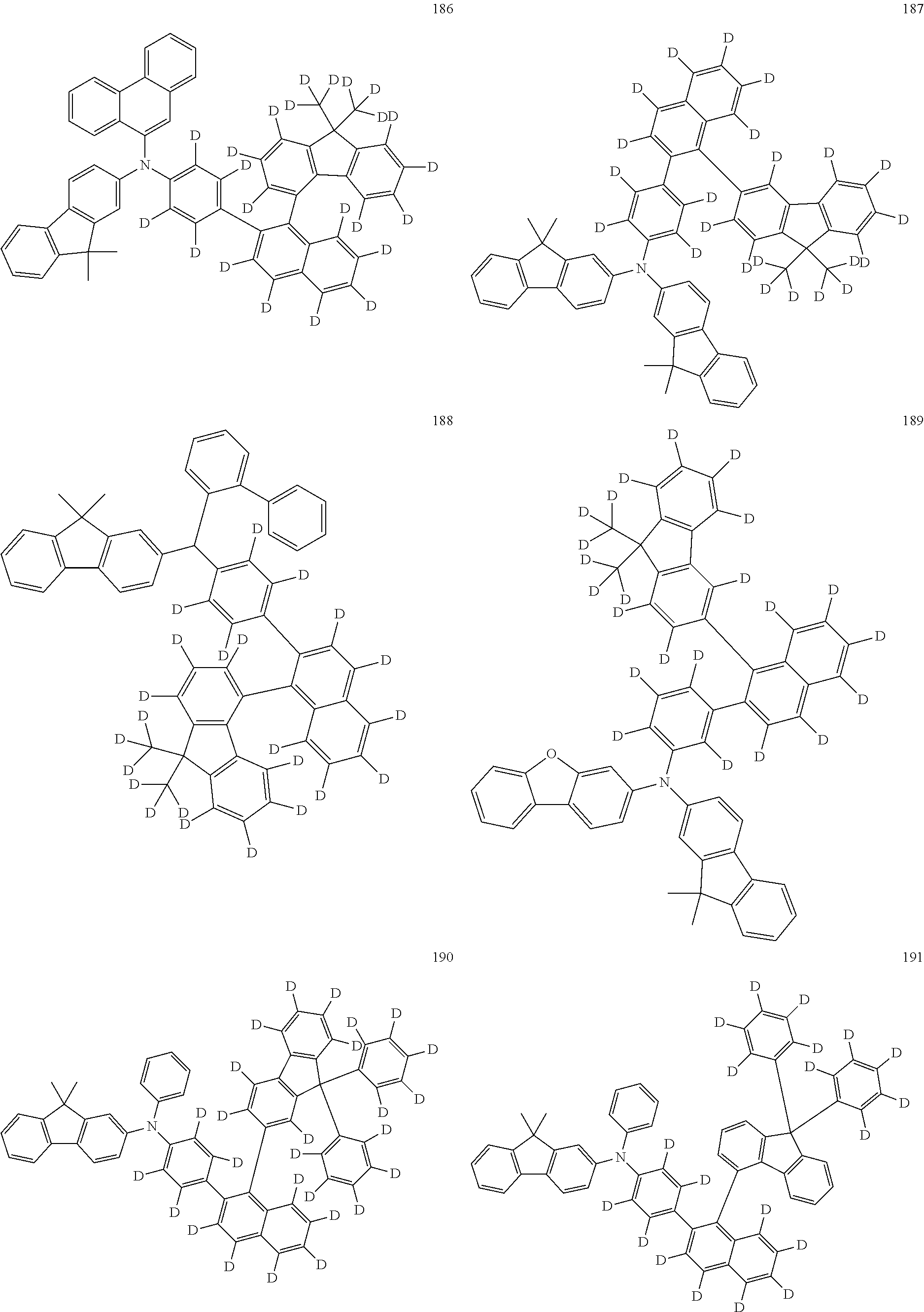

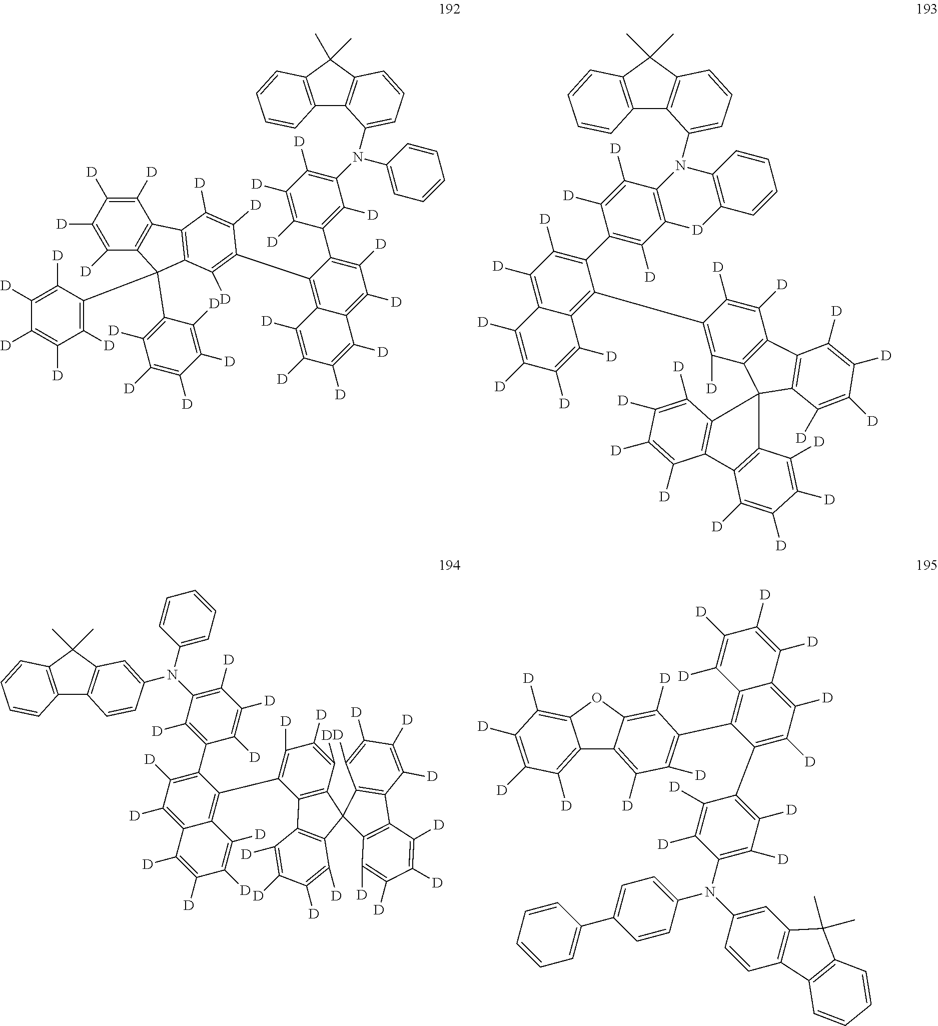

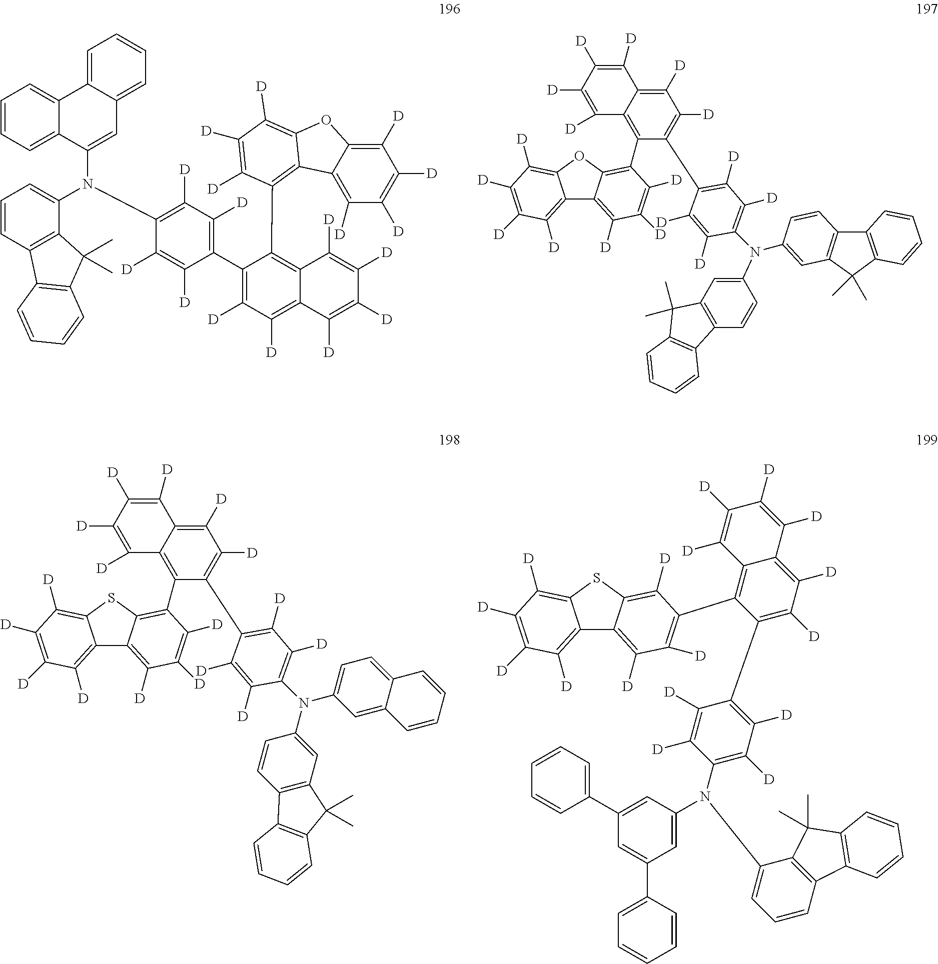

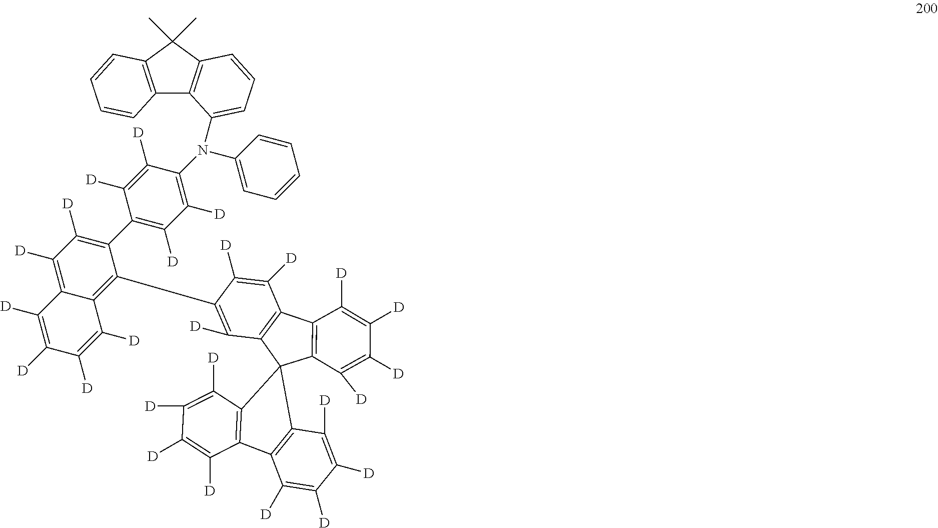 [SMILES: [2H]c1c([2H])c([2H])c2c(c1[2H])-c1c([2H])c([2H])c([2H])c([2H])c1C21c2c([2H])c([2H])c([2H])c([2H])c2-c2c([2H])c([2H])c(-c3c(-c4c([2H])c([2H])c(N(c5ccccc5)c5cccc6c5-c5ccccc5C6(C)C)c([2H])c4[2H])c([2H])c([2H])c4c([2H])c([2H])c([2H])c([2H])c34)c([2H])c21]

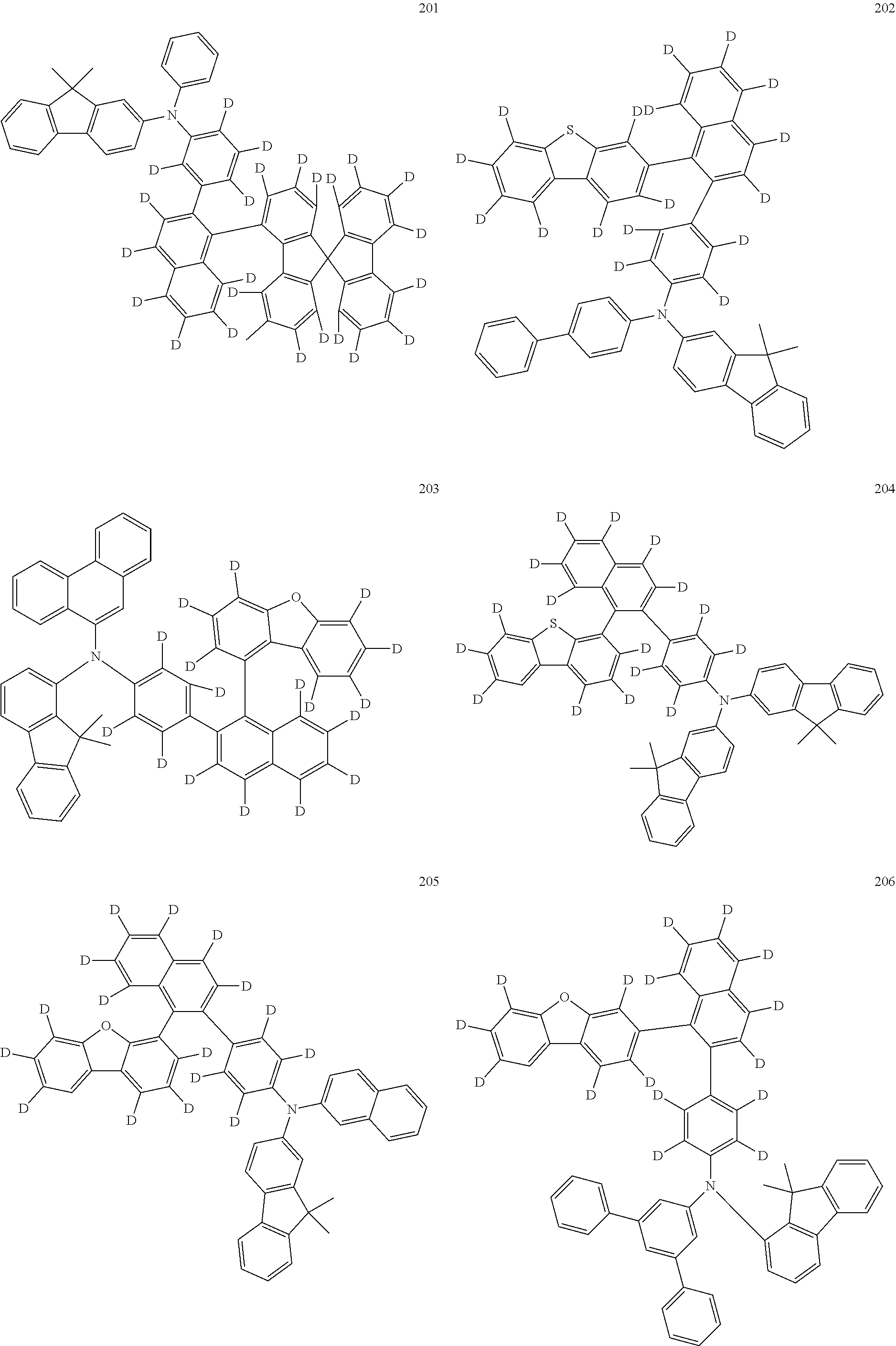

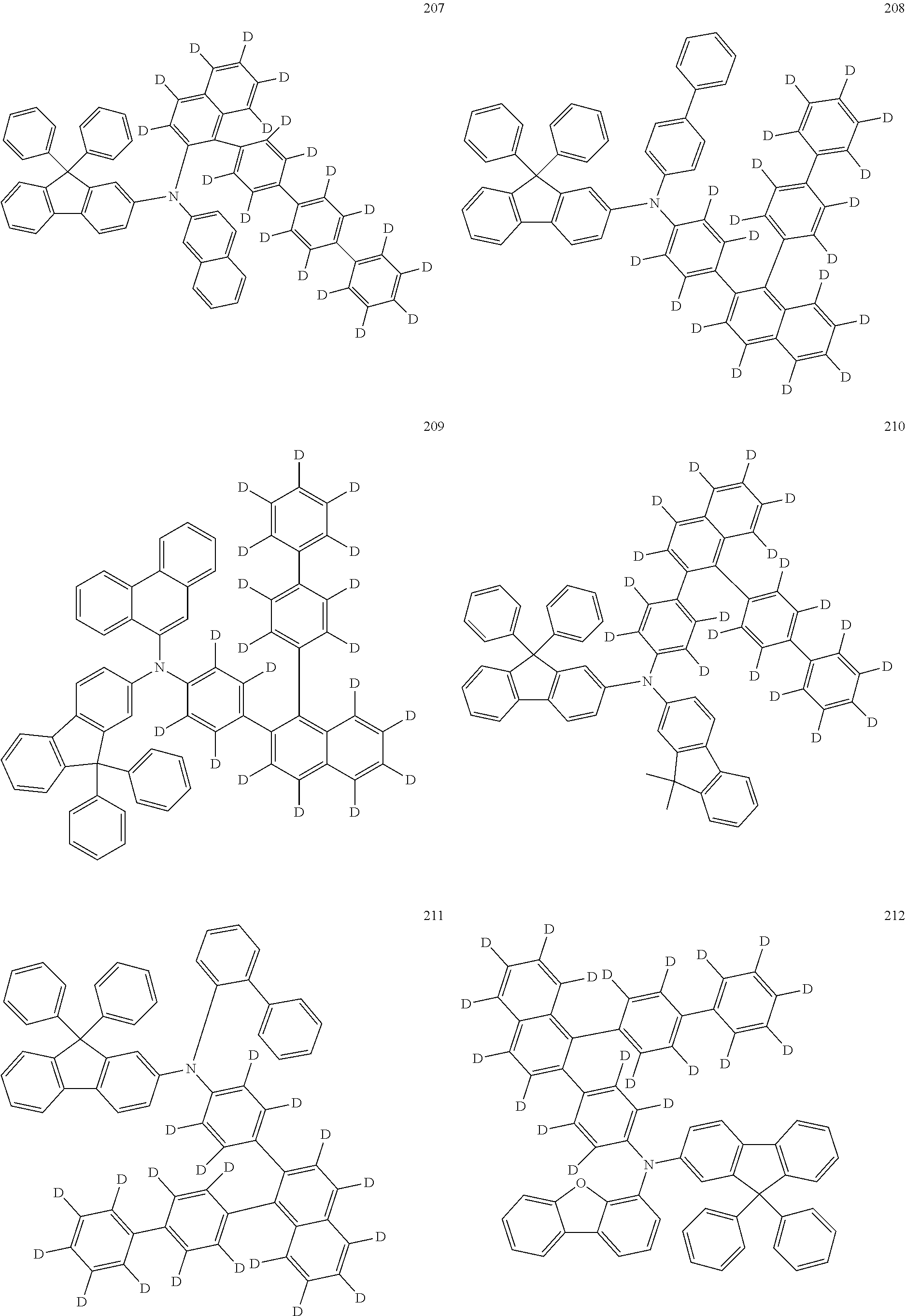

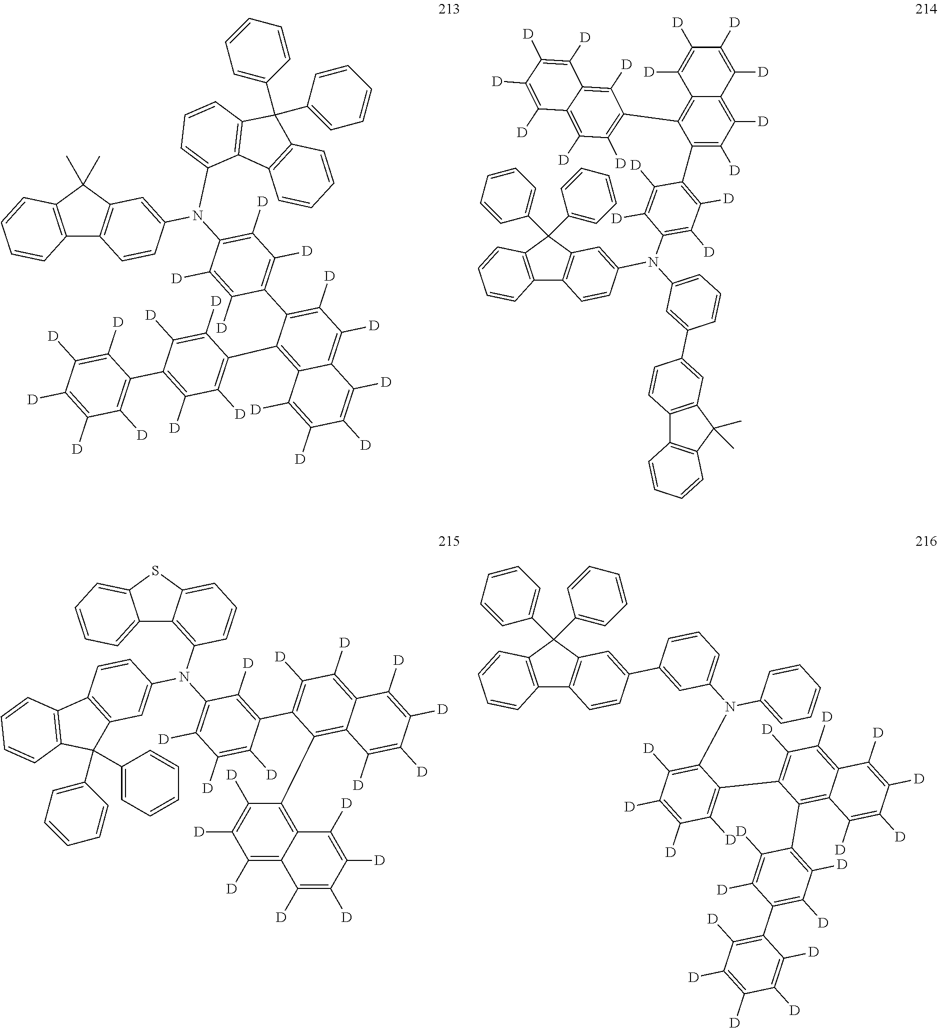

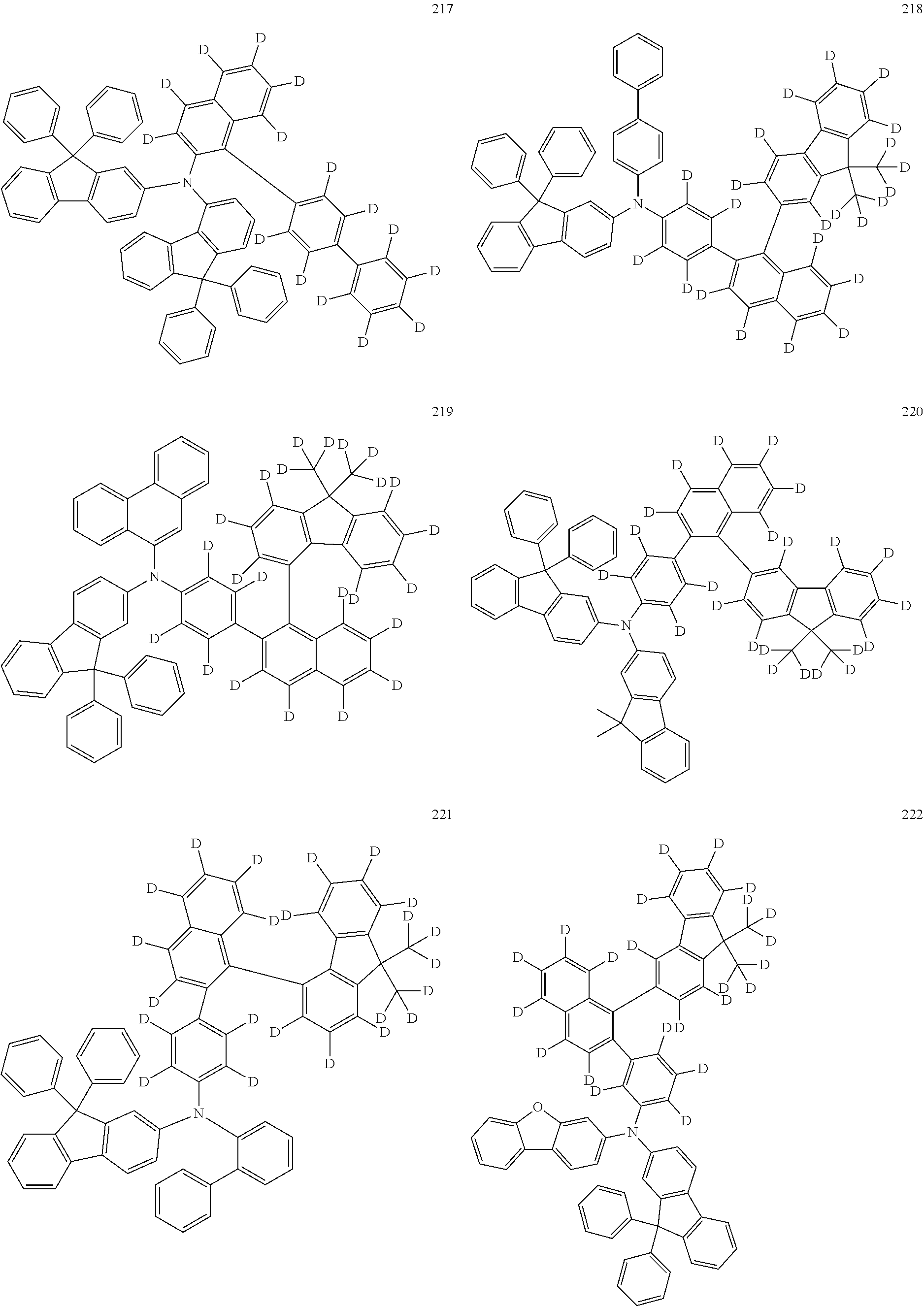

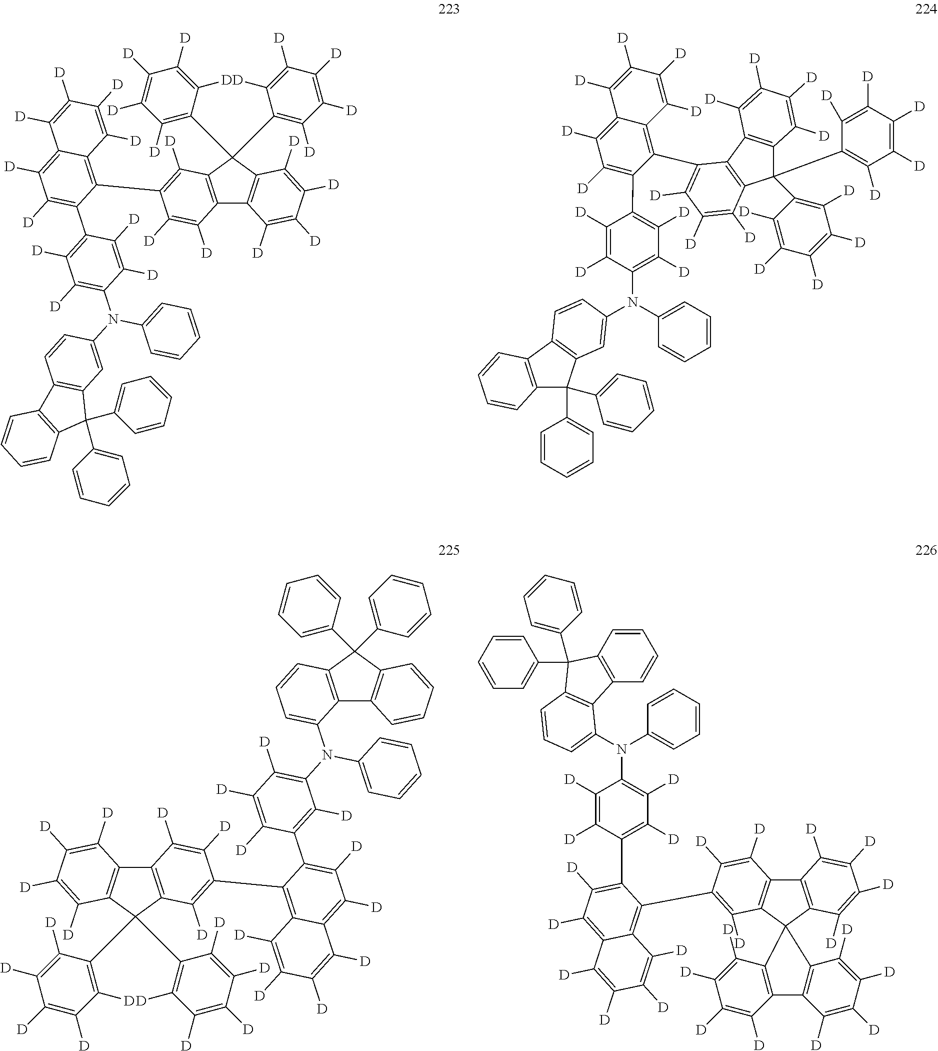

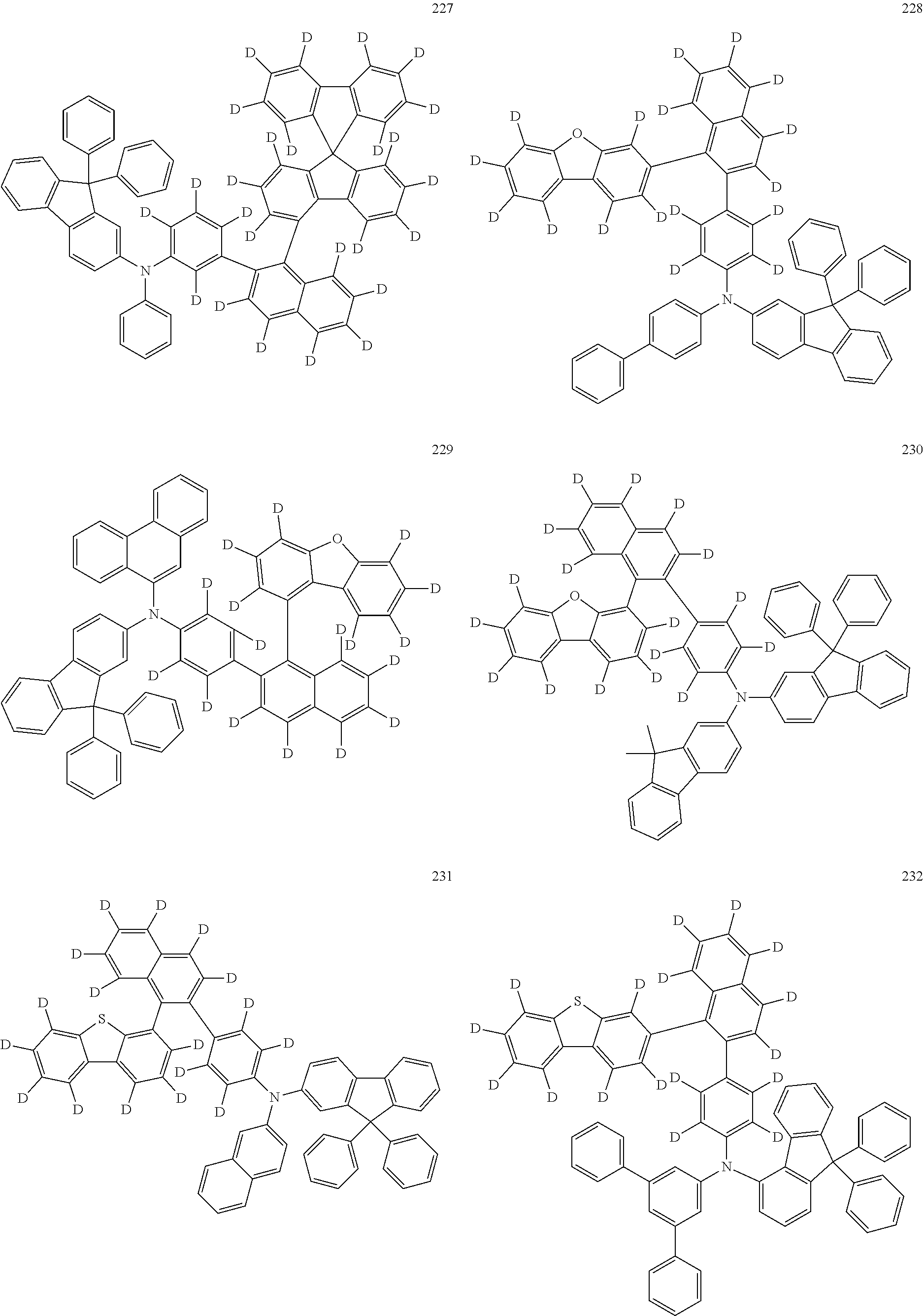

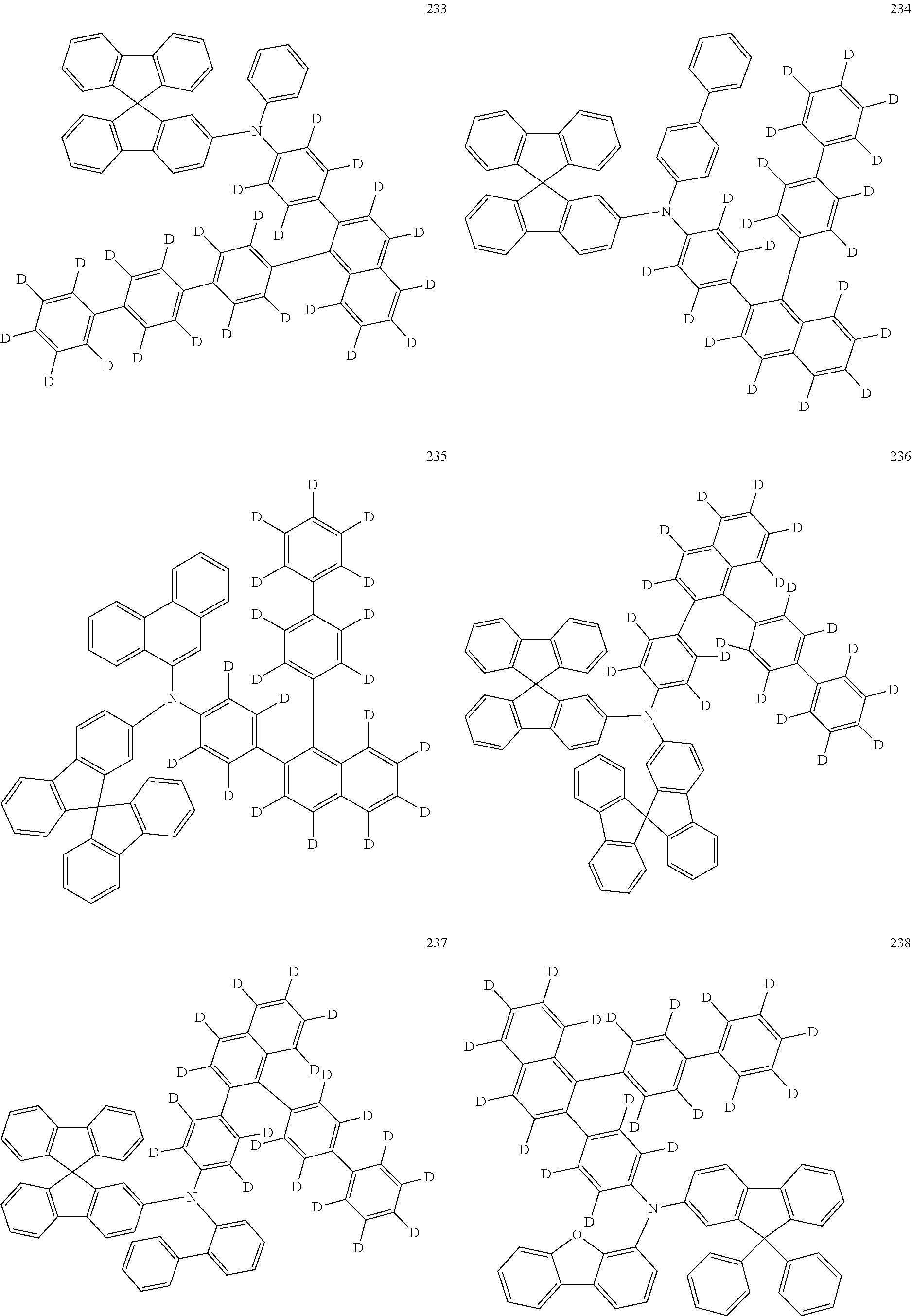

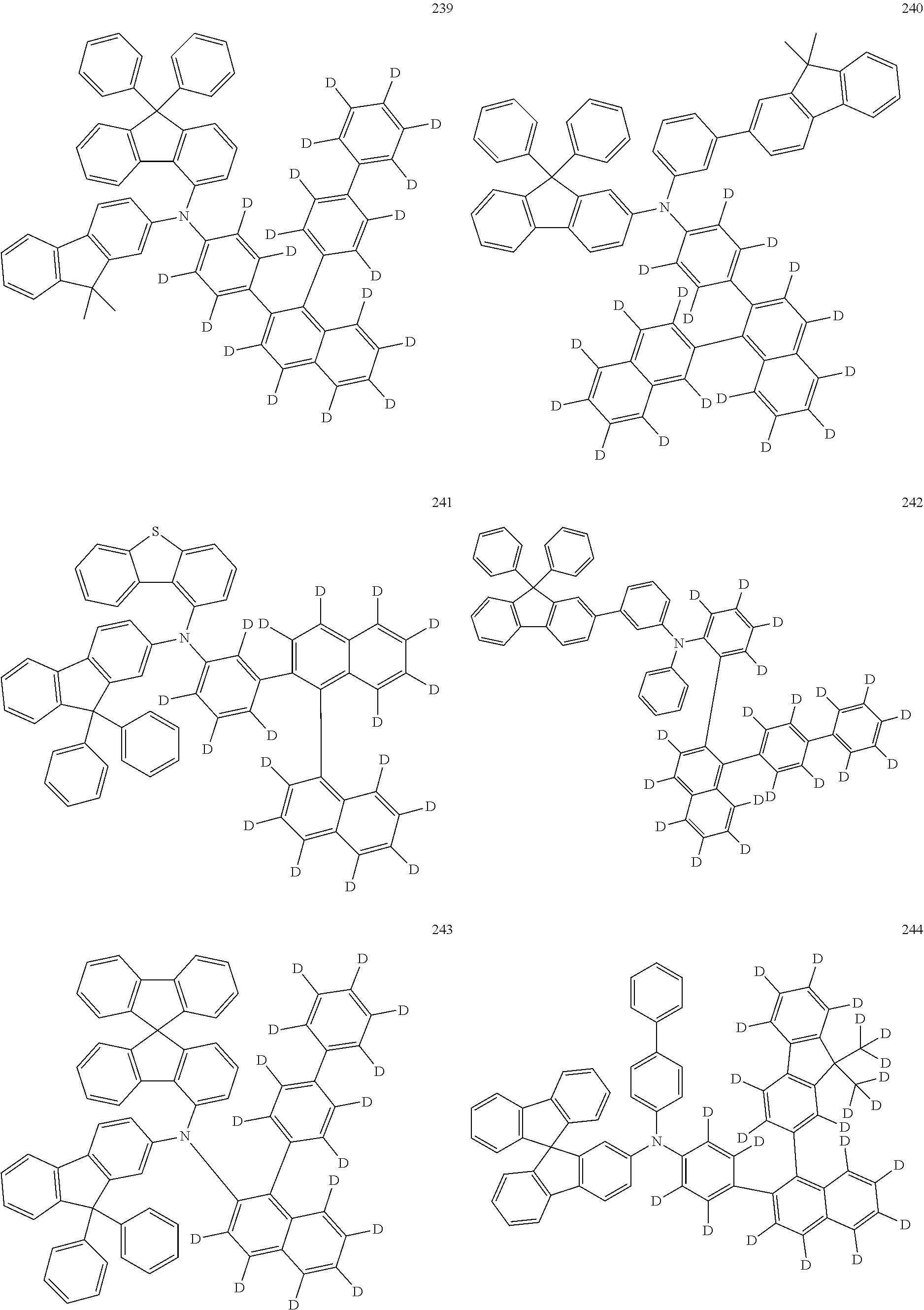

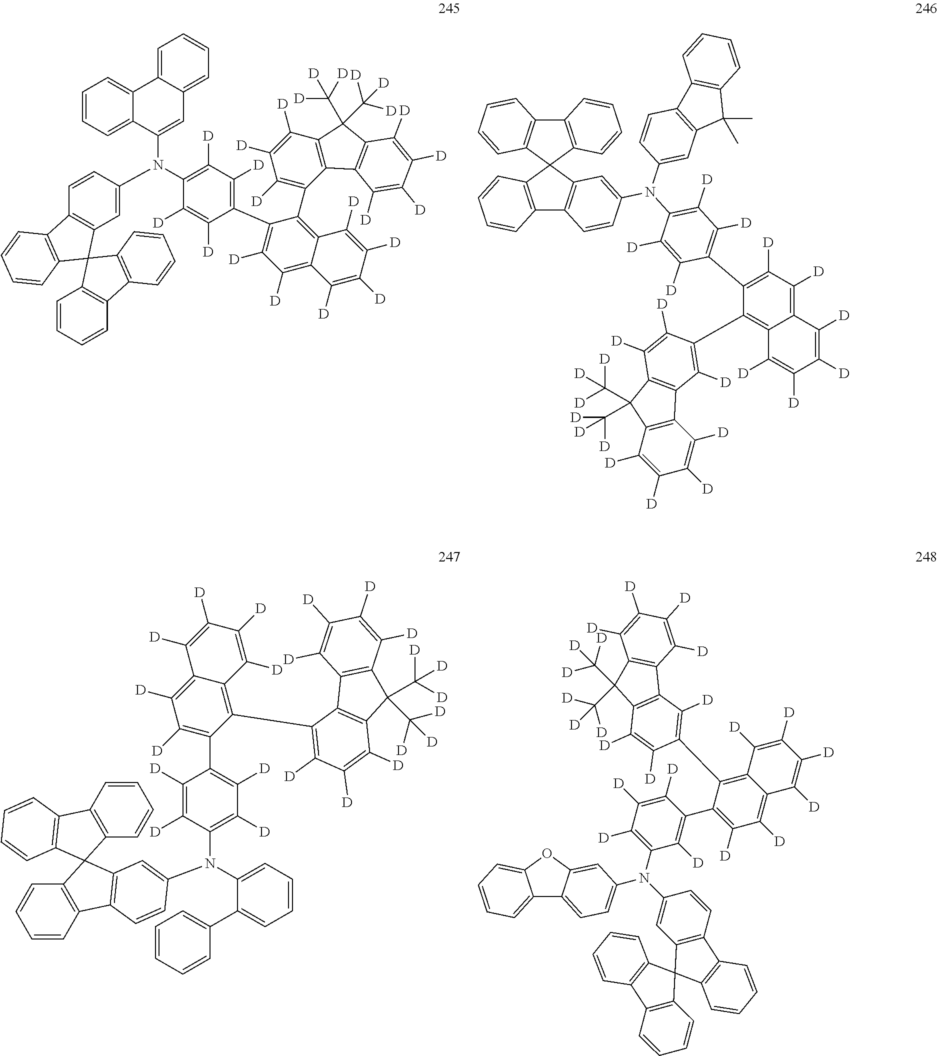

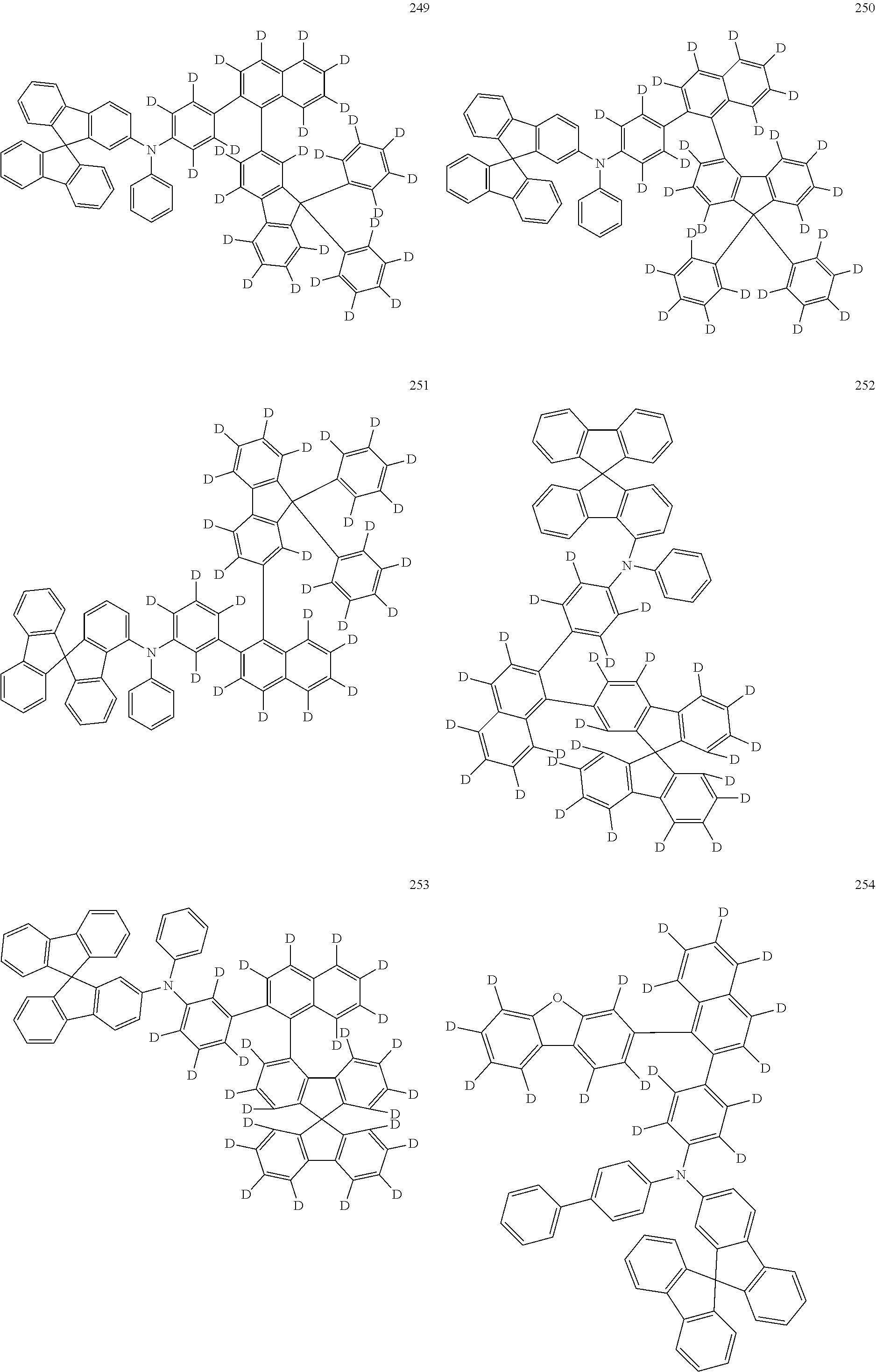

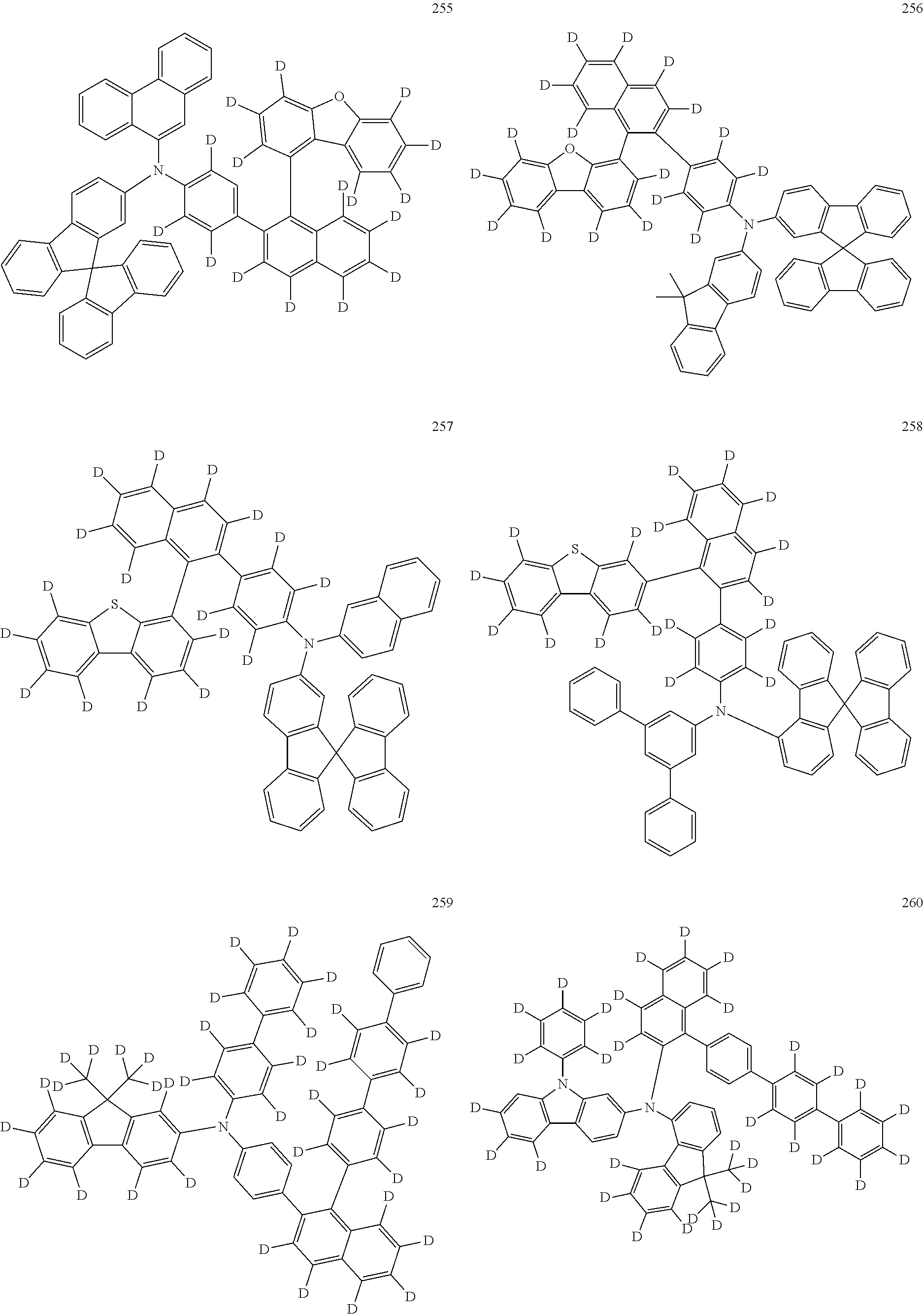

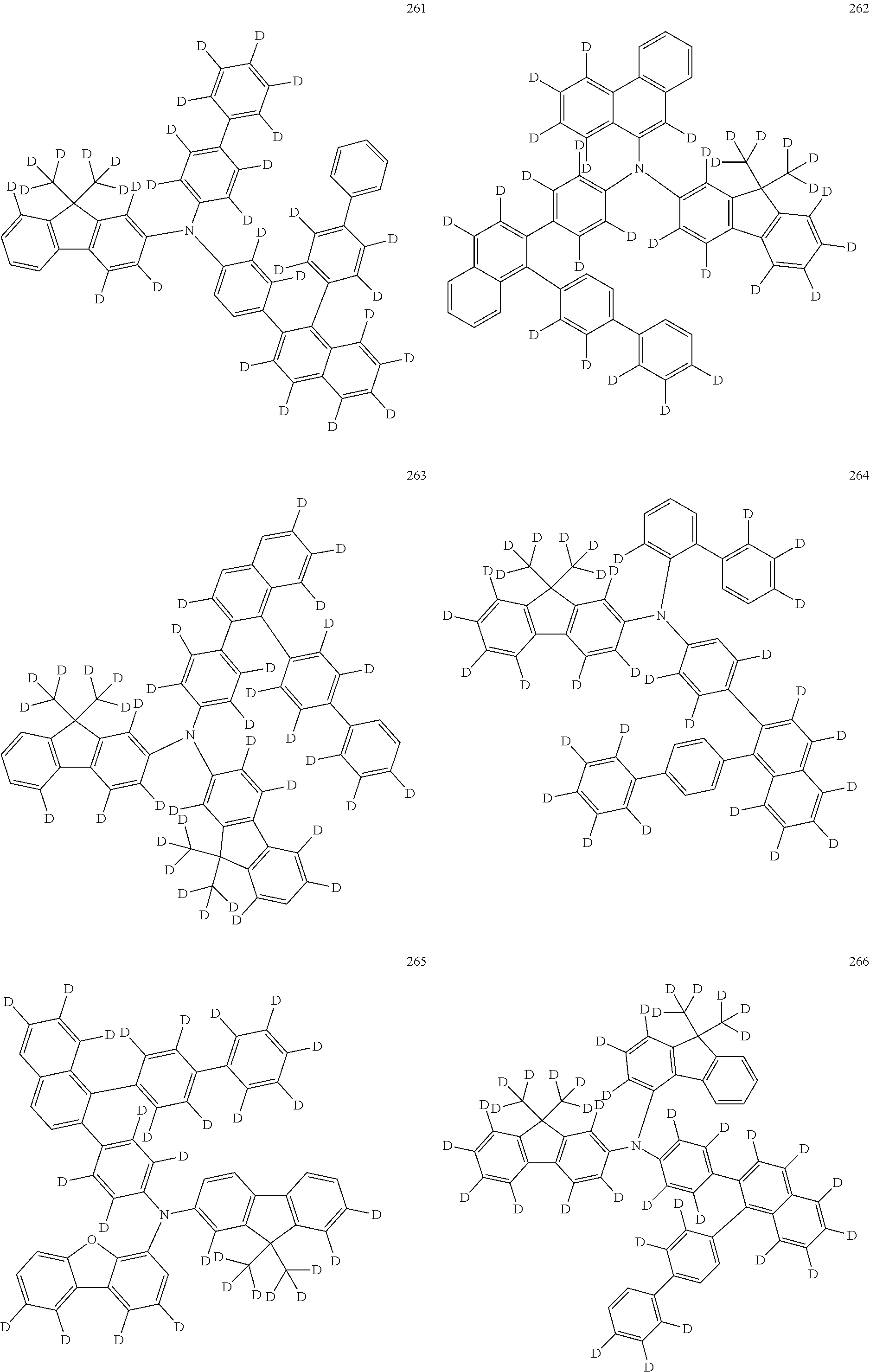

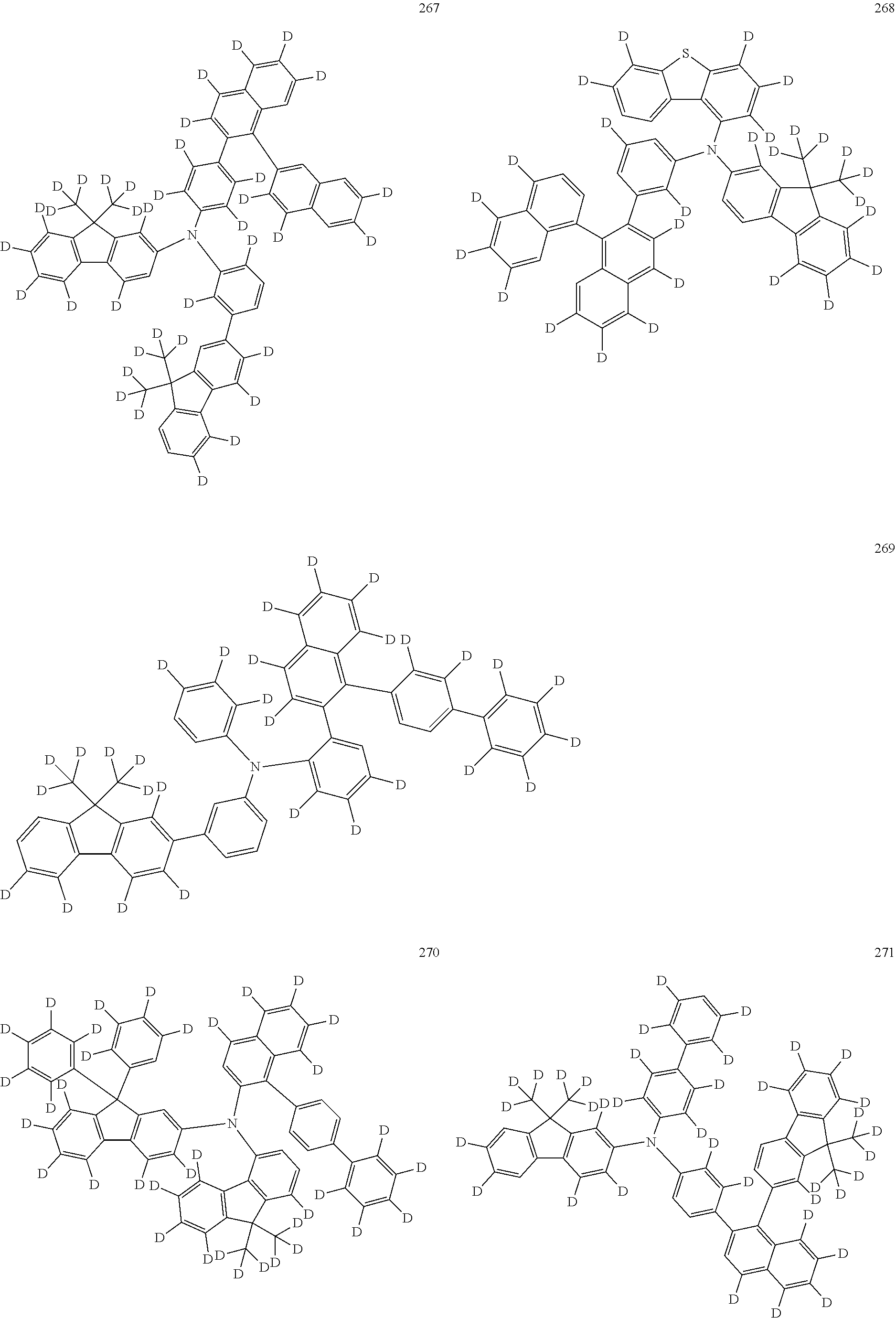

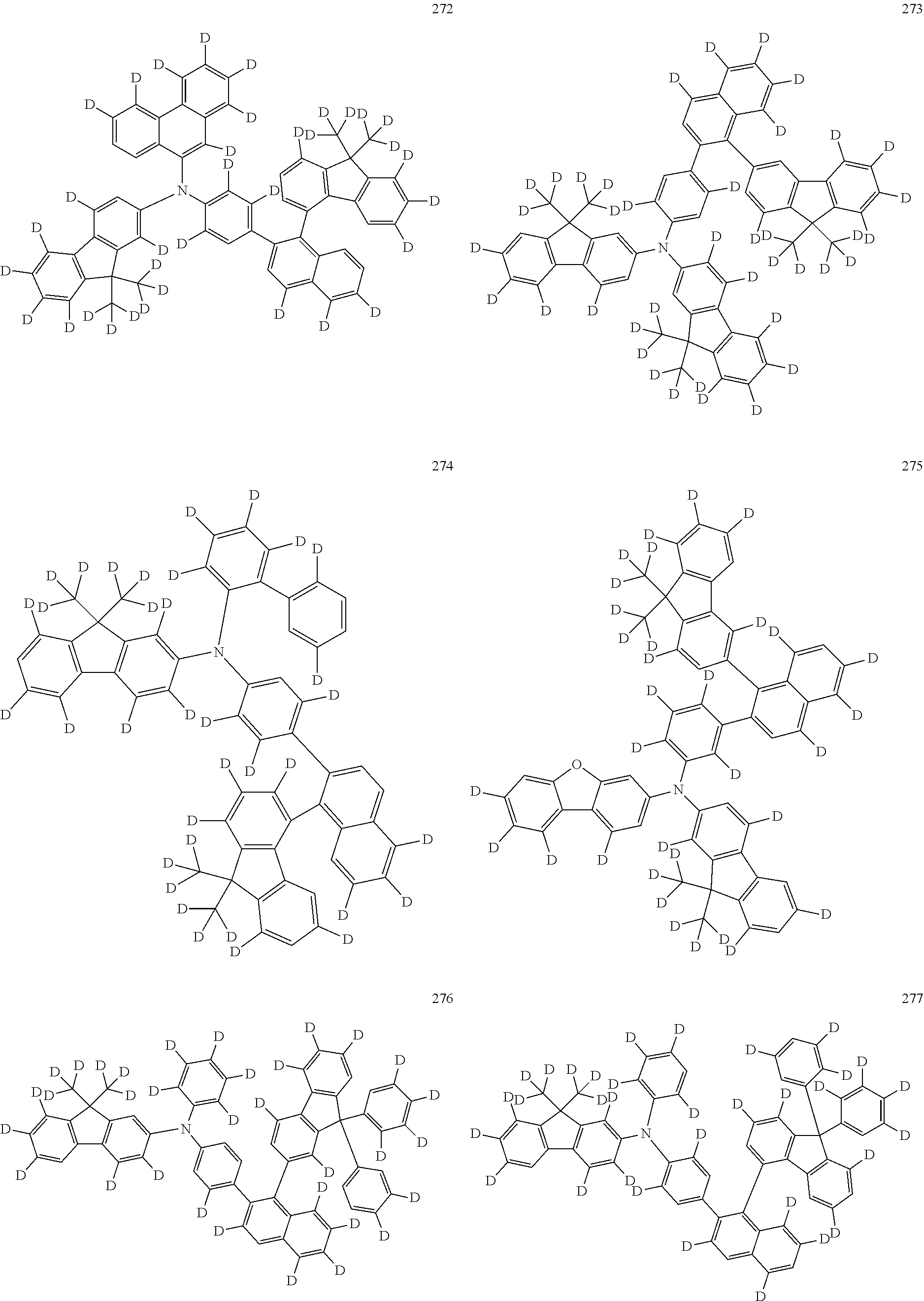

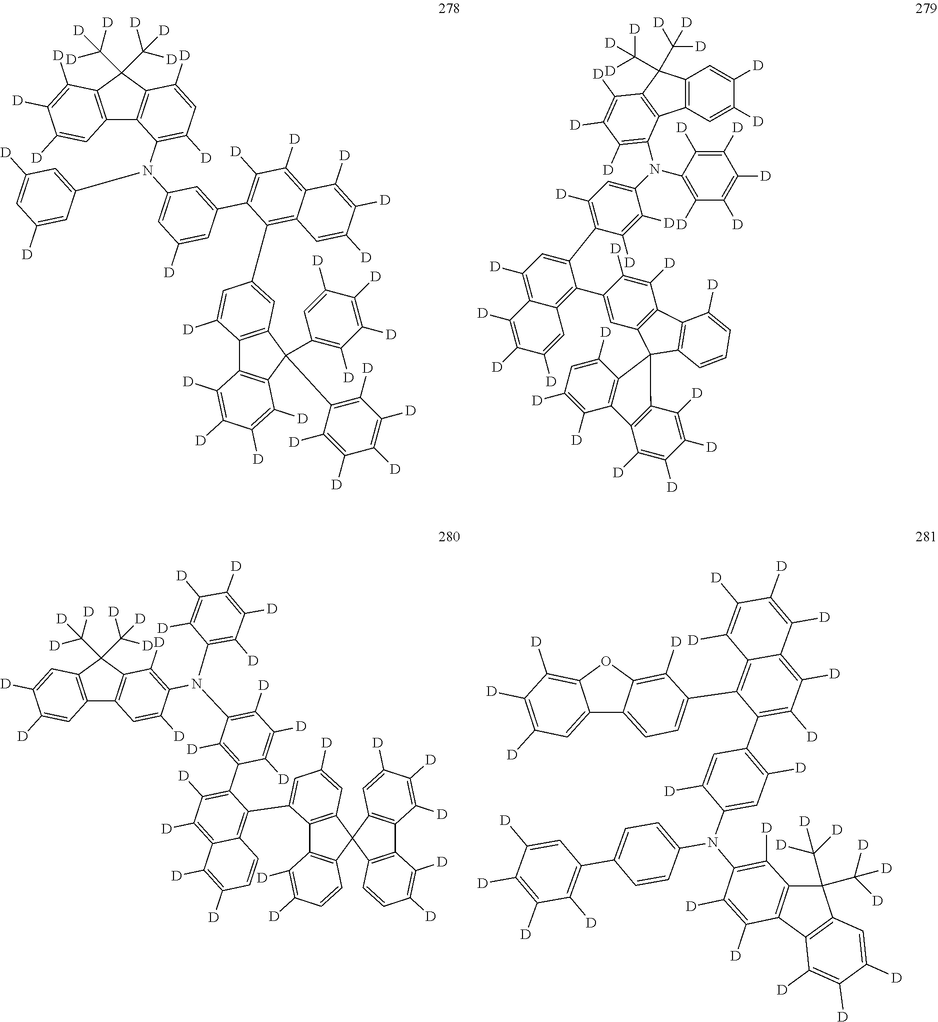

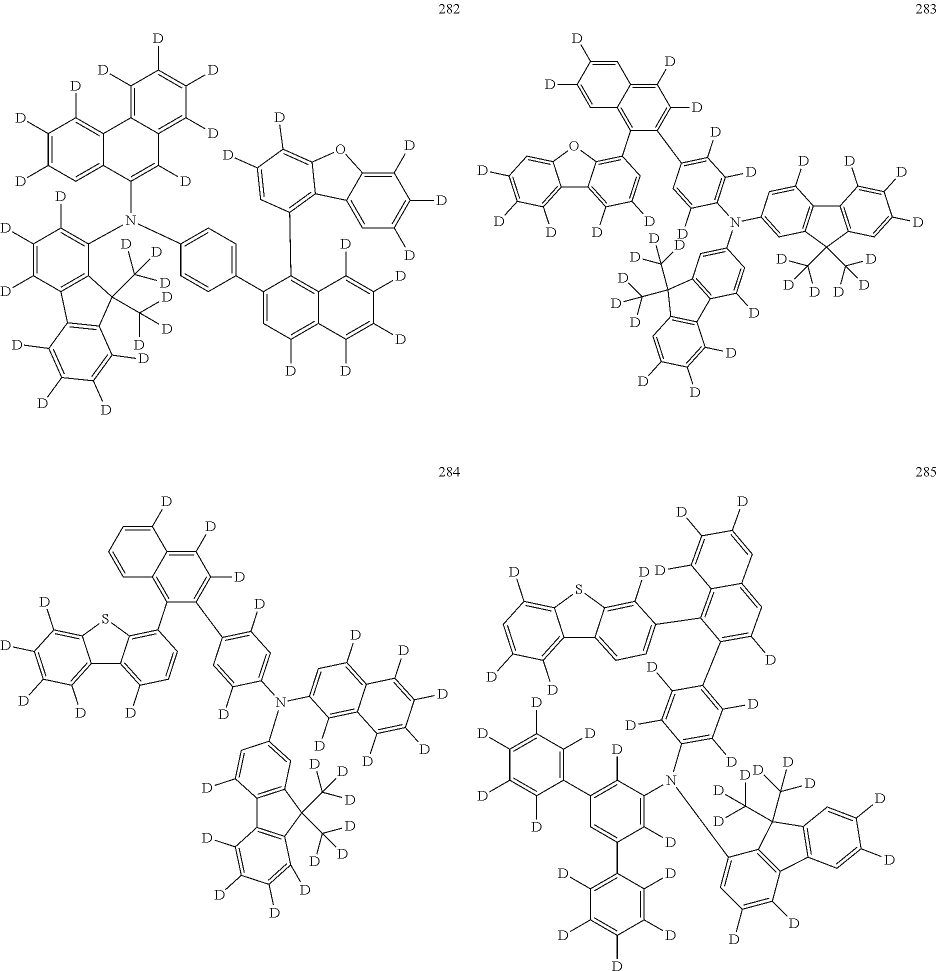

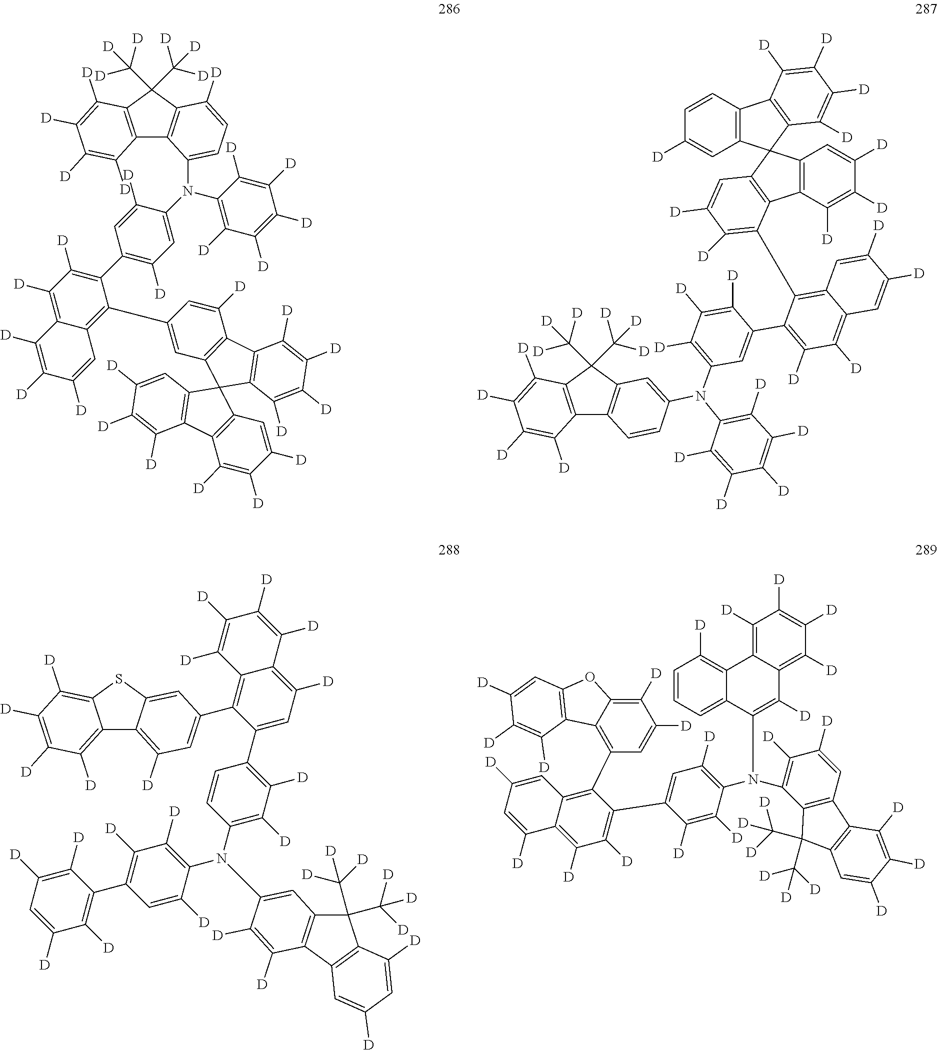

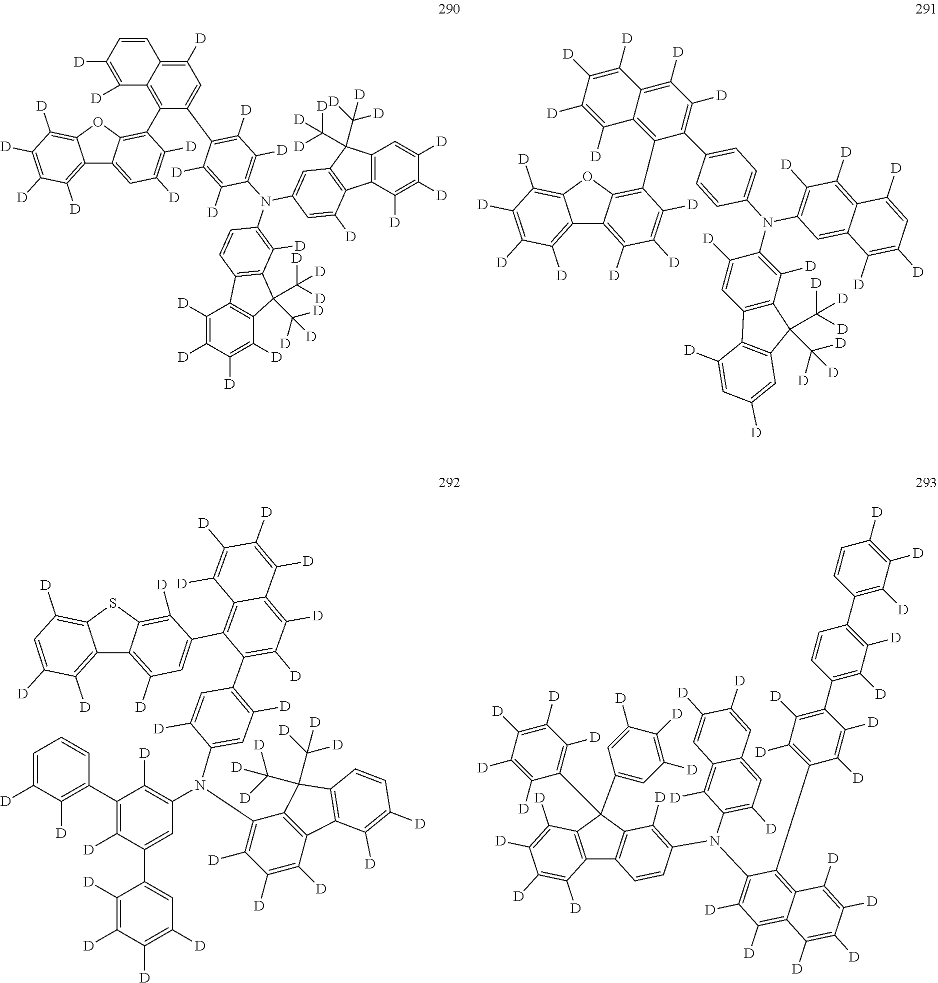

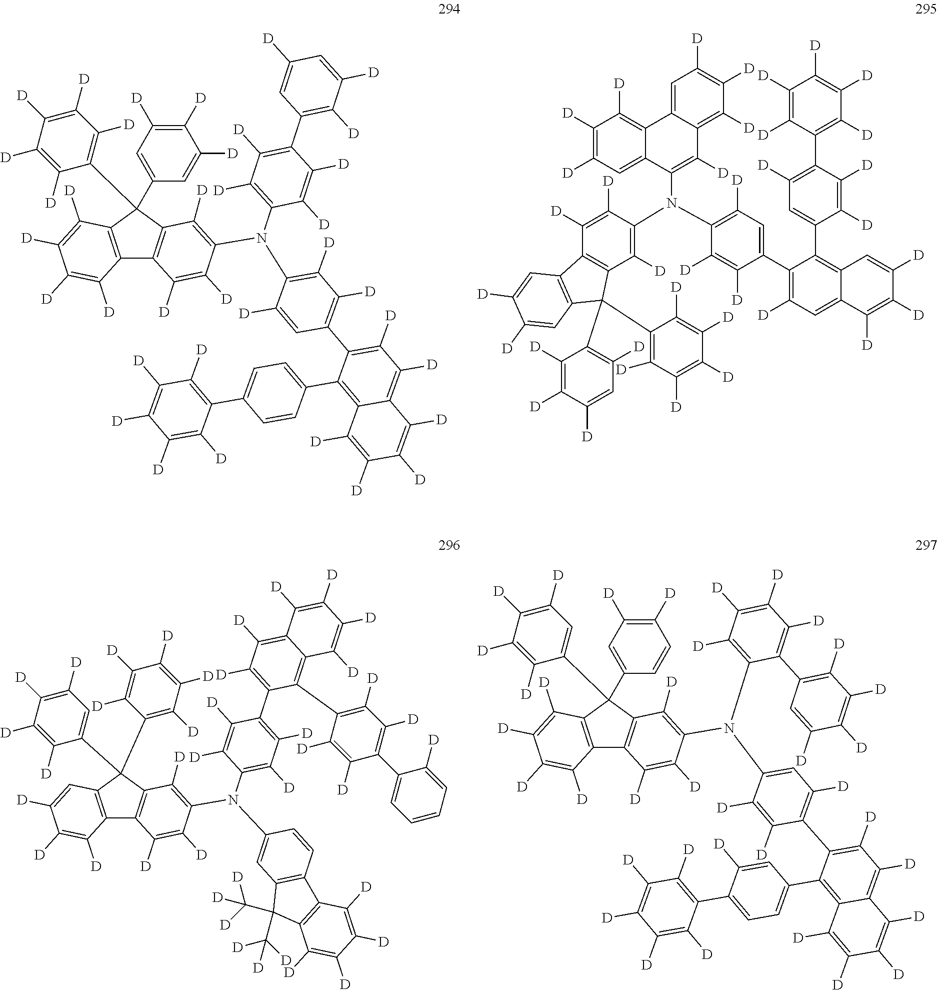

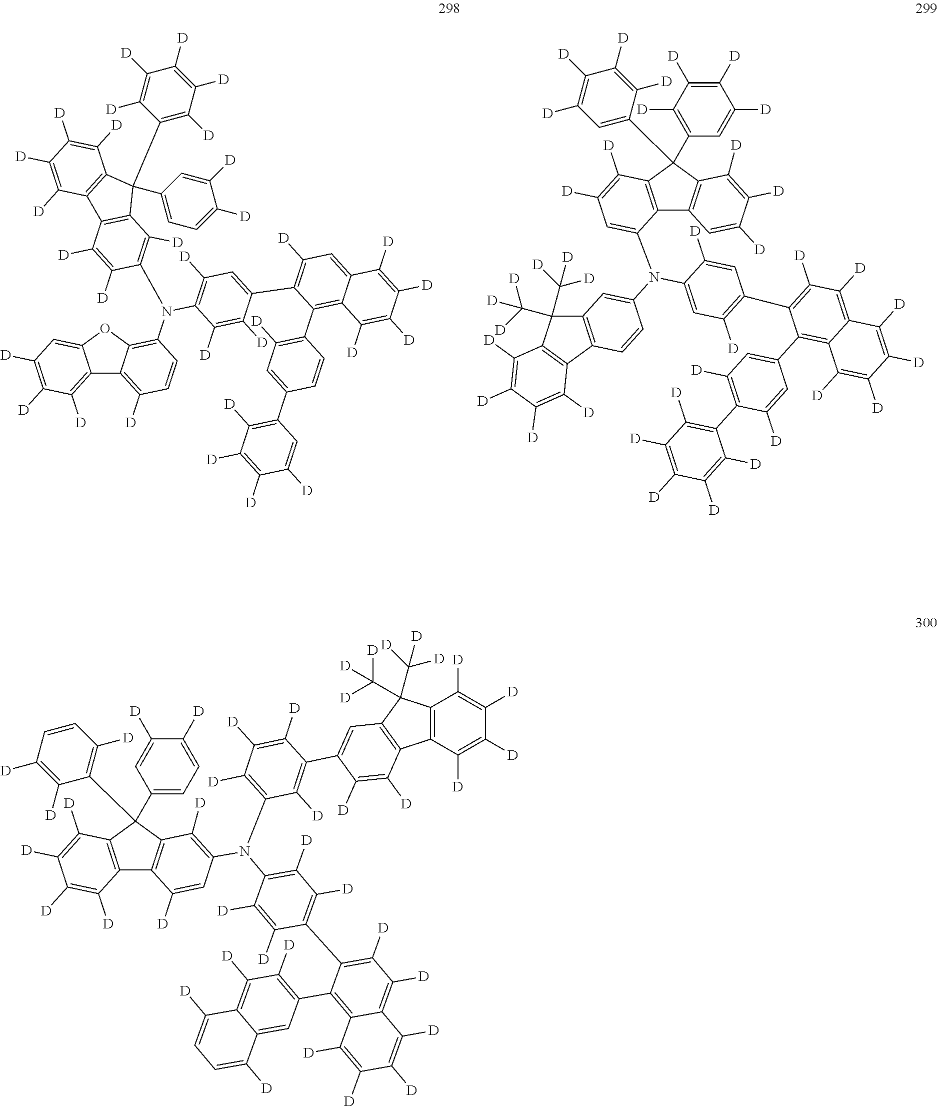

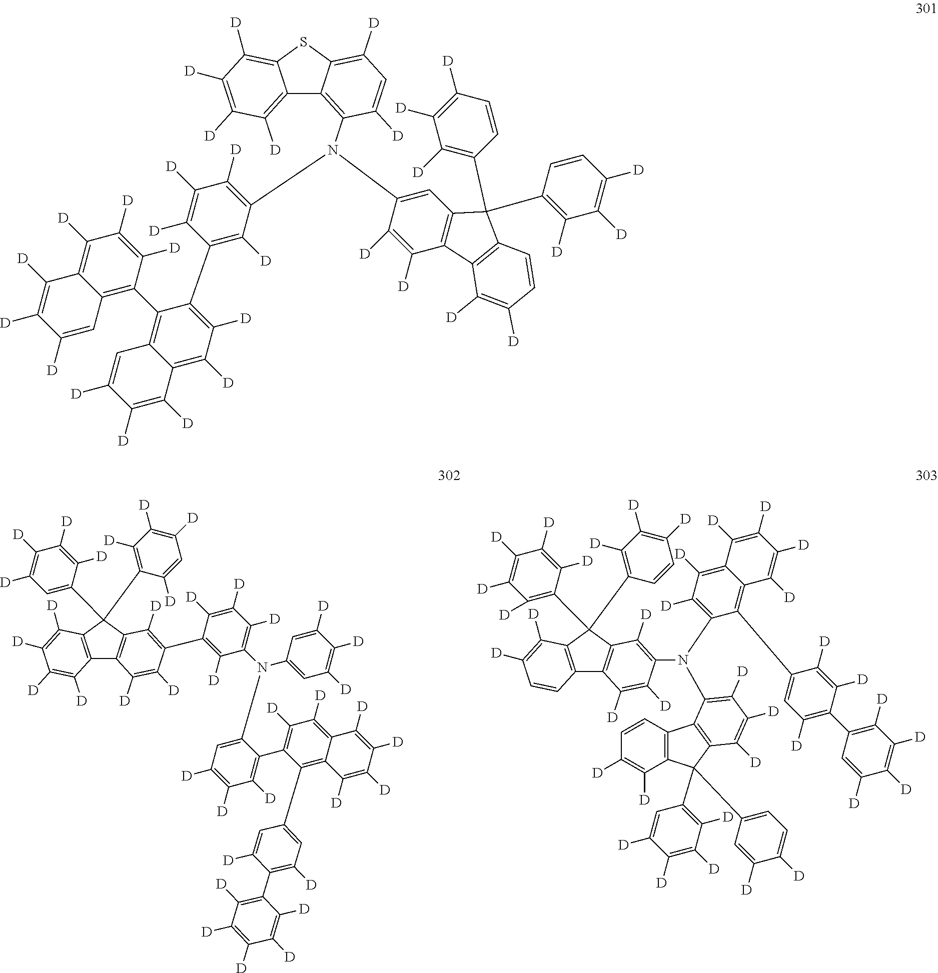

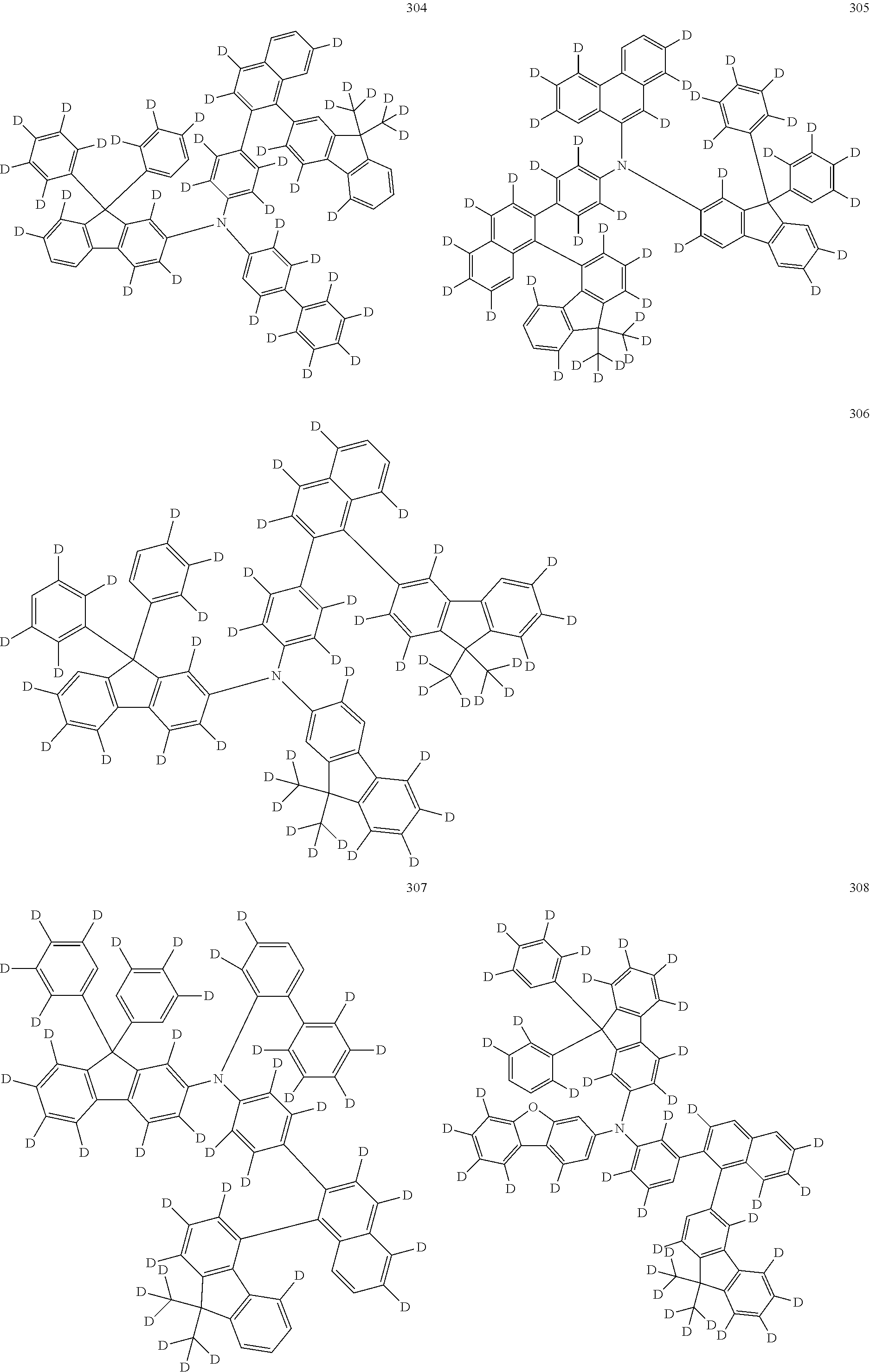

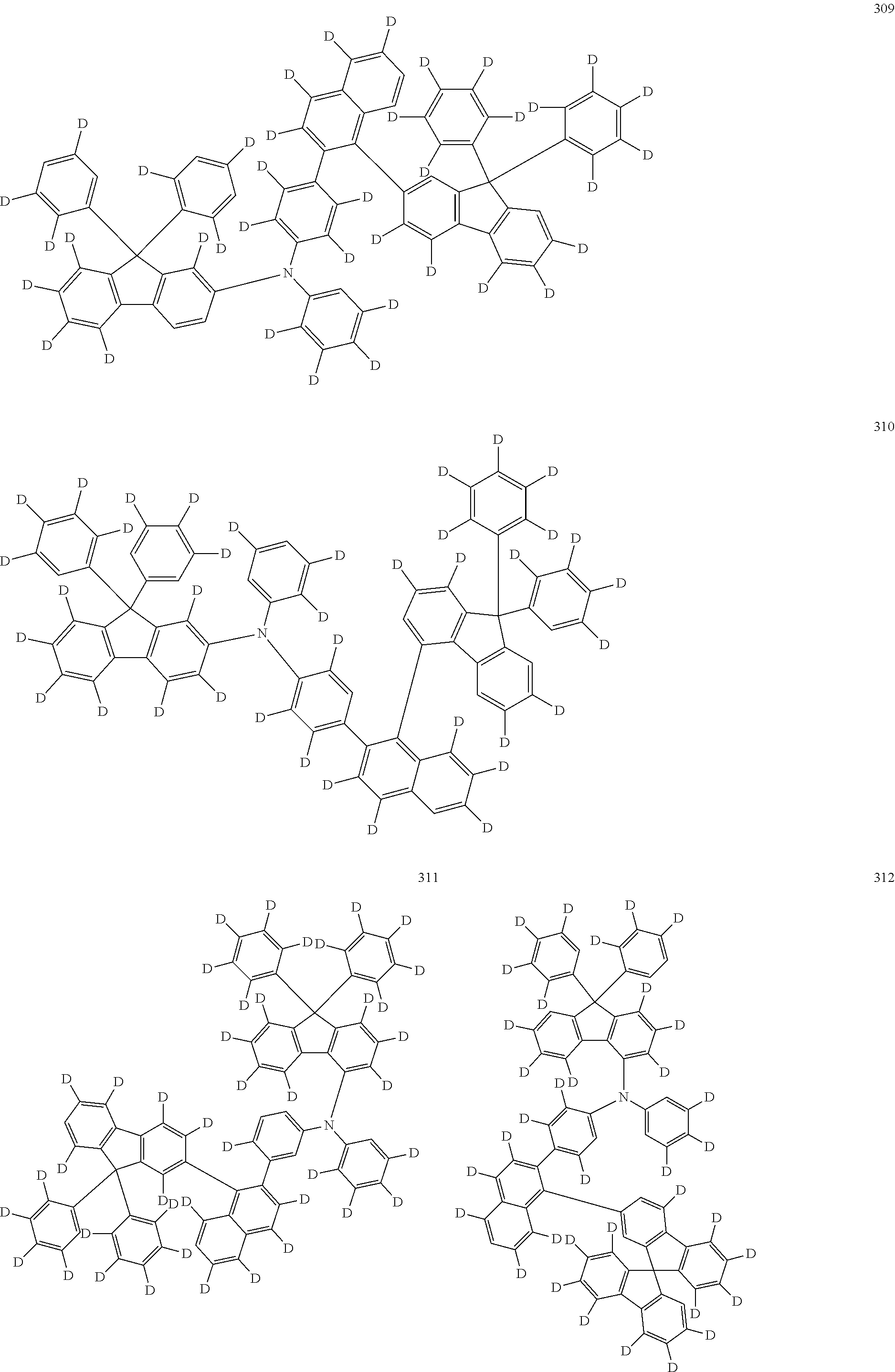

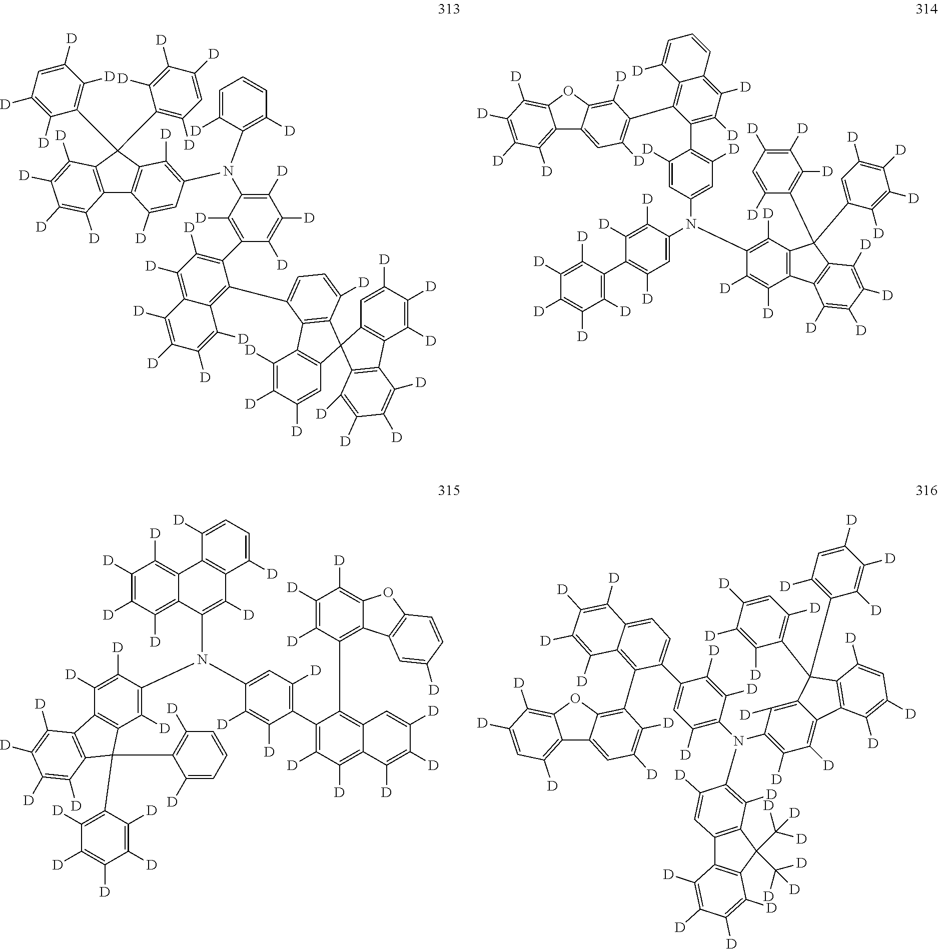

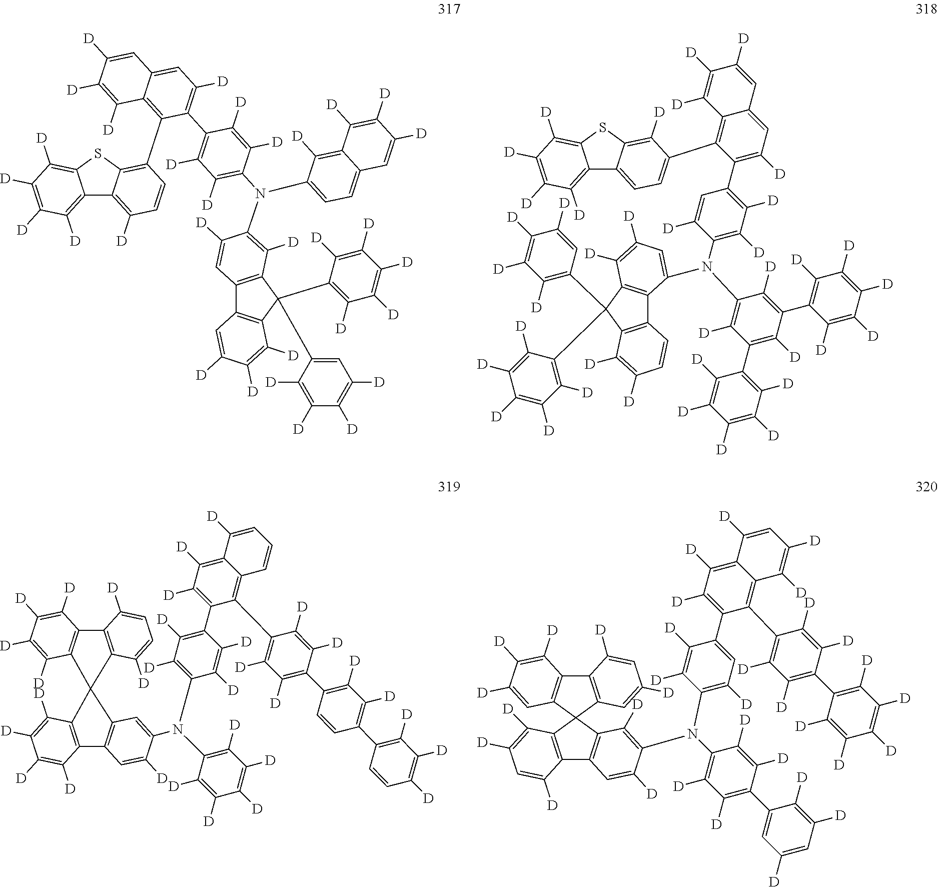

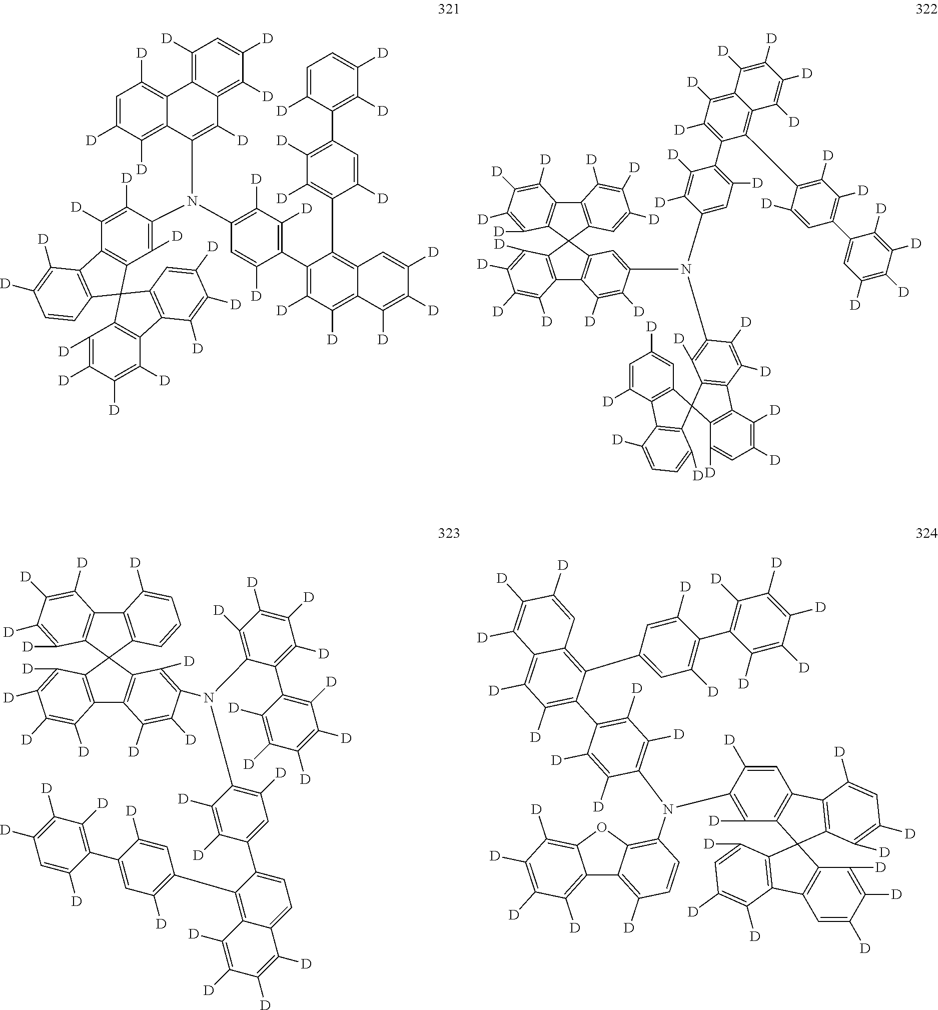

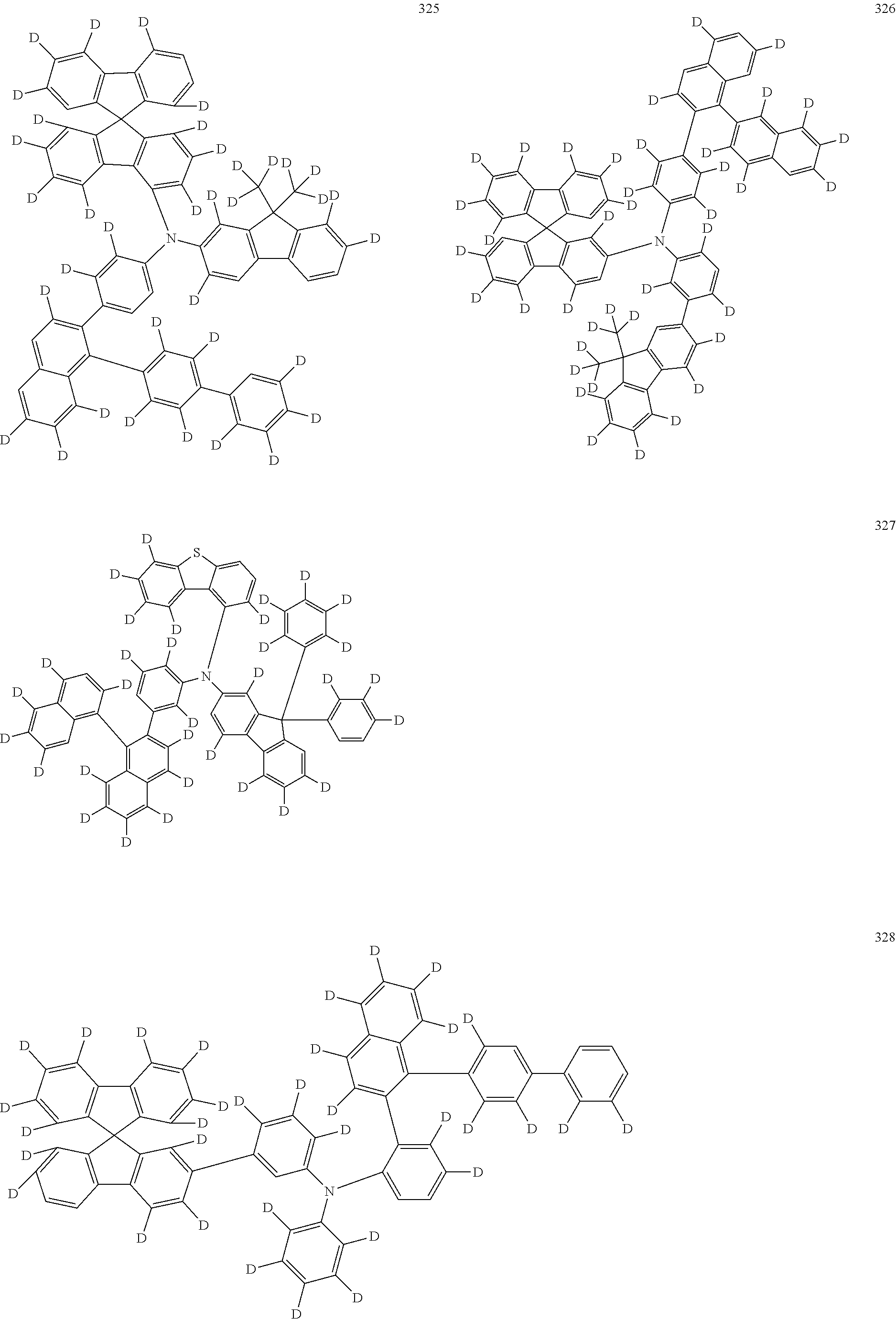

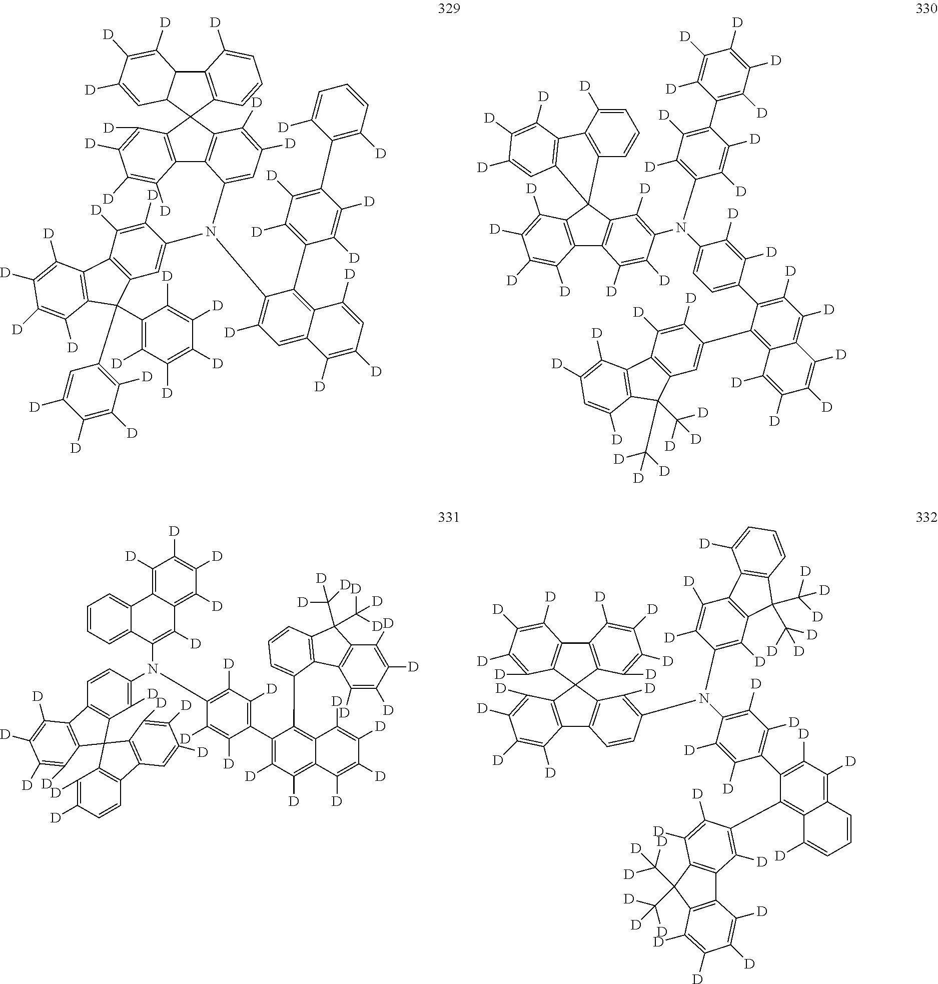

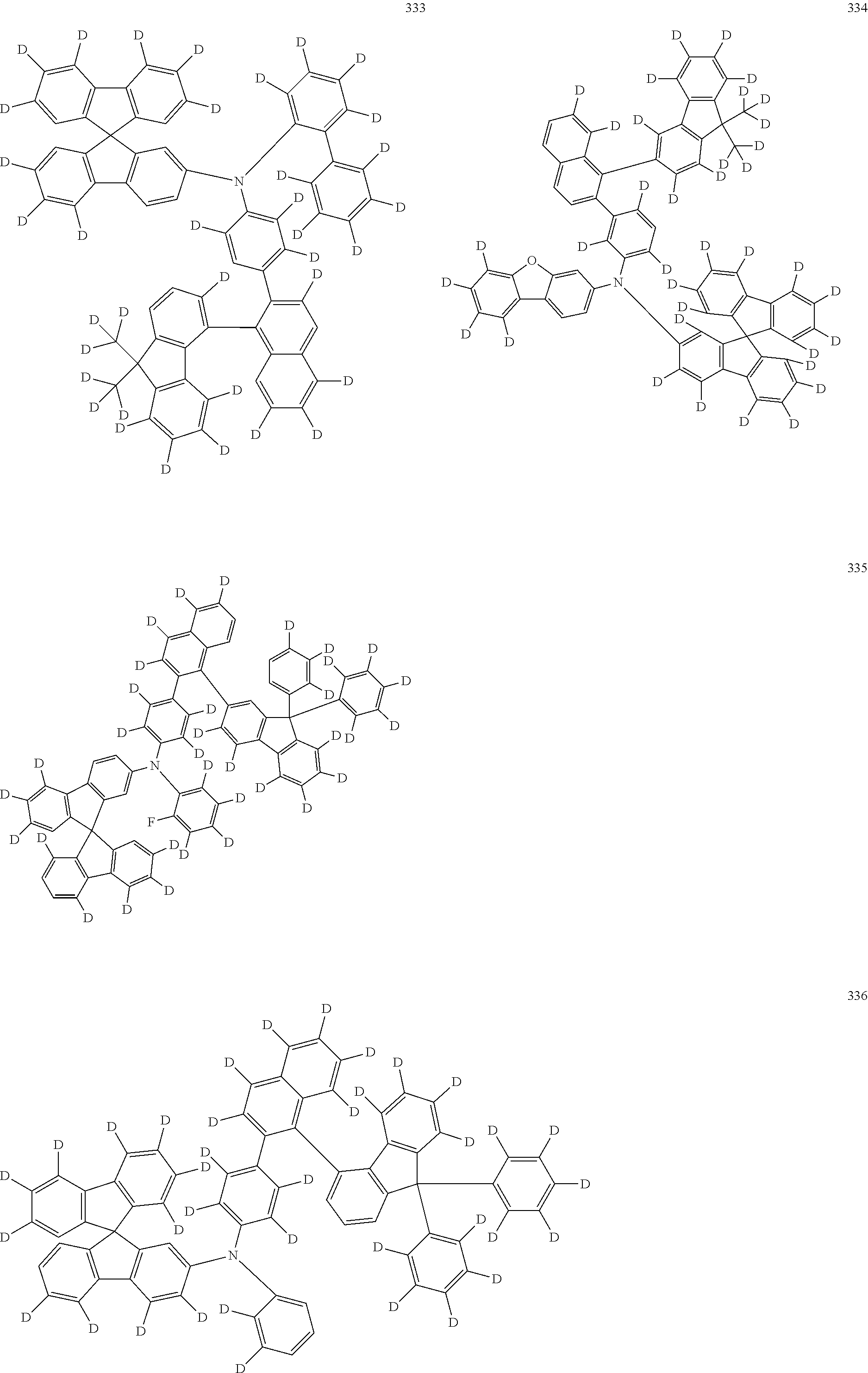

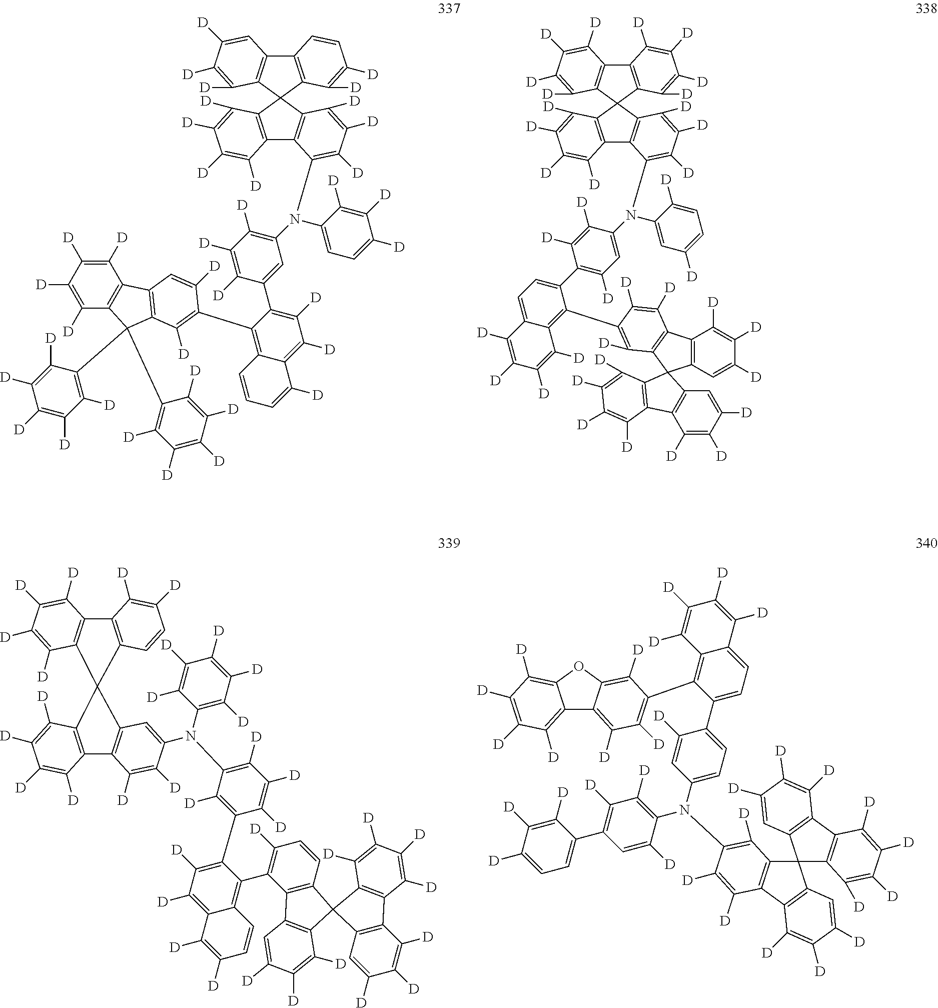

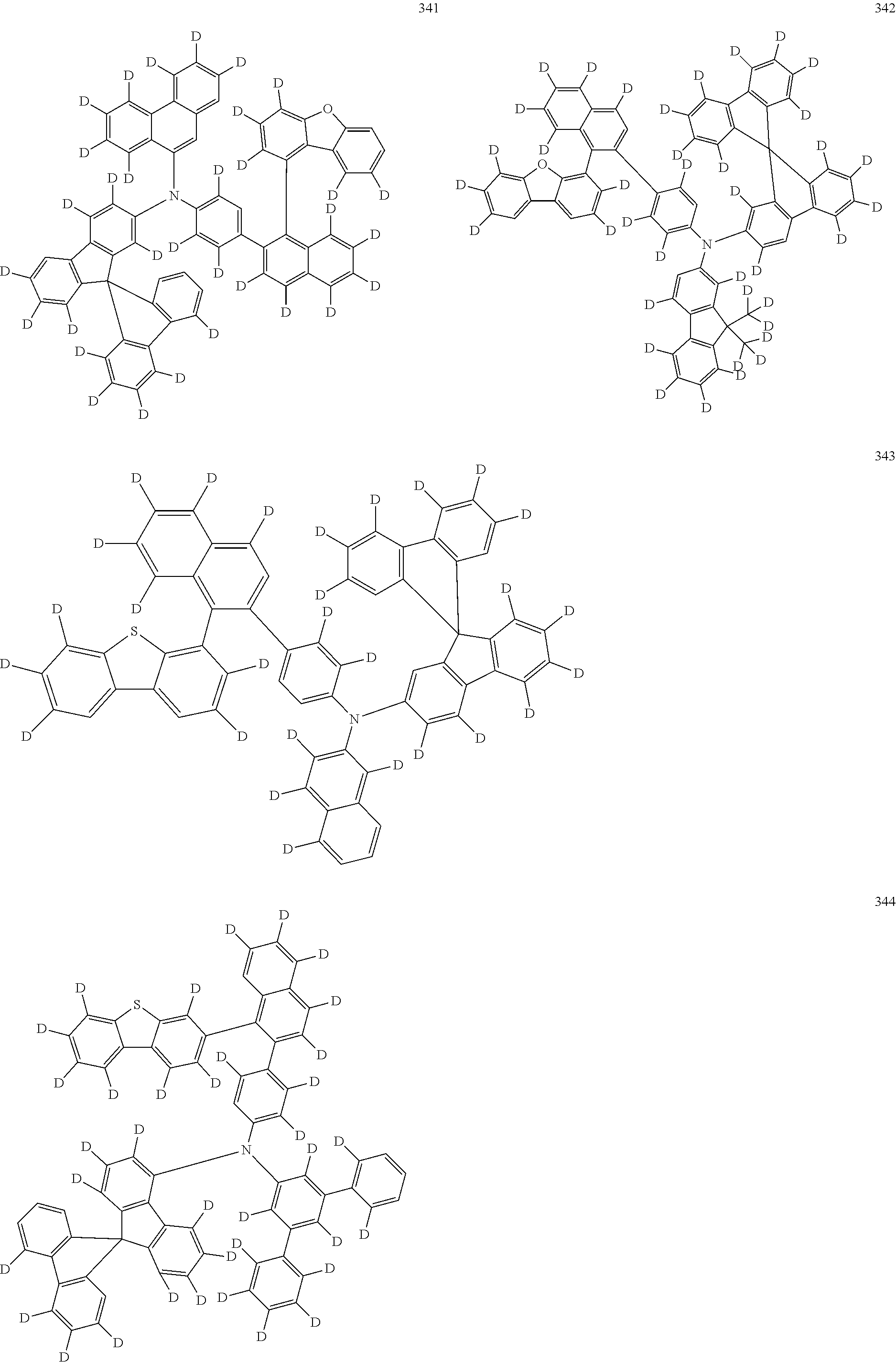

Further, various substituents may be introduced into the structure of Chemical Formula 1 to synthesize a compound having inherent characteristics of a substituent introduced. For example, it is possible to synthesize a material which satisfies conditions required for each organic material layer by introducing a substituent usually used for a hole injection layer material, a hole transport layer material, a light emitting layer material, an electron transport layer material, and a charge generation layer material, which are used for preparing an organic light emitting device, into the core structure.

In addition, it is possible to finely adjust an energy band-gap by introducing various substituents into the structure of Chemical Formula 1, and meanwhile, it is possible to improve characteristics at the interface between organic materials and diversify the use of the material.

In another exemplary embodiment of the present specification, provided is an organic light emitting device including: a first electrode; a second electrode; and an organic material layer having one or more layers provided between the first electrode and the second electrode, in which one or more layers of the organic material layer include one or more amine compounds of Chemical Formula 1.

In an exemplary embodiment of the present specification, the organic material layer may include one amine compound.

In an exemplary embodiment of the present specification, the organic material layer includes a hole transport layer, and the hole transport layer may include the amine compound.

In an exemplary embodiment of the present specification, the organic material layer includes a hole auxiliary layer, and the hole auxiliary layer may include the amine compound.

In an exemplary embodiment of the present specification, the organic material layer includes a light emitting layer, and the light emitting layer may include the amine compound as a host material of a light emitting material.

The hole auxiliary layer of the organic light emitting device according to an exemplary embodiment of the present specification means a layer that prevents electrons from migrating from the light emitting layer to the hole transport layer by matching the appropriate energy levels of the hole transport layer and the light emitting layer. The deuterium-substituted amine compound of the present invention is a material having suitable hole mobility, and when the amine compound is used in a hole auxiliary layer, it is possible to prevent the reduction of excitons formed in the light emitting layer. That is, when the amine compound is used in the hole auxiliary layer, the driving, efficiency and service life of the organic light emitting device may become excellent.

The organic material layer of the organic light emitting device of the present invention may be composed of a single-layered structure, but may be composed of a multi-layered structure in which two or more organic material layers are stacked. For example, the organic light emitting device of the present invention may have a structure including a hole injection layer, a hole transport layer, a light emitting layer, an electron transport layer, an electron injection layer, and the like as organic material layers. However, the structure of the organic light emitting device is not limited thereto, and may include a fewer number of organic material layers.

In an exemplary embodiment of the present specification, the first electrode may be a positive electrode, and the second electrode may be a negative electrode.

In another exemplary embodiment of the present specification, the first electrode may be a negative electrode, and the second electrode may be a positive electrode.

The organic light emitting device according to an exemplary embodiment of the present specification may be manufactured by typical methods and materials for manufacturing an organic light emitting device, except that an organic material layer having one or more layers is formed by using the amine compound of the above-described Chemical Formula 1.

The amine compound of Chemical Formula 1 may be formed as an organic material layer by not only a vacuum deposition method, but also a solution application method when an organic light emitting device is manufactured. Here, the solution application method means spin coating, dip coating, inkjet printing, screen printing, a spray method, roll coating, and the like, but is not limited thereto.

In an exemplary embodiment of the present specification, the organic light emitting device may be a blue organic light emitting device, and the amine compound of Chemical Formula 1 may be used as a material for the blue organic light emitting device. For example, the amine compound of Chemical Formula 1 may be included in the light emitting layer, hole transport layer or hole auxiliary layer of the blue organic light emitting device.

In another exemplary embodiment of the present specification, the organic light emitting device may be a green organic light emitting device, and the amine compound of Chemical Formula 1 may be used as a material for the green organic light emitting device. For example, the amine compound of Chemical Formula 1 may be included in the light emitting layer, hole transport layer or hole auxiliary layer of the green organic light emitting device.

In still another exemplary embodiment of the present specification, the organic light emitting device may be a red organic light emitting device, and the amine compound of Chemical Formula 1 may be used as a material for the red organic light emitting device. For example, the amine compound of Chemical Formula 1 may be included in the light emitting layer, hole transport layer or hole auxiliary layer of the red organic light emitting device.

The organic light emitting device of the present invention may further include one or more layers selected from the group consisting of a light emitting layer, a hole injection layer, a hole transport layer, an electron injection layer, an electron transport layer, an electron blocking layer, and a hole blocking layer.

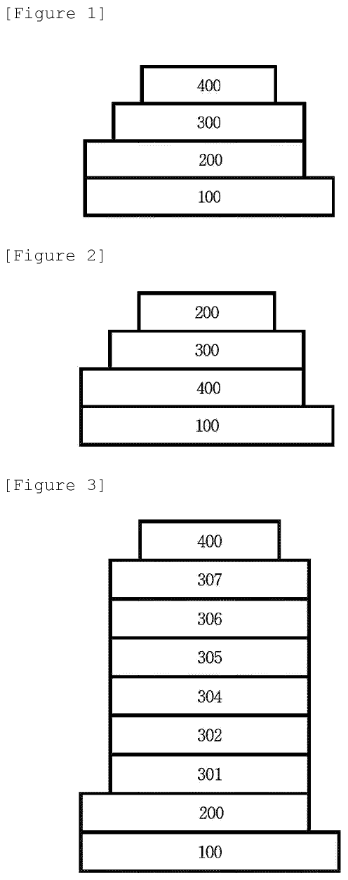

FIGS. 1 to 4 exemplify the stacking sequence of the electrodes and the organic material layer of the organic light emitting device according to an exemplary embodiment of the present specification. However, the scope of the present application is not intended to be limited by these drawings, and the structure of the organic light emitting device known in the art may also be applied to the present application.

According to FIG. 1, an organic light emitting device in which a positive electrode 200, an organic material layer 300, and a negative electrode 400 are sequentially stacked on a substrate 100 is illustrated. However, the organic light emitting device is not limited only to such a structure, and as in FIG. 2, an organic light emitting device in which a negative electrode, an organic material layer, and a positive electrode are sequentially stacked on a substrate may also be implemented.

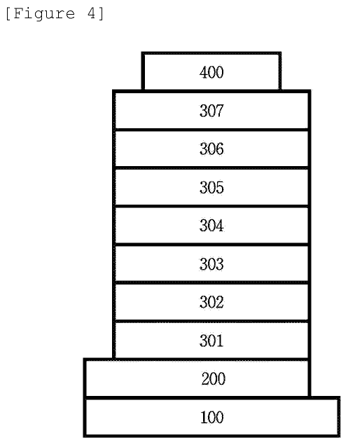

FIGS. 3 and 4 exemplify a case where an organic material layer is a multilayer. The organic light emitting device according to FIG. 3 includes a hole injection layer 301, a hole transport layer 302, a light emitting layer 304, a hole blocking layer 305, an electron transport layer 306 and an electron injection layer 307, and the organic light emitting device according to FIG. 4 includes a hole injection layer 301, a hole transport layer 302, a hole auxiliary layer 303, a light emitting layer 304, a hole blocking layer 305, an electron transport layer 306 and an electron injection layer 307. However, the scope of the present application is not limited by the stacking structure as described above, and if necessary, the other layers except for the light emitting layer may be omitted, and another necessary functional layer may be further added.

An organic material layer including the amine compound of Chemical Formula 1 may additionally include other materials, if necessary.

In the organic light emitting device according to an exemplary embodiment of the present specification, materials other than the amine compound of Chemical Formula 1 will be exemplified below, but these materials are illustrative only and are not for limiting the scope of the present application, and may be replaced with materials publicly known in the art.

As a positive electrode material, materials having a relatively high work function may be used, and a transparent conductive oxide, a metal or a conductive polymer, and the like may be used. Specific examples of the positive electrode material include: a metal such as vanadium, chromium, copper, zinc, and gold, or an alloy thereof; a metal oxide such as zinc oxide, indium oxide, indium tin oxide (ITO), and indium zinc oxide (IZO); a combination of a metal and an oxide, such as ZnO:Al or SnO₂:Sb; a conductive polymer such as poly(3-methylthiophene), poly[3,4-(ethylene-1,2-dioxy)thiophene](PEDOT), polypyrrole, and polyaniline; and the like, but are not limited thereto.

As a negative electrode material, materials having a relatively low work function may be used, and a metal, a metal oxide, or a conductive polymer, and the like may be used. Specific examples of the negative electrode material include: a metal such as magnesium, calcium, sodium, potassium, titanium, indium, yttrium, lithium, gadolinium, aluminum, silver, tin, and lead, or an alloy thereof; a multi-layer structured material, such as LiF/Al or LiO₂/Al; and the like, but are not limited thereto.

As a hole injection material, a publicly-known hole injection material may also be used, and it is possible to use, for example, a phthalocyanine compound such as copper phthalocyanine disclosed in U.S. Pat. No. 4,356,429 or starburst-type amine derivatives described in the document [Advanced Material, 6, p. 677 (1994)], for example, tris(4-carbazoyl-9-ylphenyl)amine (TCTA), 4,4′,4″-tri[phenyl(m-tolyl)amino]triphenylamine (m-MTDATA), 1,3,5-tris[4-(3-methylphenylphenylamino)phenyl]benzene (m-MTDAPB), polyaniline/dodecylbenzenesulfonic acid or poly(3,4-ethylenedioxythiophene)/poly(4-styrenesulfonate), which is a soluble conductive polymer, polyaniline/camphor sulfonic acid or polyaniline/poly(4-styrenesulfonate), and the like.

As a hole transport material, a pyrazoline derivative, an arylamine-based derivative, a stilbene derivative, a triphenyldiamine derivative, and the like may be used, and a low-molecular weight or polymer material may also be used.

As an electron transport material, it is possible to use an oxadiazole derivative, anthraquinodimethane and a derivative thereof, benzoquinone and a derivative thereof, naphthoquinone and a derivative thereof, anthraquinone and a derivative thereof, tetracyanoanthraquinodimethane and a derivative thereof, a fluorenone derivative, diphenyldicyanoethylene and a derivative thereof, a diphenoquinone derivative, a metal complex of 8-hydroxyquinoline and a derivative thereof, and the like, and a low-molecular weight material and a polymer material may also be used.

As an electron injection material, for example, LiF is representatively used in the art, but the present application is not limited thereto.

As a light emitting material, a red, green, or blue light emitting material may be used, and if necessary, two or more light emitting materials may be mixed and used. In this case, two or more light emitting materials are deposited or used as an individual supply source, or pre-mixed to be deposited and used as one supply source. Further, a fluorescent material may also be used as the light emitting material, but a phosphorescent material may also be used. As the light emitting material, it is also possible to use alone a material which emits light by combining holes and electrons each injected from a positive electrode and a negative electrode, but materials in which a host material and a dopant material are involved in light emission together may also be used.

When hosts of the light emitting material are mixed and used, the same series of hosts may also be mixed and used, and different series of hosts may also be mixed and used. For example, any two or more materials from N-type host materials or P-type host materials may be selected and used as a host material for a light emitting layer.

The organic light emitting device according to an exemplary embodiment of the present specification may be a top emission type, a bottom emission type, or a dual emission type according to the material to be used.

The amine compound according to an exemplary embodiment of the present specification may act even in organic electronic devices including organic solar cells, organic photoconductors, organic transistors, and the like, based on the principle similar to those applied to organic light emitting devices.

Hereinafter, the present specification will be described in more detail through Examples, but these Examples are provided only for exemplifying the present application, and are not intended to limit the scope of the present application.

### PREPARATION EXAMPLES

### [Preparation Example 1] Preparation of Compound 001

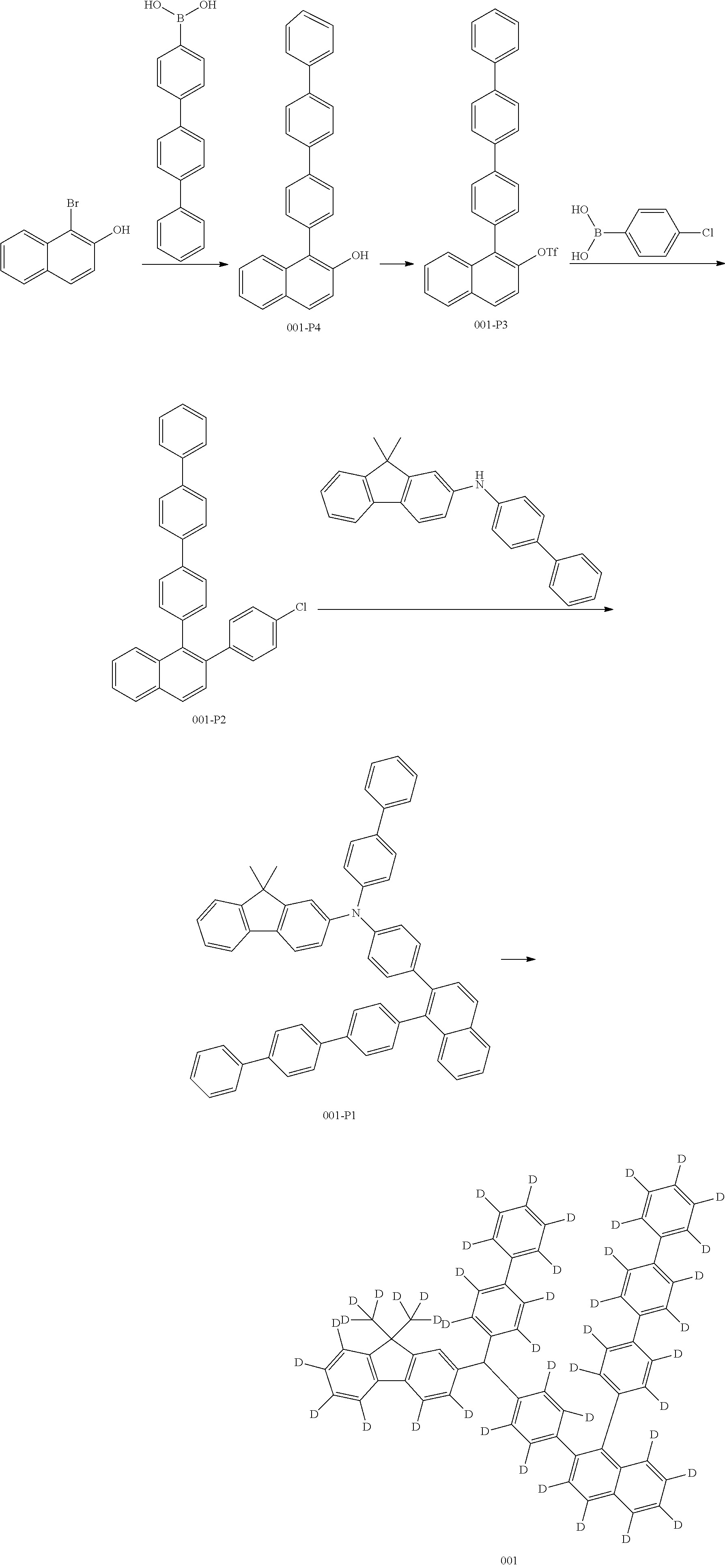

### 1) Preparation of Compound 001-P4

After a compound 1-bromonaphthalen-2-ol (100 g, 0.4483 mol) and [1,1′:4′,1″-terphenyl]-4-ylboronic acid (129.03 g, 0.4707 mol) were dissolved in 1500 ml of toluene and 300 ml of distilled water, tetrakis (triphenylphosphine)palladium(0) (Pd(PPh₃)₄) (25.90 g, 0.0224 mol) and K₂CO₃ (154.90 g, 1.1207 mol) were added thereto, and the resulting mixture was stirred under reflux for 12 hours. After the reaction solution was extracted, the organic layer was adsorbed onto silica and purified by adsorption column chromatography. The resulting product was concentrated, methanol (MeOH) was added dropwise thereto, and then the resulting solid was filtered to obtain Compound 001-P4 (150 g, 90%).
2) Preparation of Compound 001-P3

After Compound 001-P4 (150 g, 0.4027 mol) and triethylamine (67.36 mL, 0.4833 mol) were dissolved in 1500 ml of dichloromethane, trifluoromethanesulfonic anhydride (81.16 mL, 0.4833 mol) was added thereto, and the resulting mixture was stirred at room temperature for 2 hours. After the reaction was completed, the reaction solution was extracted, and then the organic layer was adsorbed onto silica and purified by adsorption column chromatography. The resulting product was concentrated, MeOH was added dropwise thereto, and then the resulting solid was filtered to obtain Compound 001-P3 (180 g, 88%).

### 3) Preparation of Compound 001-P2

After Compound 001-P3 (180 g, 0.3568 mol) and (4-chlorophenyl)boronic acid (58.58 g, 0.3746 mol) were dissolved in 1500 ml of toluene and 300 ml of distilled water, Pd(PPh₃)₄ (20.61 g, 0.0178 mol) and K₂CO₃ (123.27 g, 0.8919 mol) were added thereto, and the resulting mixture was stirred under reflux for 12 hours. After the reaction solution was extracted, the organic layer was adsorbed onto silica and purified by adsorption column chromatography. The resulting product was concentrated, MeOH was added dropwise, and then the resulting solid was filtered to obtain Compound 001-P2 (150 g, 90%).

### 4) Preparation of Compound 001-P1

After Compound 001-P2 (10 g, 0.0214 mol) and N-([1,1′-biphenyl]-4-yl)-9,9-dimethyl-9H-fluoren-2-amine) (7.74 g, 0.0214 mol) were dissolved in 100 ml of toluene, tris(dibenzylideneacetone)dipalladium(0) (Pd₂ (dba)₃) (0.98 g, 0.0011 mol), 2-dicyclohexylphosphino-2ç,4″,6″-triisopropylbiphenyl (xphos) (1.53 g, 0.0032 mol), and sodium tert-butoxide (t-BuONa) (5.14 g, 0.0535 mol) were added thereto, and the resulting mixture was stirred under reflux for 3 hours. After the reaction was completed, the reaction solution was extracted, and then the organic layer was adsorbed onto silica and purified by adsorption column chromatography to obtain Compound 001-P1 (12 g, 71%).

### 5) Preparation of Compound 001

Compound 001-P1 (12 g, 0.0152 mol), benzene-D₆ (120 ml), and trifluoromethanesulfonic acid (9.36 ml, 0.1061 mol) were put into a flask and stirred at 60° C. for 4 hours, and then 300 ml of distilled water and NaHCO₃ were added thereto to terminate the reaction. After the organic layer was extracted, moisture was removed using MgSO₄. The concentrated organic layer was allowed to pass through silica gel and purified to obtain Compound 001 (12.3 g, 97%).

A target compound was synthesized in the same manner as in Preparation Example 1, except that Compound A and Compound B in the following Table 1 were used instead of [1,1′:4′,1″-terphenyl]-4-ylboronic acid and N-([1,1′-biphenyl]-4-yl)-9,9-dimethyl-9H-fluoren-2-amine,

TABLE 1

| Compound | | | | |
|---|---|---|---|---|
| No. | Compound A | Compound B | Target compound | yield |
| 003 | 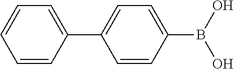 [SMILES: OB(O)c1ccc(-c2ccccc2)cc1] | 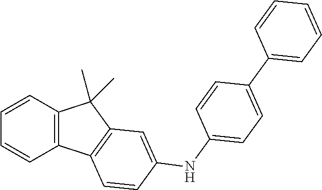 [SMILES: CC1(C)c2ccccc2-c2ccc(Nc3ccc(-c4ccccc4)cc3)cc21] | 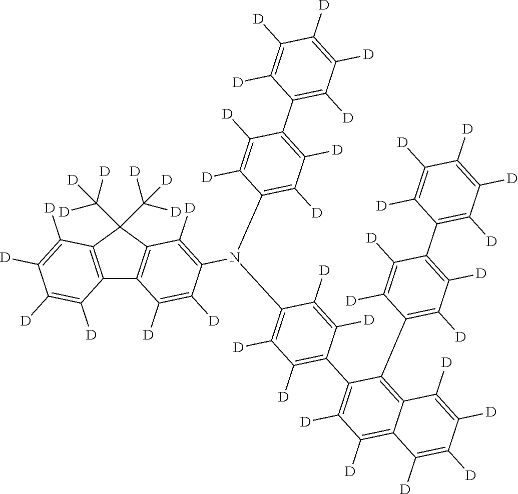 [SMILES: [2H]c1c([2H])c([2H])c(-c2c([2H])c([2H])c(-c3c(-c4c([2H])c([2H])c(N(c5c([2H])c([2H])c(-c6c([2H])c([2H])c([2H])c([2H])c6[2H])c([2H])c5[2H])c5c([2H])c([2H])c6c(c5[2H])C(C([2H])([2H])[2H])(C([2H])([2H])[2H])c5c([2H])c([2H])c([2H])c([2H])c5-6)c([2H])c4[2H])c([2H])c([2H])c4c([2H])c([2H])c([2H])c([2H])c34)c([2H])c2[2H])c([2H])c1[2H]] | 79% |
| 005 | 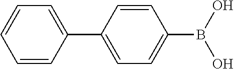 [SMILES: OB(O)c1ccc(-c2ccccc2)cc1] | 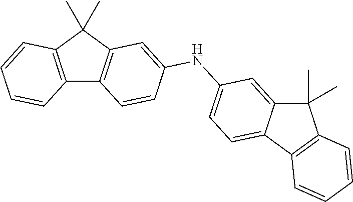 [SMILES: CC1(C)c2ccccc2-c2ccc(Nc3ccc4c(c3)C(C)(C)c3ccccc3-4)cc21] | 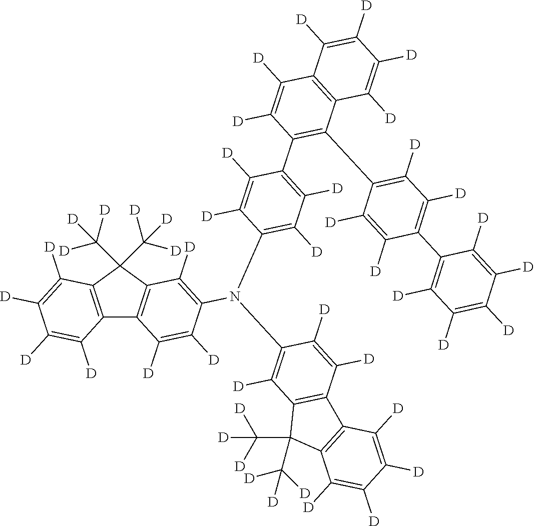 [SMILES: [2H]c1c([2H])c([2H])c(-c2c([2H])c([2H])c(-c3c(-c4c([2H])c([2H])c(N(c5c([2H])c([2H])c6c(c5[2H])C(C([2H])([2H])[2H])(C([2H])([2H])[2H])c5c([2H])c([2H])c([2H])c([2H])c5-6)c5c([2H])c([2H])c6c(c5[2H])C(C([2H])([2H])[2H])(C([2H])([2H])[2H])c5c([2H])c([2H])c([2H])c([2H])c5-6)c([2H])c4[2H])c([2H])c([2H])c4c([2H])c([2H])c([2H])c([2H])c34)c([2H])c2[2H])c([2H])c1[2H]] | 78% |
| 007 | 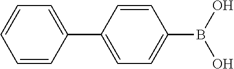 [SMILES: OB(O)c1ccc(-c2ccccc2)cc1] | 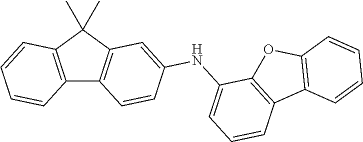 [SMILES: CC1(C)c2ccccc2-c2ccc(Nc3cccc4c3oc3ccccc34)cc21] | 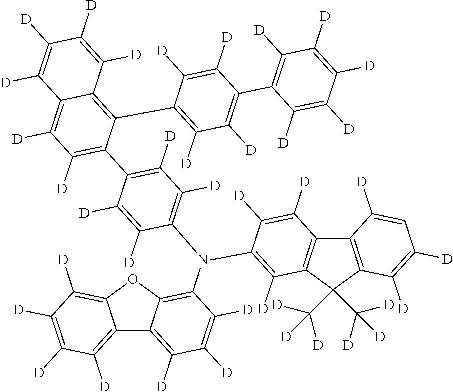 [SMILES: [2H]c1cc([2H])c2c(c1[2H])C(C([2H])([2H])[2H])(C([2H])([2H])[2H])c1c([2H])c(N(c3c([2H])c([2H])c(-c4c([2H])c([2H])c5c([2H])c([2H])c([2H])c([2H])c5c4-c4c([2H])c([2H])c(-c5c([2H])c([2H])c([2H])c([2H])c5[2H])c([2H])c4[2H])c([2H])c3[2H])c3c([2H])c([2H])c([2H])c4c3oc3c([2H])c([2H])c([2H])c([2H])c34)c([2H])c([2H])c1-2] | 72% |
| 011 | 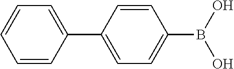 [SMILES: OB(O)c1ccc(-c2ccccc2)cc1] | 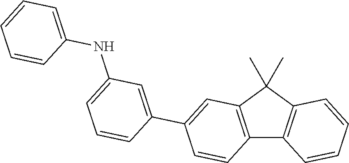 [SMILES: CC1(C)c2ccccc2-c2ccc(-c3cccc(Nc4ccccc4)c3)cc21] | 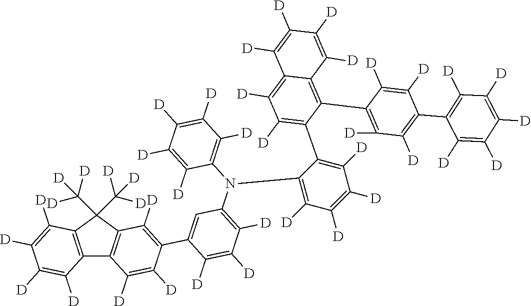 [SMILES: [2H]c1c(-c2c([2H])c([2H])c3c(c2[2H])C(C([2H])([2H])[2H])(C([2H])([2H])[2H])c2c([2H])c([2H])c([2H])c([2H])c2-3)cc(N(c2c([2H])c([2H])c([2H])c([2H])c2[2H])c2c([2H])c([2H])c([2H])c([2H])c2-c2c([2H])c([2H])c3c([2H])c([2H])c([2H])c([2H])c3c2-c2c([2H])c([2H])c(-c3c([2H])c([2H])c([2H])c([2H])c3[2H])c([2H])c2[2H])c([2H])c1[2H]] | 73% |
| 013 | 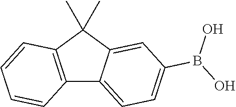 [SMILES: CC1(C)c2ccccc2-c2ccc(B(O)O)cc21] | 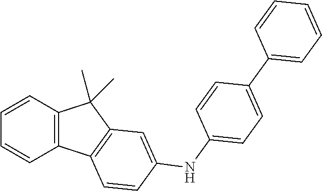 [SMILES: CC1(C)c2ccccc2-c2ccc(Nc3ccc(-c4ccccc4)cc3)cc21] | 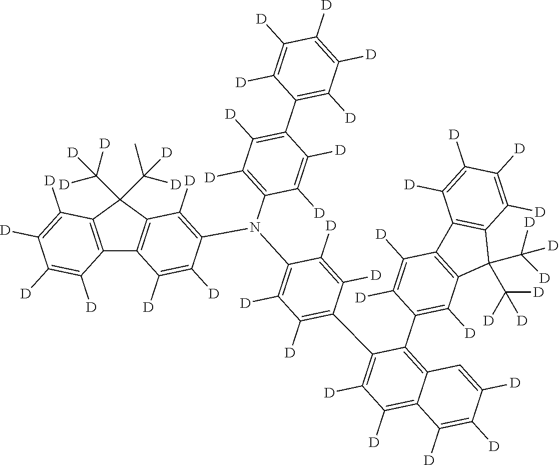 [SMILES: [2H]c1cc2c(-c3c([2H])c([2H])c4c(c3[2H])C(C([2H])([2H])[2H])(C([2H])([2H])[2H])c3c([2H])c([2H])c([2H])c([2H])c3-4)c(-c3c([2H])c([2H])c(N(c4c([2H])c([2H])c(-c5c([2H])c([2H])c([2H])c([2H])c5[2H])c([2H])c4[2H])c4c([2H])c([2H])c5c(c4[2H])C(C([2H])([2H])[2H])(C([2H])([2H])C)c4c([2H])c([2H])c([2H])c([2H])c4-5)c([2H])c3[2H])c([2H])c([2H])c2c([2H])c1[2H]] | 77% |
| 023 | 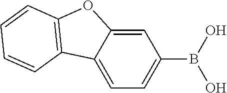 [SMILES: OB(O)c1ccc2c(c1)oc1ccccc12] | 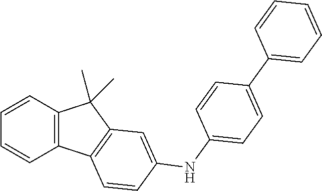 [SMILES: CC1(C)c2ccccc2-c2ccc(Nc3ccc(-c4ccccc4)cc3)cc21] | 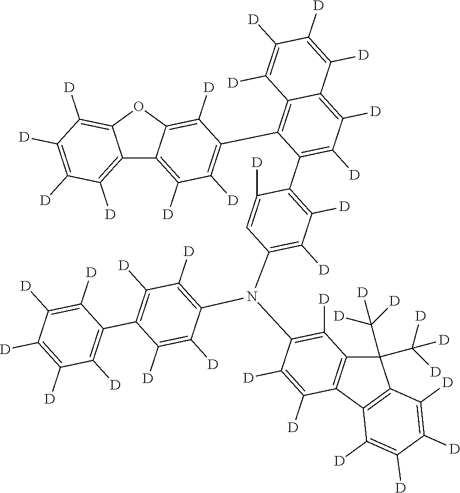 [SMILES: [2H]c1cc(N(c2c([2H])c([2H])c(-c3c([2H])c([2H])c([2H])c([2H])c3[2H])c([2H])c2[2H])c2c([2H])c([2H])c3c(c2[2H])C(C([2H])([2H])[2H])(C([2H])([2H])[2H])c2c([2H])c([2H])c([2H])c([2H])c2-3)c([2H])c([2H])c1-c1c([2H])c([2H])c2c([2H])c([2H])c([2H])c([2H])c2c1-c1c([2H])c([2H])c2c(oc3c([2H])c([2H])c([2H])c([2H])c32)c1[2H]] | 67% |
| 025 | 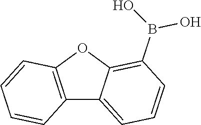 [SMILES: OB(O)c1cccc2c1oc1ccccc12] | 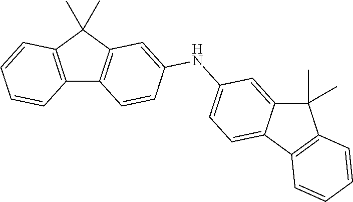 [SMILES: CC1(C)c2ccccc2-c2ccc(Nc3ccc4c(c3)C(C)(C)c3ccccc3-4)cc21] | 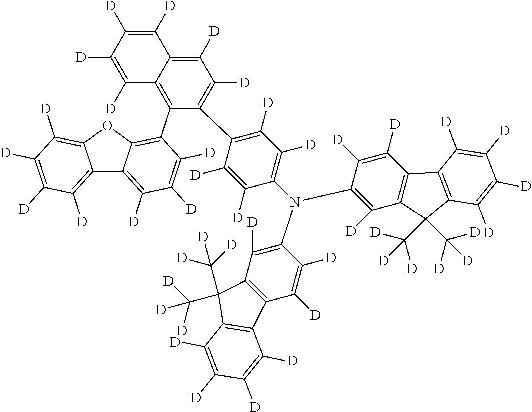 [SMILES: [2H]c1c([2H])c([2H])c2c(c1[2H])-c1c([2H])c([2H])c(N(c3c([2H])c([2H])c(-c4c([2H])c([2H])c5c([2H])c([2H])c([2H])c([2H])c5c4-c4c([2H])c([2H])c([2H])c5c4oc4c([2H])c([2H])c([2H])c([2H])c45)c([2H])c3[2H])c3c([2H])c([2H])c4c(c3[2H])C(C([2H])([2H])[2H])(C([2H])([2H])[2H])c3c([2H])c([2H])c([2H])c([2H])c3-4)c([2H])c1C2(C([2H])([2H])[2H])C([2H])([2H])[2H]] | 69% |
| 026 | 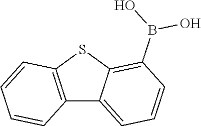 [SMILES: OB(O)c1cccc2c1sc1ccccc12] | 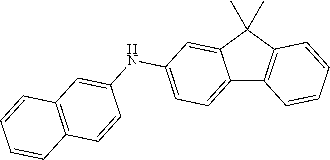 [SMILES: CC1(C)c2ccccc2-c2ccc(Nc3ccc4ccccc4c3)cc21] | 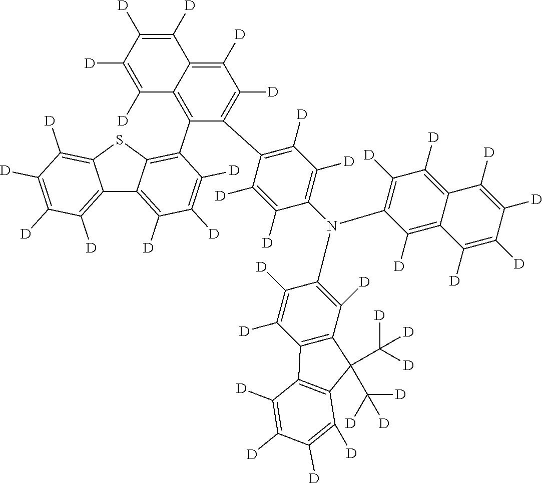 [SMILES: [2H]c1c([2H])c([2H])c2c(c1[2H])-c1c([2H])c([2H])c(N(c3c([2H])c([2H])c(-c4c([2H])c([2H])c5c([2H])c([2H])c([2H])c([2H])c5c4-c4c([2H])c([2H])c([2H])c5c4sc4c([2H])c([2H])c([2H])c([2H])c45)c([2H])c3[2H])c3c([2H])c([2H])c4c([2H])c([2H])c([2H])c([2H])c4c3[2H])c([2H])c1C2(C([2H])([2H])[2H])C([2H])([2H])[2H]] | 75% |
| 027 | 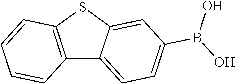 [SMILES: OB(O)c1ccc2c(c1)sc1ccccc12] | 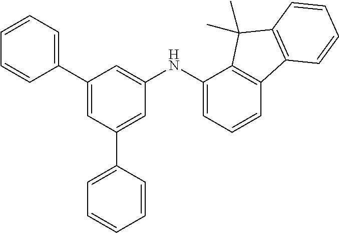 [SMILES: CC1(C)c2ccccc2-c2cccc(Nc3cc(-c4ccccc4)cc(-c4ccccc4)c3)c21] | 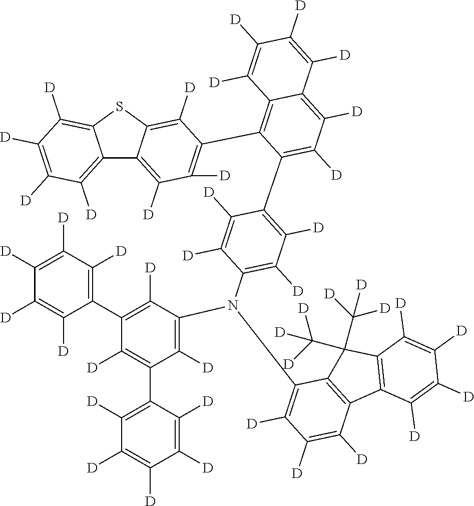 [SMILES: [2H]c1c([2H])c([2H])c(-c2c([2H])c(-c3c([2H])c([2H])c([2H])c([2H])c3[2H])c([2H])c(N(c3c([2H])c([2H])c(-c4c([2H])c([2H])c5c([2H])c([2H])c([2H])c([2H])c5c4-c4c([2H])c([2H])c5c(sc6c([2H])c([2H])c([2H])c([2H])c65)c4[2H])c([2H])c3[2H])c3c([2H])c([2H])c([2H])c4c3C(C([2H])([2H])[2H])(C([2H])([2H])[2H])c3c([2H])c([2H])c([2H])c([2H])c3-4)c2[2H])c([2H])c1[2H]] | 74% |
| 030 | 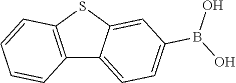 [SMILES: OB(O)c1ccc2c(c1)sc1ccccc12] | 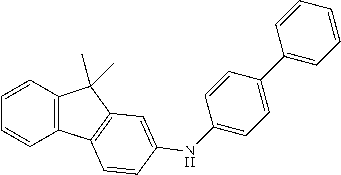 [SMILES: CC1(C)c2ccccc2-c2ccc(Nc3ccc(-c4ccccc4)cc3)cc21] | 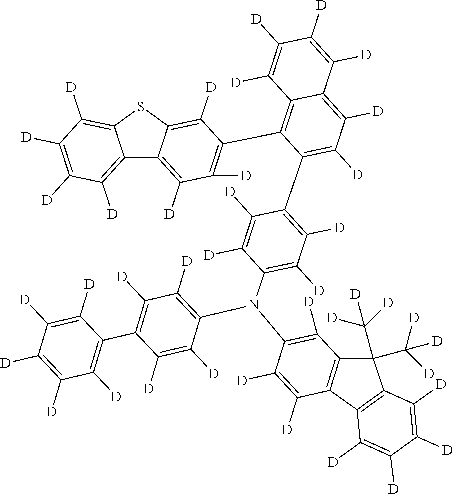 [SMILES: [2H]c1c([2H])c([2H])c(-c2c([2H])c([2H])c(N(c3c([2H])c([2H])c(-c4c([2H])c([2H])c5c([2H])c([2H])c([2H])c([2H])c5c4-c4c([2H])c([2H])c5c(sc6c([2H])c([2H])c([2H])c([2H])c65)c4[2H])c([2H])c3[2H])c3c([2H])c([2H])c4c(c3[2H])C(C([2H])([2H])[2H])(C([2H])([2H])[2H])c3c([2H])c([2H])c([2H])c([2H])c3-4)c([2H])c2[2H])c([2H])c1[2H]] | 78% |
| 259 | 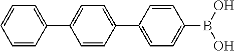 [SMILES: OB(O)c1ccc(-c2ccc(-c3ccccc3)cc2)cc1] | 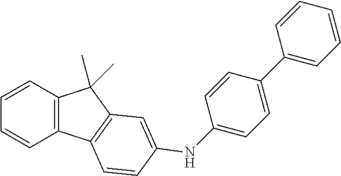 [SMILES: CC1(C)c2ccccc2-c2ccc(Nc3ccc(-c4ccccc4)cc3)cc21] | 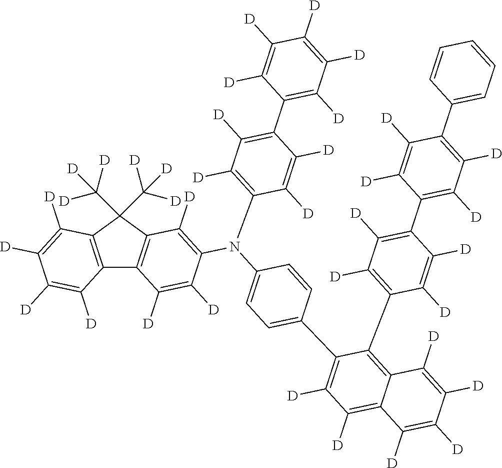 [SMILES: [2H]c1c([2H])c([2H])c(-c2c([2H])c([2H])c(N(c3ccc(-c4c([2H])c([2H])c5c([2H])c([2H])c([2H])c([2H])c5c4-c4c([2H])c([2H])c(-c5c([2H])c([2H])c(-c6ccccc6)c([2H])c5[2H])c([2H])c4[2H])cc3)c3c([2H])c([2H])c4c(c3[2H])C(C([2H])([2H])[2H])(C([2H])([2H])[2H])c3c([2H])c([2H])c([2H])c([2H])c3-4)c([2H])c2[2H])c([2H])c1[2H]] | 77% |
| 261 | 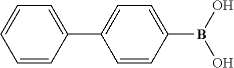 [SMILES: OB(O)c1ccc(-c2ccccc2)cc1] | 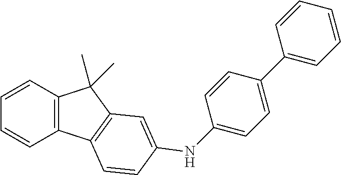 [SMILES: CC1(C)c2ccccc2-c2ccc(Nc3ccc(-c4ccccc4)cc3)cc21] | 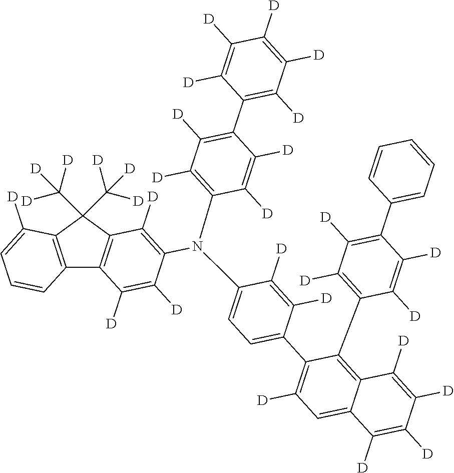 [SMILES: [2H]c1cc2c([2H])c([2H])c([2H])c([2H])c2c(-c2c([2H])c([2H])c(-c3ccccc3)c([2H])c2[2H])c1-c1ccc(N(c2c([2H])c([2H])c(-c3c([2H])c([2H])c([2H])c([2H])c3[2H])c([2H])c2[2H])c2c([2H])c([2H])c3c(c2[2H])C(C([2H])([2H])[2H])(C([2H])([2H])[2H])c2c([2H])cccc2-3)c([2H])c1[2H]] | 75% |
| 262 | 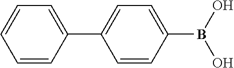 [SMILES: OB(O)c1ccc(-c2ccccc2)cc1] | 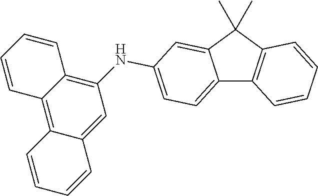 [SMILES: CC1(C)c2ccccc2-c2ccc(Nc3cc4ccccc4c4ccccc34)cc21] | 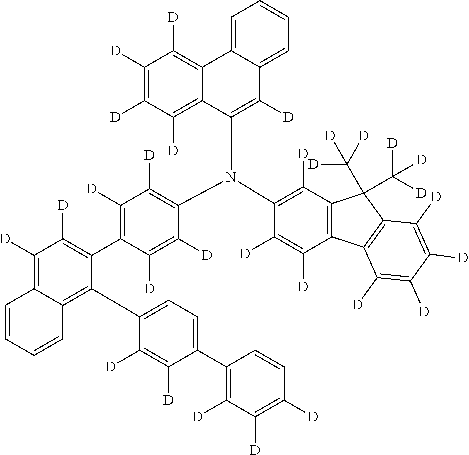 [SMILES: [2H]c1ccc(-c2ccc(-c3c(-c4c([2H])c([2H])c(N(c5c([2H])c([2H])c6c(c5[2H])C(C([2H])([2H])[2H])(C([2H])([2H])[2H])c5c([2H])c([2H])c([2H])c([2H])c5-6)c5c([2H])c6ccccc6c6c([2H])c([2H])c([2H])c([2H])c56)c([2H])c4[2H])c([2H])c([2H])c4ccccc34)c([2H])c2[2H])c([2H])c1[2H]] | 70% |
| 266 | 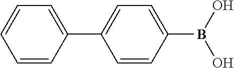 [SMILES: OB(O)c1ccc(-c2ccccc2)cc1] | 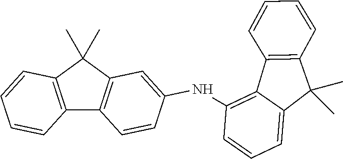 [SMILES: CC1(C)c2ccccc2-c2ccc(Nc3cccc4c3-c3ccccc3C4(C)C)cc21] | 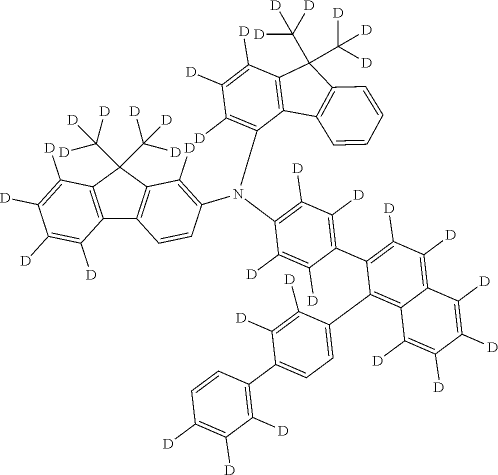 [SMILES: [2H]c1ccc(-c2ccc(-c3c(-c4c([2H])c([2H])c(N(c5ccc6c(c5[2H])C(C([2H])([2H])[2H])(C([2H])([2H])[2H])c5c([2H])c([2H])c([2H])c([2H])c5-6)c5c([2H])c([2H])c([2H])c6c5-c5ccccc5C6(C([2H])([2H])[2H])C([2H])([2H])[2H])c([2H])c4[2H])c([2H])c([2H])c4c([2H])c([2H])c([2H])c([2H])c34)c([2H])c2[2H])c([2H])c1[2H]] | 73% |
| 267 | 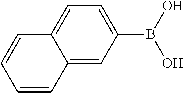 [SMILES: OB(O)c1ccc2ccccc2c1] | 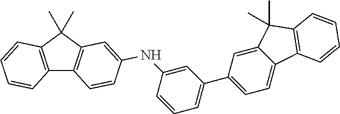 [SMILES: CC1(C)c2ccccc2-c2ccc(Nc3cccc(-c4ccc5c(c4)C(C)(C)c4ccccc4-5)c3)cc21] | 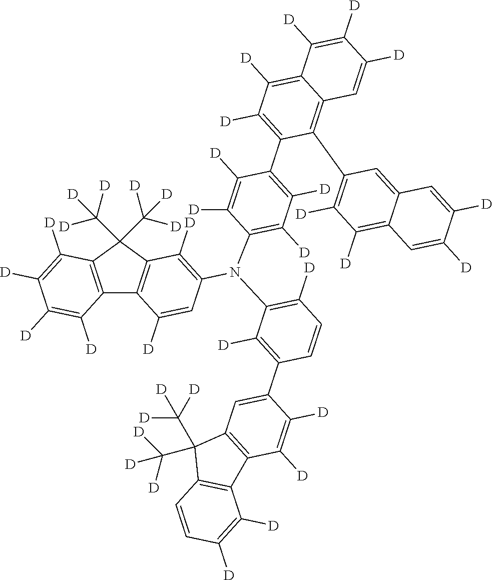 [SMILES: [2H]c1cc2cc(-c3c(-c4c([2H])c([2H])c(N(c5cc([2H])c6c(c5[2H])C(C([2H])([2H])[2H])(C([2H])([2H])[2H])c5c([2H])c([2H])c([2H])c([2H])c5-6)c5c([2H])ccc(-c6cc7c(c([2H])c6[2H])-c6c(ccc([2H])c6[2H])C7(C([2H])([2H])[2H])C([2H])([2H])[2H])c5[2H])c([2H])c4[2H])c([2H])c([2H])c4c([2H])c([2H])c([2H])cc34)c([2H])c([2H])c2cc1[2H]] | 69% |
| 281 | 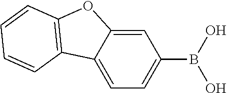 [SMILES: OB(O)c1ccc2c(c1)oc1ccccc12] | 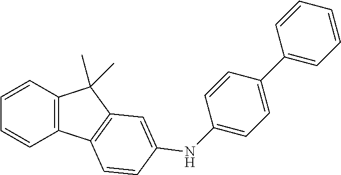 [SMILES: CC1(C)c2ccccc2-c2ccc(Nc3ccc(-c4ccccc4)cc3)cc21] | 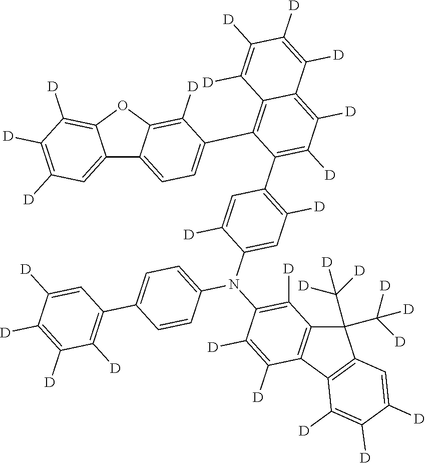 [SMILES: [2H]c1cc(N(c2ccc(-c3cc([2H])c([2H])c([2H])c3[2H])cc2)c2c([2H])c([2H])c3c(c2[2H])C(C([2H])([2H])[2H])(C([2H])([2H])[2H])c2cc([2H])c([2H])c([2H])c2-3)c([2H])cc1-c1c([2H])c([2H])c2c([2H])c([2H])c([2H])c([2H])c2c1-c1ccc2c(oc3c([2H])c([2H])c([2H])cc32)c1[2H]] | 71% |
| 288 | 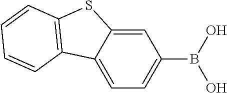 [SMILES: OB(O)c1ccc2c(c1)sc1ccccc12] | 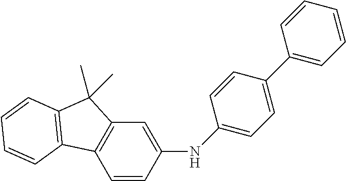 [SMILES: CC1(C)c2ccccc2-c2ccc(Nc3ccc(-c4ccccc4)cc3)cc21] | 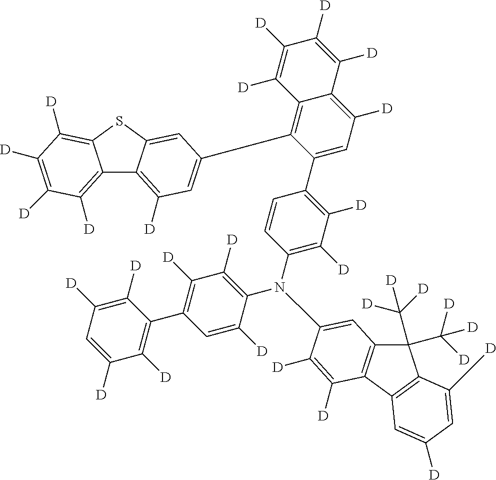 [SMILES: [2H]c1cc([2H])c2c(c1)-c1c(cc(N(c3ccc(-c4cc([2H])c5c([2H])c([2H])c([2H])c([2H])c5c4-c4cc([2H])c5c(c4)sc4c([2H])c([2H])c([2H])c([2H])c45)c([2H])c3[2H])c3c([2H])cc(-c4c([2H])c([2H])cc([2H])c4[2H])c([2H])c3[2H])c([2H])c1[2H])C2(C([2H])([2H])[2H])C([2H])([2H])[2H]] | 75% |
| 291 | 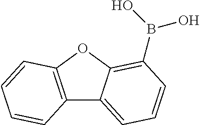 [SMILES: OB(O)c1cccc2c1oc1ccccc12] | 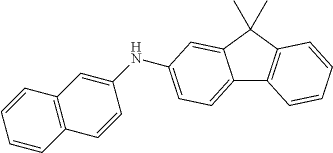 [SMILES: CC1(C)c2ccccc2-c2ccc(Nc3ccc4ccccc4c3)cc21] | 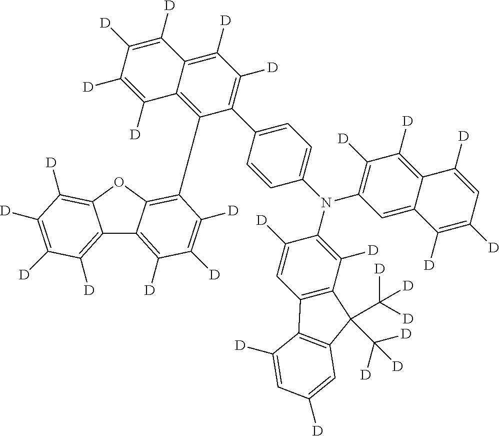 [SMILES: [2H]c1cc([2H])c2c(c1)C(C([2H])([2H])[2H])(C([2H])([2H])[2H])c1c-2cc([2H])c(N(c2ccc(-c3c([2H])c([2H])c4c([2H])c([2H])c([2H])c([2H])c4c3-c3c([2H])c([2H])c([2H])c4c3oc3c([2H])c([2H])c([2H])c([2H])c34)cc2)c2cc3c([2H])c([2H])cc([2H])c3c([2H])c2[2H])c1[2H]] | 78% |

### [Preparation Example 2] Preparation of Compound 087

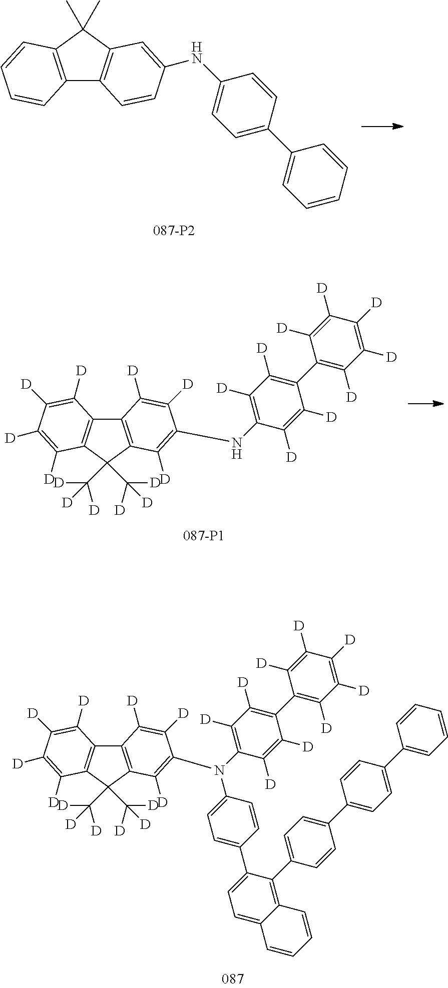 [SMILES: CC1(C)c2ccccc2-c2ccc(Nc3ccc(-c4ccccc4)cc3)cc21 ; [2H]c1c([2H])c([2H])c(-c2c([2H])c([2H])c(Nc3c([2H])c([2H])c4c(c3[2H])C(C([2H])([2H])[2H])(C([2H])([2H])[2H])c3c([2H])c([2H])c([2H])c([2H])c3-4)c([2H])c2[2H])c([2H])c1[2H] ; [2H]c1c([2H])c([2H])c(-c2c([2H])c([2H])c(N(c3ccc(-c4ccc5ccccc5c4-c4ccc(-c5ccc(-c6ccccc6)cc5)cc4)cc3)c3c([2H])c([2H])c4c(c3[2H])C(C([2H])([2H])[2H])(C([2H])([2H])[2H])c3c([2H])c([2H])c([2H])c([2H])c3-4)c([2H])c2[2H])c([2H])c1[2H]]

### 1) Preparation of Compound 087-P1

N-([1,1′-biphenyl]-4-yl)-9,9-dimethyl-9H-fluoren-2-amine (10 g, 0.0277 mol), benzene-D₆ (100 ml, 10 T), and trifluoromethanesulfonic acid (17.10 ml, 0.1936 mol) were put into a flask and stirred at 60° C. for 4 hours, and then 300 ml of distilled water and NaHCO₃ were added thereto to terminate the reaction. After the organic layer was extracted, moisture was removed using MgSO₄. The concentrated organic layer was allowed to pass through silica gel and purified to obtain Compound 087-P1 (10 g, 94%).

### 2) Preparation of Compound 087

After Compound 087-P1 (10 g, 0.0261 mol) and a compound 1-([1,1′:4′,1″-terphenyl]-4-yl)-2-(4-chlorophenyl)naphthalene (12.17 g, 0.0261 mol) were dissolved in 100 ml of toluene, Pd₂(dba)₃ (1.19 g, 0.0013 mol), xphos (1.86 g, 0.0039 mol), and t-BuONa (6.26 g, 0.0652 mol) were added thereto, and the resulting mixture was stirred under reflux for 3 hours. After the reaction was completed, the reaction solution was extracted, and then the organic layer was adsorbed onto silica and purified by adsorption column chromatography to obtain Compound 087 (18 g, 85%).

A target compound was synthesized in the same manner as in Preparation Example 2, except that Compound C and Compound D in the following Table 2 were used instead of N-([1,1′-biphenyl]-4-yl)-9,9-dimethyl-9H-fluoren-2-amine and chlorophenyl)naphthalene, respectively, in Preparation Example 2.

TABLE 2

| Compound | | | | |
|---|---|---|---|---|
| No. | Compound C | Compound D | Target compound | yield |
| 088 | 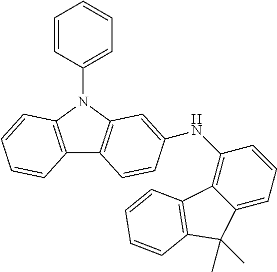 [SMILES: CC1(C)c2ccccc2-c2c(Nc3ccc4c5ccccc5n(-c5ccccc5)c4c3)cccc21] | 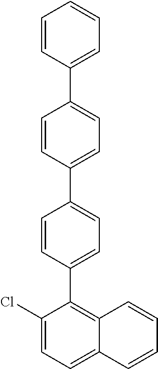 [SMILES: Clc1ccc2ccccc2c1-c1ccc(-c2ccc(-c3ccccc3)cc2)cc1] | 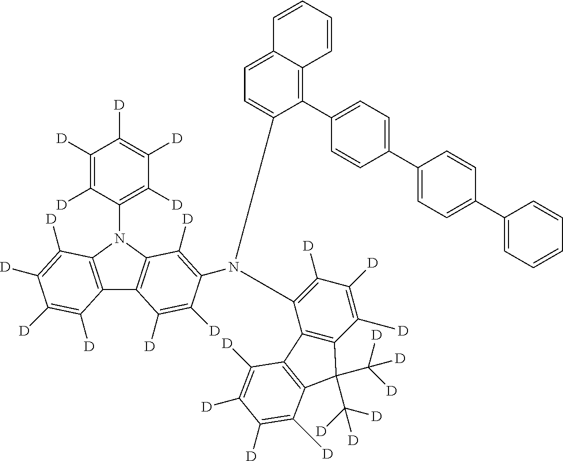 [SMILES: [2H]c1c([2H])c([2H])c(-n2c3c([2H])c([2H])c([2H])c([2H])c3c3c([2H])c([2H])c(N(c4ccc5ccccc5c4-c4ccc(-c5ccc(-c6ccccc6)cc5)cc4)c4c([2H])c([2H])c([2H])c5c4-c4c([2H])c([2H])c([2H])c([2H])c4C5(C([2H])([2H])[2H])C([2H])([2H])[2H])c([2H])c32)c([2H])c1[2H]] | 76% |
| 089 | 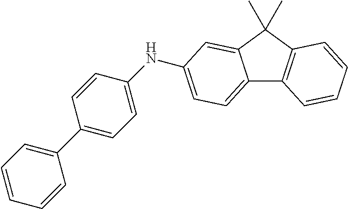 [SMILES: CC1(C)c2ccccc2-c2ccc(Nc3ccc(-c4ccccc4)cc3)cc21] | 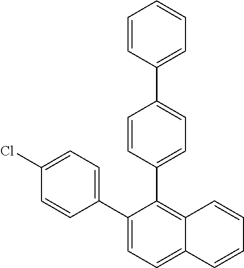 [SMILES: Clc1ccc(-c2ccc3ccccc3c2-c2ccc(-c3ccccc3)cc2)cc1] | 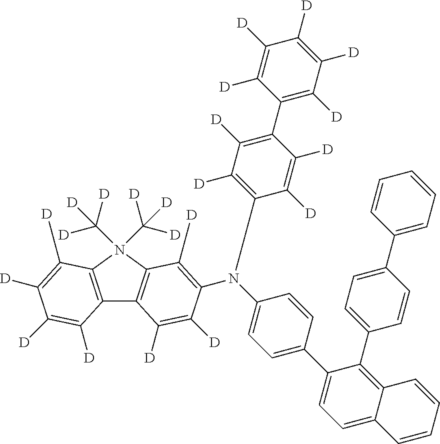 | 71% |
| 090 | 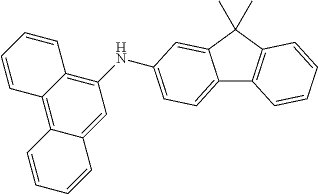 [SMILES: CC1(C)c2ccccc2-c2ccc(Nc3cc4ccccc4c4ccccc34)cc21] | 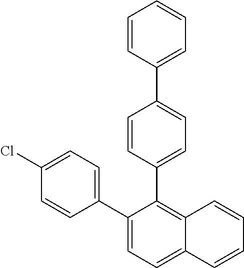 [SMILES: Clc1ccc(-c2ccc3ccccc3c2-c2ccc(-c3ccccc3)cc2)cc1] | 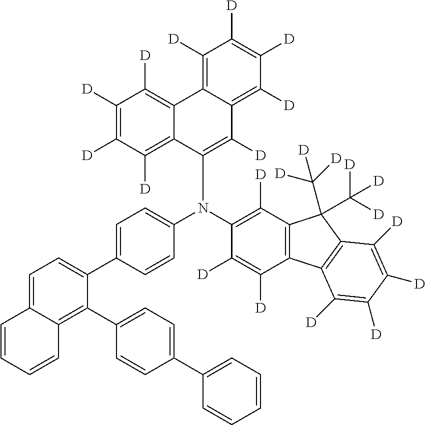 [SMILES: [2H]c1c([2H])c([2H])c2c(c1[2H])-c1c([2H])c([2H])c(N(c3ccc(-c4ccc5ccccc5c4-c4ccc(-c5ccccc5)cc4)cc3)c3c([2H])c4c([2H])c([2H])c([2H])c([2H])c4c4c([2H])c([2H])c([2H])c([2H])c34)c([2H])c1C2(C([2H])([2H])[2H])C([2H])([2H])[2H]] | 72% |
| 091 | 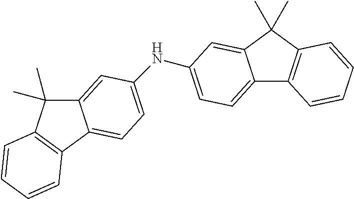 [SMILES: CC1(C)c2ccccc2-c2ccc(Nc3ccc4c(c3)C(C)(C)c3ccccc3-4)cc21] | 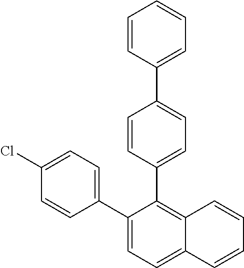 [SMILES: Clc1ccc(-c2ccc3ccccc3c2-c2ccc(-c3ccccc3)cc2)cc1] | 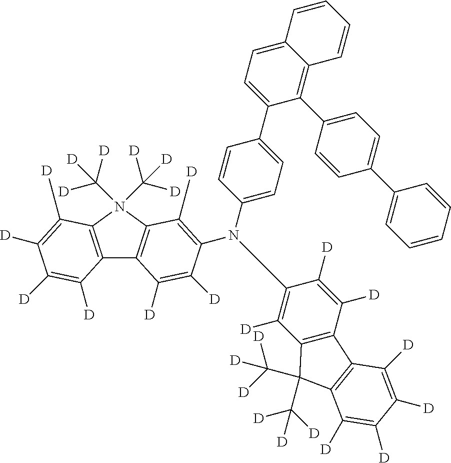 | 78% |
| 093 | 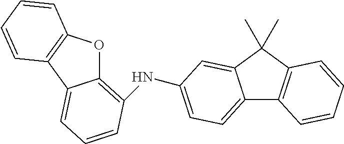 [SMILES: CC1(C)c2ccccc2-c2ccc(Nc3cccc4c3oc3ccccc34)cc21] | 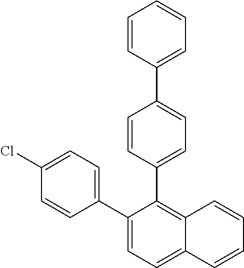 [SMILES: Clc1ccc(-c2ccc3ccccc3c2-c2ccc(-c3ccccc3)cc2)cc1] | 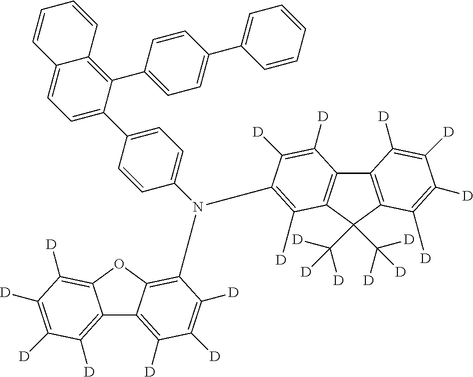 [SMILES: [2H]c1c([2H])c([2H])c2c(c1[2H])-c1c([2H])c([2H])c(N(c3ccc(-c4ccc5ccccc5c4-c4ccc(-c5ccccc5)cc4)cc3)c3c([2H])c([2H])c([2H])c4c3oc3c([2H])c([2H])c([2H])c([2H])c34)c([2H])c1C2(C([2H])([2H])[2H])C([2H])([2H])[2H]] | 79% |
| 098 | 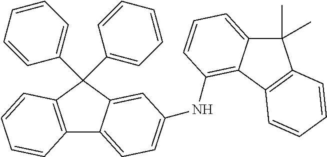 [SMILES: CC1(C)c2ccccc2-c2c(Nc3ccc4c(c3)C(c3ccccc3)(c3ccccc3)c3ccccc3-4)cccc21] | 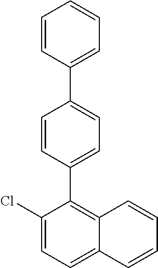 [SMILES: Clc1ccc2ccccc2c1-c1ccc(-c2ccccc2)cc1] | 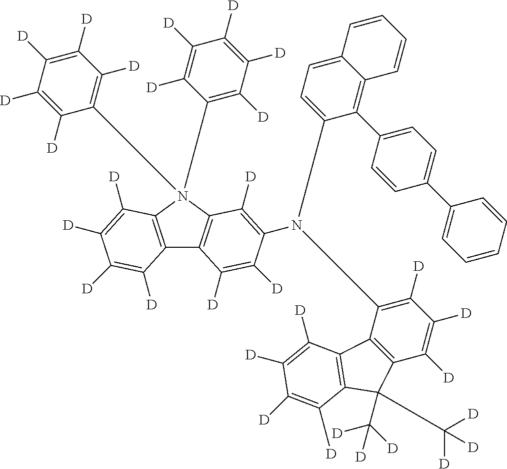 | 70% |
| 099 | 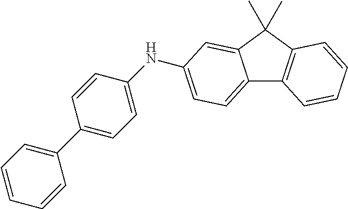 [SMILES: CC1(C)c2ccccc2-c2ccc(Nc3ccc(-c4ccccc4)cc3)cc21] | 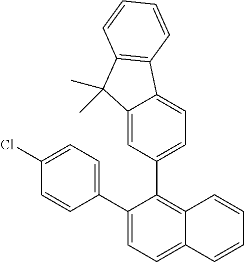 [SMILES: CC1(C)c2ccccc2-c2ccc(-c3c(-c4ccc(Cl)cc4)ccc4ccccc34)cc21] | 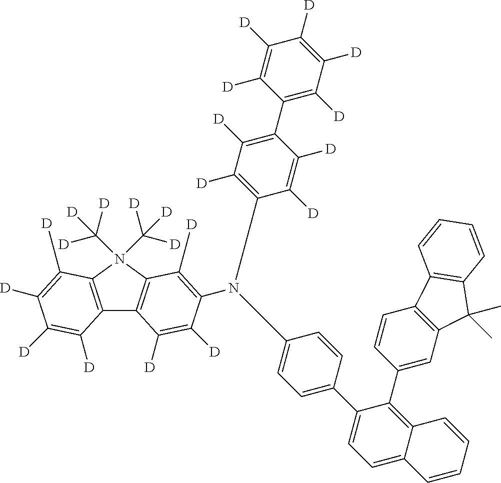 | 75% |
| 109 | 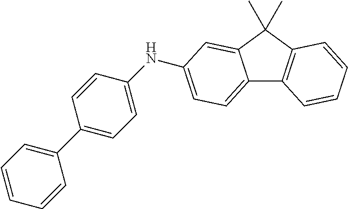 [SMILES: CC1(C)c2ccccc2-c2ccc(Nc3ccc(-c4ccccc4)cc3)cc21] | 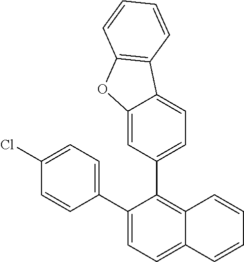 [SMILES: Clc1ccc(-c2ccc3ccccc3c2-c2ccc3c(c2)oc2ccccc23)cc1] | 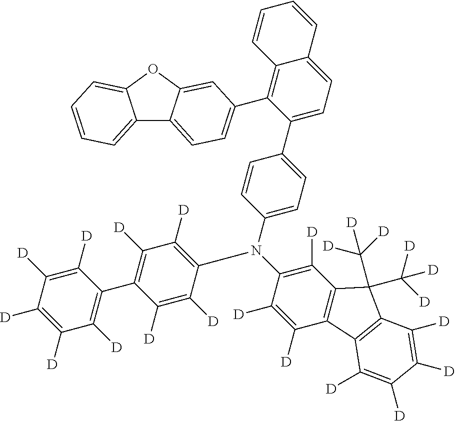 [SMILES: [2H]c1c([2H])c([2H])c(-c2c([2H])c([2H])c(N(c3ccc(-c4ccc5ccccc5c4-c4ccc5c(c4)oc4ccccc45)cc3)c3c([2H])c([2H])c4c(c3[2H])C(C([2H])([2H])[2H])(C([2H])([2H])[2H])c3c([2H])c([2H])c([2H])c([2H])c3-4)c([2H])c2[2H])c([2H])c1[2H]] | 78% |
| 111 | 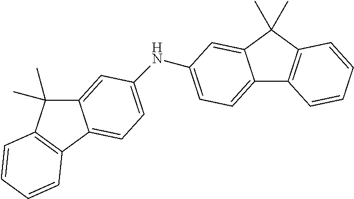 [SMILES: CC1(C)c2ccccc2-c2ccc(Nc3ccc4c(c3)C(C)(C)c3ccccc3-4)cc21] | 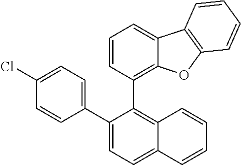 [SMILES: Clc1ccc(-c2ccc3ccccc3c2-c2cccc3c2oc2ccccc23)cc1] | 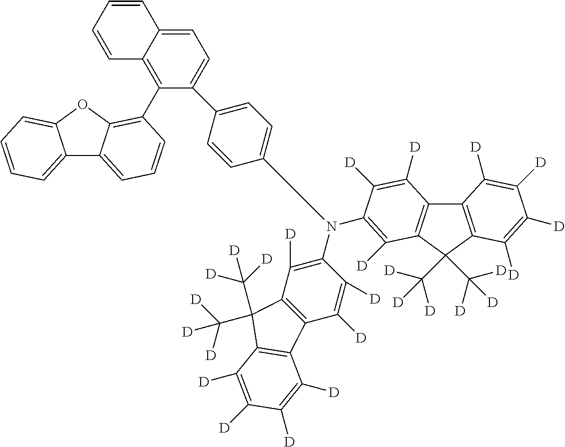 [SMILES: [2H]c1c([2H])c([2H])c2c(c1[2H])-c1c([2H])c([2H])c(N(c3ccc(-c4ccc5ccccc5c4-c4cccc5c4oc4ccccc45)cc3)c3c([2H])c([2H])c4c(c3[2H])C(C([2H])([2H])[2H])(C([2H])([2H])[2H])c3c([2H])c([2H])c([2H])c([2H])c3-4)c([2H])c1C2(C([2H])([2H])[2H])C([2H])([2H])[2H]] | 74% |
| 112 | 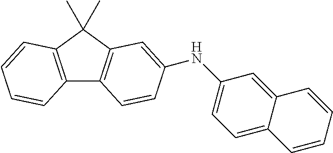 [SMILES: CC1(C)c2ccccc2-c2ccc(Nc3ccc4ccccc4c3)cc21] | 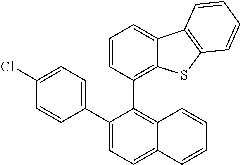 [SMILES: Clc1ccc(-c2ccc3ccccc3c2-c2cccc3c2sc2ccccc23)cc1] | 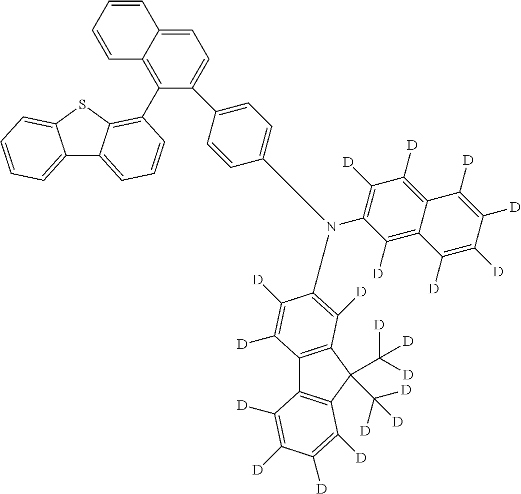 [SMILES: [2H]c1c([2H])c([2H])c2c(c1[2H])-c1c([2H])c([2H])c(N(c3ccc(-c4ccc5ccccc5c4-c4cccc5c4sc4ccccc45)cc3)c3c([2H])c([2H])c4c([2H])c([2H])c([2H])c([2H])c4c3[2H])c([2H])c1C2(C([2H])([2H])[2H])C([2H])([2H])[2H]] | 73% |
| 116 | 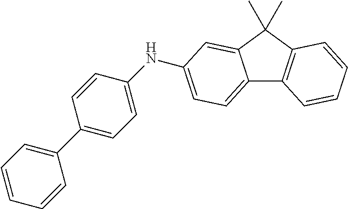 [SMILES: CC1(C)c2ccccc2-c2ccc(Nc3ccc(-c4ccccc4)cc3)cc21] | 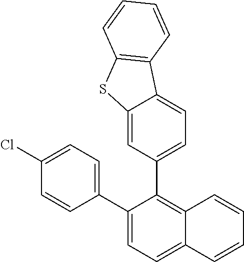 [SMILES: Clc1ccc(-c2ccc3ccccc3c2-c2ccc3c(c2)sc2ccccc23)cc1] | 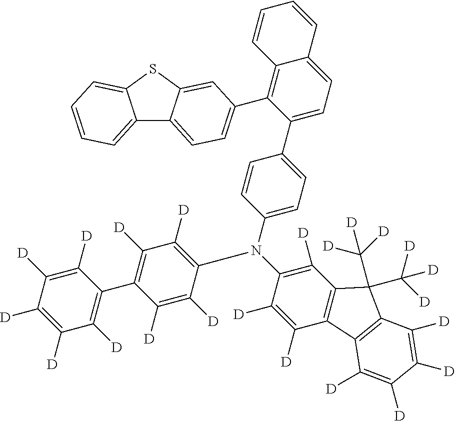 [SMILES: [2H]c1c([2H])c([2H])c(-c2c([2H])c([2H])c(N(c3ccc(-c4ccc5ccccc5c4-c4ccc5c(c4)sc4ccccc45)cc3)c3c([2H])c([2H])c4c(c3[2H])C(C([2H])([2H])[2H])(C([2H])([2H])[2H])c3c([2H])c([2H])c([2H])c([2H])c3-4)c([2H])c2[2H])c([2H])c1[2H]] | 77% |
| 173 | 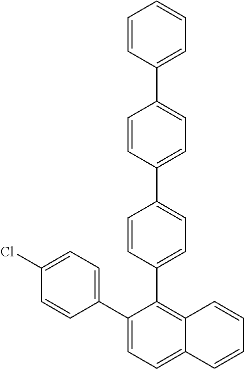 [SMILES: Clc1ccc(-c2ccc3ccccc3c2-c2ccc(-c3ccc(-c4ccccc4)cc3)cc2)cc1] | 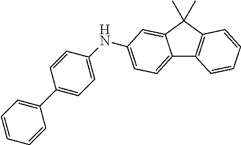 [SMILES: CC1(C)c2ccccc2-c2ccc(Nc3ccc(-c4ccccc4)cc3)cc21] | 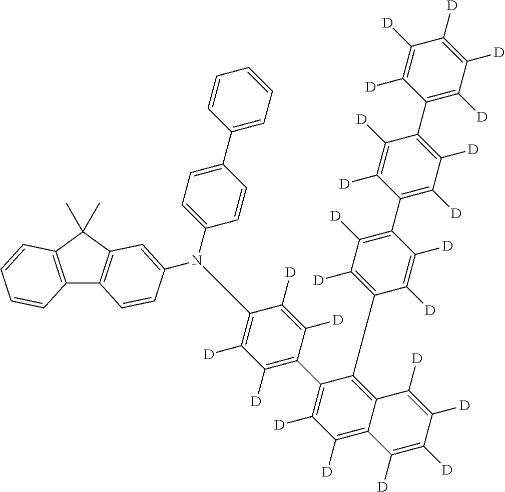 [SMILES: [2H]c1c([2H])c([2H])c(-c2c([2H])c([2H])c(-c3c([2H])c([2H])c(-c4c(-c5c([2H])c([2H])c(N(c6ccc(-c7ccccc7)cc6)c6ccc7c(c6)C(C)(C)c6ccccc6-7)c([2H])c5[2H])c([2H])c([2H])c5c([2H])c([2H])c([2H])c([2H])c45)c([2H])c3[2H])c([2H])c2[2H])c([2H])c1[2H]] | 71% |
| 175 | 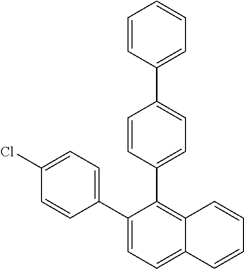 [SMILES: Clc1ccc(-c2ccc3ccccc3c2-c2ccc(-c3ccccc3)cc2)cc1] | 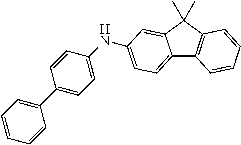 [SMILES: CC1(C)c2ccccc2-c2ccc(Nc3ccc(-c4ccccc4)cc3)cc21] | 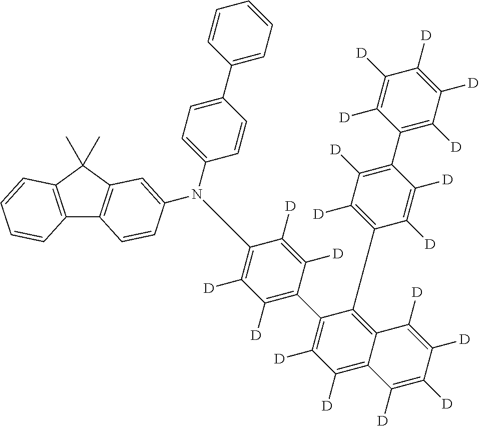 [SMILES: [2H]c1c([2H])c([2H])c(-c2c([2H])c([2H])c(-c3c(-c4c([2H])c([2H])c(N(c5ccc(-c6ccccc6)cc5)c5ccc6c(c5)C(C)(C)c5ccccc5-6)c([2H])c4[2H])c([2H])c([2H])c4c([2H])c([2H])c([2H])c([2H])c34)c([2H])c2[2H])c([2H])c1[2H]] | 71% |
| 176 | 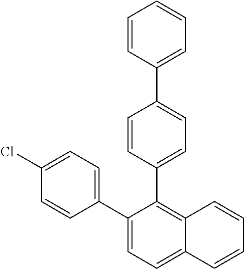 [SMILES: Clc1ccc(-c2ccc3ccccc3c2-c2ccc(-c3ccccc3)cc2)cc1] | 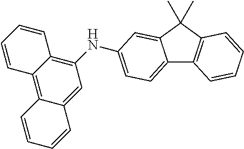 [SMILES: CC1(C)c2ccccc2-c2ccc(Nc3cc4ccccc4c4ccccc34)cc21] | 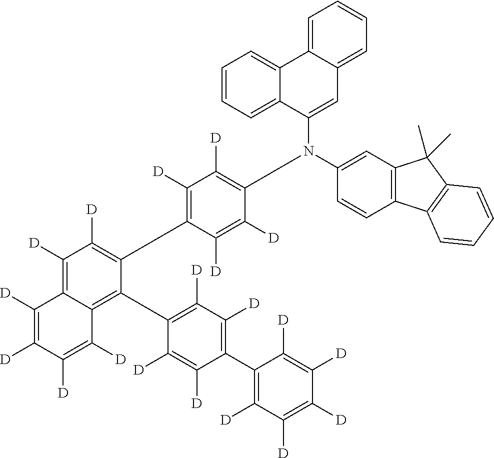 [SMILES: [2H]c1c([2H])c([2H])c(-c2c([2H])c([2H])c(-c3c(-c4c([2H])c([2H])c(N(c5ccc6c(c5)C(C)(C)c5ccccc5-6)c5cc6ccccc6c6ccccc56)c([2H])c4[2H])c([2H])c([2H])c4c([2H])c([2H])c([2H])c([2H])c34)c([2H])c2[2H])c([2H])c1[2H]] | 74% |
| 177 | 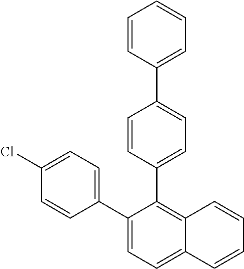 [SMILES: Clc1ccc(-c2ccc3ccccc3c2-c2ccc(-c3ccccc3)cc2)cc1] | 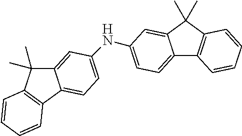 [SMILES: CC1(C)c2ccccc2-c2ccc(Nc3ccc4c(c3)C(C)(C)c3ccccc3-4)cc21] | 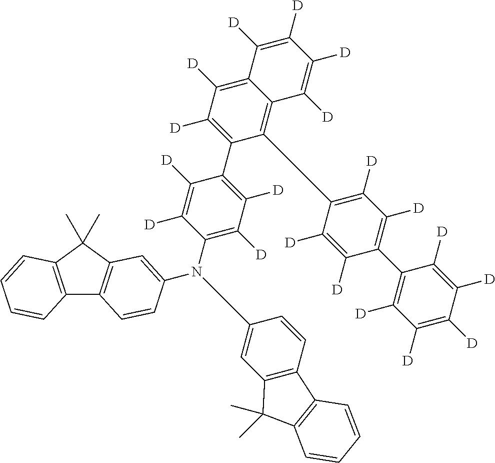 [SMILES: [2H]c1c([2H])c([2H])c(-c2c([2H])c([2H])c(-c3c(-c4c([2H])c([2H])c(N(c5ccc6c(c5)C(C)(C)c5ccccc5-6)c5ccc6c(c5)C(C)(C)c5ccccc5-6)c([2H])c4[2H])c([2H])c([2H])c4c([2H])c([2H])c([2H])c([2H])c34)c([2H])c2[2H])c([2H])c1[2H]] | 70% |
| 179 | 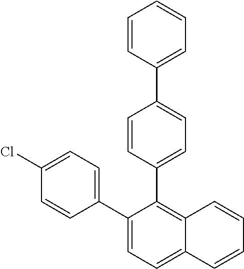 [SMILES: Clc1ccc(-c2ccc3ccccc3c2-c2ccc(-c3ccccc3)cc2)cc1] | 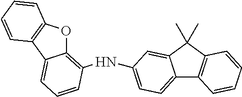 [SMILES: CC1(C)c2ccccc2-c2ccc(Nc3cccc4c3oc3ccccc34)cc21] | 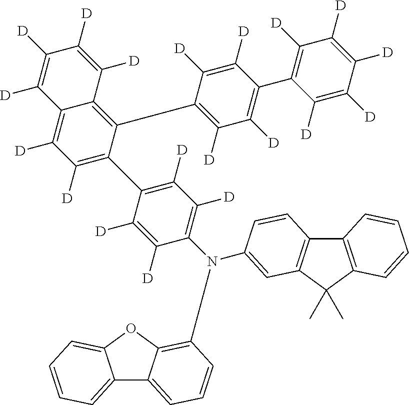 [SMILES: [2H]c1c([2H])c([2H])c(-c2c([2H])c([2H])c(-c3c(-c4c([2H])c([2H])c(N(c5ccc6c(c5)C(C)(C)c5ccccc5-6)c5cccc6c5oc5ccccc56)c([2H])c4[2H])c([2H])c([2H])c4c([2H])c([2H])c([2H])c([2H])c34)c([2H])c2[2H])c([2H])c1[2H]] | 77% |
| 182 | 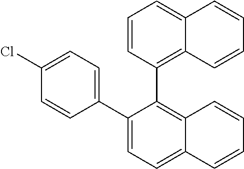 [SMILES: Clc1ccc(-c2ccc3ccccc3c2-c2cccc3ccccc23)cc1] | 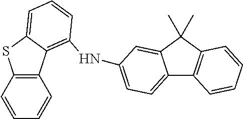 [SMILES: CC1(C)c2ccccc2-c2ccc(Nc3cccc4sc5ccccc5c34)cc21] | 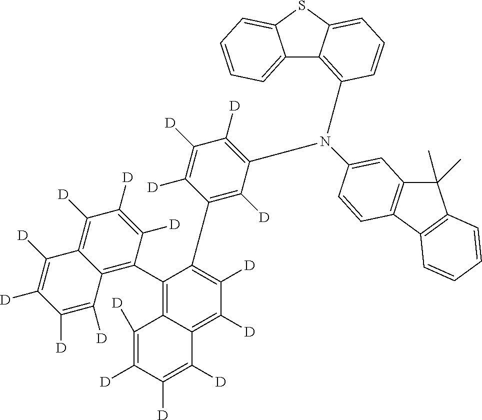 [SMILES: [2H]c1c([2H])c(-c2c([2H])c([2H])c3c([2H])c([2H])c([2H])c([2H])c3c2-c2c([2H])c([2H])c([2H])c3c([2H])c([2H])c([2H])c([2H])c23)c([2H])c(N(c2ccc3c(c2)C(C)(C)c2ccccc2-3)c2cccc3sc4ccccc4c23)c1[2H]] | 72% |
| 184 | 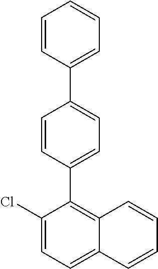 [SMILES: Clc1ccc2ccccc2c1-c1ccc(-c2ccccc2)cc1] | 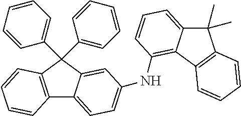 [SMILES: CC1(C)c2ccccc2-c2c(Nc3ccc4c(c3)C(c3ccccc3)(c3ccccc3)c3ccccc3-4)cccc21] | 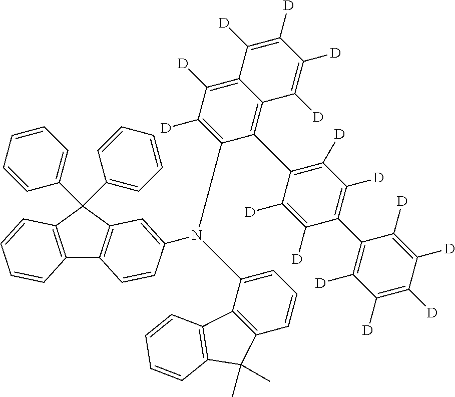 [SMILES: [2H]c1c([2H])c([2H])c(-c2c([2H])c([2H])c(-c3c(N(c4ccc5c(c4)C(c4ccccc4)(c4ccccc4)c4ccccc4-5)c4cccc5c4-c4ccccc4C5(C)C)c([2H])c([2H])c4c([2H])c([2H])c([2H])c([2H])c34)c([2H])c2[2H])c([2H])c1[2H]] | 73% |
| 185 | 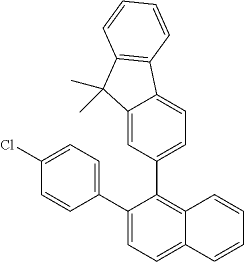 [SMILES: CC1(C)c2ccccc2-c2ccc(-c3c(-c4ccc(Cl)cc4)ccc4ccccc34)cc21] | 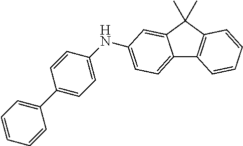 [SMILES: CC1(C)c2ccccc2-c2ccc(Nc3ccc(-c4ccccc4)cc3)cc21] | 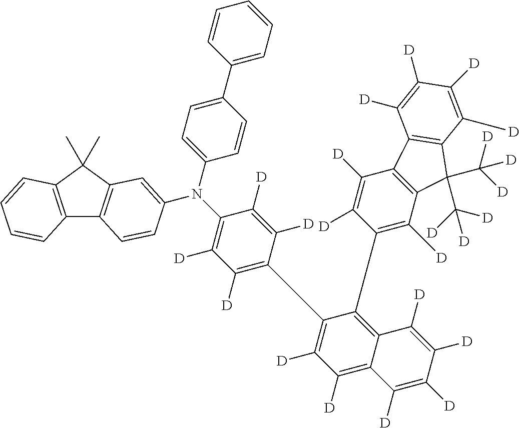 [SMILES: [2H]c1c([2H])c([2H])c2c(c1[2H])-c1c([2H])c([2H])c(-c3c(-c4c([2H])c([2H])c(N(c5ccc(-c6ccccc6)cc5)c5ccc6c(c5)C(C)(C)c5ccccc5-6)c([2H])c4[2H])c([2H])c([2H])c4c([2H])c([2H])c([2H])c([2H])c34)c([2H])c1C2(C([2H])([2H])[2H])C([2H])([2H])[2H]] | 71% |
| 189 | 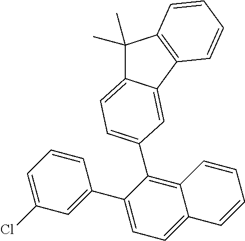 [SMILES: CC1(C)c2ccccc2-c2cc(-c3c(-c4cccc(Cl)c4)ccc4ccccc34)ccc21] | 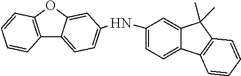 [SMILES: CC1(C)c2ccccc2-c2ccc(Nc3ccc4c(c3)oc3ccccc34)cc21] | 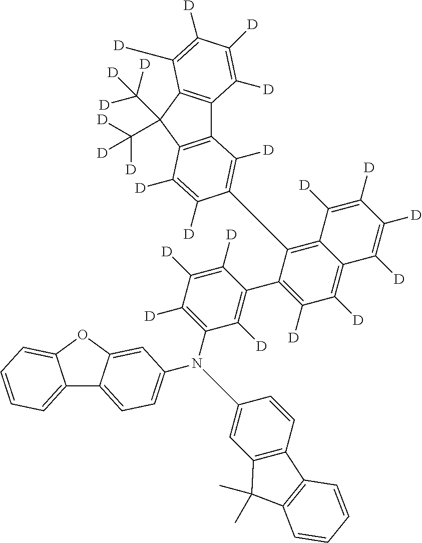 [SMILES: [2H]c1c([2H])c([2H])c2c(c1[2H])-c1c([2H])c(-c3c(-c4c([2H])c([2H])c([2H])c(N(c5ccc6c(c5)C(C)(C)c5ccccc5-6)c5ccc6c(c5)oc5ccccc56)c4[2H])c([2H])c([2H])c4c([2H])c([2H])c([2H])c([2H])c34)c([2H])c([2H])c1C2(C([2H])([2H])[2H])C([2H])([2H])[2H]] | 9% |
| 195 | 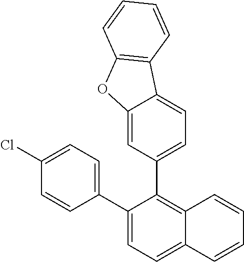 [SMILES: Clc1ccc(-c2ccc3ccccc3c2-c2ccc3c(c2)oc2ccccc23)cc1] | 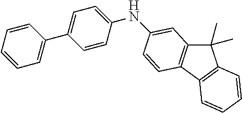 [SMILES: CC1(C)c2ccccc2-c2ccc(Nc3ccc(-c4ccccc4)cc3)cc21] | 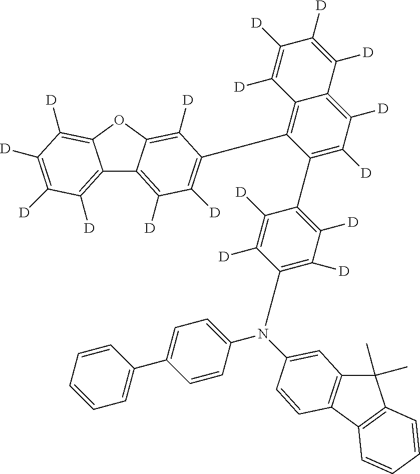 [SMILES: [2H]c1c([2H])c(N(c2ccc(-c3ccccc3)cc2)c2ccc3c(c2)C(C)(C)c2ccccc2-3)c([2H])c([2H])c1-c1c([2H])c([2H])c2c([2H])c([2H])c([2H])c([2H])c2c1-c1c([2H])c([2H])c2c(oc3c([2H])c([2H])c([2H])c([2H])c32)c1[2H]] | 71% |
| 199 | 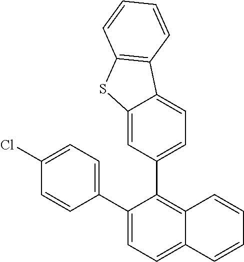 [SMILES: Clc1ccc(-c2ccc3ccccc3c2-c2ccc3c(c2)sc2ccccc23)cc1] | 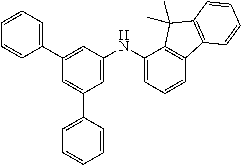 [SMILES: CC1(C)c2ccccc2-c2cccc(Nc3cc(-c4ccccc4)cc(-c4ccccc4)c3)c21] | 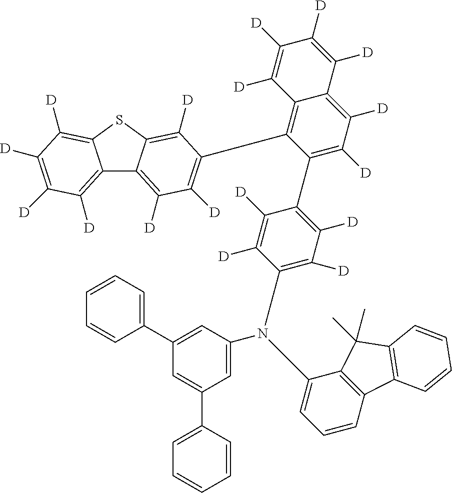 [SMILES: [2H]c1c([2H])c(N(c2cc(-c3ccccc3)cc(-c3ccccc3)c2)c2cccc3c2C(C)(C)c2ccccc2-3)c([2H])c([2H])c1-c1c([2H])c([2H])c2c([2H])c([2H])c([2H])c([2H])c2c1-c1c([2H])c([2H])c2c(sc3c([2H])c([2H])c([2H])c([2H])c32)c1[2H]] | 73% |
| 205 | 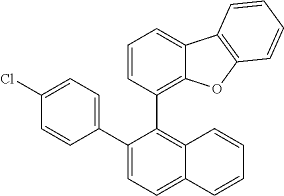 [SMILES: Clc1ccc(-c2ccc3ccccc3c2-c2cccc3c2oc2ccccc23)cc1] | 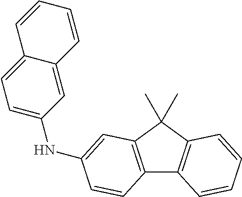 [SMILES: CC1(C)c2ccccc2-c2ccc(Nc3ccc4ccccc4c3)cc21] | 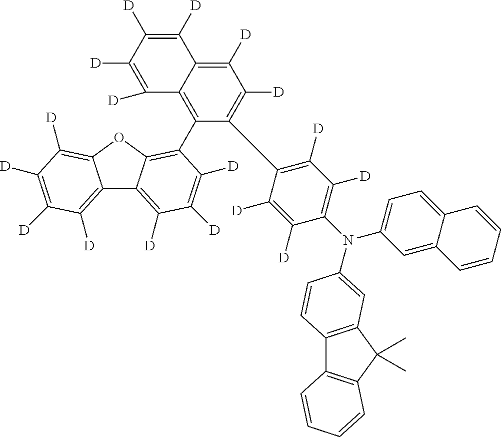 [SMILES: [2H]c1c([2H])c(N(c2ccc3c(c2)C(C)(C)c2ccccc2-3)c2ccc3ccccc3c2)c([2H])c([2H])c1-c1c([2H])c([2H])c2c([2H])c([2H])c([2H])c([2H])c2c1-c1c([2H])c([2H])c([2H])c2c1oc1c([2H])c([2H])c([2H])c([2H])c12] | 70% |

Compounds were prepared in the same manner as in the Preparation Examples 1 and 2, and the synthesis confirmation results thereof are shown in Tables 3 and 4. Table 3 shows the measured values of ¹H NMR(CDCl₃, 300 MHz), and Table 4 shows the measured values of a field desorption mass spectrometer (FD-MS).

TABLE 3

| Compound | ¹H NMR (CDCl₃, 300 MHz) |
|---|---|
| 001 | No proton |
| 003 | No proton |
| 005 | No proton |
| 007 | No proton |
| 011 | No proton |
| 013 | No proton |
| 023 | No proton |
| 025 | No proton |
| 026 | No proton |
| 027 | No proton |
| 030 | No proton |
| 087 | δ = 8.93 (1H, d), 8.31-8.26 (2H, m), 7.75-7.72 (3H, |
| | m), 7.55-7.37 (9H, m), 7.25 (8H, s) |
| 088 | δ = 8.80 (1H, d), 7.97 (1H, d), 7.89 (1H, d), 7.76- |
| | 7.75 (3H, m), 7.49-7.41 (3H, m), 7.25-7.20 (10H, |
| | m) |
| 089 | δ = 8.93 (1H, d), 8.31-8.26 (2H, m), 7.75-7.72 (3H, |
| | m), 7.55-7.37 (9H, m), 7.25 (4H, s) |
| 090 | δ = 8.93 (1H, d), 8.31-8.26 (2H, m), 7.75-7.72 (3H, |
| | m), 7.55-7.37 (9H, m), 7.25 (4H, s) |
| 091 | δ = 8.93 (1H, d), 8.31-8.26 (2H, m), 7.75-7.72 (3H, |
| | m), 7.55-7.37 (9H, m), 7.25 (4H, s) |
| 093 | δ = 8.93 (1H, d), 8.31-8.26 (2H, m), 7.75-7.72 (3H, |
| | m), 7.55-7.37 (9H, m), 7.25 (4H, s) |
| 098 | δ = 8.80 (1H, d), 7.97 (1H, d), 7.89 (1H, d), 7.76- |
| | 7.75 (3H, m), 7.49-7.41 (3H, m), 7.25-7.20 (10H, |
| | m) |
| 099 | δ = 8.93 (1H, d), 8.31-8.26 (2H, m), 8.09 (1H, d), |
| | 7.90-7.89(2H, m), 7.78-7.72 (2H, m), 7.75-7.74 |
| | (4H, m), 7.40-7.37 (4H, m), 7.28 (1H, m), 1.69 |
| | (6H, s) |
| 109 | δ = 8.93 (1H, d), 8.31-8.26 (2H, m), 8.03-7.98 (2H, |
| | m), 7.82-7.72 (3H, m), 7.55-7.54 (4H, m), 7.40- |
| | 7.31 (5H, m) |
| 111 | δ = 8.93 (1H, d), 8.31-8.26 (2H, m), 8.08-7.98 (3H, |
| | m), 7.72 (1H, d), 7.55-7.51 (5H, m), 7.40-7.31 |
| | (5H, m) |
| 112 | δ = 8.93 (1H, d), 8.55 (1H, d), 8.45 (1H, d), 8.32- |
| | 8.26 (3H, m), 7.93 (1H, d), 7.72-7.70 (2H, m), |
| | 7.55-7.37 (8H, m) |
| 116 | δ = 8.93 (1H, d), 8.45 (1H, d), 8.31-8.17 (5H, m), |
| | 7.93 (1H, d), 7.72 (1H, d), 7.56-7.37 (8H, m) |
| 173 | δ = 7.90-7.86 (2H, m), 7.75 (2H, d), 7.55-7.28 |
| | (11H, m), 7.16 (1H, d), 1.69 (6H, s) |
| 175 | δ = 7.90-7.86 (2H, m), 7.75 (2H, d), 7.55-7.28 |
| | (11H, m), 7.16 (1H, d), 1.69 (6H, s) |
| 176 | δ = 8.98 (1H, d), 8.84 (1H, d), 8.11 (1H, d), 7.90- |
| | 7.86 (3H, m), 7.68-7.49 (6H, m), 7.38-7.28 (3H, |
| | m), 7.16 (1H, d), 1.69 (6H, s) |
| 177 | δ = 7.90-7.86 (4H, m), 7.55 (2H, d), 7.38-7.28 (6H, |
| | m), 7.16 (2H, d), 1.69 (12H, s) |
| 179 | δ = 7.98 (1H, d), 7.90-7.86 (2H, d), 7.64 (1H, d), |
| | 7.55-7.54 (2H, m), 7.39-7.28 (6H, m), 7.16 (1H, |
| | d), 6.97 (1H, d), 1.69 (6H, s) |
| 182 | δ = 8.45 (1H, d), 7.93-7.86 (3H, m), 7.59-7.28 (9H, |
| | d), 7.16 (1H, d), 1.69 (6H, s) |
| 184 | δ = 7.90-7.86 (3H, m), 7.55 (2H, d), 7.38-7.10 |
| | (19H, m), 1.69 (6H, s) |
| 185 | δ = 7.90-7.86 (2H, m), 7.75 (2H, d), 7.55-7.28 |
| | (11H, m), 7.16 (1H, d), 1.69 (6H, s) |
| 189 | δ = 8.03-7.98 (2H, m), 7.90-7.80 (3H, m), 7.55-7.54 |
| | (2H, m), 7.39-7.28 (5H, m), 7.16 (1H, d), 6.91 |
| | (1H, d), 1.69 (6H, s) |
| 195 | δ = 7.90-7.86 (2H, m), 7.75 (2H, d), 7.55-7.28 |
| | (11H, m), 7.16 (1H, d), 1.69 (6H, s) |
| 199 | δ = 7.90 (1H, d), 7.75 (4H, m), 7.60-7.28 (14H, m), |
| | 7.06 (1H, d), 1.69 (6H, s) |
| 205 | δ = 7.90-7.86 (2H, m), 7.78-7.71 (2H, m), 7.55-7.54 |
| | (2H, m), 7.45-7.28 (6H, m), 7.16-7.11 (2H, m), |
| | 1.69 (6H, s) |
| 259 | δ = 7.55-7.37 (9H, m) |
| 261 | δ = 8.28 (1H, s), 7.90-7.86 (2H, m), 7.73 (1H, d), |
| | 7.51-7.37 (6H, m), 7.16 (1H, m), 7.06 (1H, d) |
| 262 | δ = 8.98-8.93 (2H, m), 8.23 (1H, d), 8.11 (1H, d), |
| | 7.96 (2H, s), 7.77-7.47 (6H, m) |
| 266 | δ = 7.96-7.86 (4H, m), 7.77 (1H, d), 7.52 (1H, d), |
| | 7.33 (1H, d), 7.16 (1H, m), 7.06 (1H, m), 6.94 |
| | (1H, d) |
| 267 | δ = 8.29 (1H, s), 8.15 (1H, s), 8.07-8.05 (2H, d), |
| | 7.67 (1H, s), 7.48-7.43 (2H, m), 7.33 (1H, d), 7.06 |
| | (1H, d), 6.94 (1H, s) |
| 281 | δ = 8.03 (1H, d), 7.82 (1H, d), 7.72-7.55 (5H, m), |
| | 7.37-7.30 (4H, m) |
| 288 | δ = 8.33 (1H, s), 7.80-7.62 (5H, m), 7.43-7.37 (2H, |
| | m), 7.11-7.06 (2H, m) |
| 291 | δ = 7.58-7.54 (4H, m), 7.37-7.33 (3H, m), 7.16 (1H, |
| | s), 6.83 (1H, s) |

TABLE 4

| Compound | FD-MS |
|---|---|
| 001 | m/z = 836.64 |
| | (C₆₁D₄₅N = 837.31) |
| 003 | m/z = 756.58 |
| | (C₅₅D₄₁N = 757.19) |
| 005 | m/z = 800.64 |
| | (C₅₈D₄₅N = 801.28) |
| 007 | m/z = 768.55 |
| | (C₅₅D₃₉NO = 769.16) |
| 011 | m/z = 756.58 |
| | (C₅₅D₄₁N = 757.19) |
| 013 | m/z = 800.64 |
| | (C₅₈D₄₅N = 801.28) |
| 023 | m/z = 768.55 |
| | (C₅₅D₃₉NO = 769.16) |
| 025 | m/z = 812.60 |
| | (C₅₈D₄₃NO = 813.25) |
| 026 | m/z = 756.50 |
| | (C₅₃D₃₇NO = 757.17) |
| 027 | m/z = 864.58 |
| | (C₆₁D₄₃NS = 865.34) |
| 030 | m/z = 784.53 |
| | (C₅₅D₃₉NS = 785.22) |
| 087 | m/z = 813.49 |
| | (C₆₁H₂₃D₂₂N = 814.17) |
| 088 | m/z = 829.51 |
| | (C₆₁H₁₉D₂₅N₂ = 830.19) |
| 089 | m/z = 737.46 |
| | (C₅₅H₁₉D₂₂N = 738.07) |
| 090 | m/z = 761.46 |
| | (C₅₇H₁₉D₂₂N = 762.10) |
| 091 | m/z = 781.52 |
| | (C₅₈H₁₉D₂₆N = 782.16) |
| 093 | m/z = 749.43 |
| | (C₅₅H₁₉D₂₀NO = 750.05) |
| 098 | m/z = 833.54 |
| | (C₆₂H₁₅D₃₀N = 834.23) |
| 099 | m/z = 777.49 |
| | (C₅₈H₂₃D₂₂N = 778.14) |
| 109 | m/z = 751.44 |
| | (C₅₅H₁₇D₂₂NO = 752.06) |
| 111 | m/z = 795.50 |
| | (C₅₈H₁₇D₂₆NO = 796.15) |
| 112 | m/z = 739.39 |
| | (C₅₃H₁₇D₂₀NS = 740.07) |
| 116 | m/z = 767.42 |
| | (C₅₅H₁₇D₂₂NS = 768.12) |
| 173 | m/z = 814.50 |
| | (C₆₁H₂₂D₂₃N = 815.18) |
| 175 | m/z = 734.44 |
| | (C₅₅H₂₂D₁₉N = 735.06) |
| 176 | m/z = 758.44 |
| | (C₅₇H₂₂D₁₉N = 759.08) |
| 177 | m/z = 774.47 |
| | (C₅₈H₂₆D₁₉N = 775.12) |
| 179 | m/z = 748.42 |
| | (C₅₅H₂₀D₁₉N = 749.04) |
| 182 | m/z = 736.37 |
| | (C₅₃H₂₀D₁₇NS = 737.05) |
| 184 | m/z = 818.45 |
| | (C₆₂H₃₀D₁₅N = 819.14) |
| 185 | m/z = 778.50 |
| | (C₅₈H₂₂D₂₃N = 779.15) |
| 189 | m/z = 792.48 |
| | (C₅₈H₂₀D₂₃NO = 793.13) |
| 195 | m/z = 746.41 |
| | (C₅₅H₂₂D₁₇NO = 747.03) |
| 199 | m/z = 838.42 |
| | (C₆₁H₂₆D₁₇NS = 839.19) |
| 205 | m/z = 720.39 |
| | (C₅₃H₂₀D₁₇NO = 720.99) |
| 259 | m/z = 827.58 |
| | (C₆₁H₉D₃₆N = 828.26) |
| 261 | m/z = 744.51 |
| | (C₅₅H₁₂D₂₉N = 745.12) |
| 262 | m/z = 768.51 |
| | (C₅₇H₁₂D₂₉N = 769.14) |
| 266 | m/z = 790.57 |
| | (C₅₈H₁₀D₃₅N = 791.22) |
| 267 | m/z = 842.60 |
| | (C₆₂H₁₀D₃₇N = 843.29) |
| 281 | m/z = 757.48 |
| | (C₅₅H₁₁D₂₈NO = 758.09) |
| 288 | m/z = 774.46 |
| | (C₅₅H₁₀D₂₉NS = 775.16) |
| 291 | m/z = 731.46 |
| | (C₅₃H₉D₂₈NO = 732.06) |

### Experimental Example 1

### (1) Manufacture of Organic Light Emitting Device

### Comparative Example 1

A glass substrate, in which ITO was thinly coated to have a thickness of 1,500 Å, was ultrasonically washed with distilled water. When the washing with distilled water was finished, the glass substrate was ultrasonically washed with a solvent such as acetone, methanol, and isopropyl alcohol, dried and then was subjected to UVO treatment for 5 minutes using UV in a UV cleaning machine. Thereafter, the substrate was transferred to a plasma washing machine (PT), and then was subjected to plasma treatment in a vacuum state for an ITO work function and in order to remove a residual film, and was transferred to a thermal deposition apparatus for organic deposition.

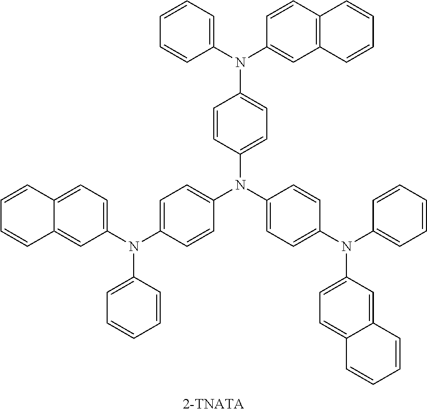 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1]

Subsequently, air in the chamber was evacuated until the degree of vacuum in the chamber reached 10⁻⁶ torr, and then a hole injection layer having a thickness of 600 Å was deposited on the ITO substrate by applying current to the cell to evaporate 2-TNATA. A hole transport layer having a thickness of 300 Å was deposited on the hole injection layer by placing the following N,N′-bis(α-naphthyl)-N,N′-diphenyl-4,4′-diamine (NPB) in another cell in the vacuum deposition apparatus and applying current to the cell to evaporate NPB.

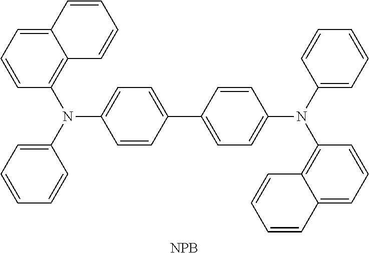 [SMILES: c1ccc(N(c2ccc(-c3ccc(N(c4ccccc4)c4cccc5ccccc45)cc3)cc2)c2cccc3ccccc23)cc1 ; "NPB"]

A light emitting layer was thermally vacuum deposited thereon as follows. The light emitting layer was deposited by depositing a compound of 9-[4-(4,6-diphenyl-1,3,5-triazin-2-yl)phenyl]-9′-phenyl-3,3′-bi-9H-carbazole as a host to have a thickness of 400 Å and doping the deposited layer with a green phosphorescent dopant Ir(ppy)₃ at 7%. Thereafter, bathocuproine (BCP) as a hole blocking layer was deposited to have a thickness of 60 Å, and Alq₃ as an electron transport layer was deposited to have a thickness of 200 Å thereon. Finally, an organic light emitting device was manufactured by depositing lithium fluoride (LiF) to have a thickness of 10 Å on the electron transport layer to form an electron injection layer, and then depositing an aluminum (Al) negative electrode to have a thickness of 1,200 Å on the electron injection layer to form a negative electrode.

Meanwhile, all the organic compounds required for manufacturing an organic light emitting device were subjected to vacuum sublimed purification under 10⁻⁸ to 10⁻⁶ torr for each material, and used for the manufacture of the organic light emitting device.

### Examples 1 to 43 and Comparative Examples 2 to 13

Organic light emitting devices of Examples 1 to 34 and Comparative Examples 2 to 13 were manufactured in the same manner as in the manufacturing method of the organic light emitting device of Comparative Example 1, except that the compounds in the following Table 5 were used instead of Compound NPB used during the forming of the hole transport layer in Comparative Example 1.

### (2) Driving Voltage and Light Emitting Efficiency of Organic Light Emitting Device

For the organic light emitting devices of Examples 1 to 43 and Comparative Examples 1 to 13 manufactured as above, electroluminescent light emission (EL) characteristics were measured using M7000 manufactured by McScience Inc., and with the measurement results, a service life T₉₀ (unit: h, hour), which was the time when the luminance became 90% compared to the initial luminance when the standard luminance was 6,000 cd/m², was measured using a service life measurement apparatus (M6000) manufactured by McScience Inc.

The characteristics of the organic light emitting devices of the present invention shown with the measurement results are shown in the following Table 5.

TABLE 5

| | | Driving | | Service |
|---|---|---|---|---|
| | | voltage | Efficiency | life |
| | Compound | (V) | (cd/A) | (T₉₀) |
| Comparative | NPB | 4.61 | 99.47 | 125 |
| Example 1 | | | | |
| Comparative | Comparative | 3.91 | 138.6 | 231 |
| Example 2 | Compound A | | | |
| Comparative | Comparative | 4.48 | 132.8 | 222 |
| Example 3 | Compound B | | | |
| Comparative | Comparative | 3.62 | 137.5 | 238 |
| Example 4 | Compound C | | | |
| Comparative | Comparative | 3.94 | 138.5 | 242 |
| Example 5 | Compound D | | | |
| Comparative | Comparative | 4.52 | 136.7 | 241 |
| Example 6 | Compound E | | | |
| Comparative | Comparative | 4.33 | 133.9 | 238 |
| Example 7 | Compound F | | | |
| Comparative | Comparative | 3.89 | 131.1 | 236 |
| Example 8 | Compound G | | | |
| Comparative | Comparative | 4.66 | 135.4 | 233 |
| Example 9 | Compound H | | | |
| Comparative | Comparative | 4.40 | 131.6 | 242 |
| Example 10 | Compound I | | | |
| Comparative | Comparative | 3.98 | 135.5 | 242 |
| Example 11 | Compound J | | | |
| Comparative | Comparative | 4.37 | 132.4 | 243 |
| Example 12 | Compound K | | | |
| Comparative | Comparative | 4.32 | 131.3 | 241 |
| Example 13 | Compound L | | | |
| Example 1 | Compound 1 | 3.73 | 142.6 | 247 |
| Example 2 | Compound 3 | 3.63 | 145.7 | 258 |
| Example 3 | Compound 5 | 4.24 | 133.8 | 253 |
| Example 4 | Compound 7 | 3.98 | 135.7 | 259 |
| Example 5 | Compound 11 | 4.05 | 135.4 | 246 |
| Example 6 | Compound 13 | 3.40 | 139.9 | 259 |
| Example 7 | Compound 23 | 3.99 | 137.1 | 251 |
| Example 8 | Compound 25 | 4.14 | 138.9 | 244 |
| Example 9 | Compound 26 | 3.93 | 138.6 | 248 |
| Example 10 | Compound 27 | 3.55 | 141.6 | 250 |
| Example 11 | Compound 30 | 4.23 | 130.8 | 247 |
| Example 12 | Compound 87 | 3.94 | 134.5 | 244 |
| Example 13 | Compound 88 | 3.98 | 139.6 | 250 |
| Example 14 | Compound 89 | 3.61 | 143.4 | 257 |
| Example 15 | Compound 90 | 4.28 | 133.8 | 248 |
| Example 16 | Compound 91 | 3.84 | 133.7 | 246 |
| Example 17 | Compound 93 | 4.11 | 130.6 | 248 |
| Example 18 | Compound 98 | 3.66 | 138.4 | 246 |
| Example 19 | Compound 99 | 3.71 | 141.6 | 255 |
| Example 20 | Compound 109 | 3.81 | 142.5 | 254 |
| Example 21 | Compound 111 | 3.96 | 135.5 | 249 |
| Example 22 | Compound 112 | 3.71 | 138.4 | 252 |
| Example 23 | Compound 116 | 3.62 | 144.3 | 249 |
| Example 24 | Compound 173 | 4.20 | 141.7 | 250 |
| Example 25 | Compound 175 | 3.67 | 144.2 | 259 |
| Example 26 | Compound 176 | 3.80 | 139.8 | 245 |
| Example 27 | Compound 177 | 3.75 | 136.9 | 258 |
| Example 28 | Compound 179 | 3.91 | 137.7 | 248 |
| Example 29 | Compound 182 | 4.49 | 140.1 | 247 |
| Example 30 | Compound 184 | 4.33 | 138.5 | 249 |
| Example 31 | Compound 185 | 3.69 | 142.27 | 252 |
| Example 32 | Compound 189 | 4.24 | 138.9 | 248 |
| Example 33 | Compound 195 | 4.05 | 131.8 | 250 |
| Example 34 | Compound 199 | 3.81 | 135.5 | 252 |
| Example 35 | Compound 205 | 3.99 | 131.2 | 248 |
| Example 36 | Compound 259 | 4.03 | 134.6 | 246 |
| Example 37 | Compound 261 | 3.65 | 147.5 | 261 |
| Example 38 | Compound 262 | 3.87 | 148.4 | 253 |
| Example 39 | Compound 266 | 3.52 | 130.9 | 246 |
| Example 40 | Compound 267 | 4.13 | 138.7 | 261 |
| Example 41 | Compound 281 | 3.91 | 133.5 | 249 |
| Example 42 | Compound 288 | 3.81 | 131.6 | 250 |
| Example 43 | Compound 291 | 4.10 | 140.1 | 249 |

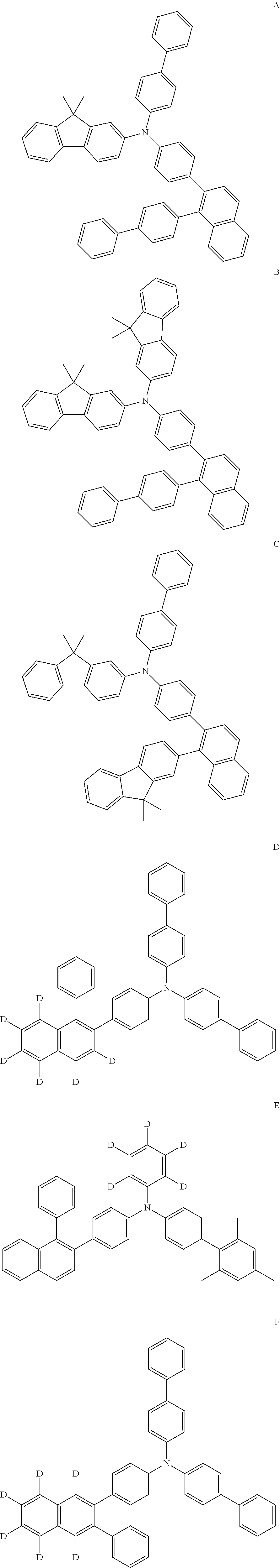

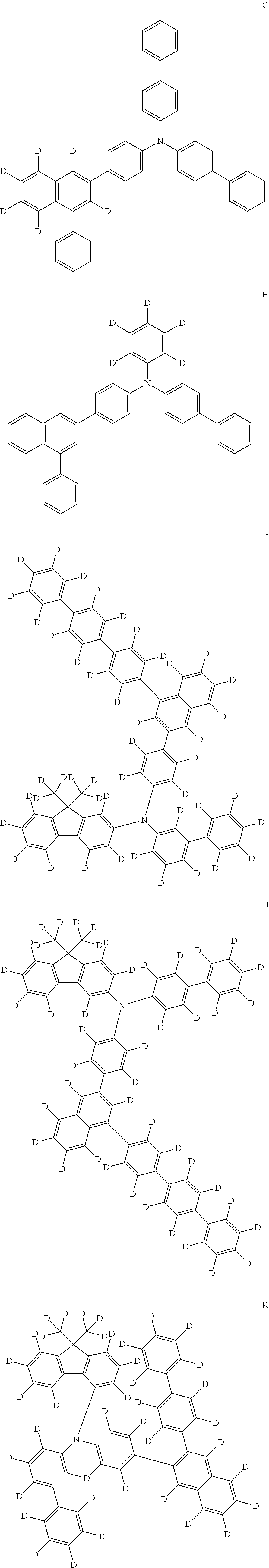

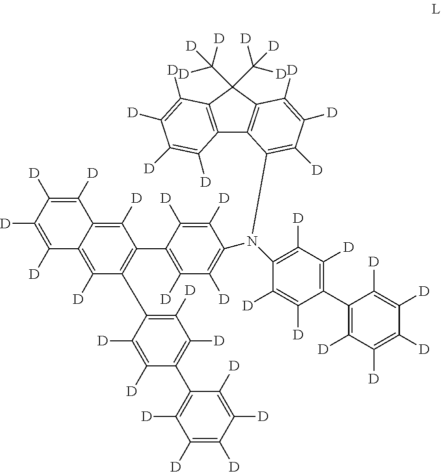 [SMILES: [2H]c1c([2H])c([2H])c(-c2c([2H])c([2H])c(-c3c(-c4c([2H])c([2H])c(N(c5c([2H])c([2H])c(-c6c([2H])c([2H])c([2H])c([2H])c6[2H])c([2H])c5[2H])c5c([2H])c([2H])c([2H])c6c5-c5c([2H])c([2H])c([2H])c([2H])c5C6(C([2H])([2H])[2H])C([2H])([2H])[2H])c([2H])c4[2H])c([2H])c4c([2H])c([2H])c([2H])c([2H])c4c3[2H])c([2H])c2[2H])c([2H])c1[2H]]

Although the compounds of Example 2 and Comparative Example 2 have the same compound skeleton, there is a difference in whether or not the compound is substituted with deuterium, and when the driving voltages, efficiencies and service lives are compared, it can be seen that Example 2 has low driving voltage, high efficiency, and long service life compared to Comparative Example 1.

Similarly between Example 3 and Comparative Example 3 and between Example 6 and Comparative Example 4, there is only a difference in whether or not the compound is substituted with deuterium, and it can be confirmed that Example 3 and Example 6 are all excellent in terms of driving voltage, efficiency and service life, and in particular, the service life was improved by about 14% and 9%, respectively.

In the case of Comparative Examples 5 to 9, a portion of the compound is substituted with deuterium, but the compound does not include a fluorene structure, and the naphthalene substitution position is different from that of the present invention, so it can be seen that Comparative Examples 5 to 9 have a shorter service life than that of the Examples.

Although Comparative Compounds I to L used in Comparative Examples 10 to 13 include deuterium, the compounds are compounds in which the substituent position of naphthalene is different from that of the compound of the present invention, and it can be confirmed that the service life of Comparative Examples 10 to 13 is lower than that of the Examples.

### Experimental Example 2

### (1) Manufacture of Organic Light Emitting Device

### Examples 44 to 86 and Comparative Examples 14 to 25

A glass substrate thinly coated with indium tin oxide (ITO) having a thickness of 1,500 Å was ultrasonically washed with distilled water. When the washing with distilled water was finished, the glass substrate was ultrasonically washed with a solvent such as acetone, methanol, and isopropyl alcohol, dried and then was subjected to UVO treatment for 5 minutes using UV in a UV cleaning machine. Thereafter, the substrate was transferred to a plasma washing machine (PT), and then was subjected to plasma treatment in a vacuum state in order to increase an ITO work function and remove a residual film, and was transferred to a thermal deposition apparatus for organic deposition.

Subsequently, air in the chamber was evacuated until the degree of vacuum in the chamber reached 10⁻⁶ torr, and then a hole injection layer having a thickness of 600 Å was deposited on the ITO substrate by applying current to the cell to evaporate 2-TNATA. A hole transport layer having a thickness of 300 Å was deposited on the hole injection layer by placing the following N,N′-bis(α-naphthyl)-N,N′-diphenyl-4,4′-diamine (NPB) in another cell in the vacuum deposition apparatus and applying current to the cell to evaporate NPB. Thereafter, a compound shown in the following Table 6 was deposited to have a thickness of 100 Å as a hole auxiliary layer.

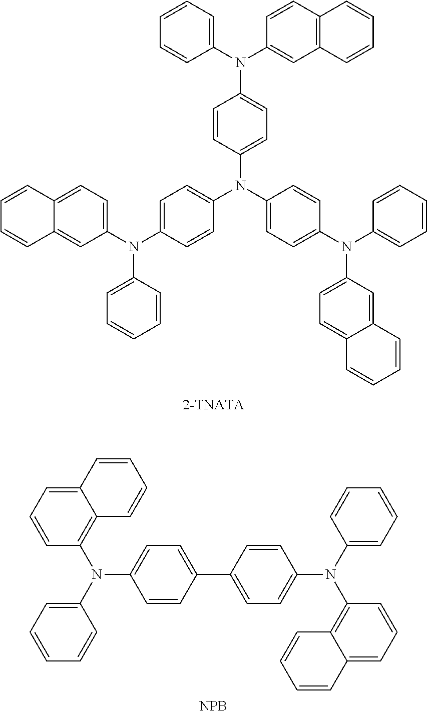 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1 ; c1ccc(N(c2ccc(-c3ccc(N(c4ccccc4)c4cccc5ccccc45)cc3)cc2)c2cccc3ccccc23)cc1 ; "NPB"]

A light emitting layer was thermally vacuum deposited thereon as follows. The light emitting layer was deposited by depositing a compound of 9-[4-(4,6-diphenyl-1,3,5-triazin-2-yl)phenyl]-9′-phenyl-3,3′-bi-9H-carbazole as a host to have a thickness of 400 Å and doping the deposited layer with [Ir(ppy)₃] as a green phosphorescent dopant by 7% of the deposited thickness of the light emitting layer. Thereafter, bathocuproine (BCP) was deposited as a hole blocking layer to have a thickness of 60 Å, and Alq₃ was deposited as an electron transport layer to have a thickness of 200 Å thereon. Finally, an organic light emitting device was manufactured by depositing lithium fluoride (LiF) to have a thickness of 10 Å on the electron transport layer to form an electron injection layer, and then depositing an aluminum (Al) negative electrode to have a thickness of 1,200 Å on the electron injection layer to form a negative electrode.

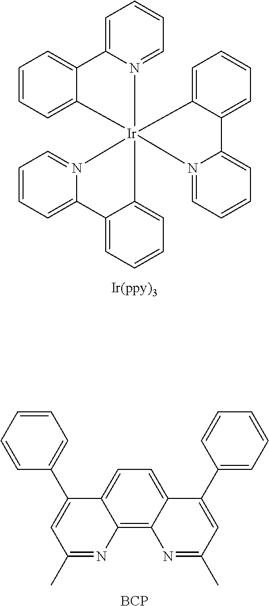

Meanwhile, all the organic compounds required for manufacturing an organic light emitting device were subjected to vacuum sublimed purification under 10⁻⁸ to 10⁻⁶ torr for each material, and used for the manufacture of the organic light emitting device.

### (2) Driving Voltage and Light Emitting Efficiency of Organic Light Emitting Device

For the organic light emitting devices of Examples 44 to 86 and Comparative Examples 14 to 25 manufactured as above, electroluminescent light emission (EL) characteristics were measured using M7000 manufactured by McScience Inc., and with the measurement results, a service life T₉₀ (unit: h, hour), which was the time when the luminance became 90% compared to the initial luminance when the standard luminance was 6,000 cd/m², was measured using a service life measurement apparatus (M6000) manufactured by McScience Inc.

The characteristics of the organic light emitting devices of the present invention shown with the measurement results are shown in the following Table 6.

TABLE 6

| | | Driving | | Service |
|---|---|---|---|---|
| | | voltage | Efficiency | life |
| | Compound | (V) | (cd/A) | (T₉₀) |
| Comparative | Comparative | 5.77 | 6.9 | 52 |
| Example 14 | Compound A | | | |
| Comparative | Comparative | 5.23 | 7.3 | 56 |
| Example 15 | Compound B | | | |
| Comparative | Comparative | 5.79 | 6.9 | 49 |
| Example 16 | Compound C | | | |
| Comparative | Comparative | 5.76 | 7.2 | 50 |
| Example 17 | Compound D | | | |
| Comparative | Comparative | 5.66 | 7.0 | 52 |
| Example 18 | Compound E | | | |
| Comparative | Comparative | 5.86 | 6.9 | 56 |
| Example 19 | Compound F | | | |
| Comparative | Comparative | 5.95 | 7.0 | 55 |
| Example 20 | Compound G | | | |
| Comparative | Comparative | 5.90 | 7.1 | 57 |
| Example 21 | Compound H | | | |
| Comparative | Comparative | 5.69 | 6.8 | 55 |
| Example 22 | Compound I | | | |
| Comparative | Comparative | 5.74 | 6.7 | 53 |
| Example 23 | Compound J | | | |
| Comparative | Comparative | 5.79 | 6.5 | 57 |
| Example 24 | Compound K | | | |
| Comparative | Comparative | 5.77 | 6.8 | 56 |
| Example 25 | Compound L | | | |
| Example 44 | Compound 1 | 5.33 | 7.5 | 70 |
| Example 45 | Compound 3 | 5.32 | 8.2 | 76 |
| Example 46 | Compound 5 | 5.24 | 8.0 | 74 |
| Example 47 | Compound 7 | 5.37 | 7.9 | 75 |
| Example 48 | Compound 11 | 5.50 | 7.4 | 68 |
| Example 49 | Compound 13 | 5.46 | 7.3 | 71 |
| Example 50 | Compound 23 | 5.61 | 7.8 | 69 |
| Example 51 | Compound 25 | 5.57 | 7.7 | 70 |
| Example 52 | Compound 26 | 5.30 | 7.9 | 74 |
| Example 53 | Compound 27 | 5.27 | 8.0 | 75 |
| Example 54 | Compound 30 | 5.35 | 8.1 | 69 |
| Example 55 | Compound 87 | 5.41 | 7.6 | 69 |
| Example 56 | Compound 88 | 5.40 | 7.9 | 70 |
| Example 57 | Compound 89 | 5.27 | 7.8 | 75 |
| Example 58 | Compound 90 | 5.33 | 7.8 | 75 |
| Example 59 | Compound 91 | 5.39 | 7.7 | 74 |
| Example 60 | Compound 93 | 5.29 | 7.9 | 72 |
| Example 61 | Compound 98 | 5.44 | 8.0 | 73 |
| Example 62 | Compound 99 | 5.30 | 7.9 | 75 |
| Example 63 | Compound 109 | 5.47 | 7.9 | 69 |
| Example 64 | Compound 111 | 5.51 | 8.1 | 71 |
| Example 65 | Compound 112 | 5.23 | 7.6 | 73 |
| Example 66 | Compound 116 | 5.37 | 7.7 | 71 |
| Example 67 | Compound 173 | 5.29 | 7.5 | 74 |
| Example 68 | Compound 175 | 5.30 | 7.5 | 72 |
| Example 69 | Compound 176 | 5.48 | 7.6 | 73 |
| Example 70 | Compound 177 | 5.31 | 7.9 | 76 |
| Example 71 | Compound 179 | 5.38 | 7.8 | 69 |
| Example 72 | Compound 182 | 5.47 | 8.1 | 69 |
| Example 73 | Compound 184 | 5.43 | 7.9 | 71 |
| Example 74 | Compound 185 | 5.28 | 7.8 | 75 |
| Example 75 | Compound 189 | 5.31 | 7.6 | 73 |
| Example 76 | Compound 195 | 5.34 | 7.8 | 71 |
| Example 77 | Compound 199 | 5.26 | 8.0 | 74 |
| Example 78 | Compound 205 | 5.39 | 7.5 | 75 |
| Example 79 | Compound 259 | 5.40 | 7.6 | 72 |
| Example 80 | Compound 261 | 5.22 | 7.9 | 76 |
| Example 81 | Compound 262 | 5.24 | 7.9 | 69 |
| Example 82 | Compound 266 | 5.30 | 8.2 | 67 |
| Example 83 | Compound 267 | 5.29 | 7.8 | 73 |
| Example 84 | Compound 281 | 5.32 | 7.5 | 72 |
| Example 85 | Compound 288 | 5.33 | 7.6 | 72 |
| Example 86 | Compound 291 | 5.35 | 7.7 | 70 |

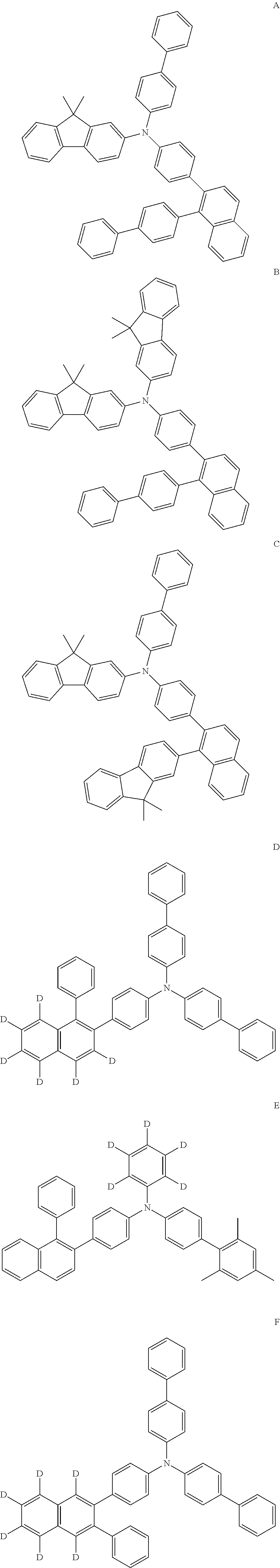

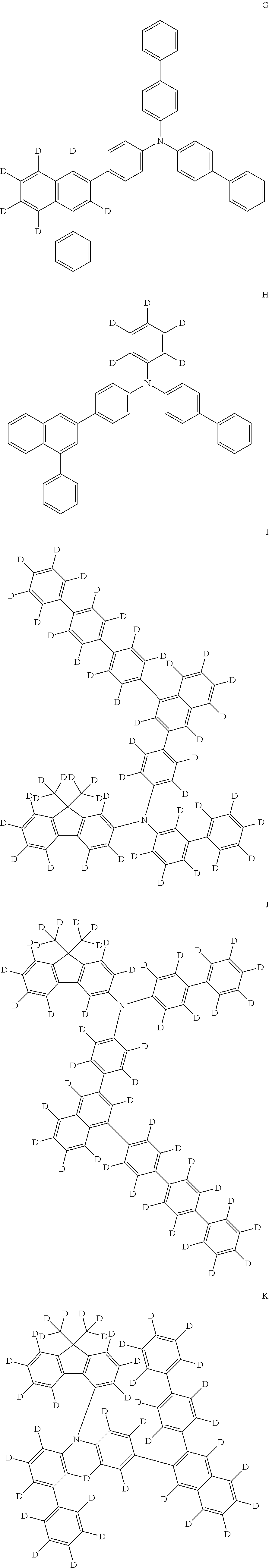

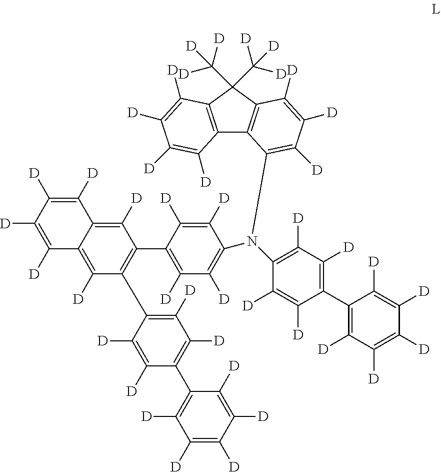 [SMILES: [2H]c1c([2H])c([2H])c(-c2c([2H])c([2H])c(-c3c(-c4c([2H])c([2H])c(N(c5c([2H])c([2H])c(-c6c([2H])c([2H])c([2H])c([2H])c6[2H])c([2H])c5[2H])c5c([2H])c([2H])c([2H])c6c5-c5c([2H])c([2H])c([2H])c([2H])c5C6(C([2H])([2H])[2H])C([2H])([2H])[2H])c([2H])c4[2H])c([2H])c4c([2H])c([2H])c([2H])c([2H])c4c3[2H])c([2H])c2[2H])c([2H])c1[2H]]

When the compound according to the present application is used for an organic light emitting device, the driving voltage of the device could be lowered, the light efficiency of the device could be improved, and the service life characteristics of the device could be improved due to the thermal stability of the compound.

Specifically, although the compounds of Example 45 and Comparative Example 14 have the same compound skeleton, there is a difference in whether or not the compound is substituted with deuterium, and when the driving voltages, efficiencies and service lives are compared, it can be seen that the Example 45 has low driving voltage, high efficiency, and service life improved by about 42% compared to Comparative Example 14.

Similarly between Example 46 and Comparative Example 15 and between Example 49 and Comparative Example 16, there is only a difference in whether or not the compound is substituted with deuterium, and it can be confirmed that Examples 46 and 49 are all excellent in terms of efficiency and service life compared to Comparative Examples 15 and 16, and in particular, the service life in Example 46 and Example 49 was improved by about 32% and 45% compared to Comparative Example 15 and Comparative Example 16, respectively.

In the case of Comparative Examples 17 to 21, a portion of the compound is substituted with deuterium, but the compound does not include a fluorene structure, and the naphthalene substitution position is different from that of the present invention, so it can be seen that Comparative Examples 17 to 21 have a shorter service life than that of the Examples.

Although Comparative Compounds I to L used in Comparative Examples 22 to 25 include deuterium, the compounds are compounds in which the substituent position of naphthalene is different from that of the compound of the present invention, and it can be confirmed that the service life of Comparative Examples 22 to 25 is lower than that of the Examples.

The deuterium content of the compound of the present invention satisfies more than 0% and 100% or less, and since the single bond dissociation energy of carbon and deuterium is higher than the single bond dissociation energy of carbon and hydrogen, the stability of the entire molecules is enhanced, so that it could be confirmed that there was an effect that the service life of the device was improved.

### EXPLANATION OF REFERENCE NUMERALS AND SYMBOLS

100: Substrate
200: Positive electrode
300: Organic material layer
301: Hole injection layer
302: Hole transport layer
303: Hole auxiliary layer
304: Light emitting layer
305: Hole blocking layer
306: Electron transport layer
307: Electron injection layer
400: Negative electrode

1. An amine compound represented by the following Chemical Formula 1 and having a deuterium content of more than 0% and 100% or less: 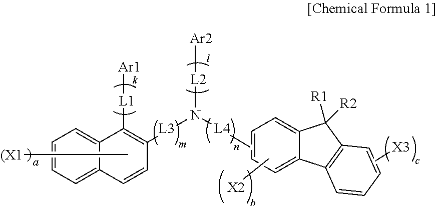
wherein, in Chemical Formula 1,
L1 to L4 are the same as or different from each other, and are each independently a direct bond; a substituted or unsubstituted arylene group having 6 to 60 carbon atoms; or a substituted or unsubstituted heteroarylene group having 2 to 60 carbon atoms,
Ar1 is a phenyl group substituted with deuterium; a substituted or unsubstituted aryl group having 8 to 60 carbon atoms; or a substituted or unsubstituted heteroaryl group having 2 to 60 carbon atoms,
Ar2 is a substituted or unsubstituted aryl group having 6 to 60 carbon atoms; or a substituted or unsubstituted heteroaryl group having 2 to 60 carbon atoms,
R1 and R2 are the same as or different from each other, and are each independently a substituted or unsubstituted alkyl group having 1 to 60 carbon atoms; a substituted or unsubstituted aryl group having 6 to 60 carbon atoms; or a substituted or unsubstituted heteroaryl group having 2 to 60 carbon atoms, or may be bonded to each other to form a substituted or unsubstituted hydrocarbon ring having 3 to 60 carbon atoms; or a substituted or unsubstituted hetero ring having 2 to 60 carbon atoms,
X1 is hydrogen; or deuterium,
X2 and X3 are the same as or different from each other, and are each independently hydrogen; or deuterium; a substituted or unsubstituted aryl group having 6 to 60 carbon atoms; or a substituted or unsubstituted heteroaryl group having 2 to 60 carbon atoms,
k, l, m and n are each an integer from 1 to 3,
a is an integer from 0 to 6,
b is an integer from 0 to 3,
c is an integer from 0 to 4, and
when k, l, m, n, a, b, and c are each 2 or higher, substituents in the parenthesis are the same as or different from each other.

2. The amine compound of claim 1, wherein at least one of the following Structures A and B of Chemical Formula 1 comprises deuterium: 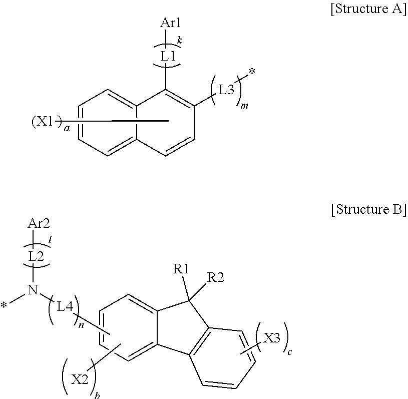
in Structures A and B, the definition of each substituent is the same as that in Chemical Formula 1, and 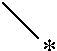 [SMILES: *C]
is a position where Structures A and B are bonded to each other.

3. The amine compound of claim 1, wherein at least one of Ar1 and Ar2 is a phenyl group substituted with deuterium; a biphenyl group substituted with deuterium; a terphenyl group substituted with deuterium; a naphthyl group substituted with deuterium; a phenanthrenyl group substituted with deuterium; a dimethylfluorenyl group substituted with deuterium; a diphenylfluorenyl group substituted with deuterium; a spirobifluorenyl group substituted with deuterium; a 9-phenyl-9H-carbazole group substituted with deuterium; a dibenzofuran group substituted with deuterium; or a dibenzothiophene group substituted with deuterium.

4. The amine compound of claim 1, wherein R1 and R2 are the same as or different from each other, and are each independently a substituted or unsubstituted alkyl group having 1 to 10 carbon atoms; or a substituted or unsubstituted aryl group having 6 to 20 carbon atoms, or are bonded to each other to form a substituted or unsubstituted hydrocarbon ring having 3 to 30 carbon atoms.

5. The amine compound of claim 1, wherein a deuterium content of Chemical Formula 1 is 20% to 100%.

6. The amine compound of claim 1, wherein Chemical Formula 1 is represented by any one of the following compounds:

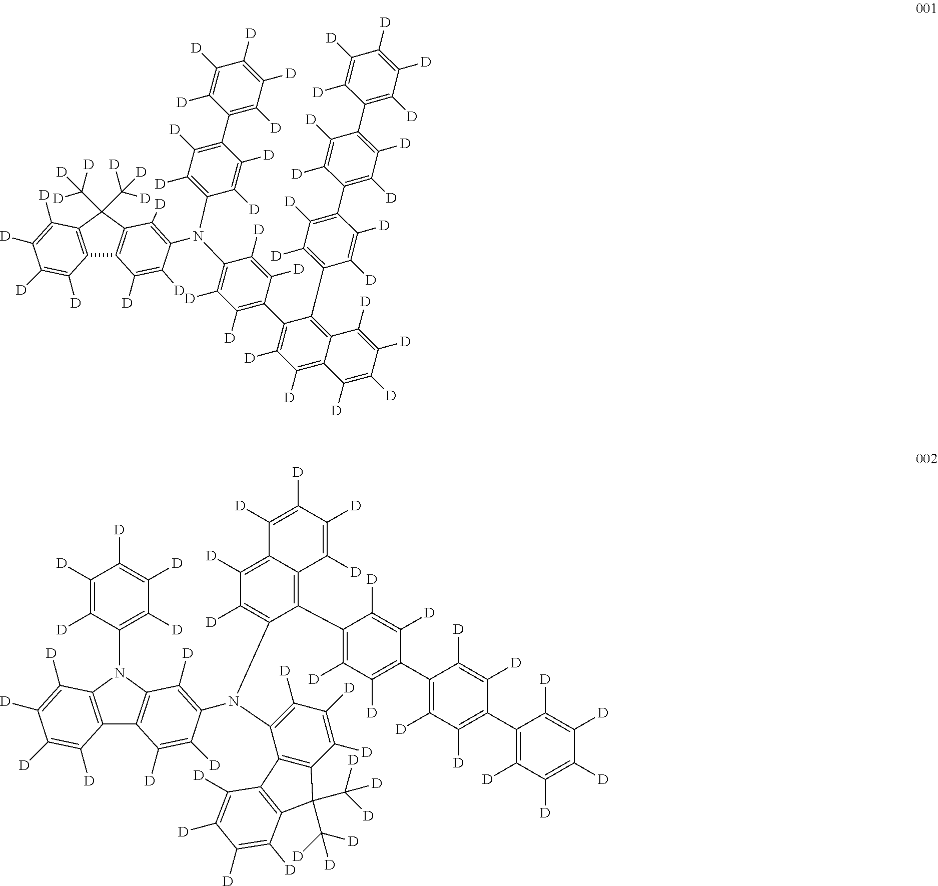

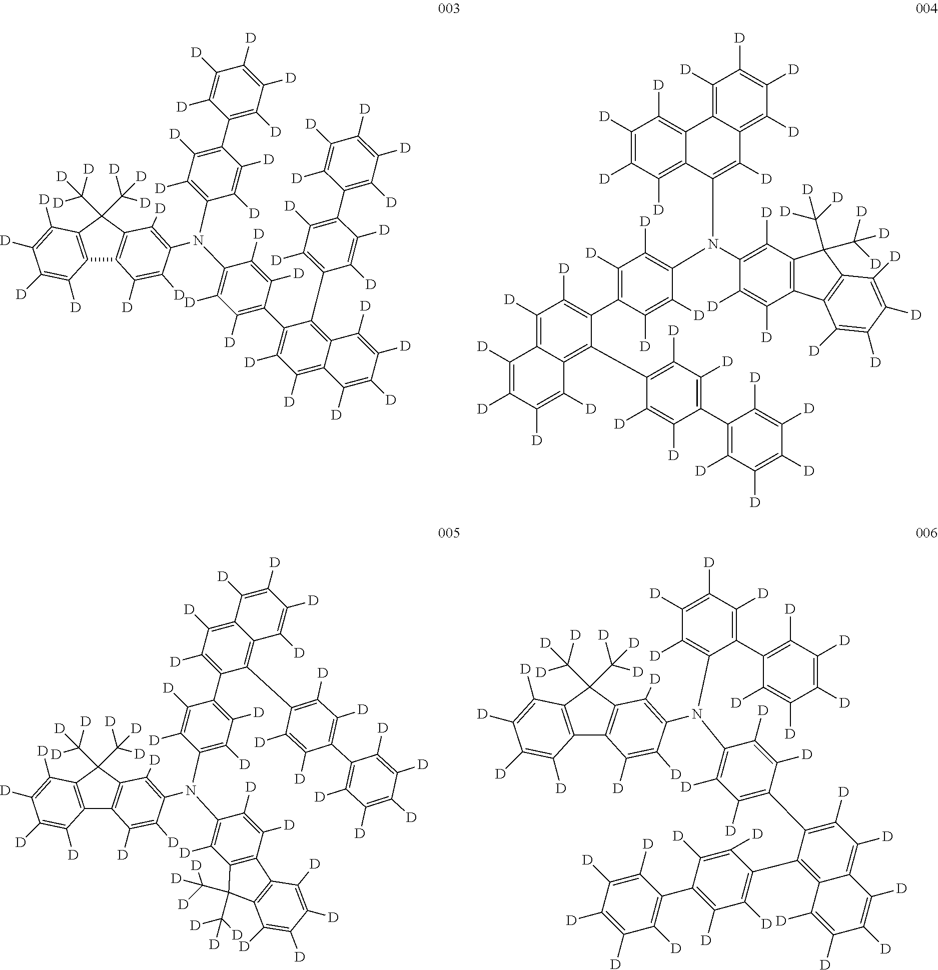

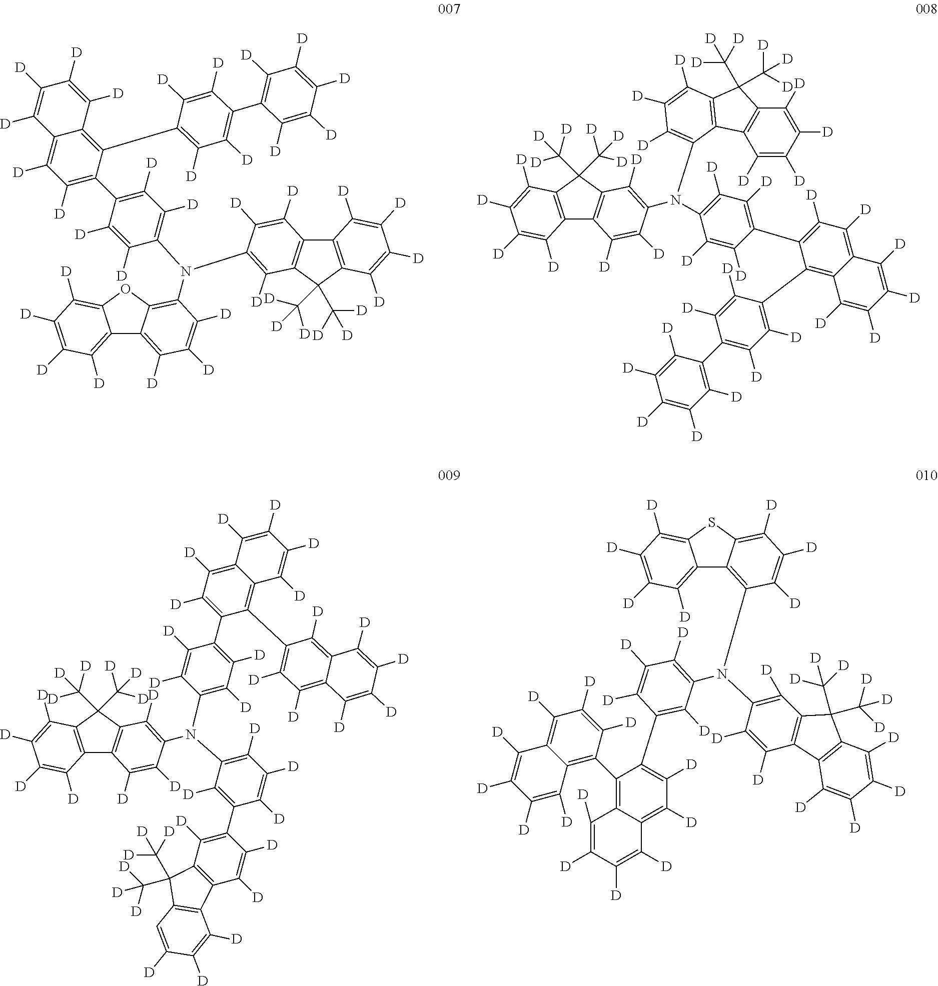

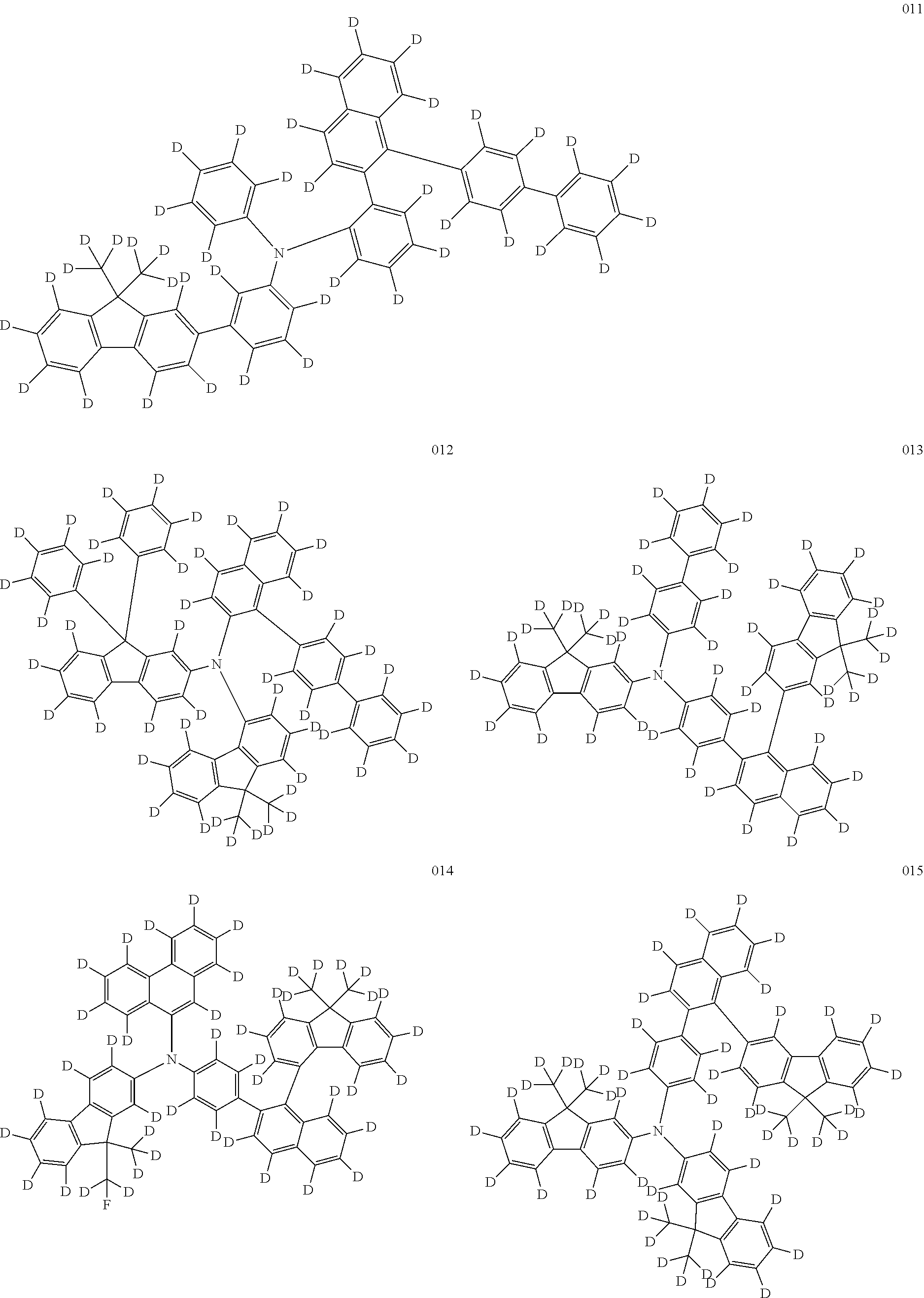

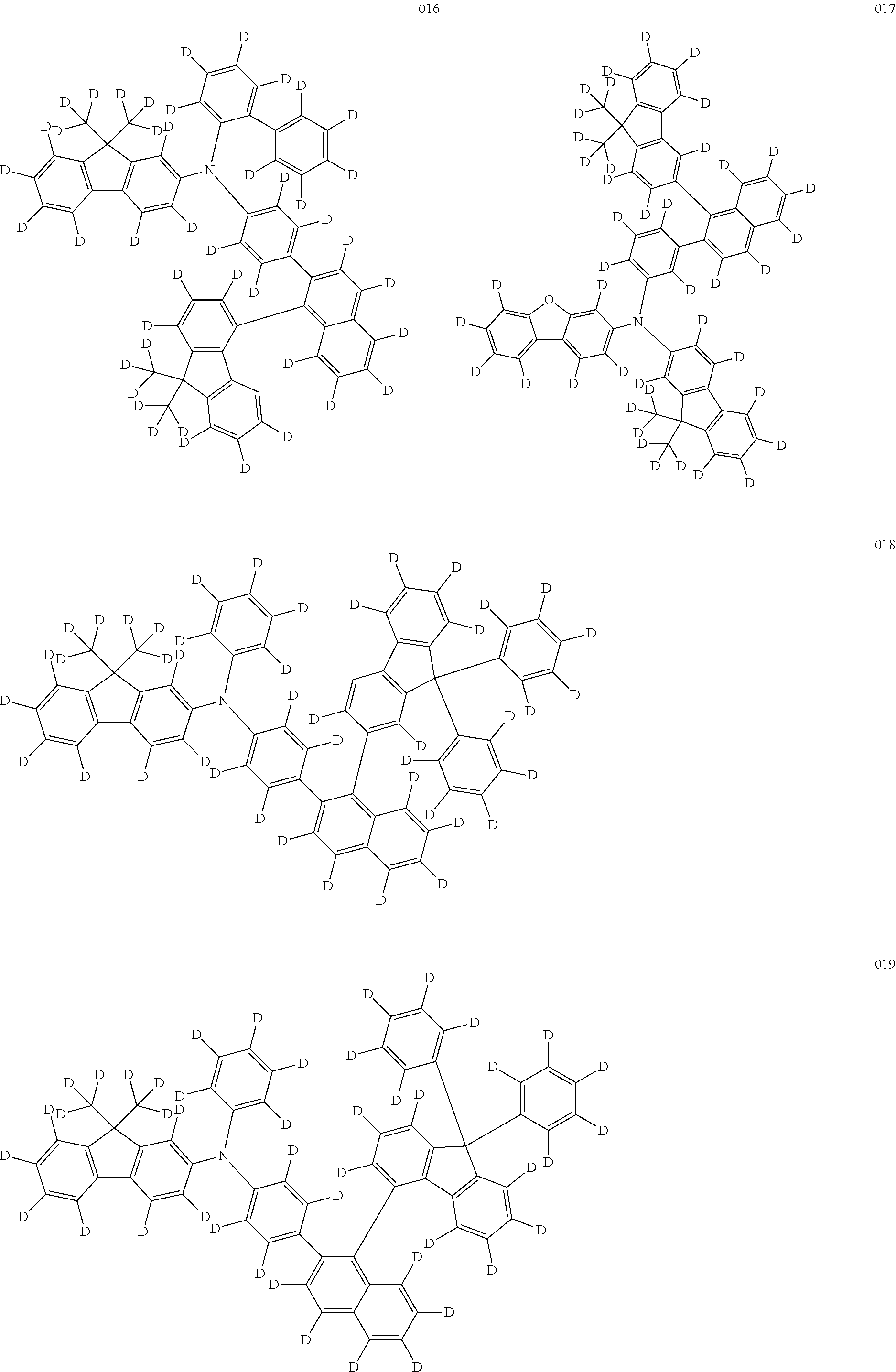

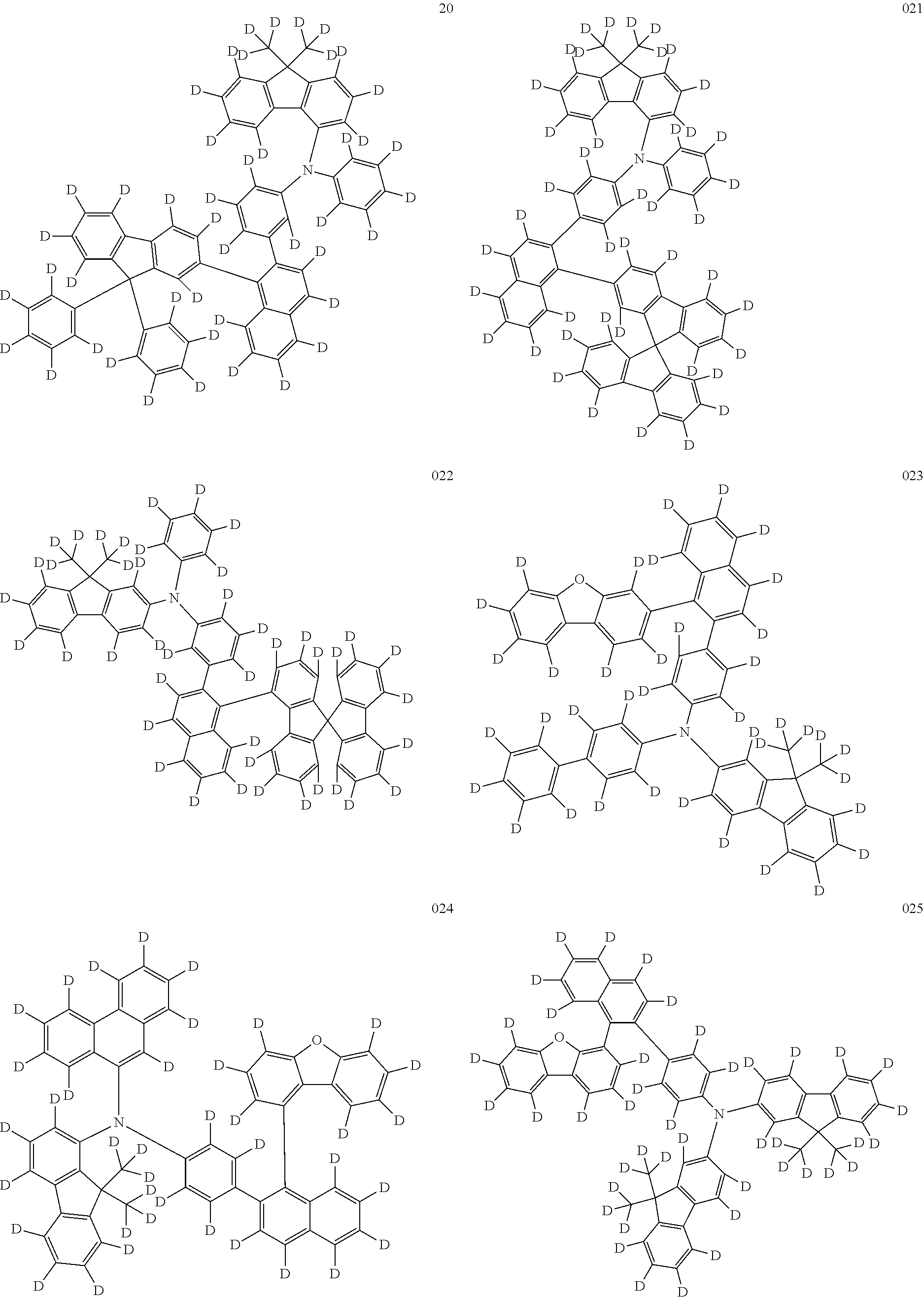

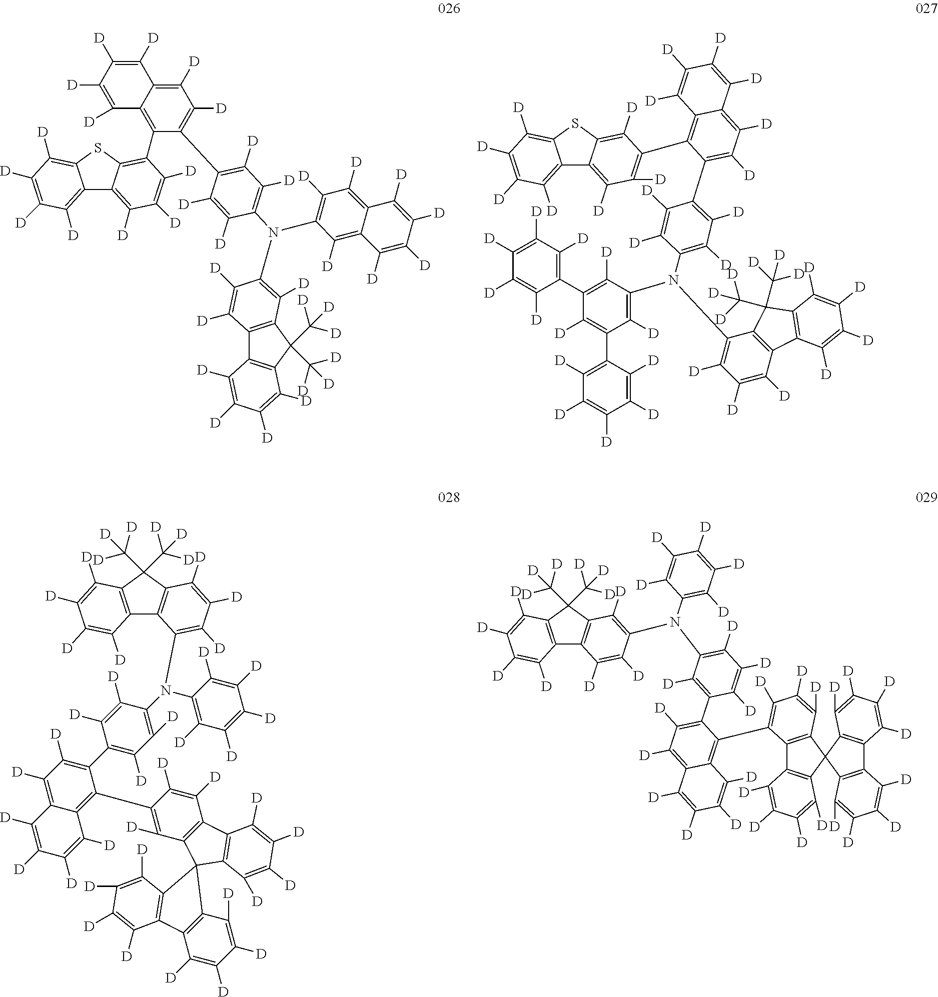

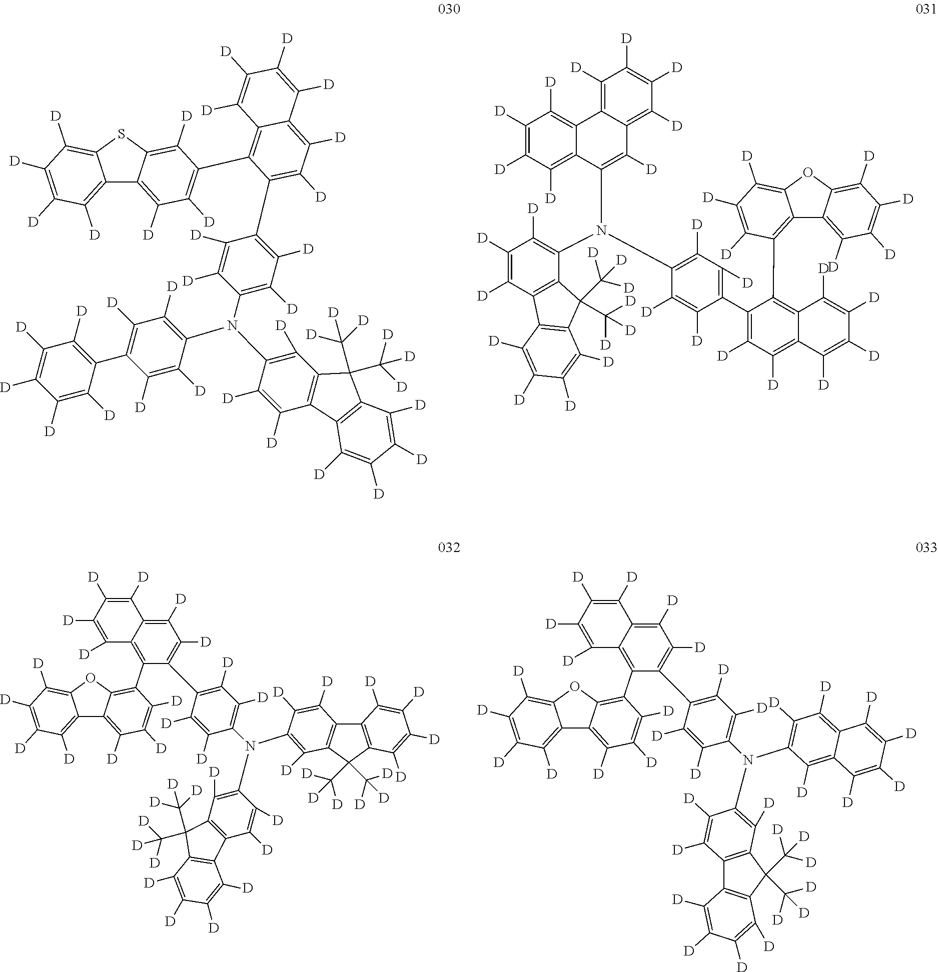

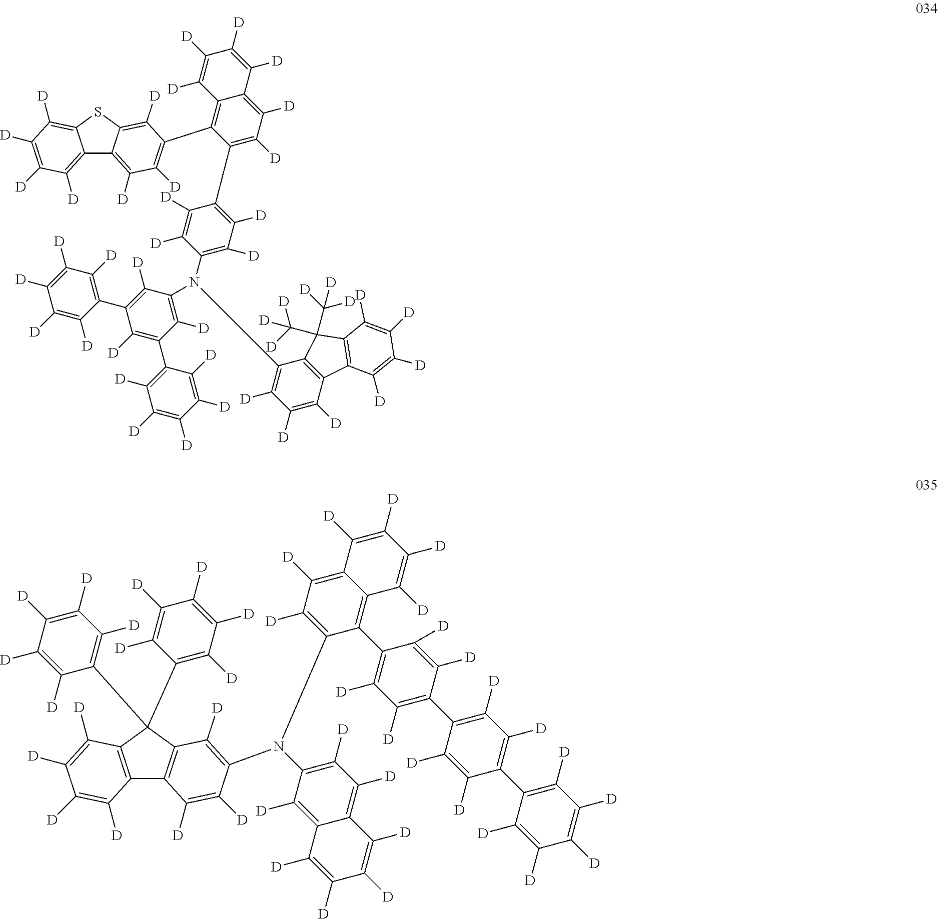

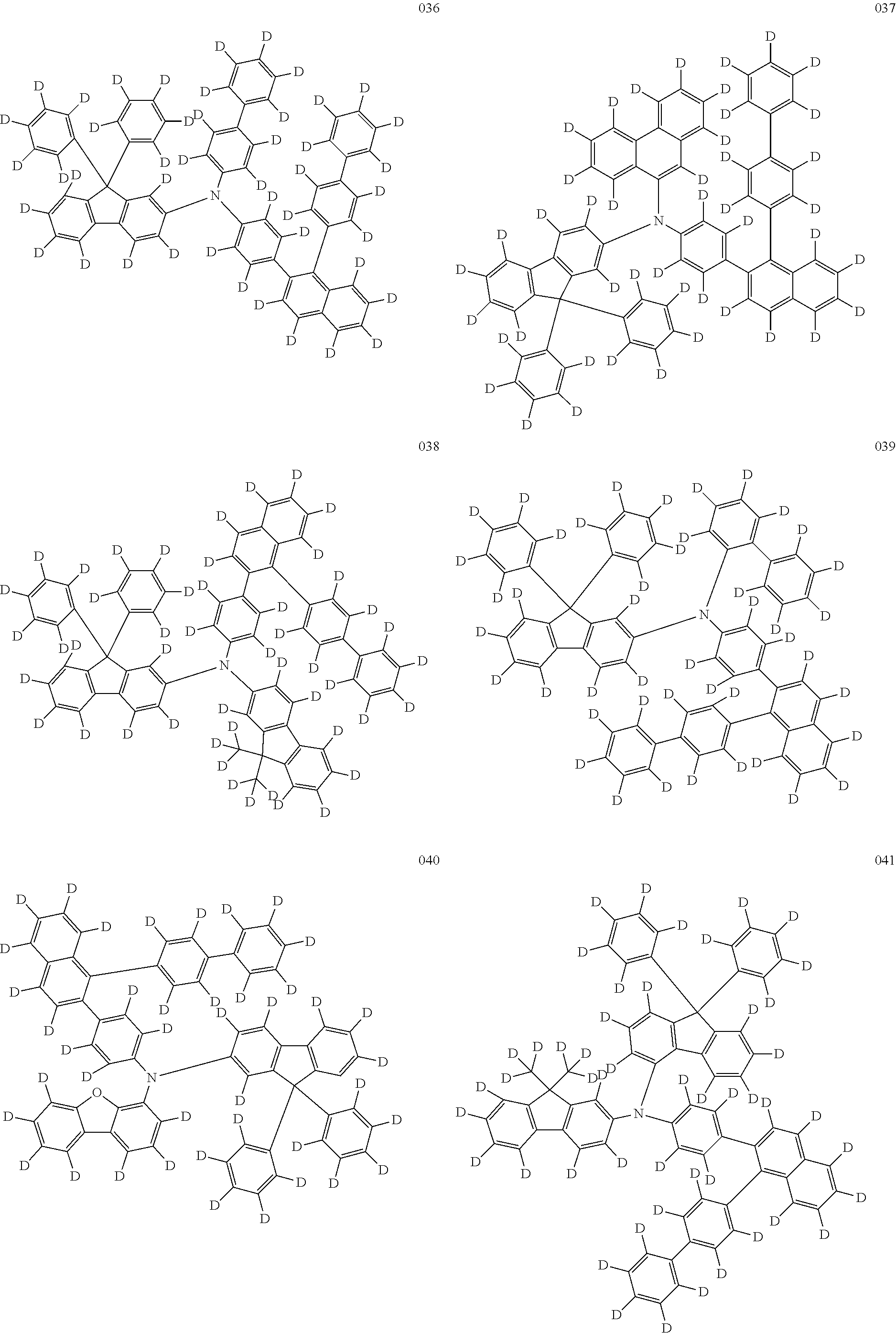

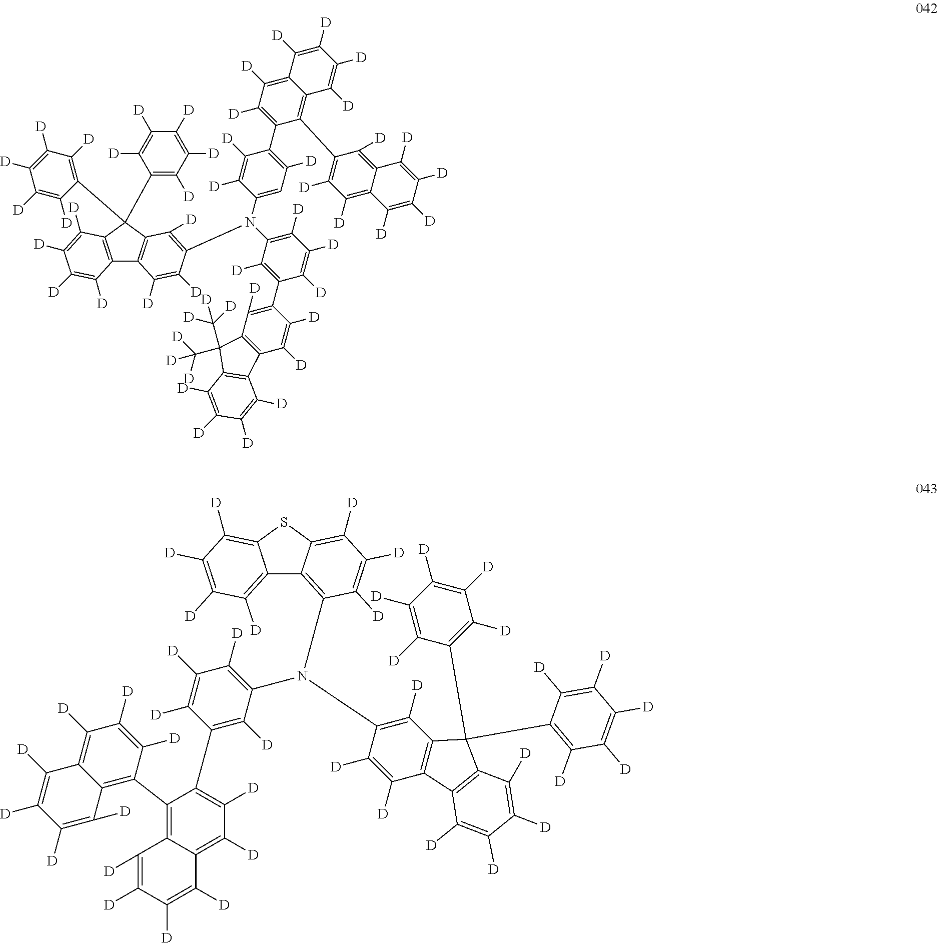

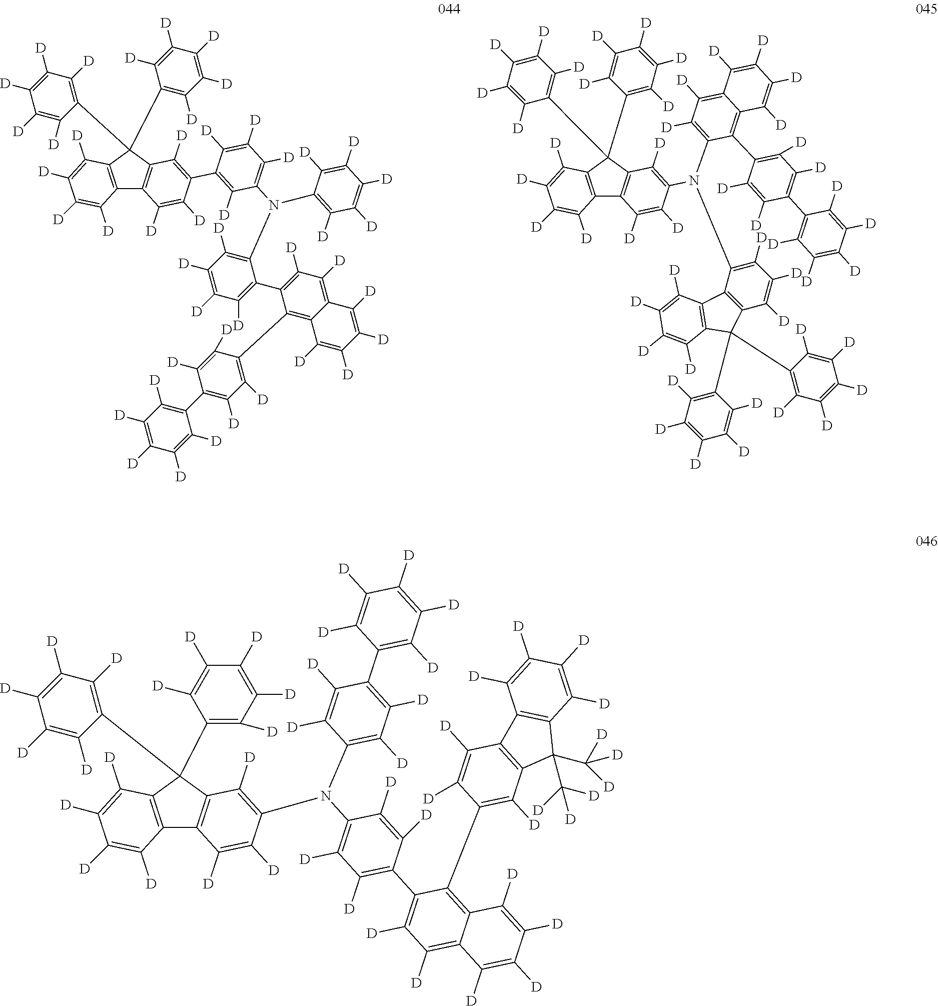

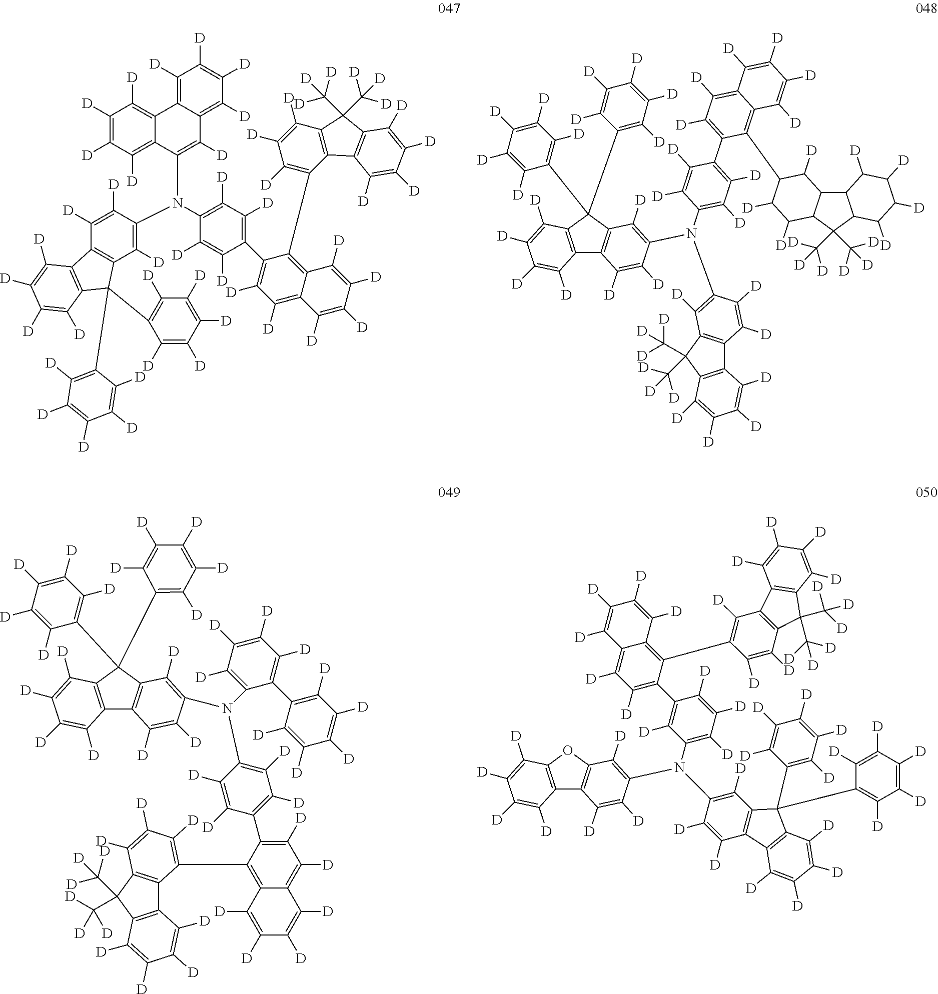

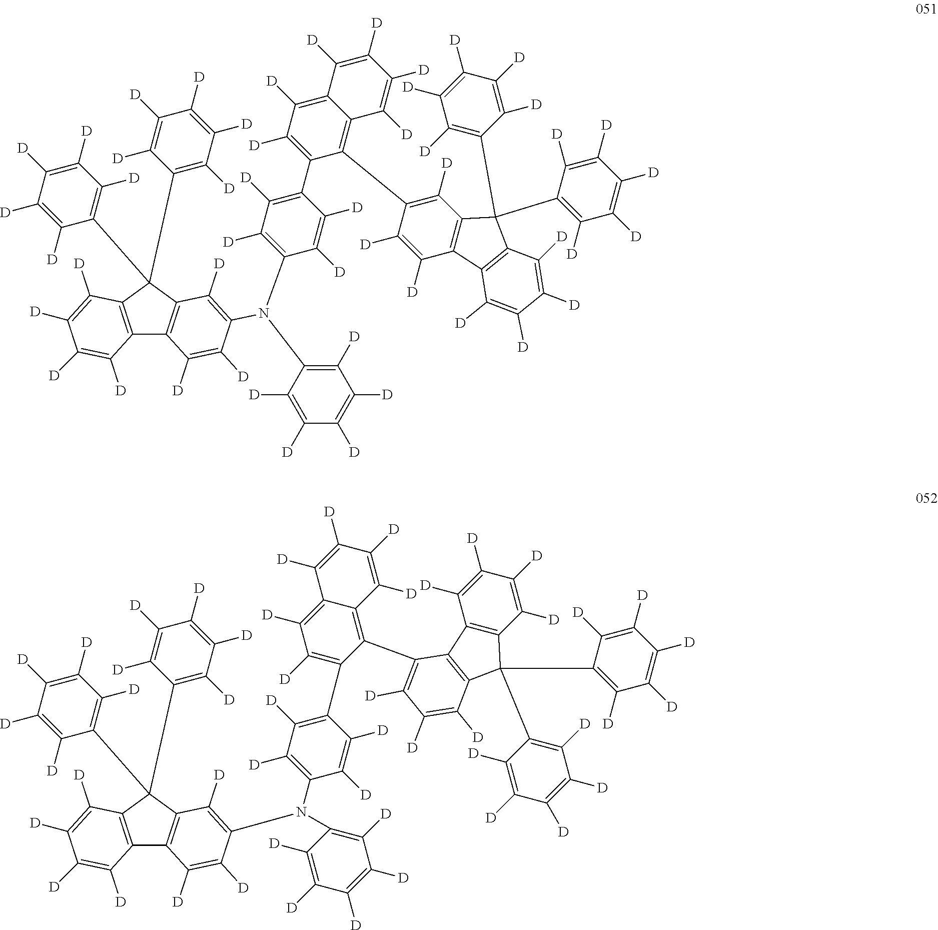

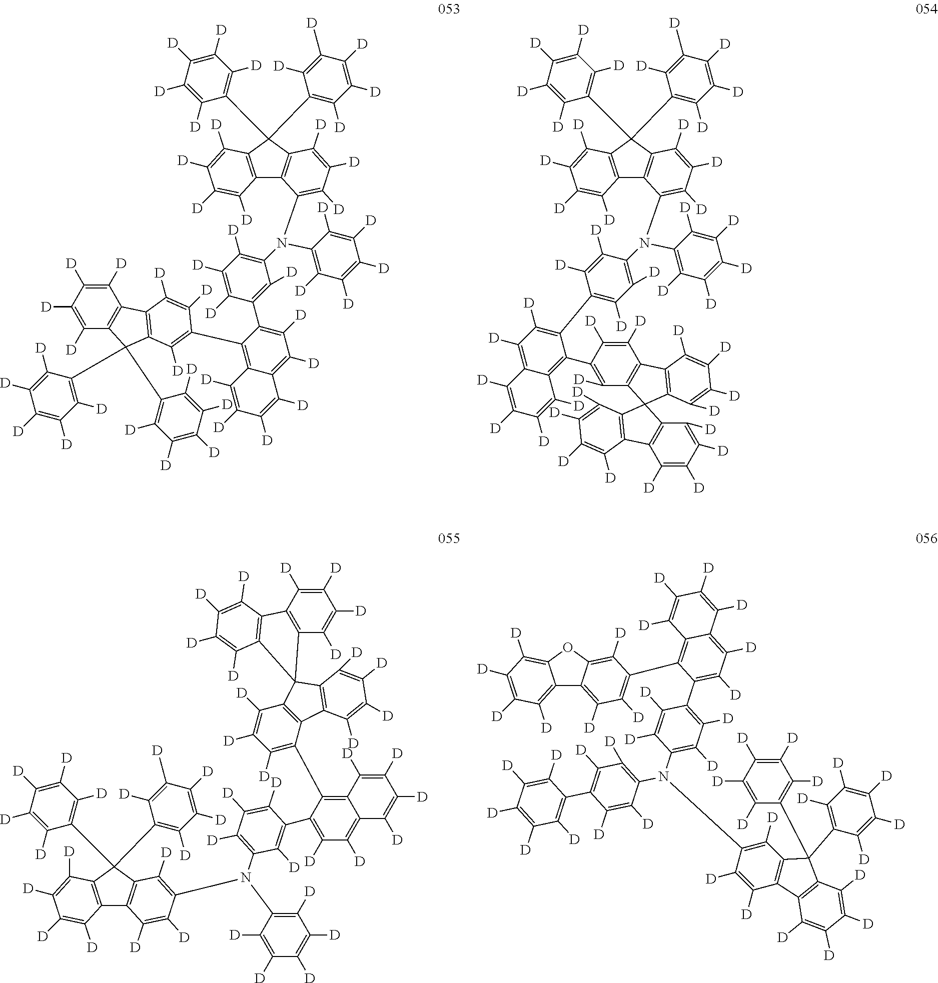

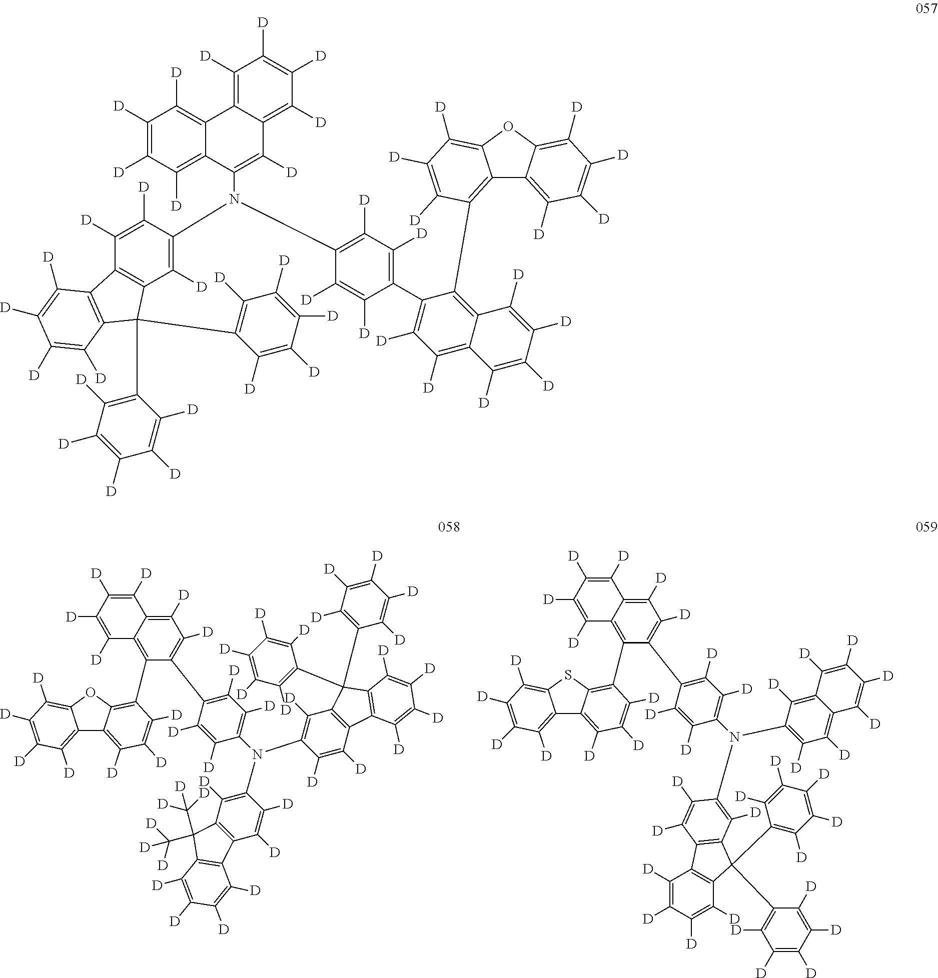

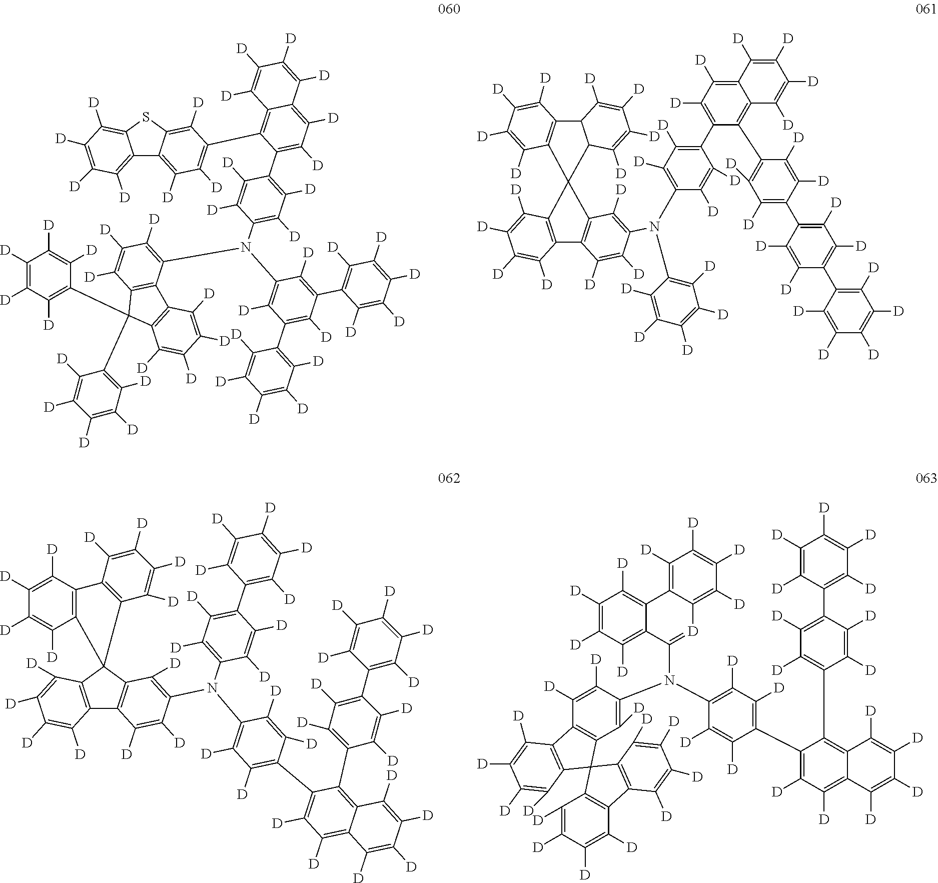

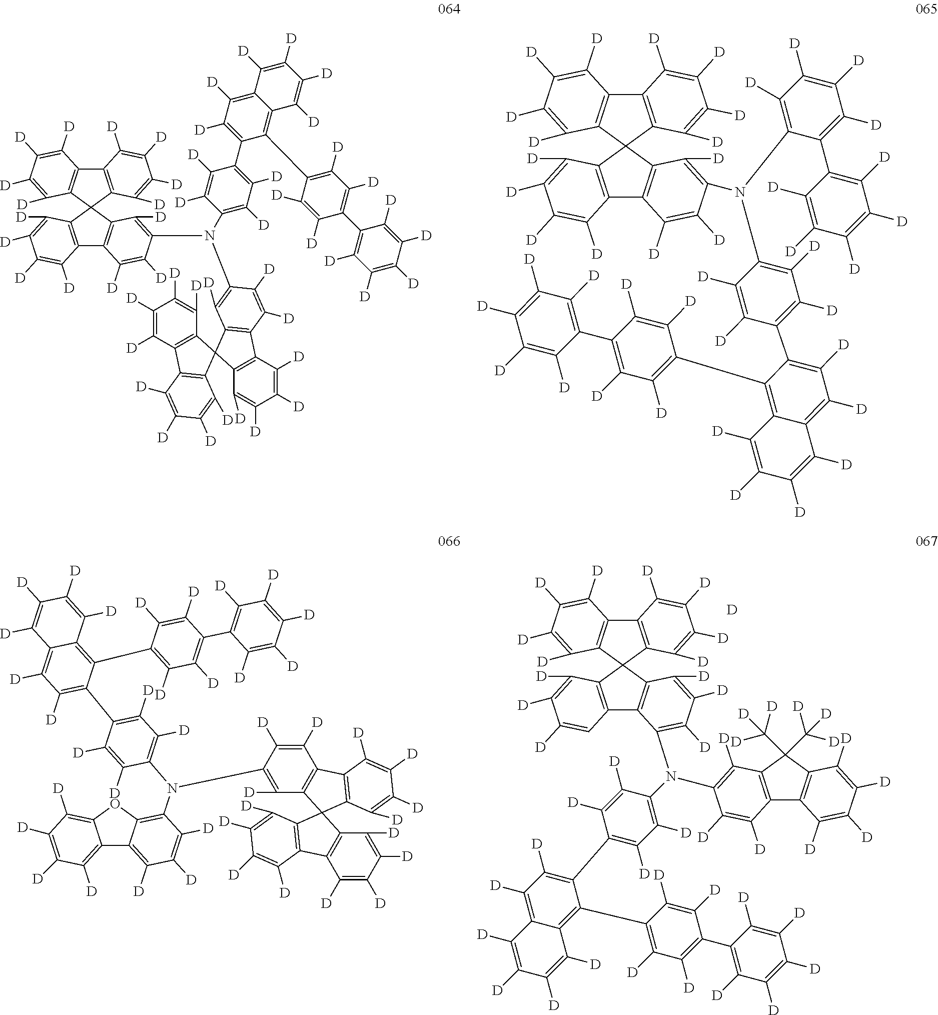

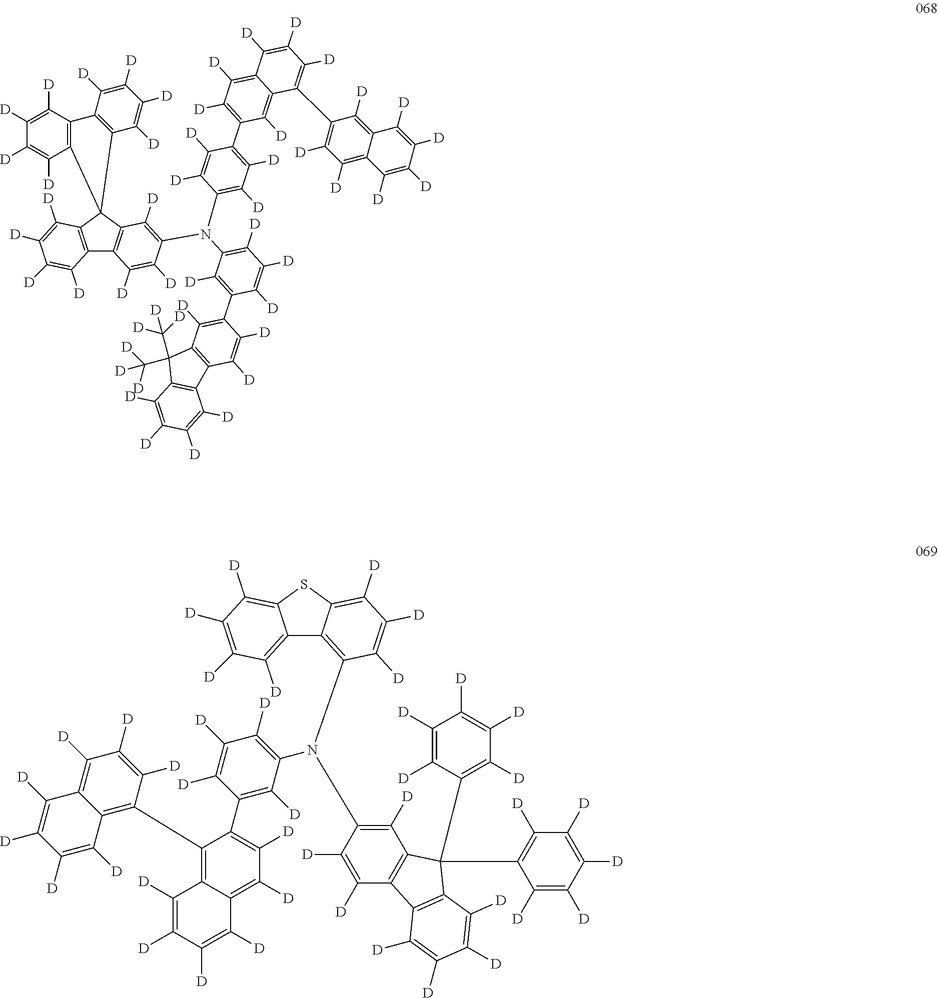

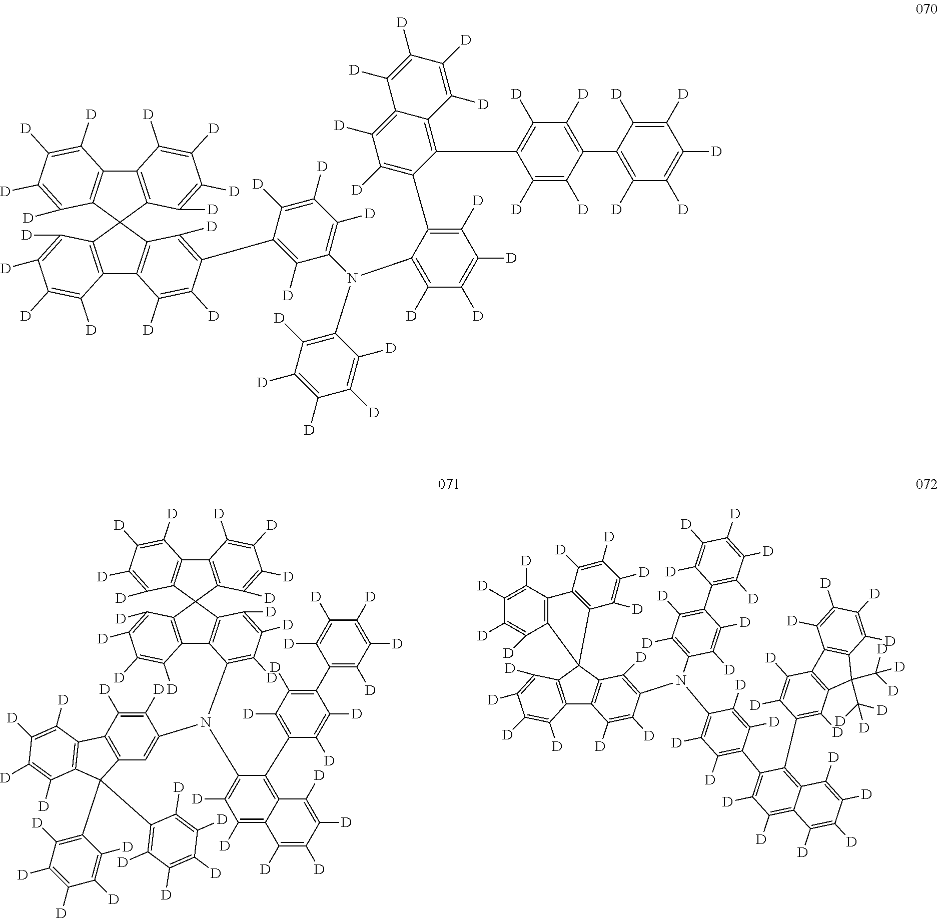

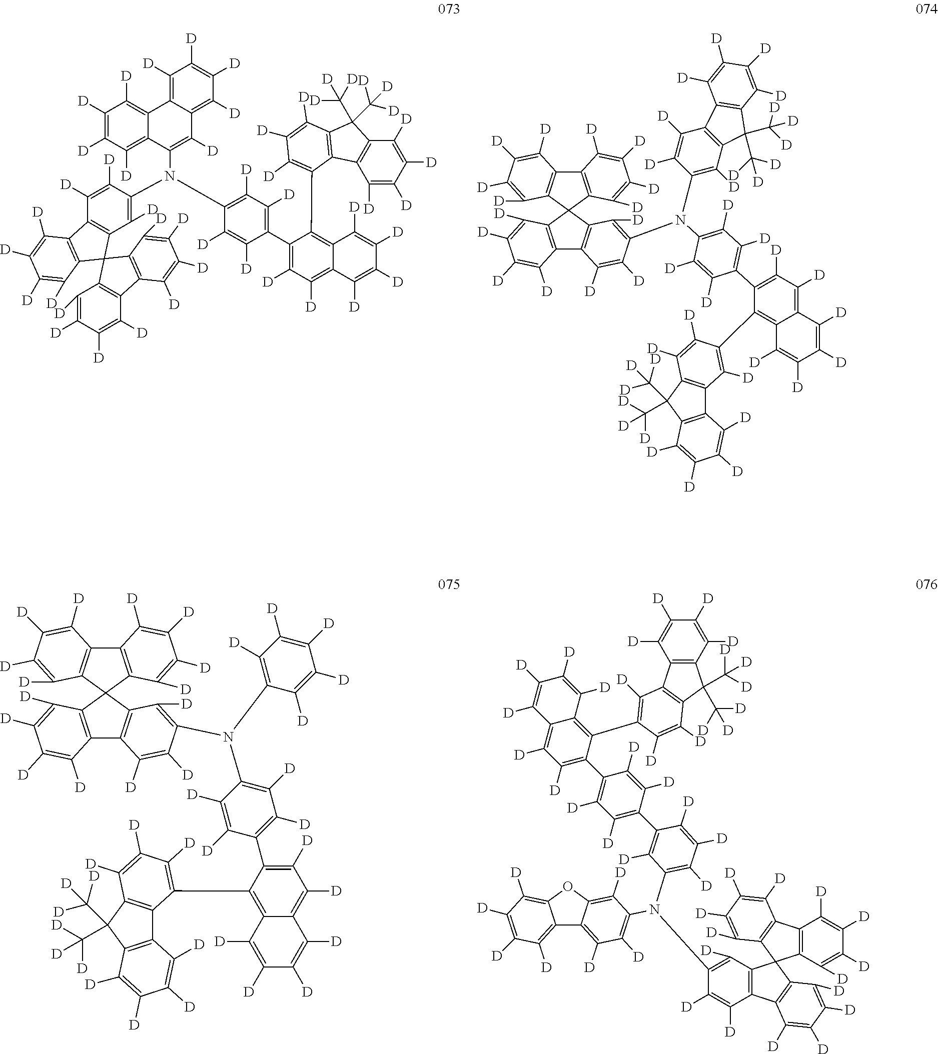

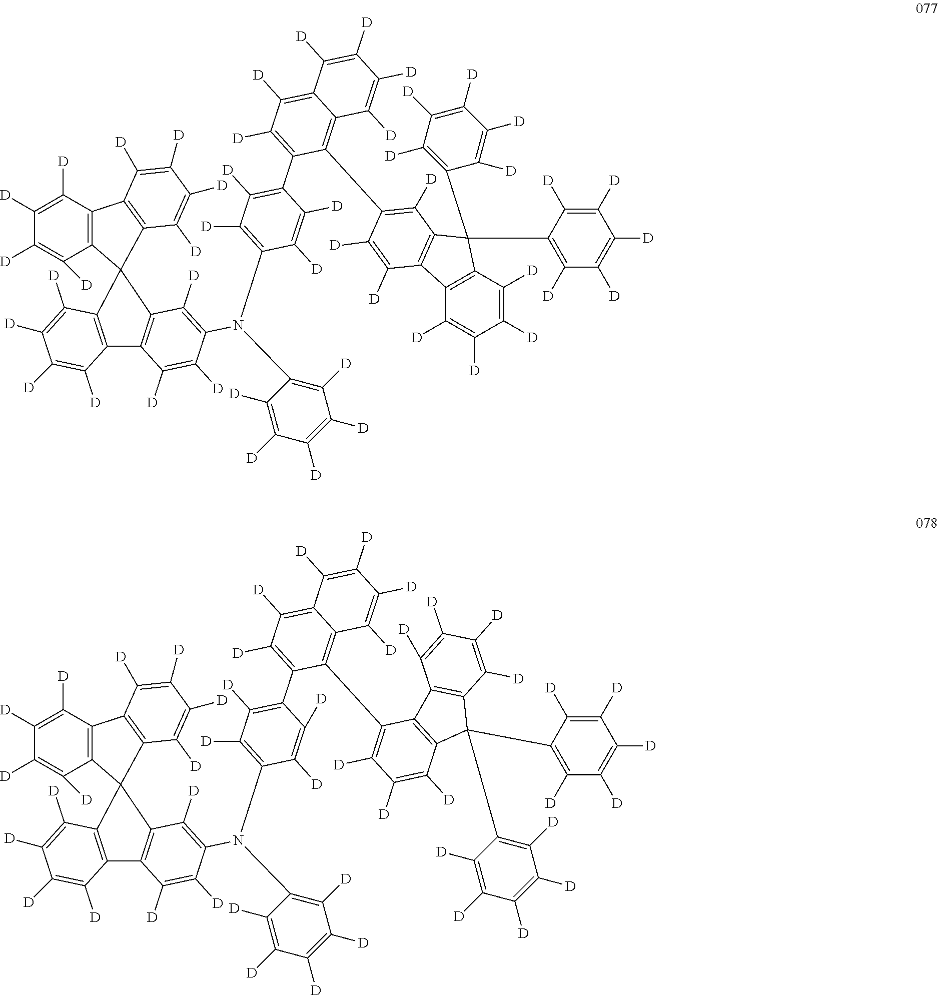

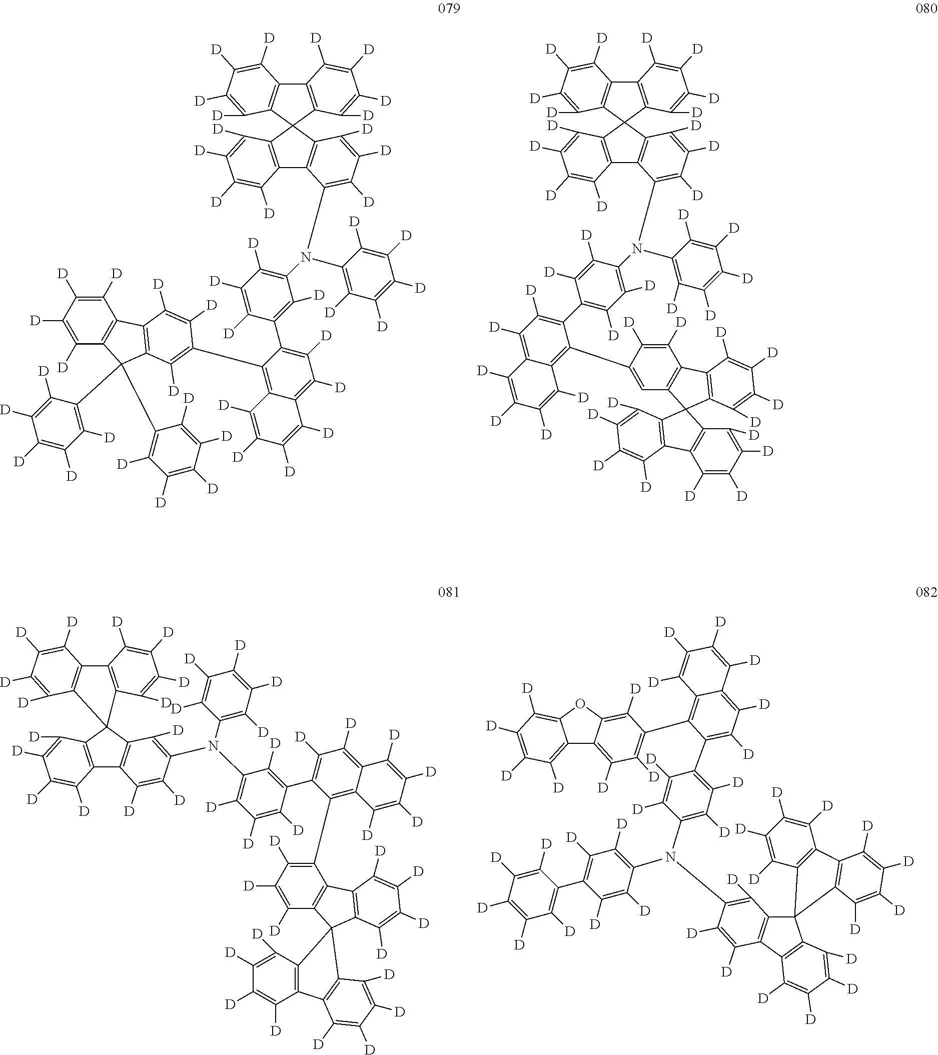

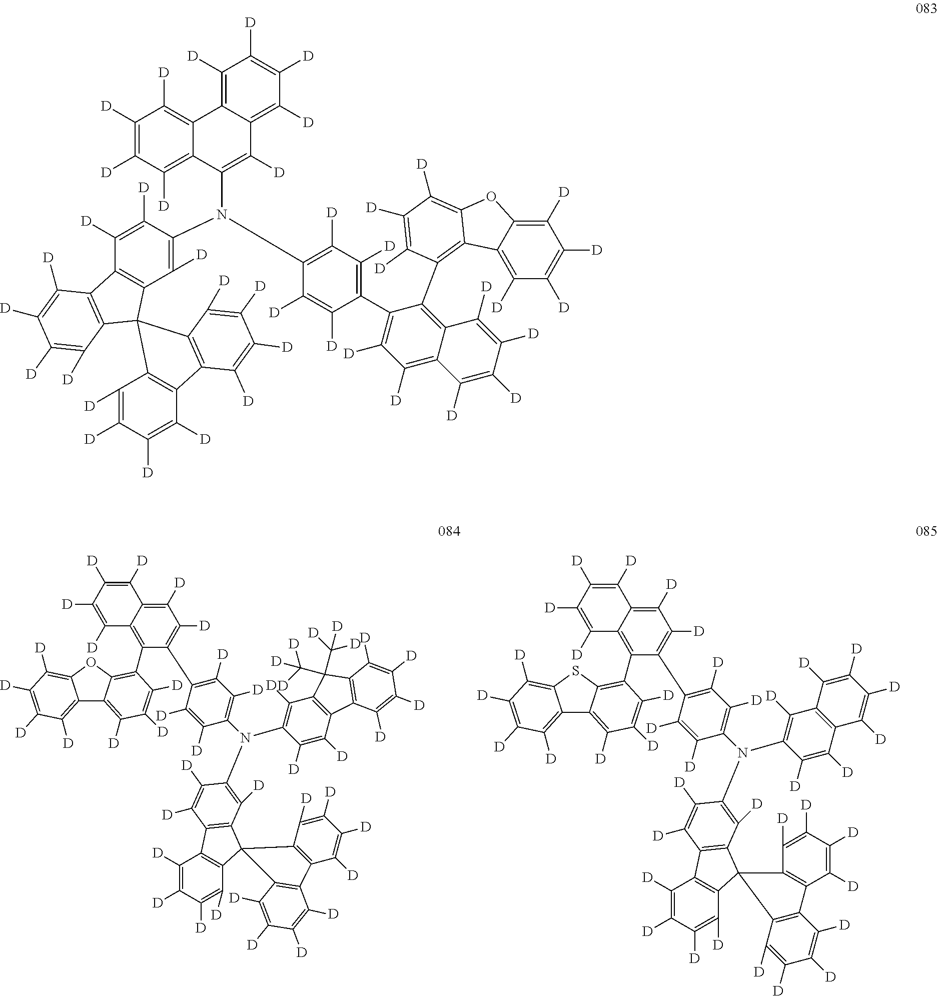

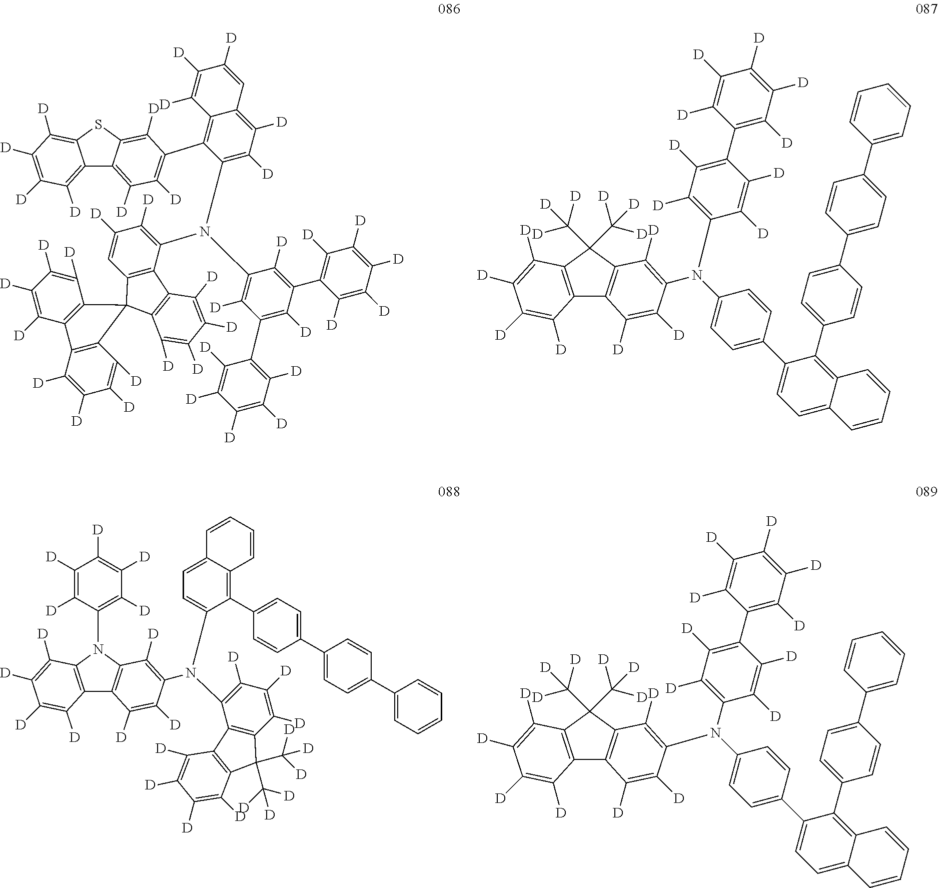

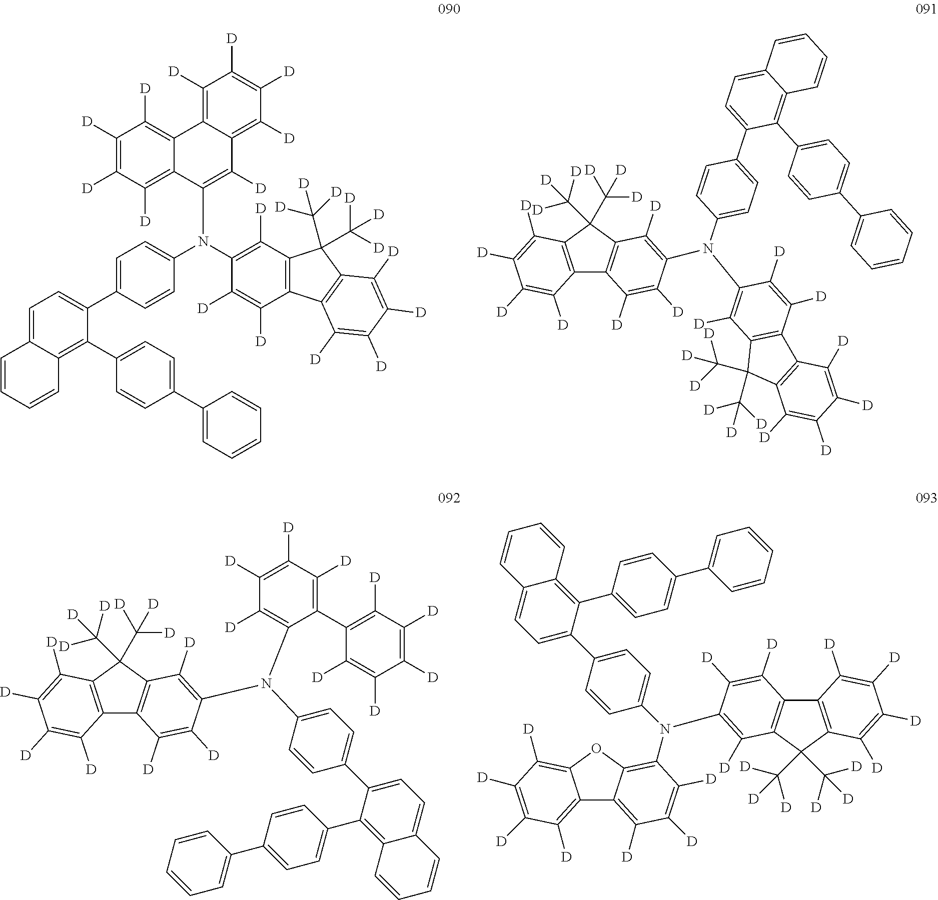

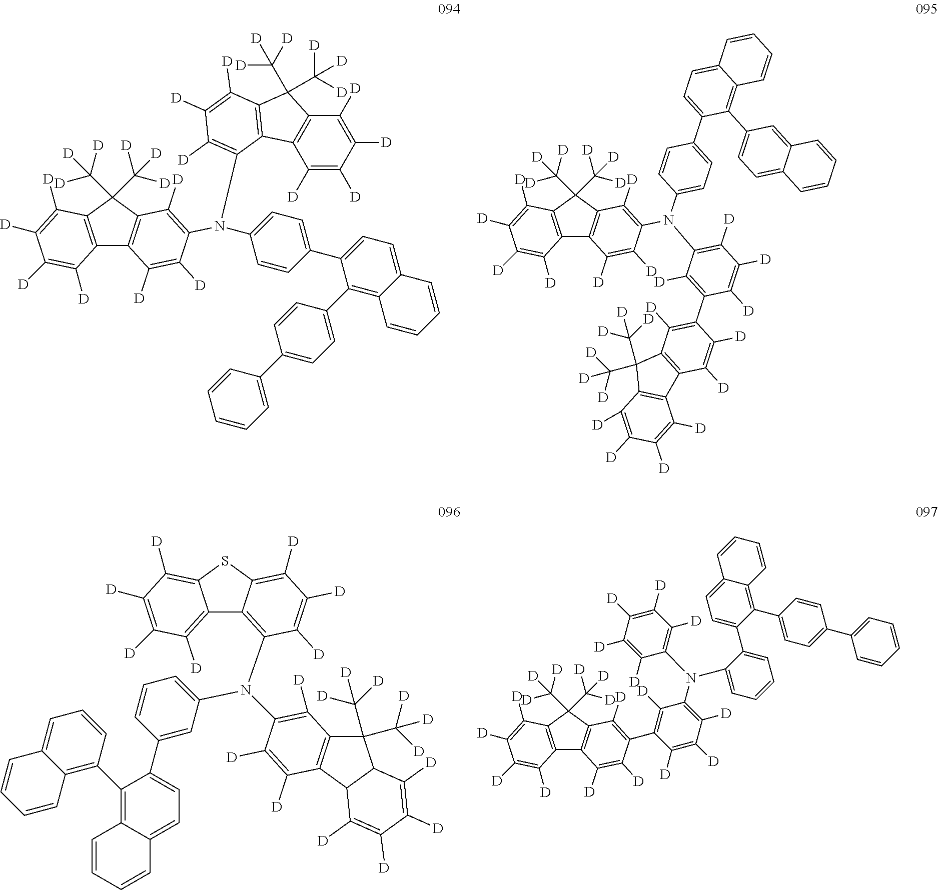

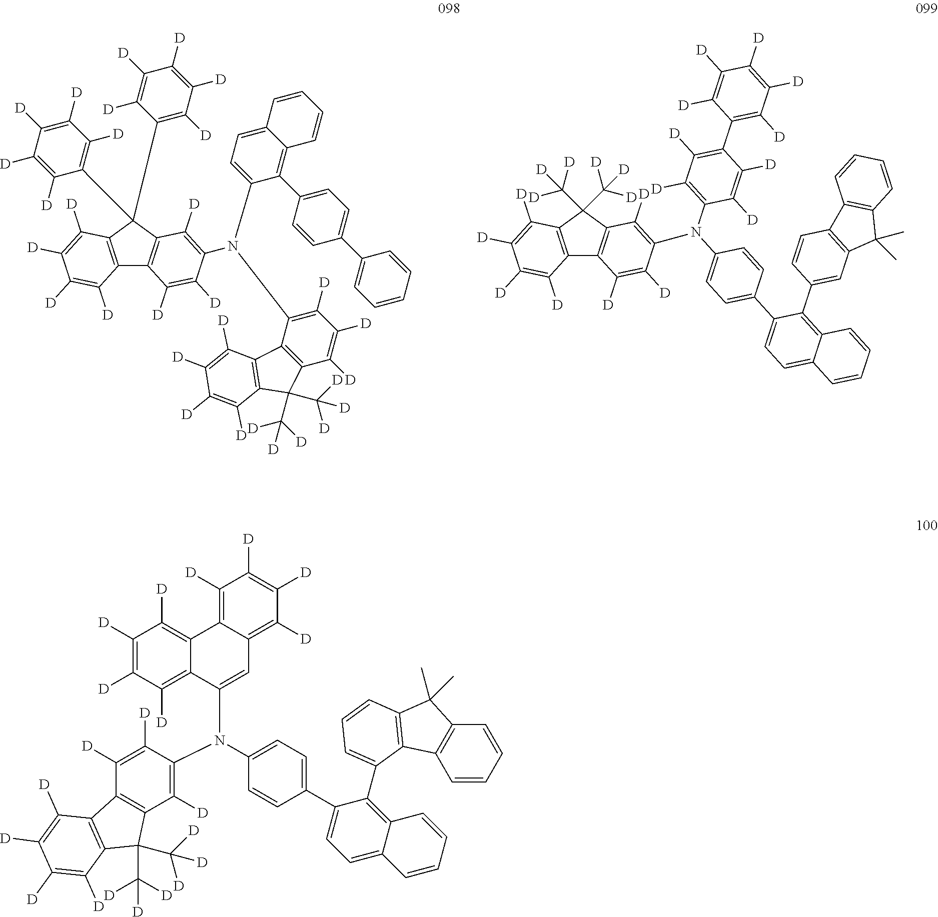

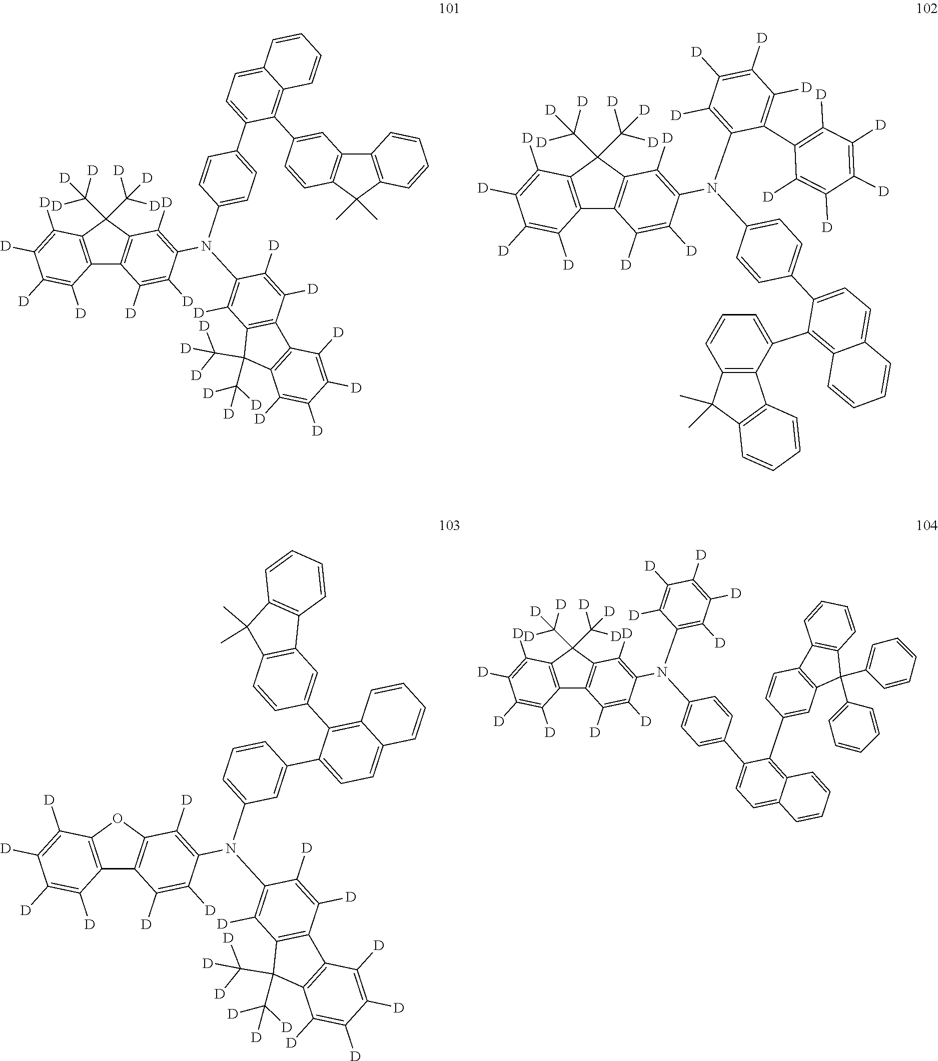

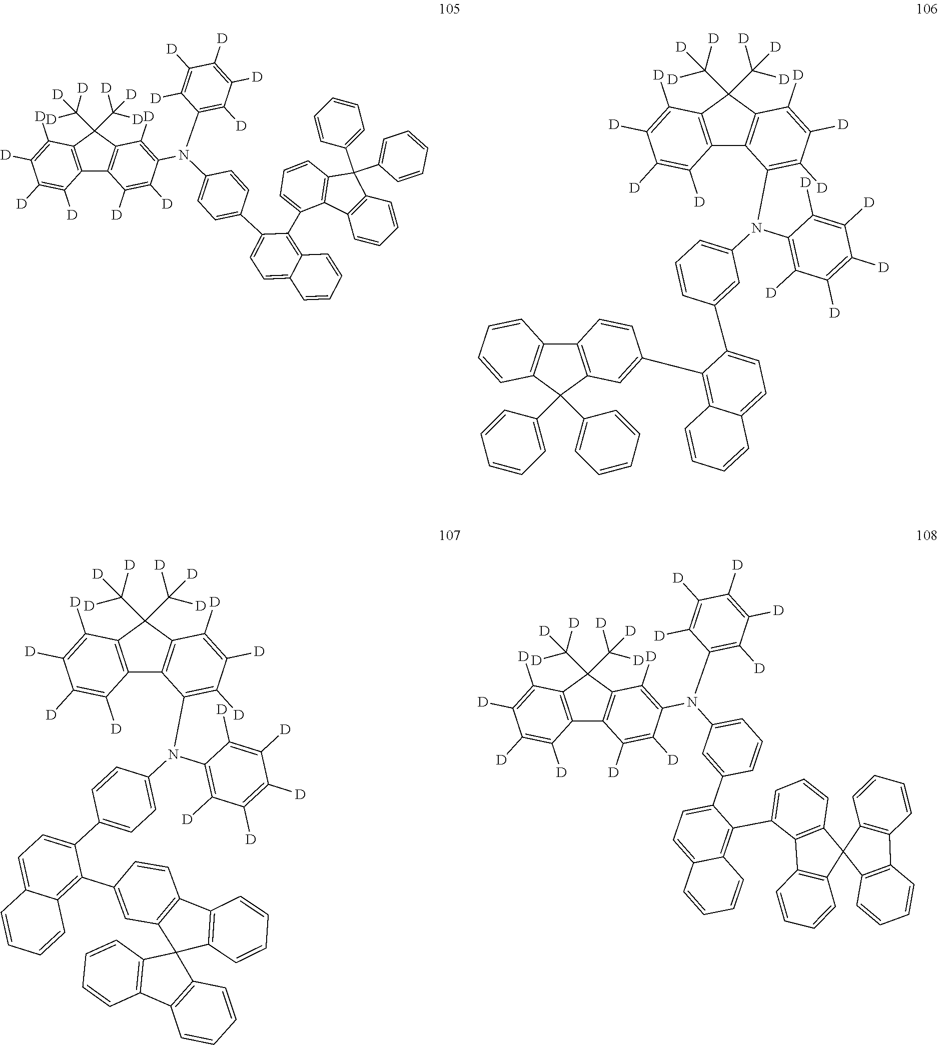

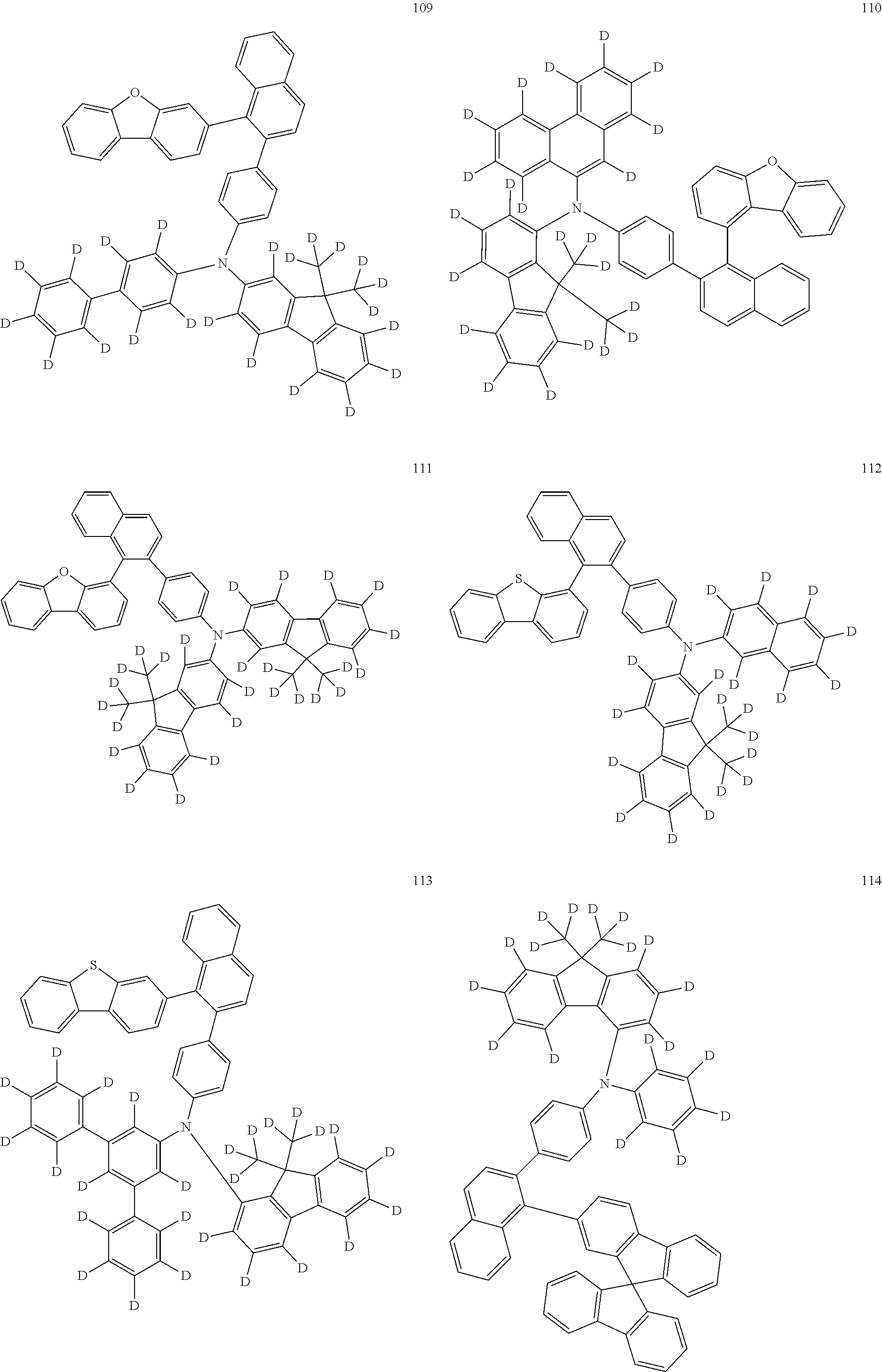

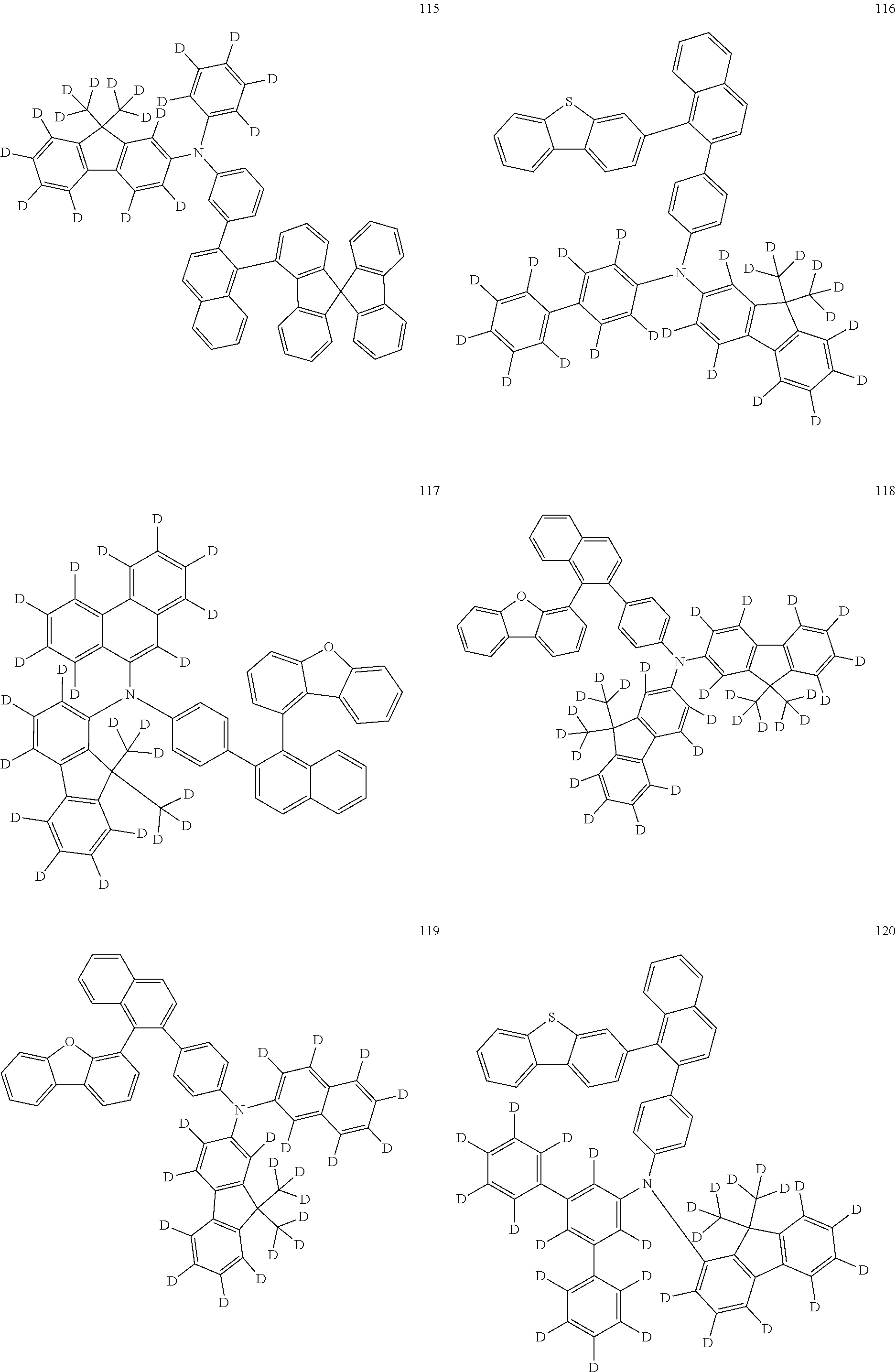

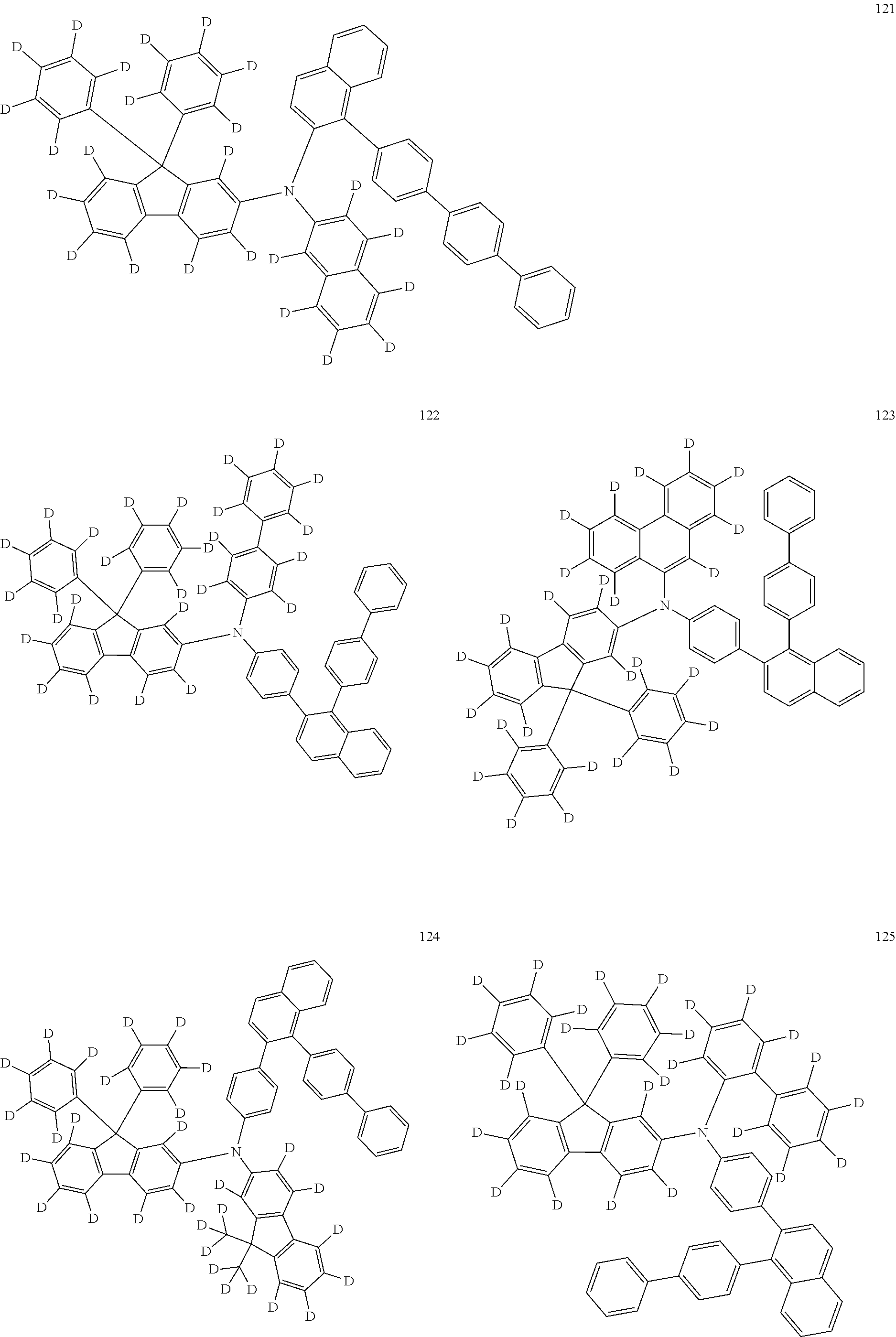

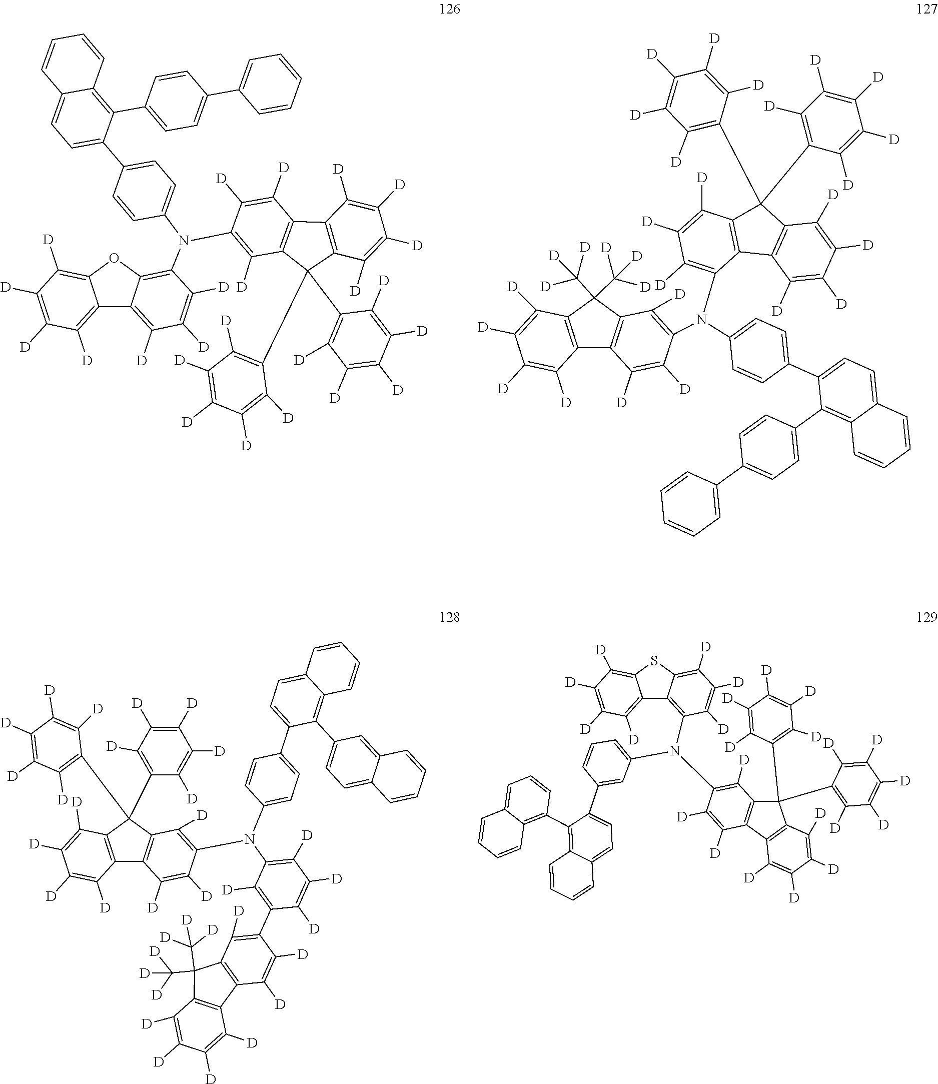

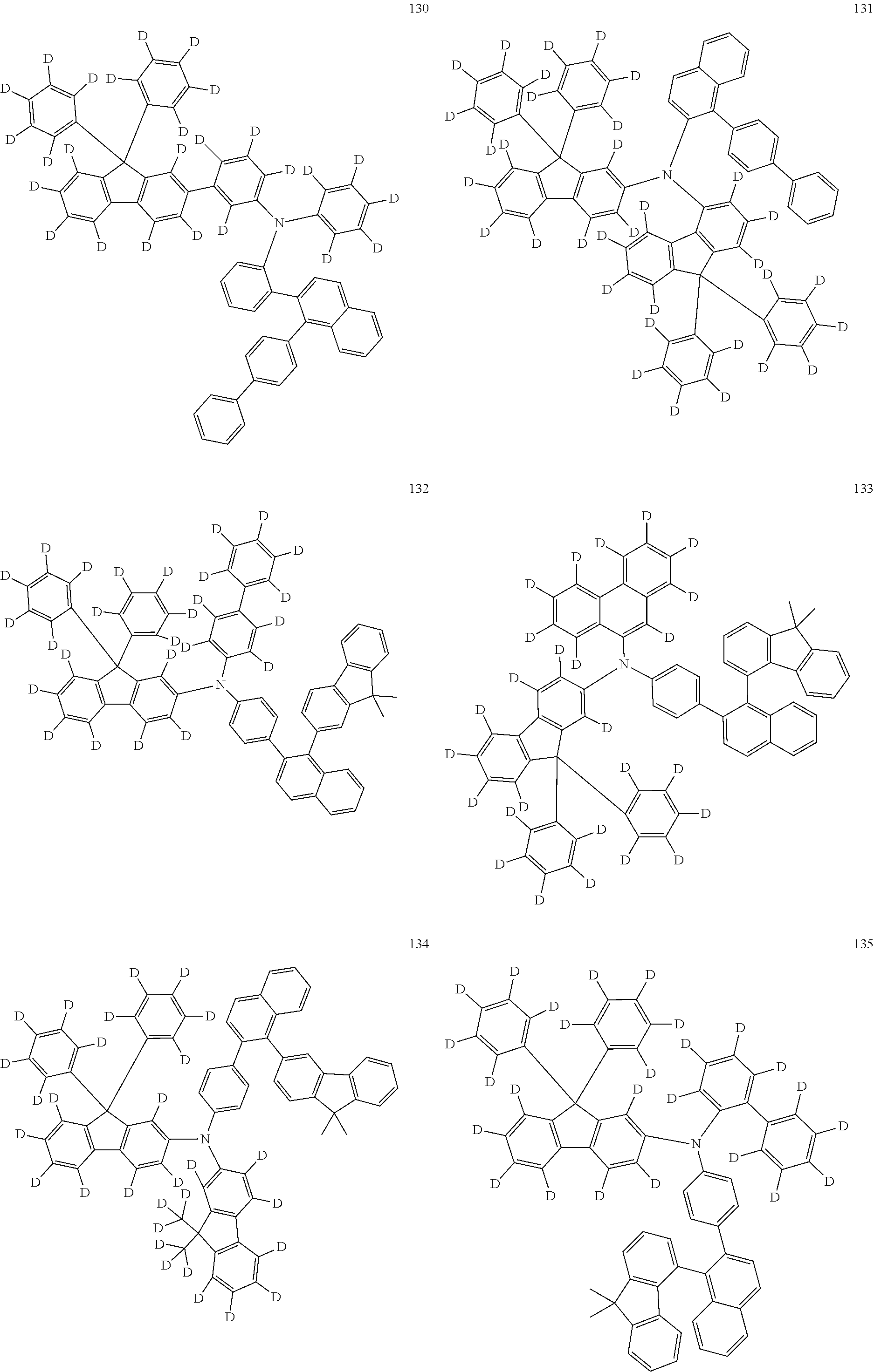

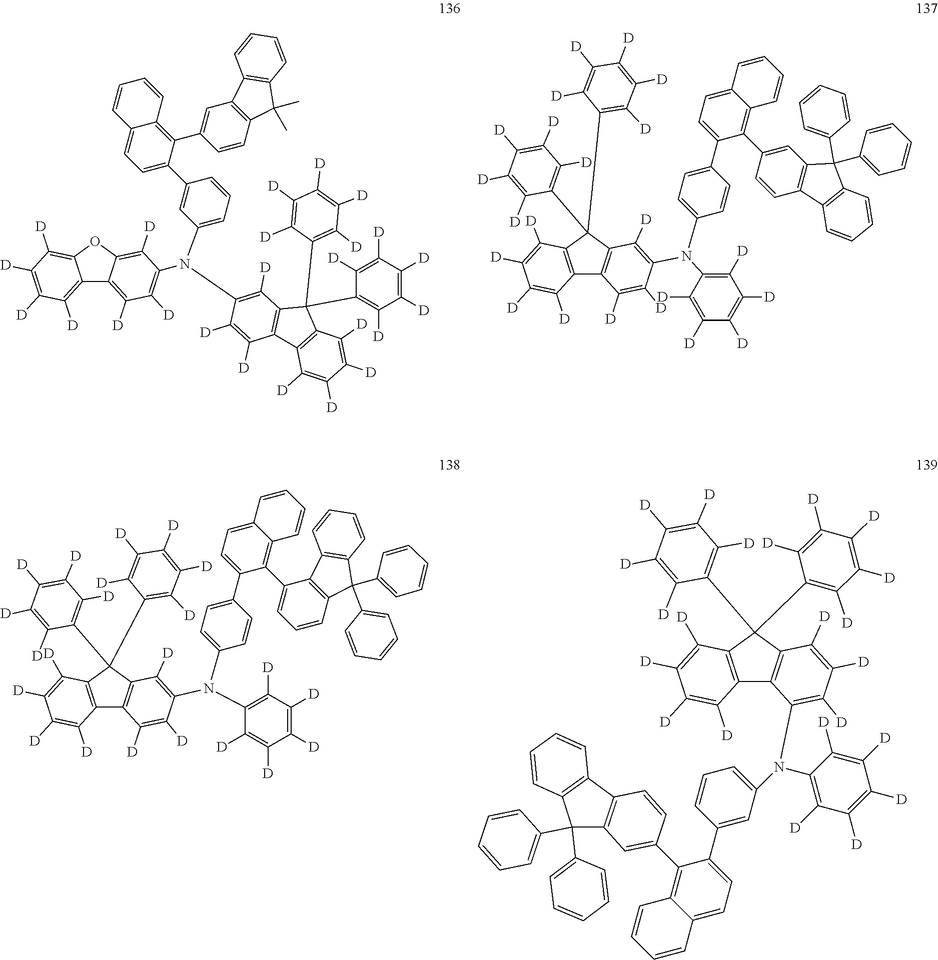

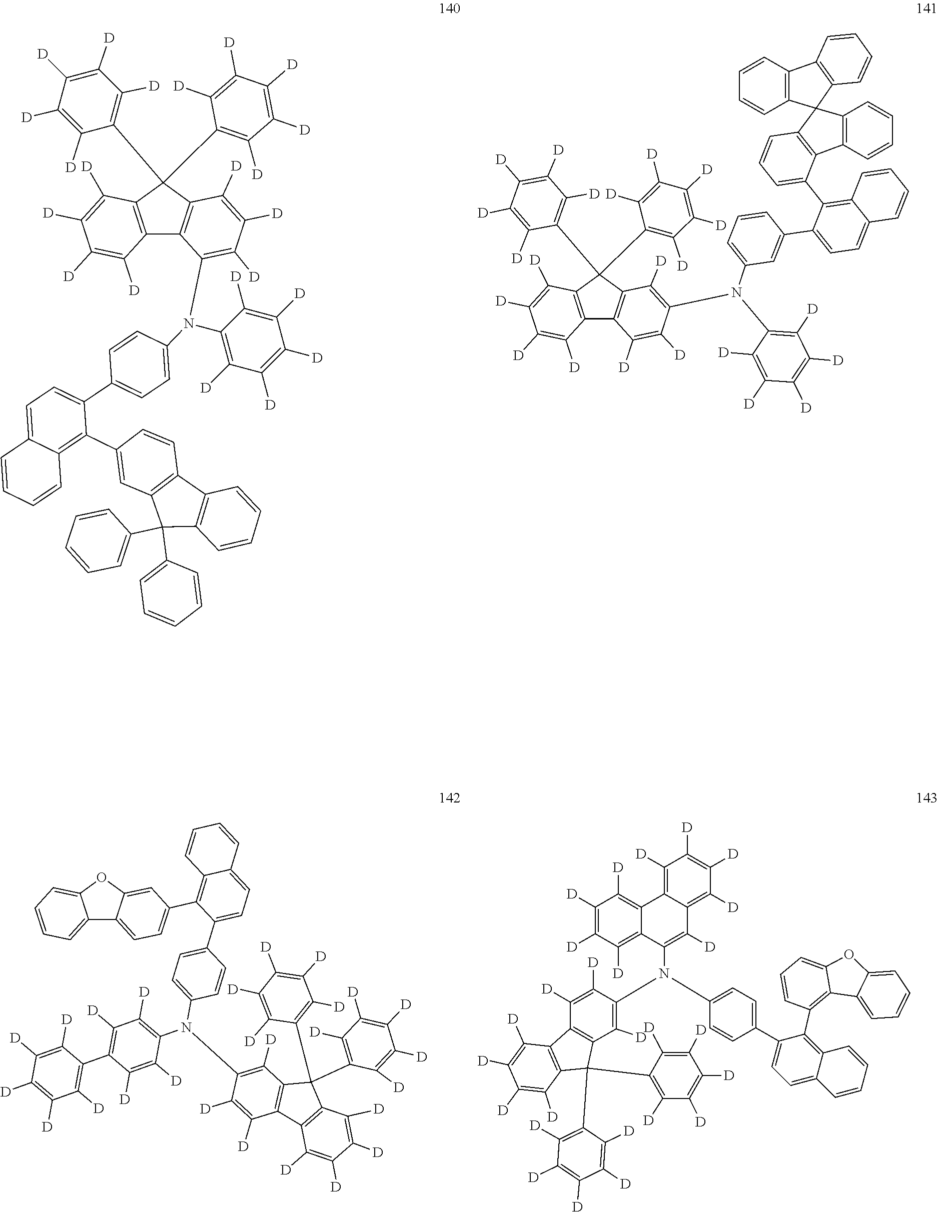

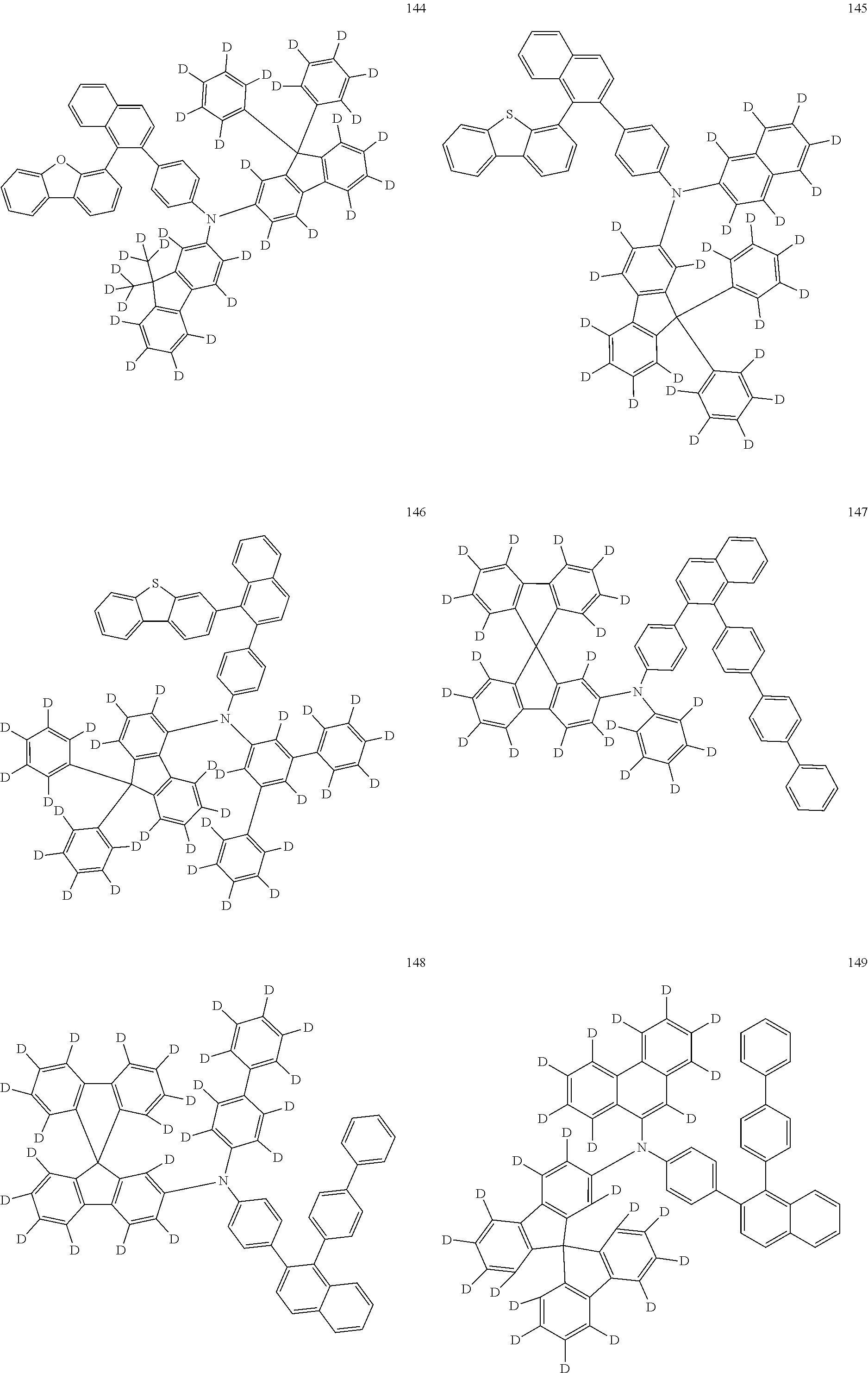

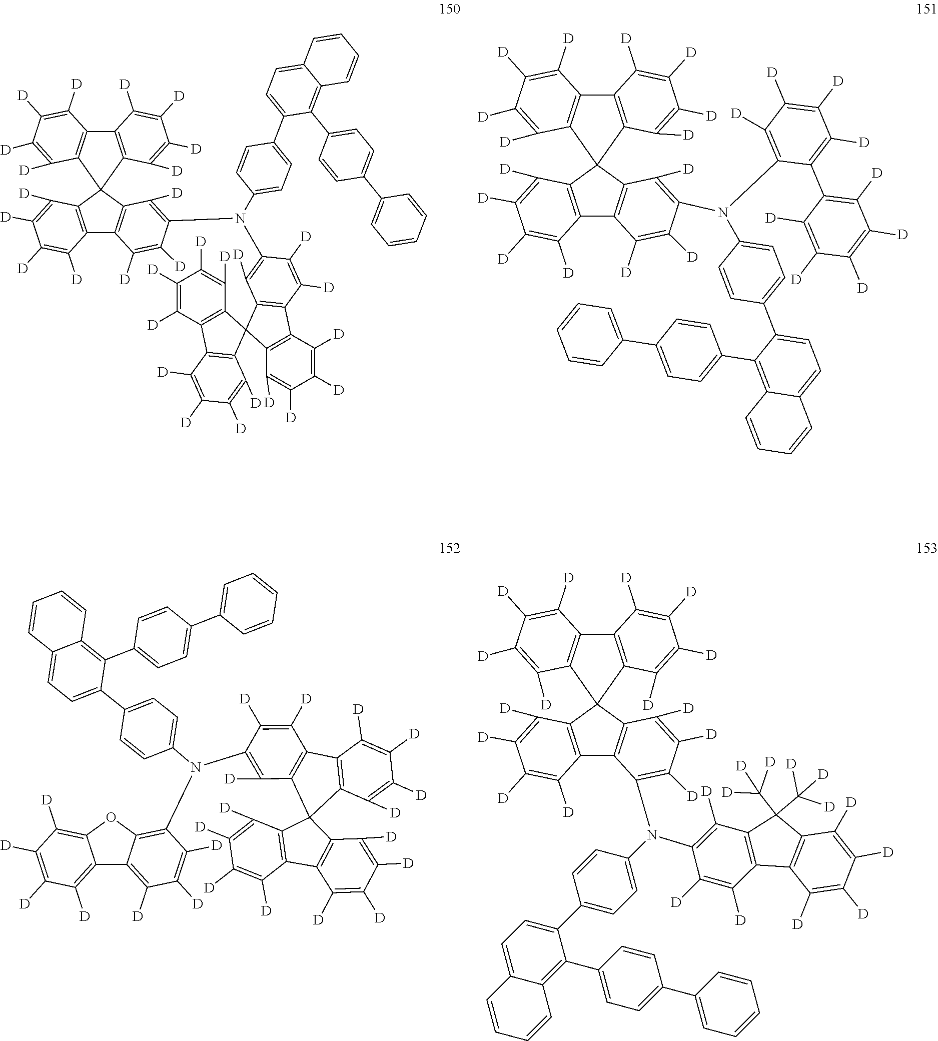

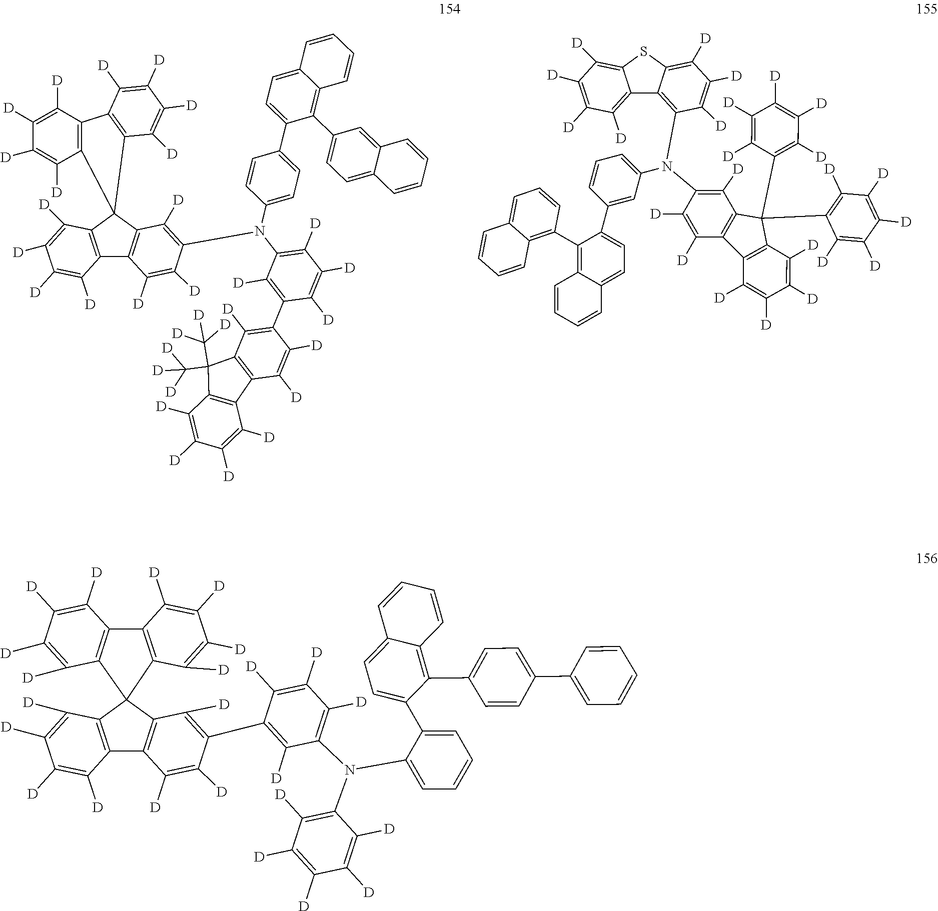

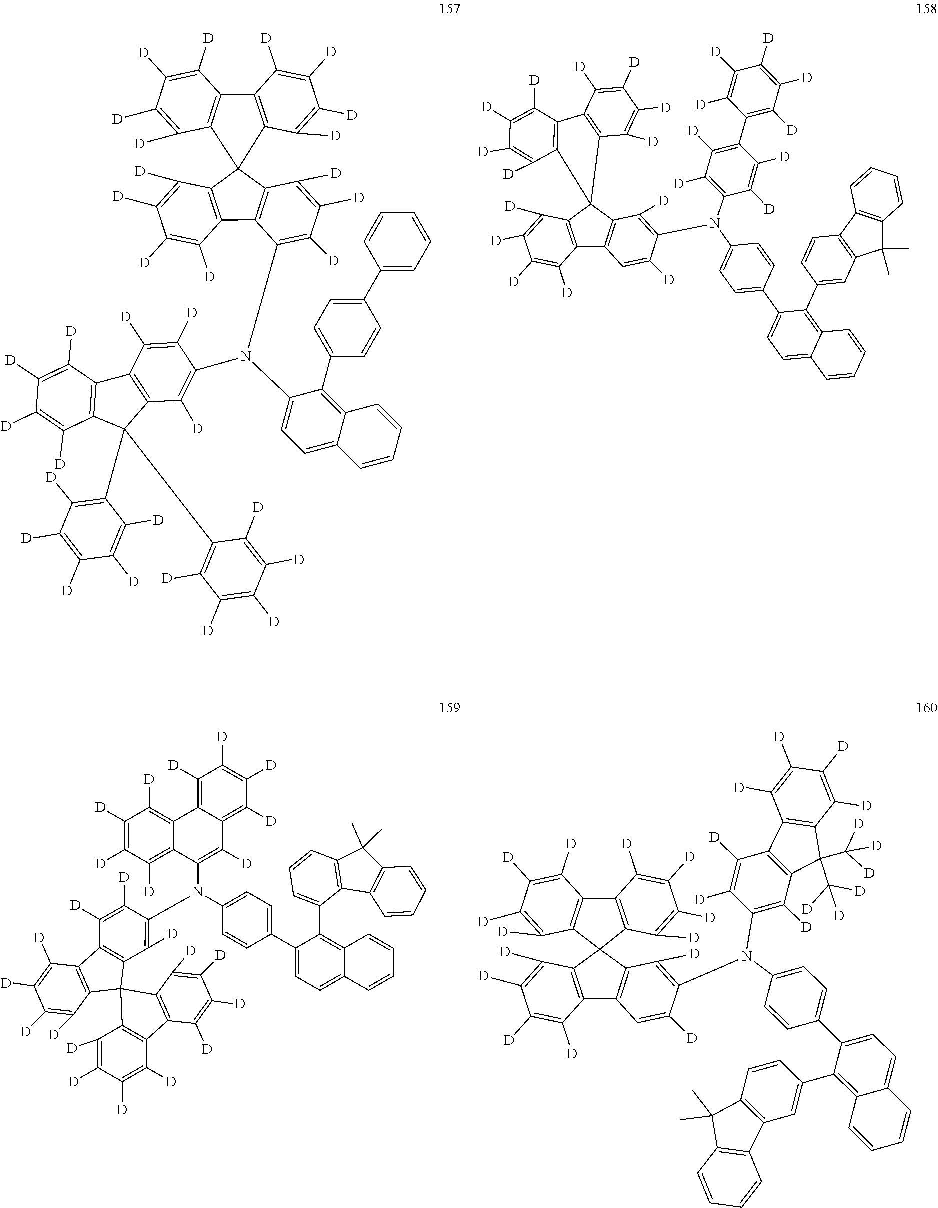

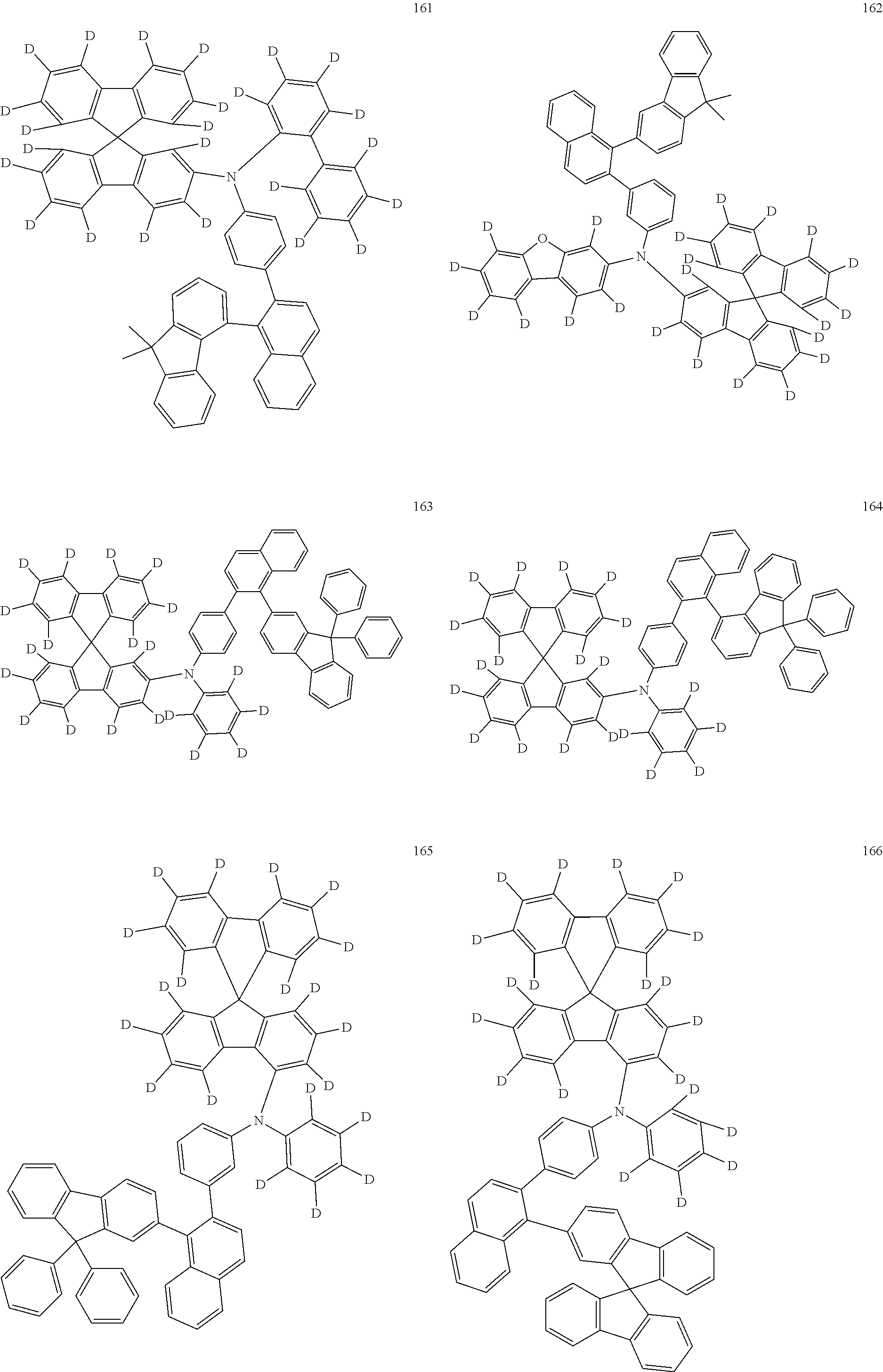

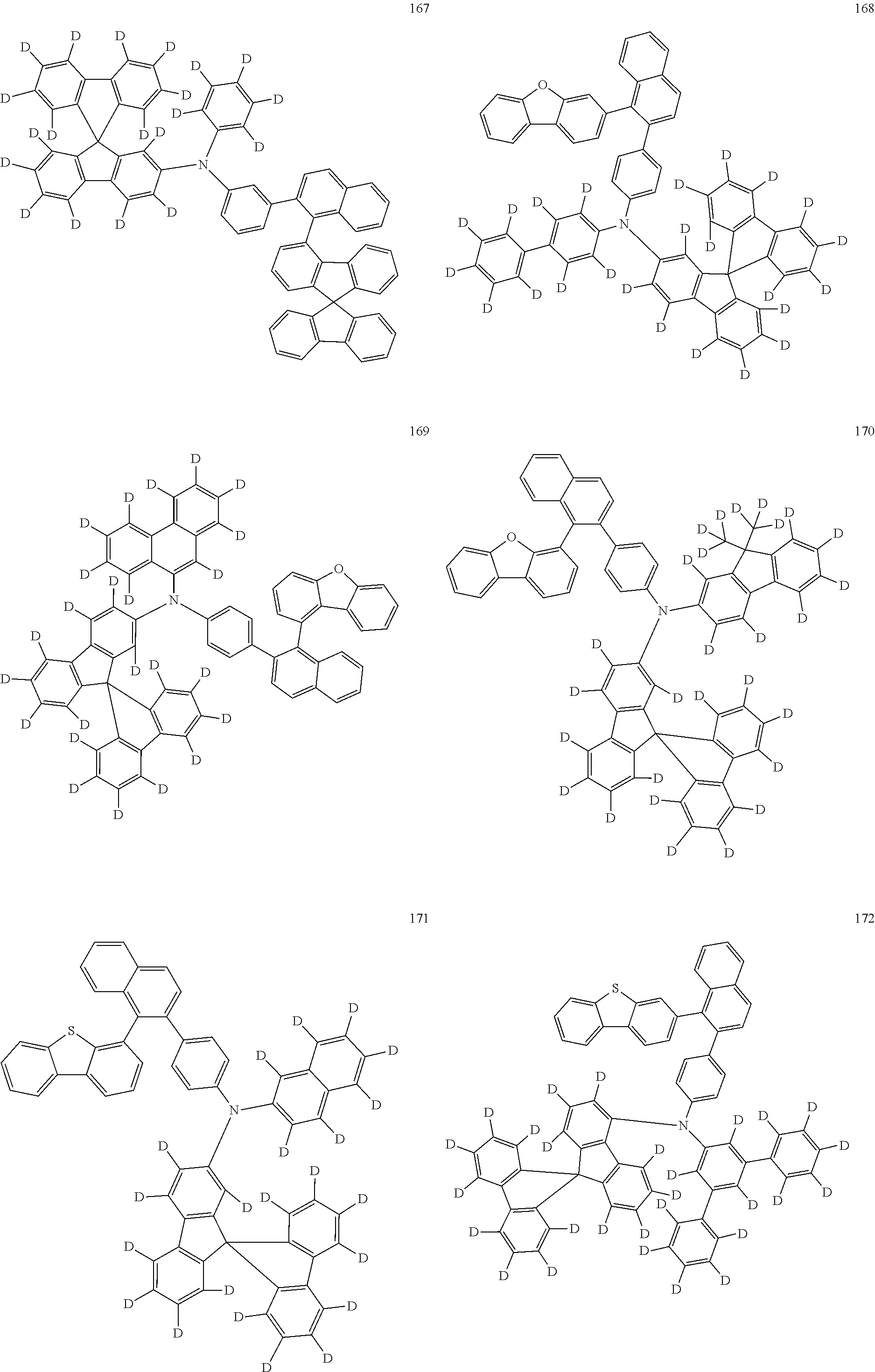

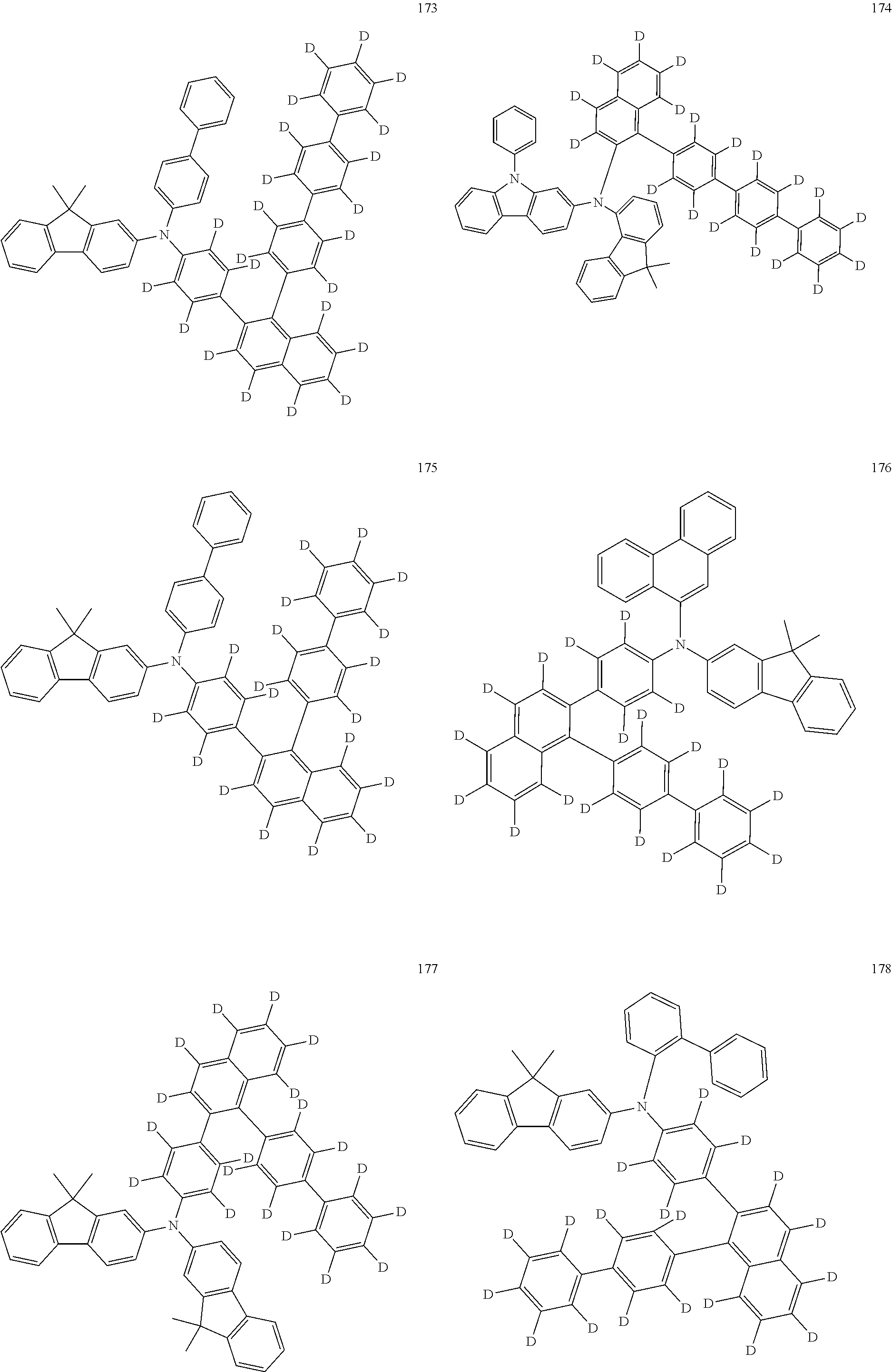

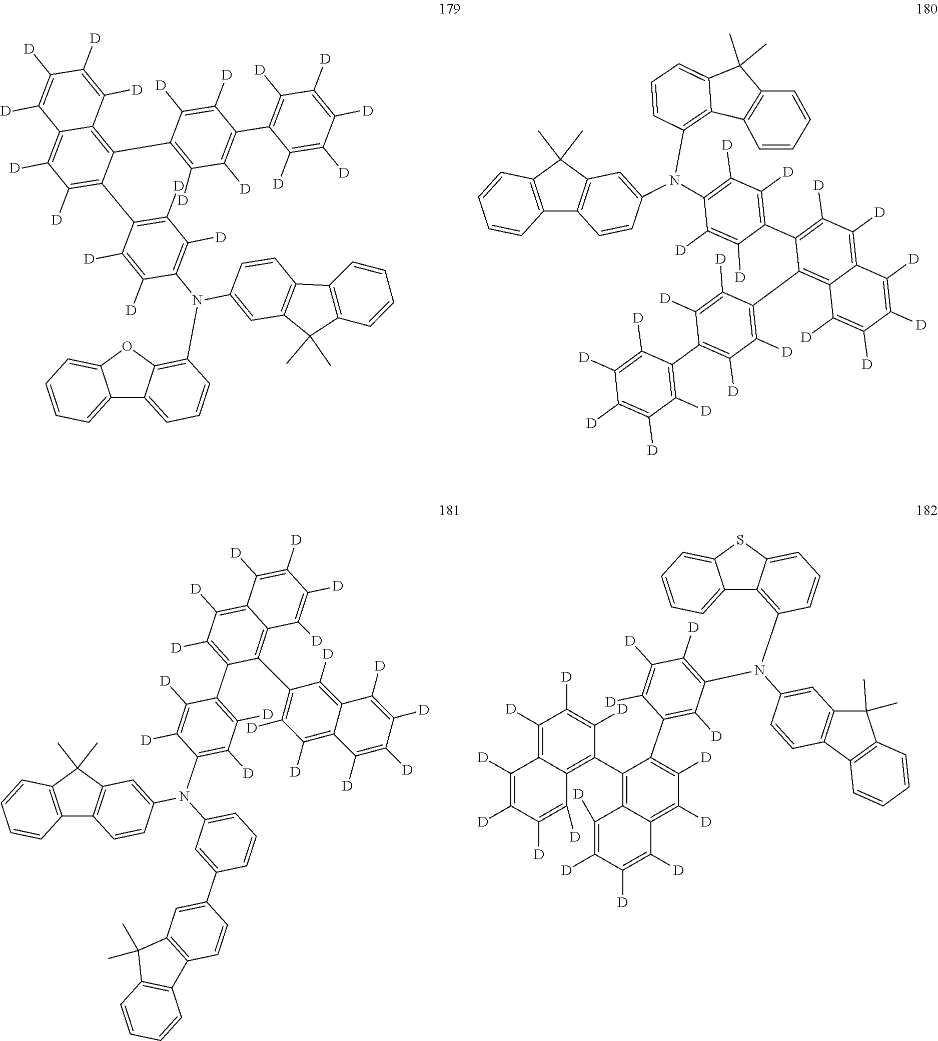

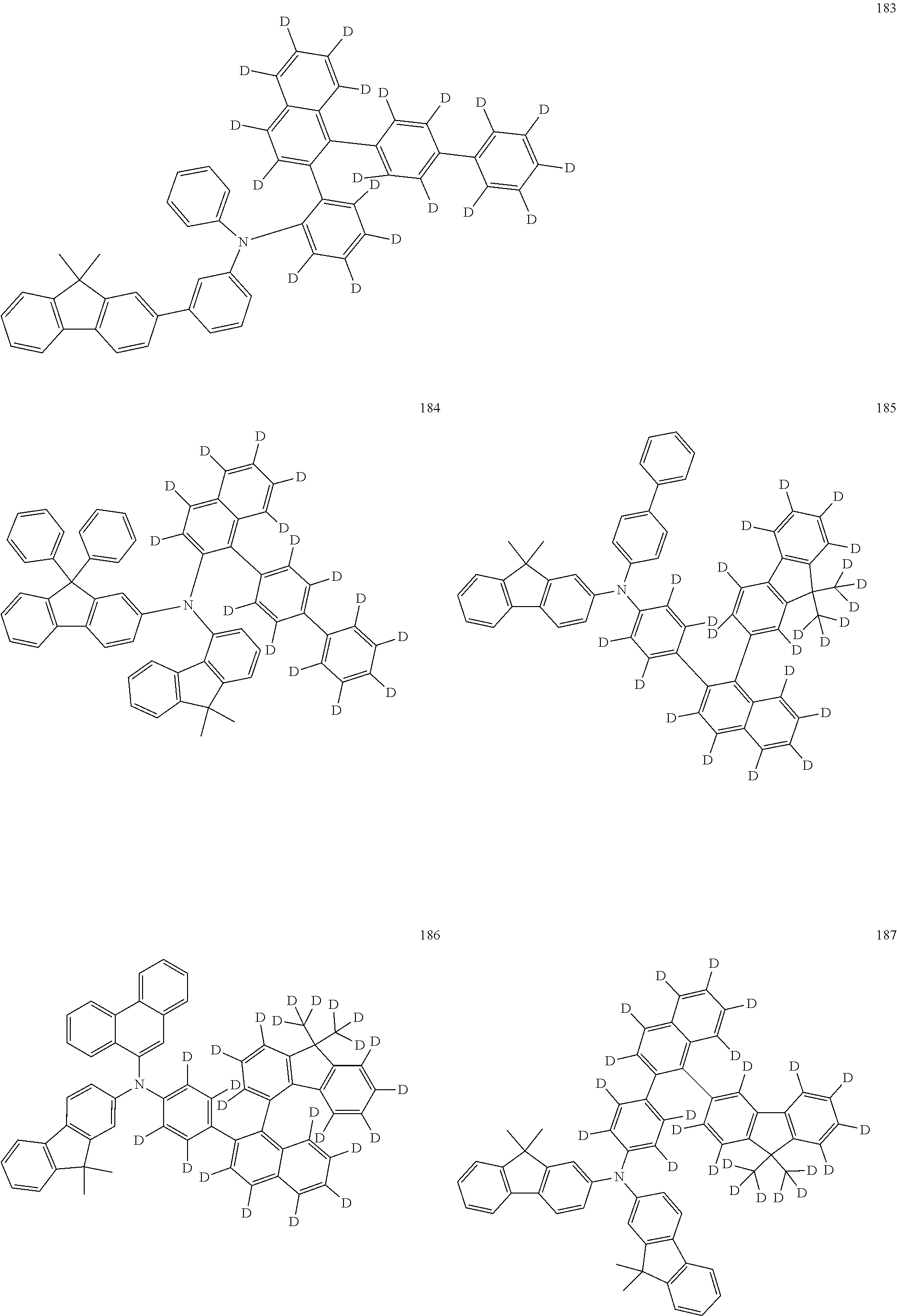

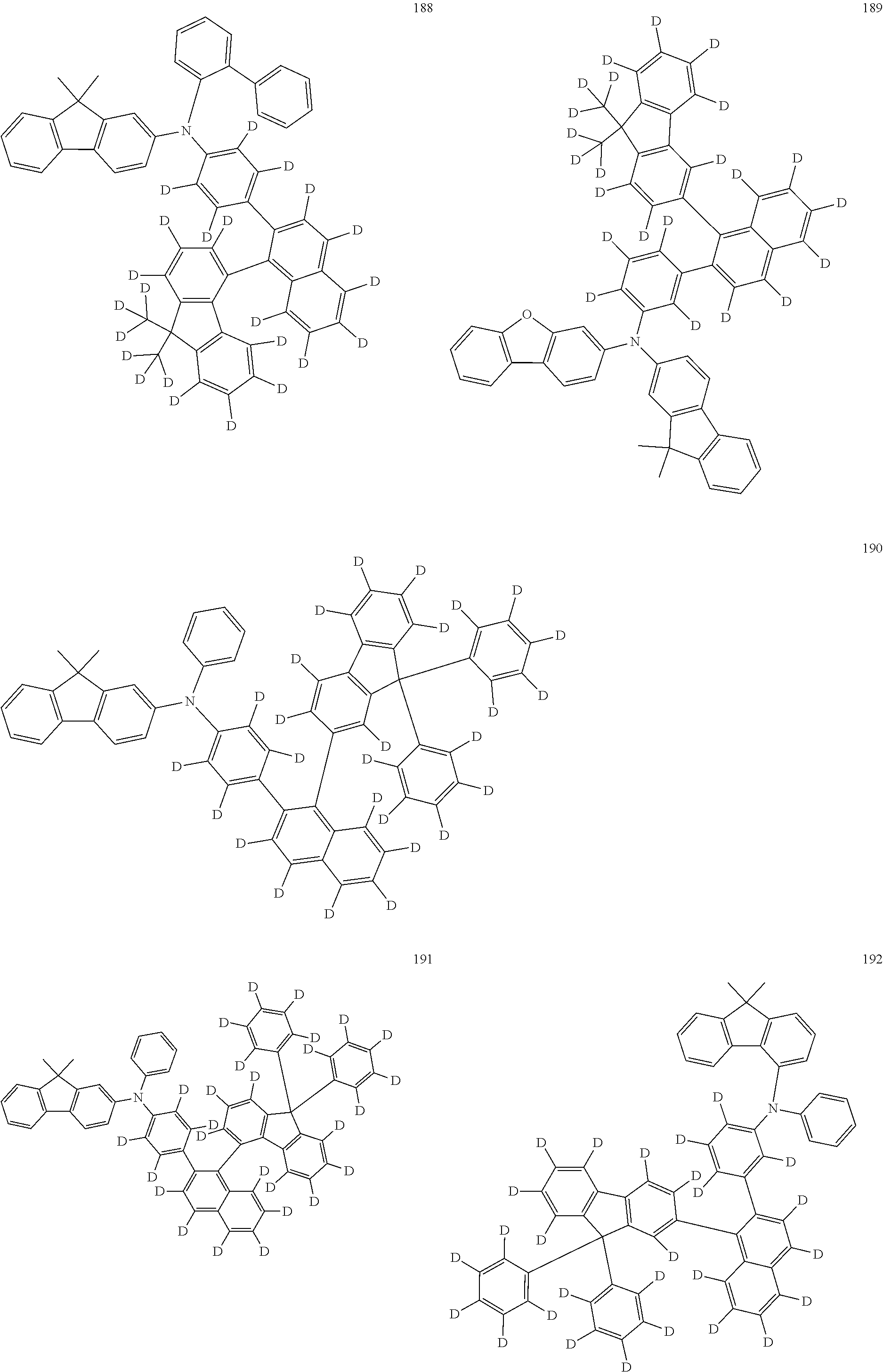

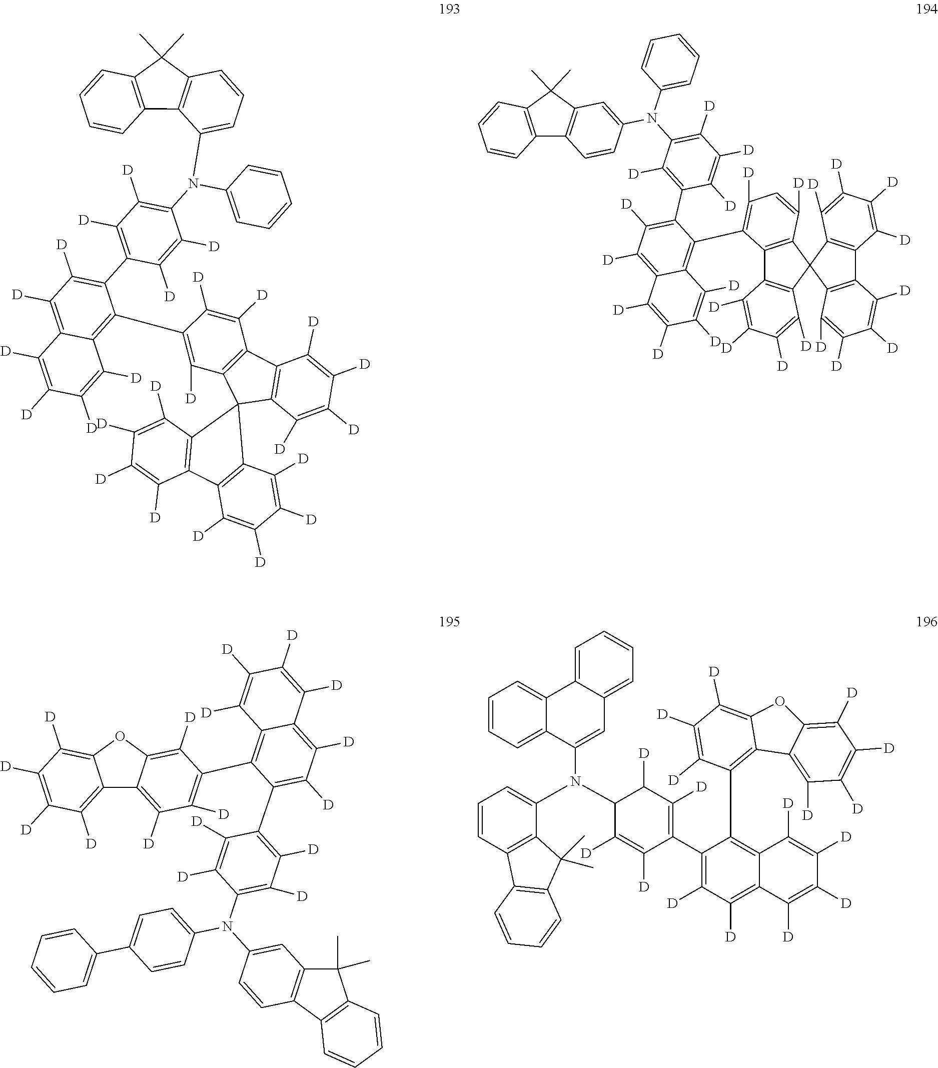

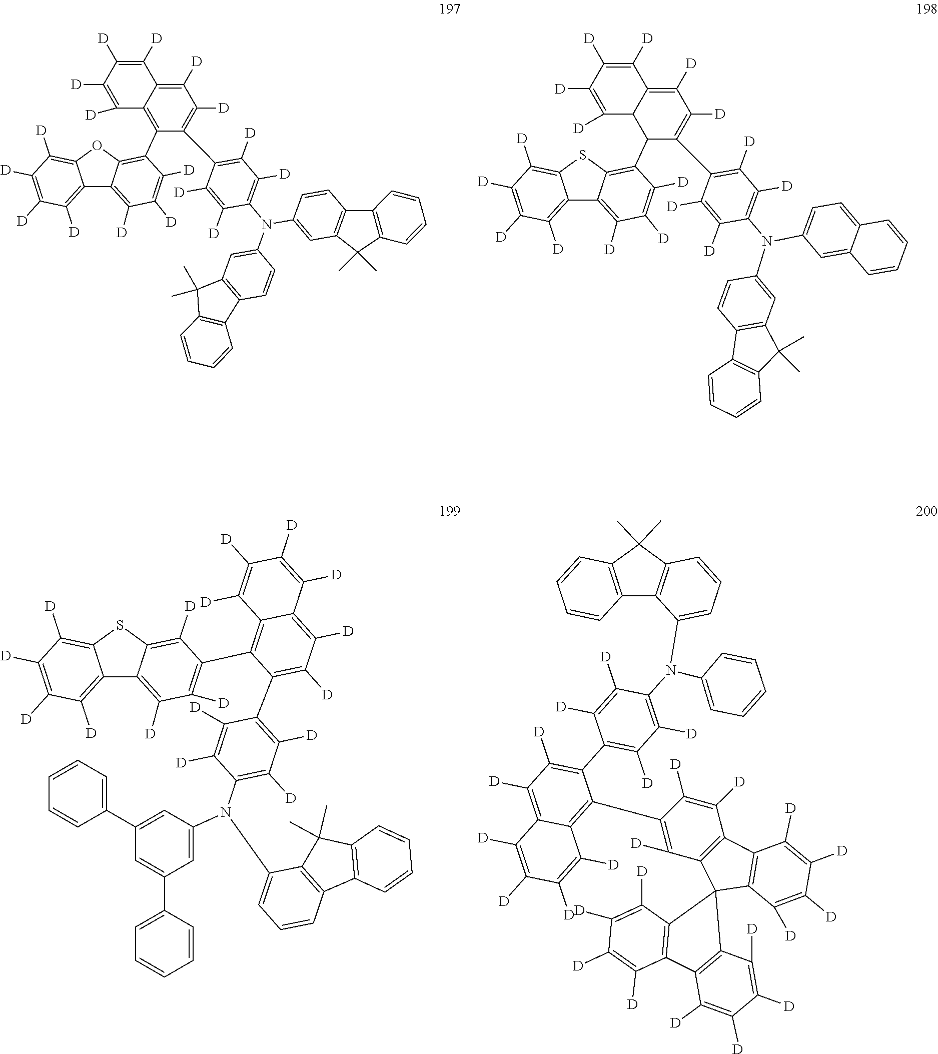

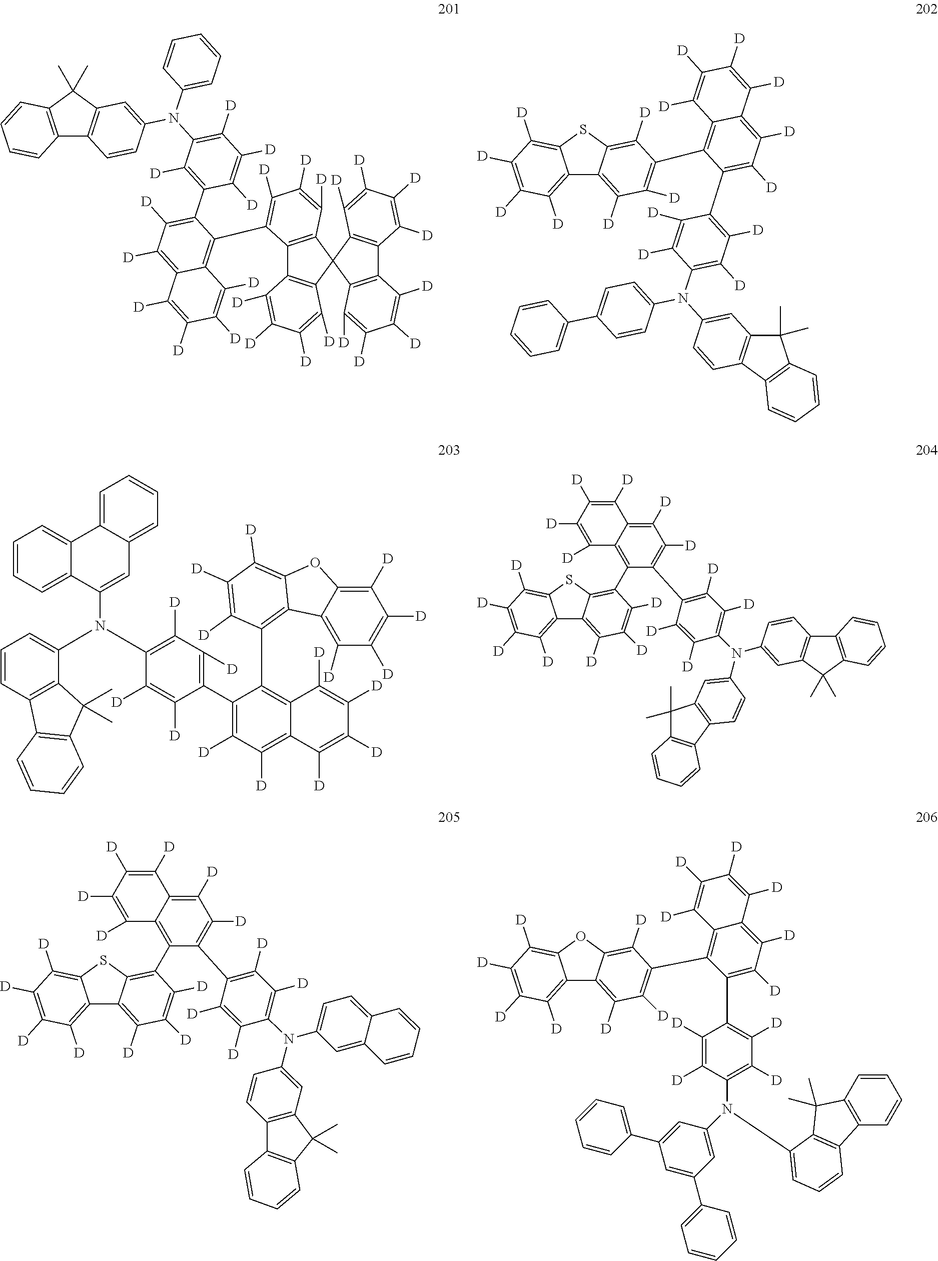

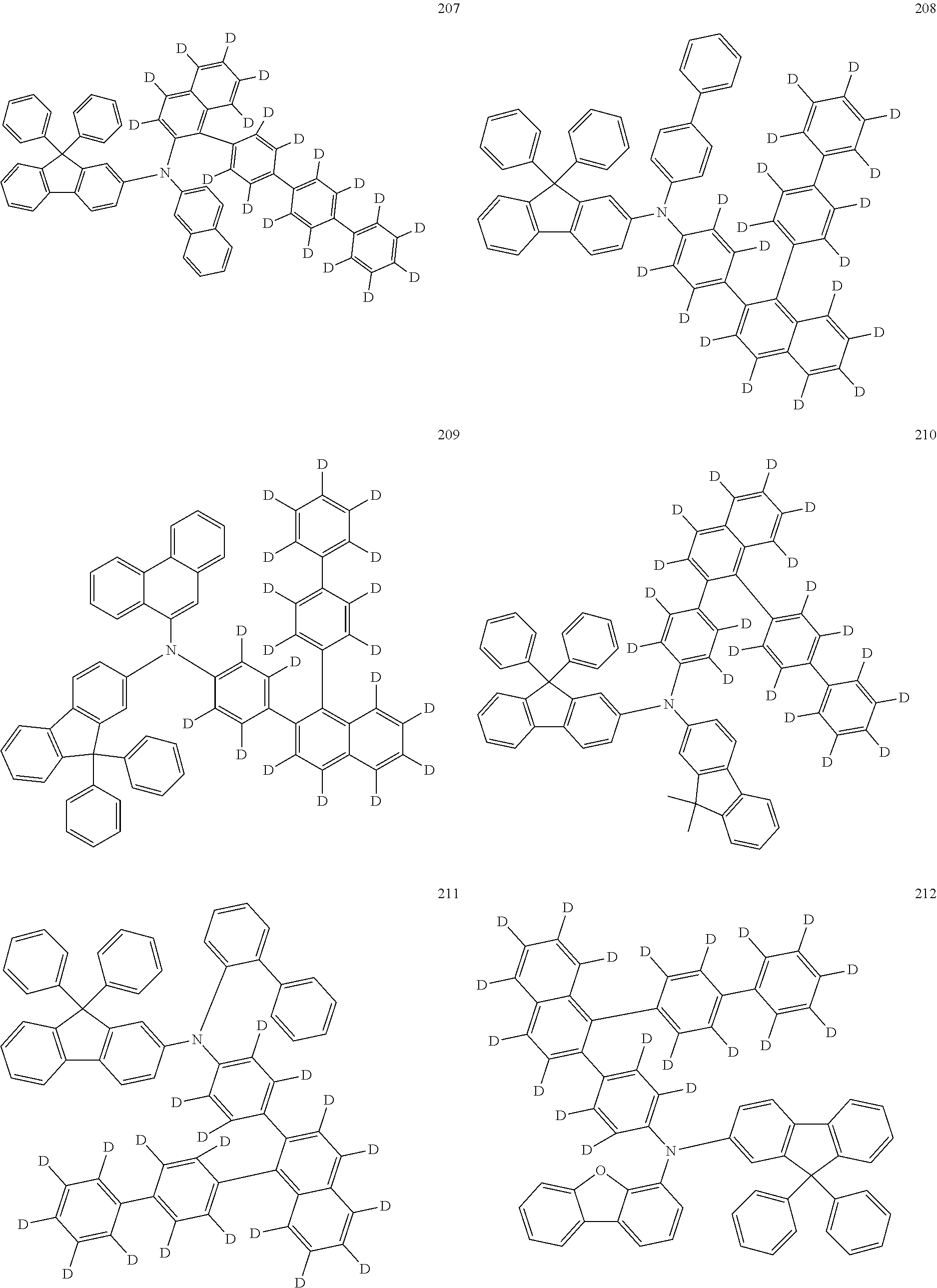

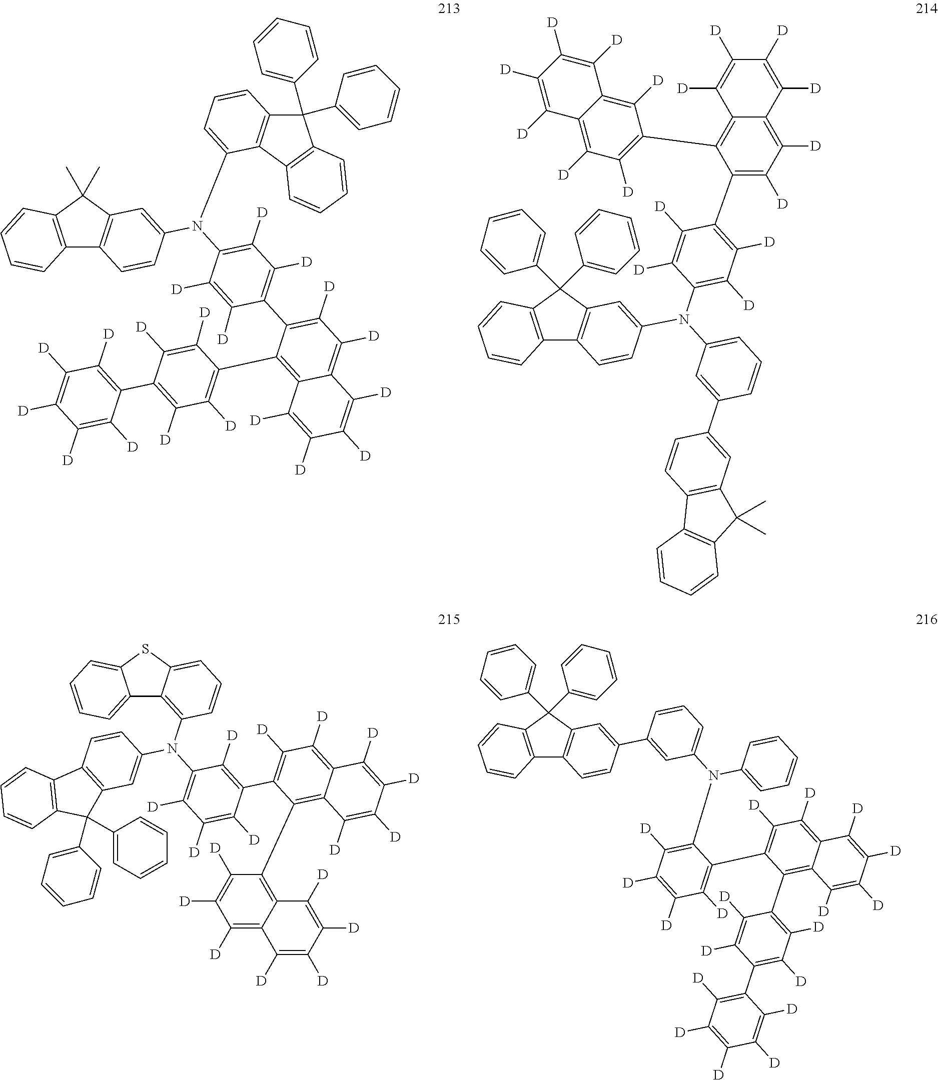

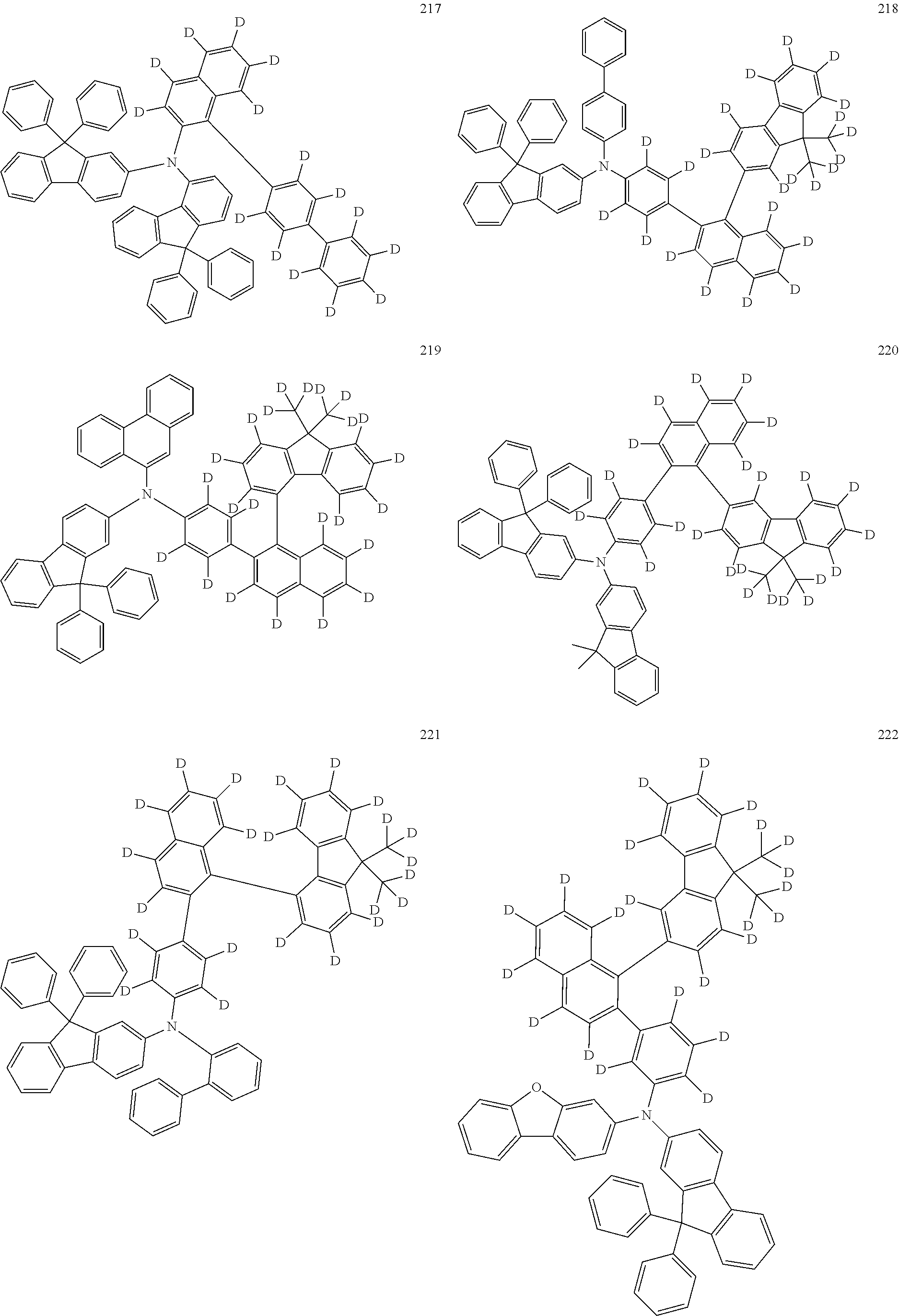

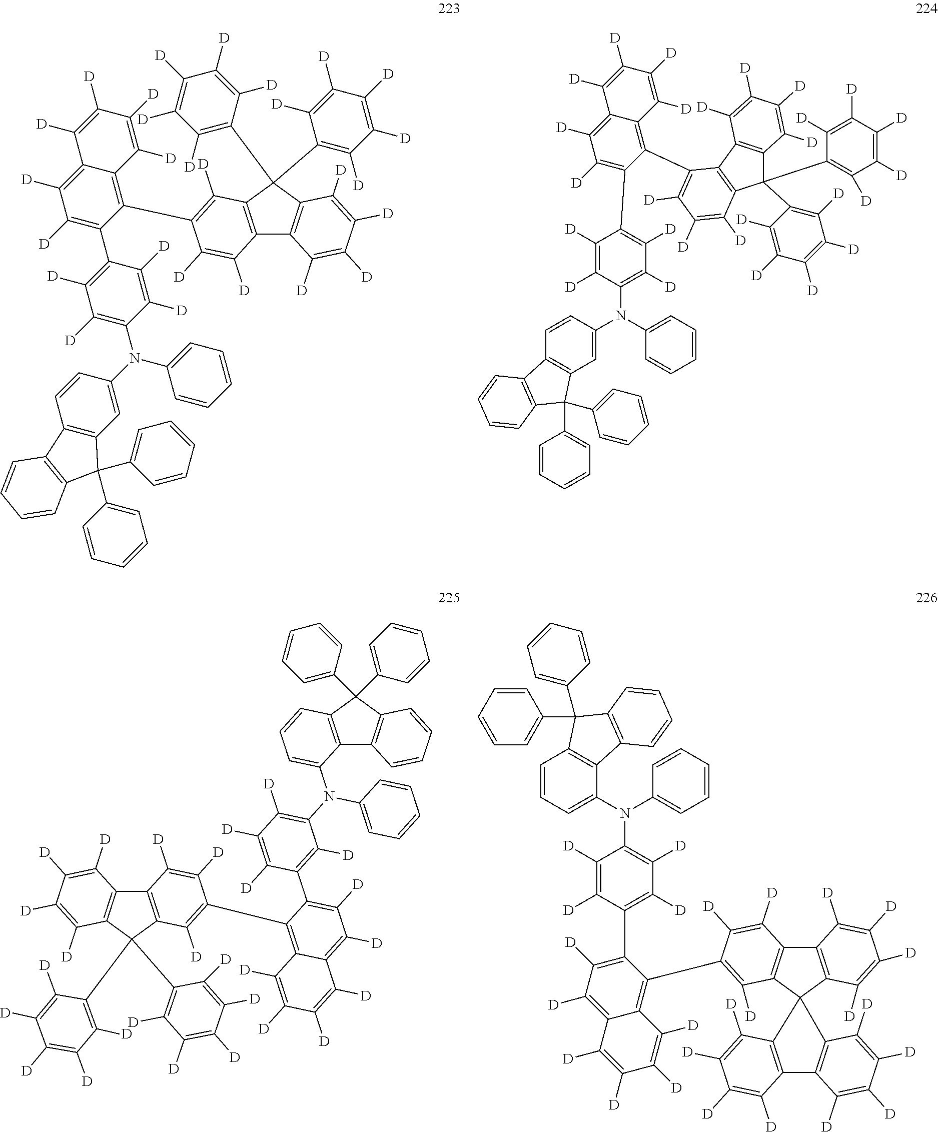

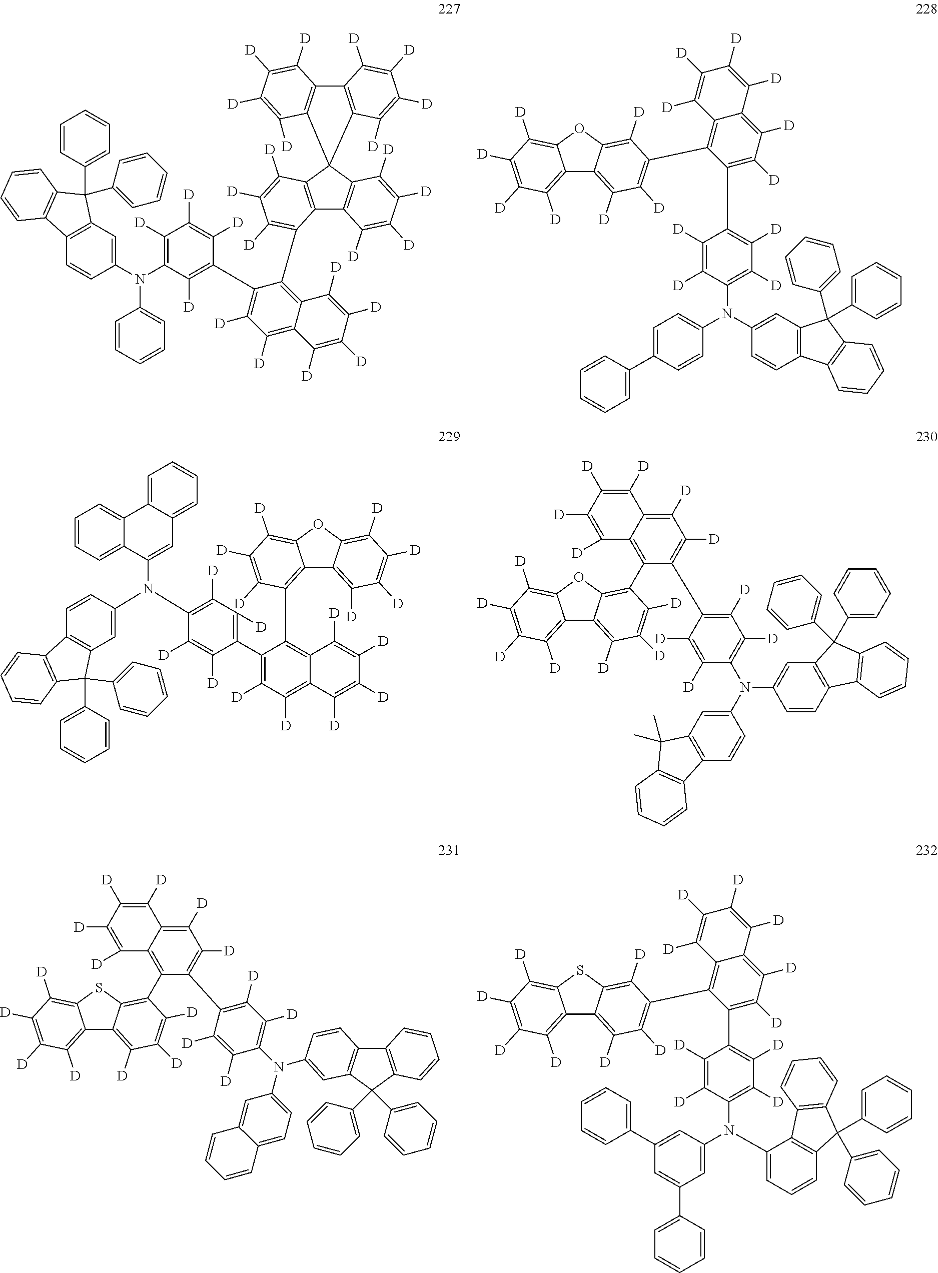

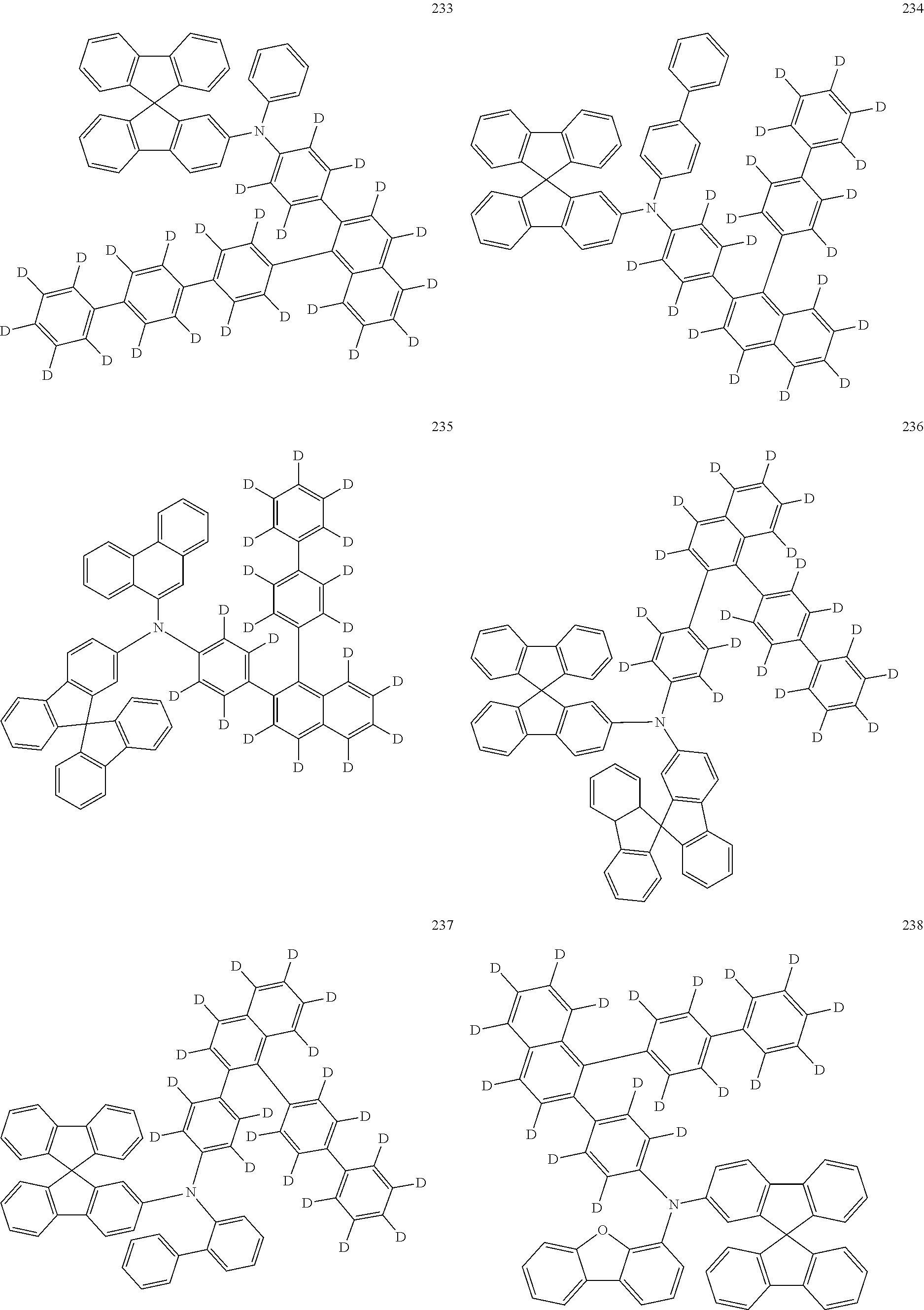

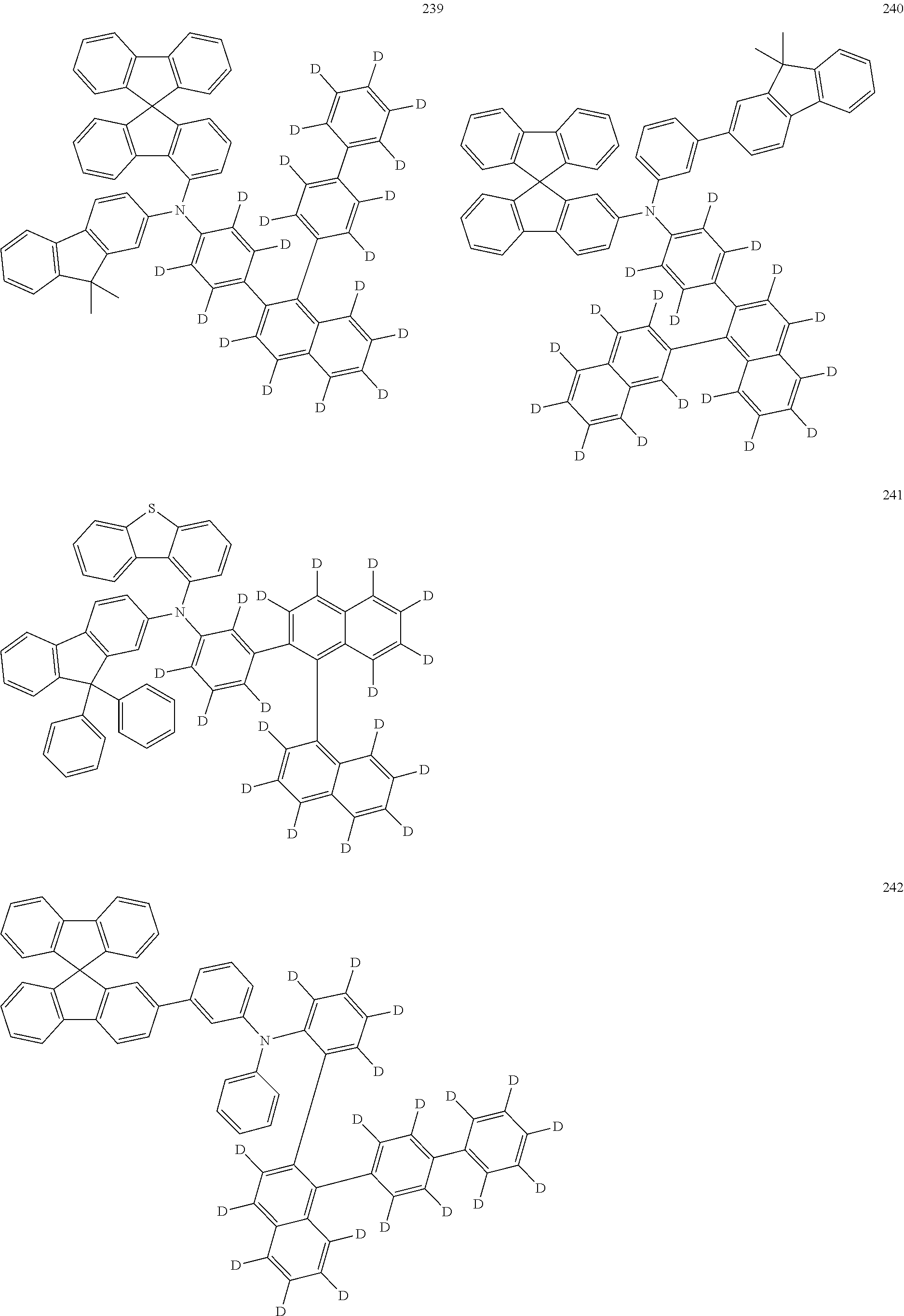

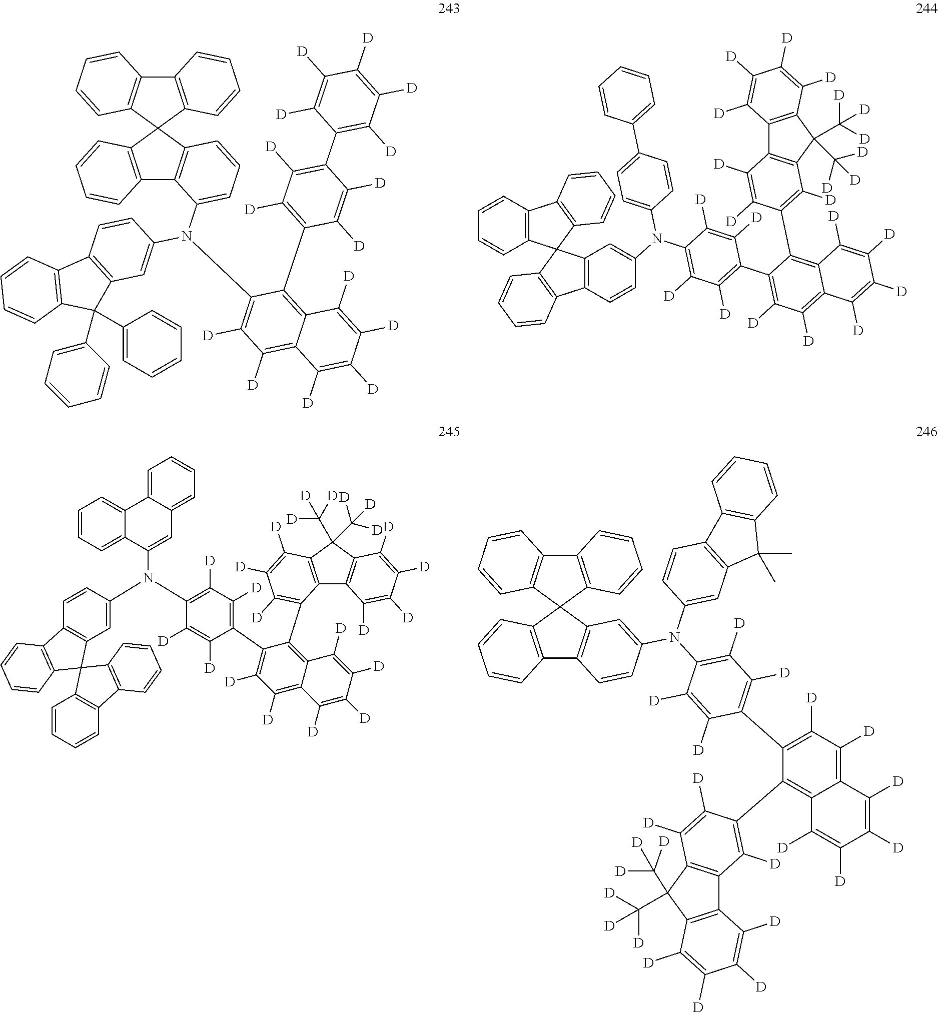

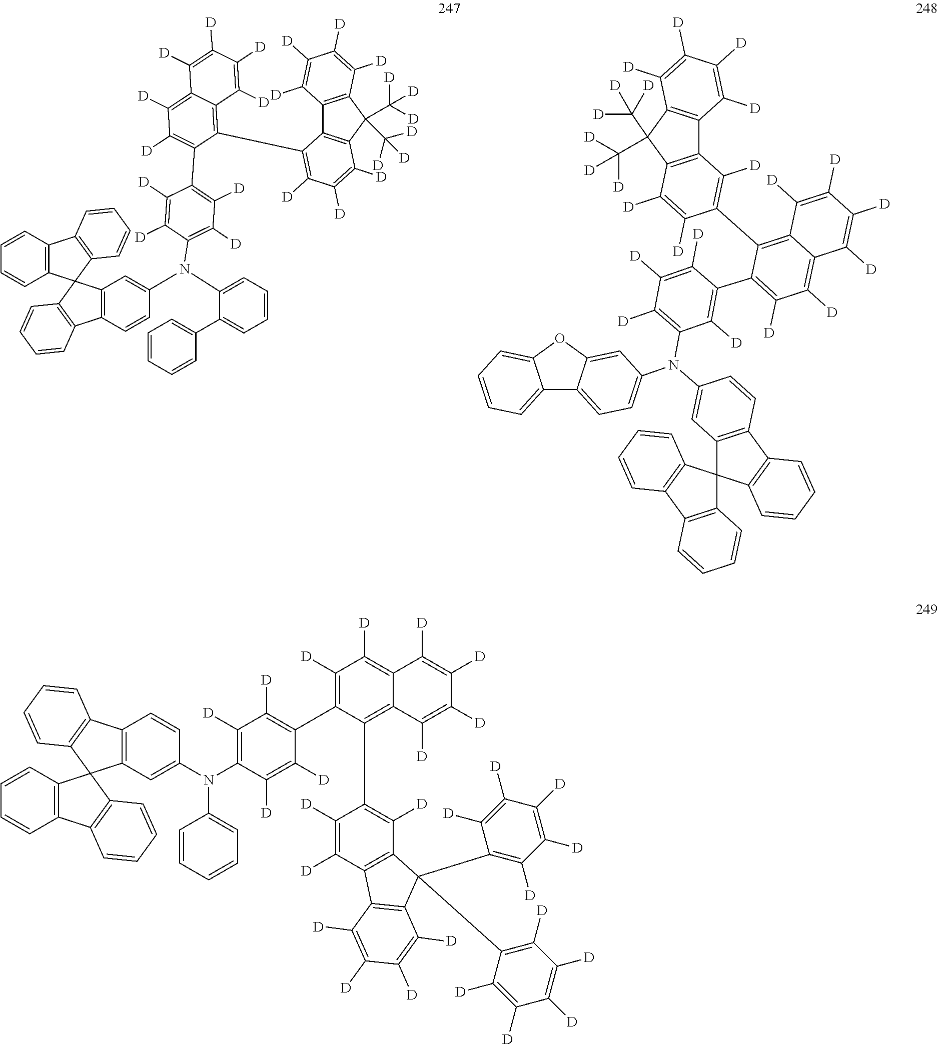

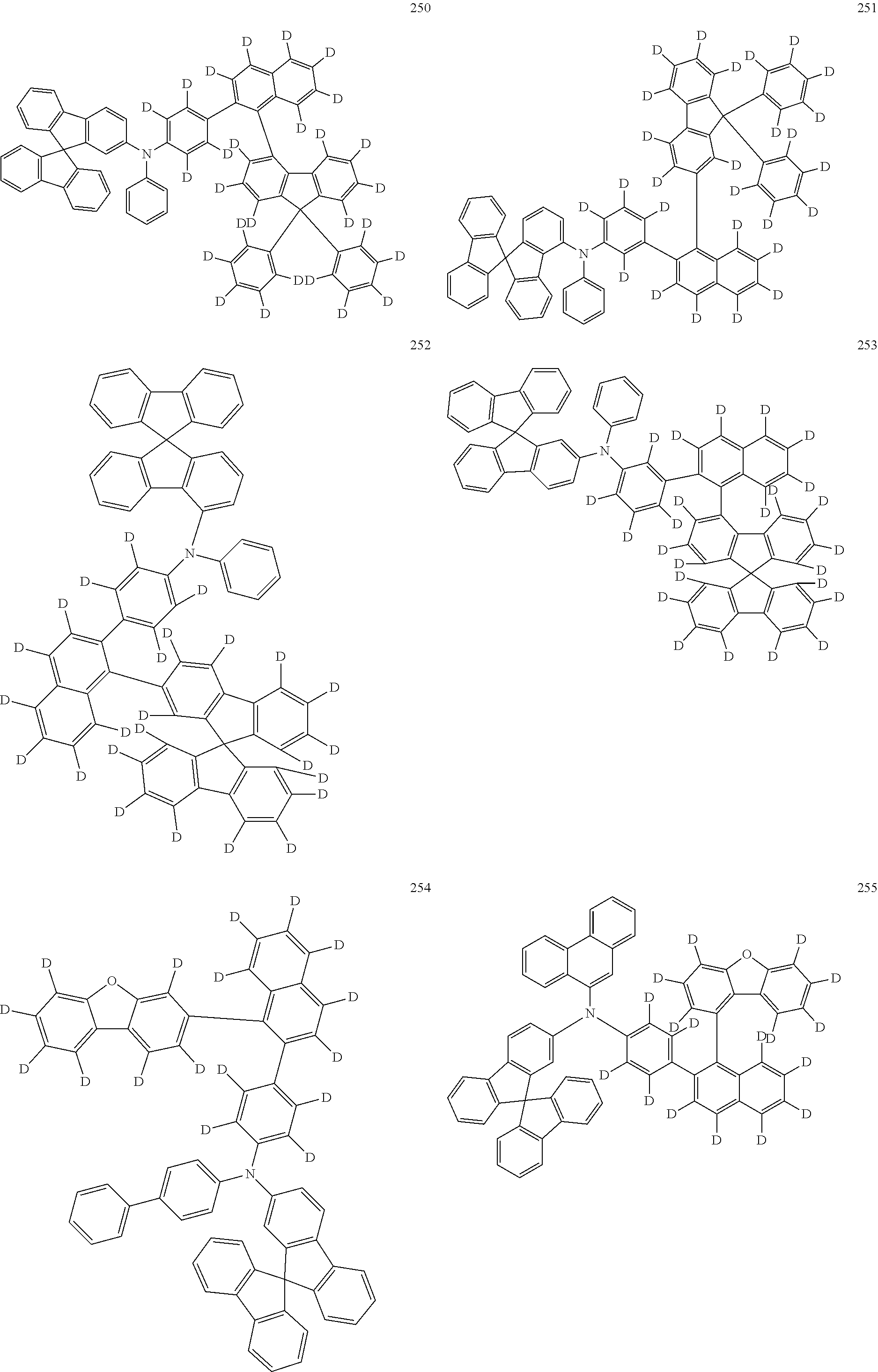

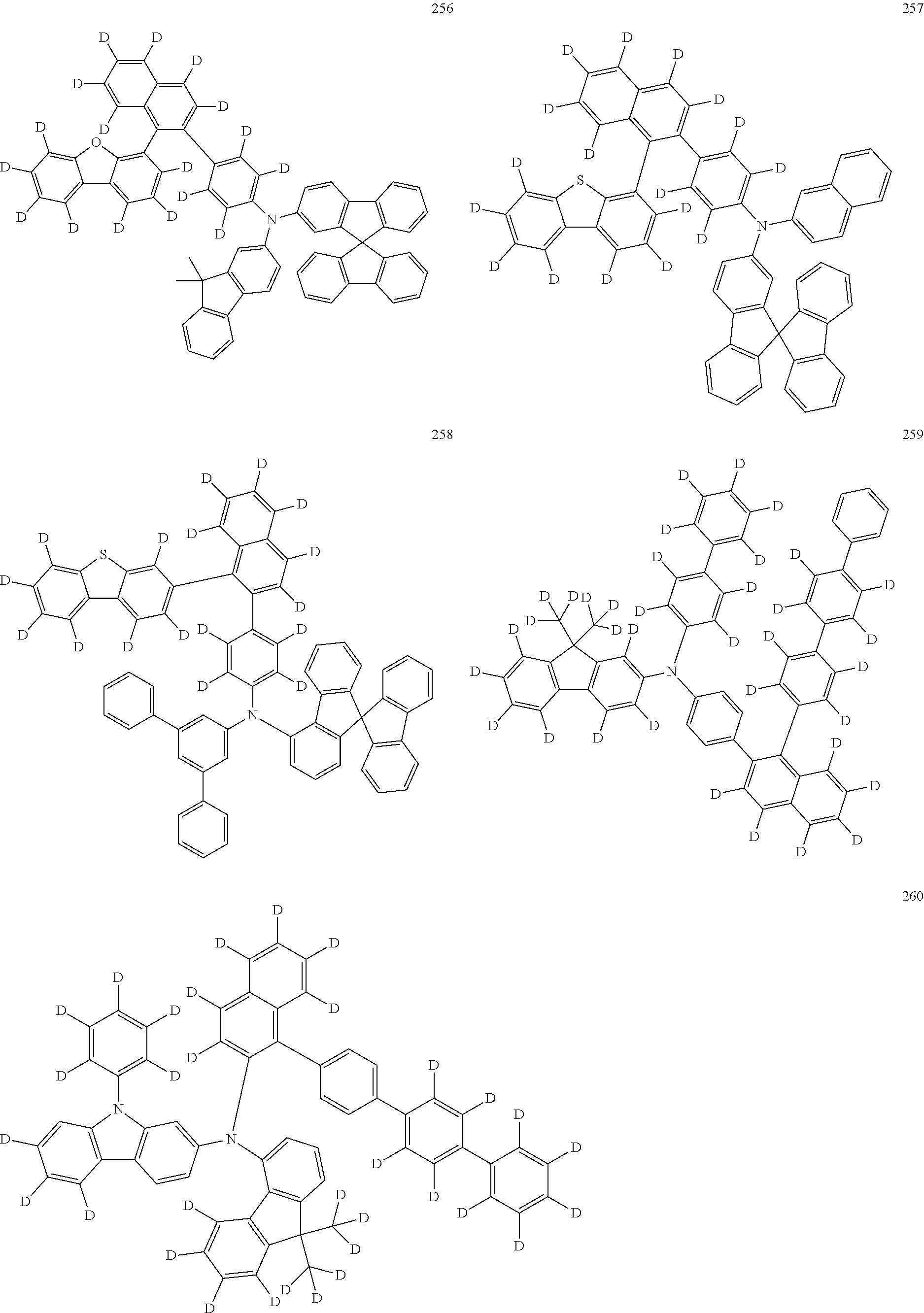

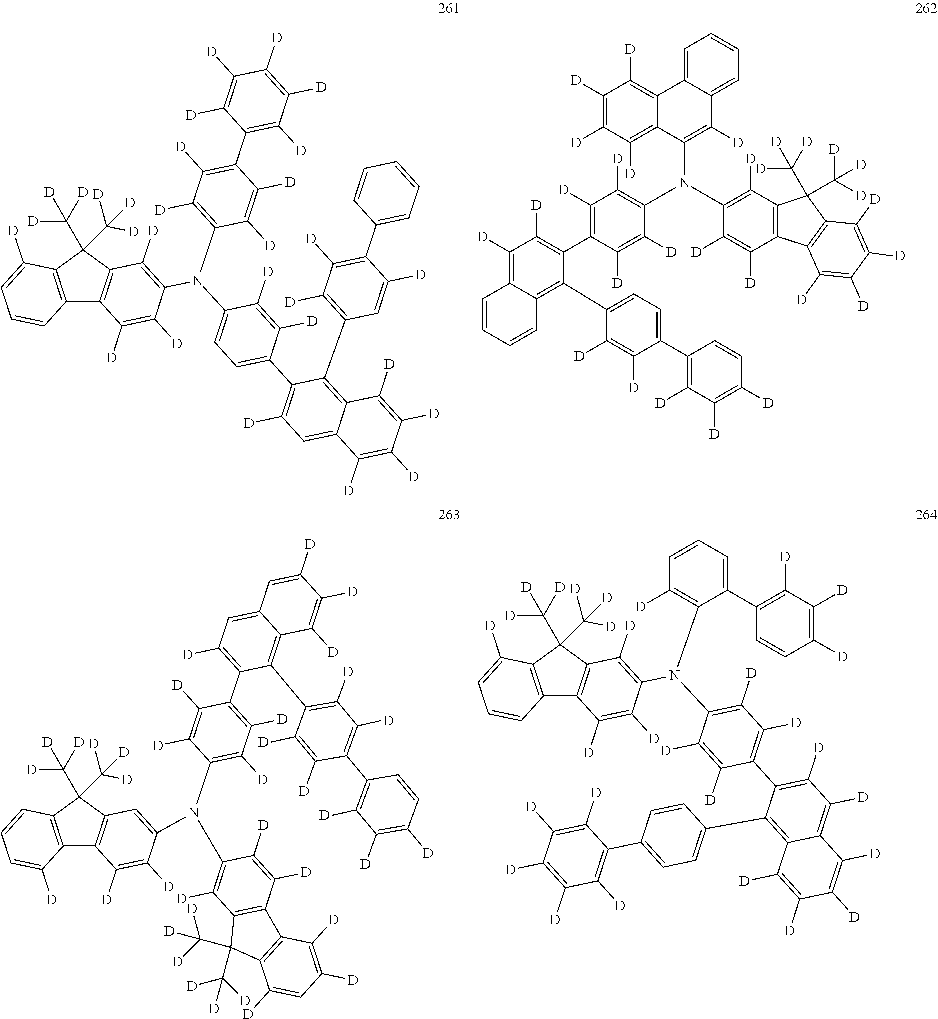

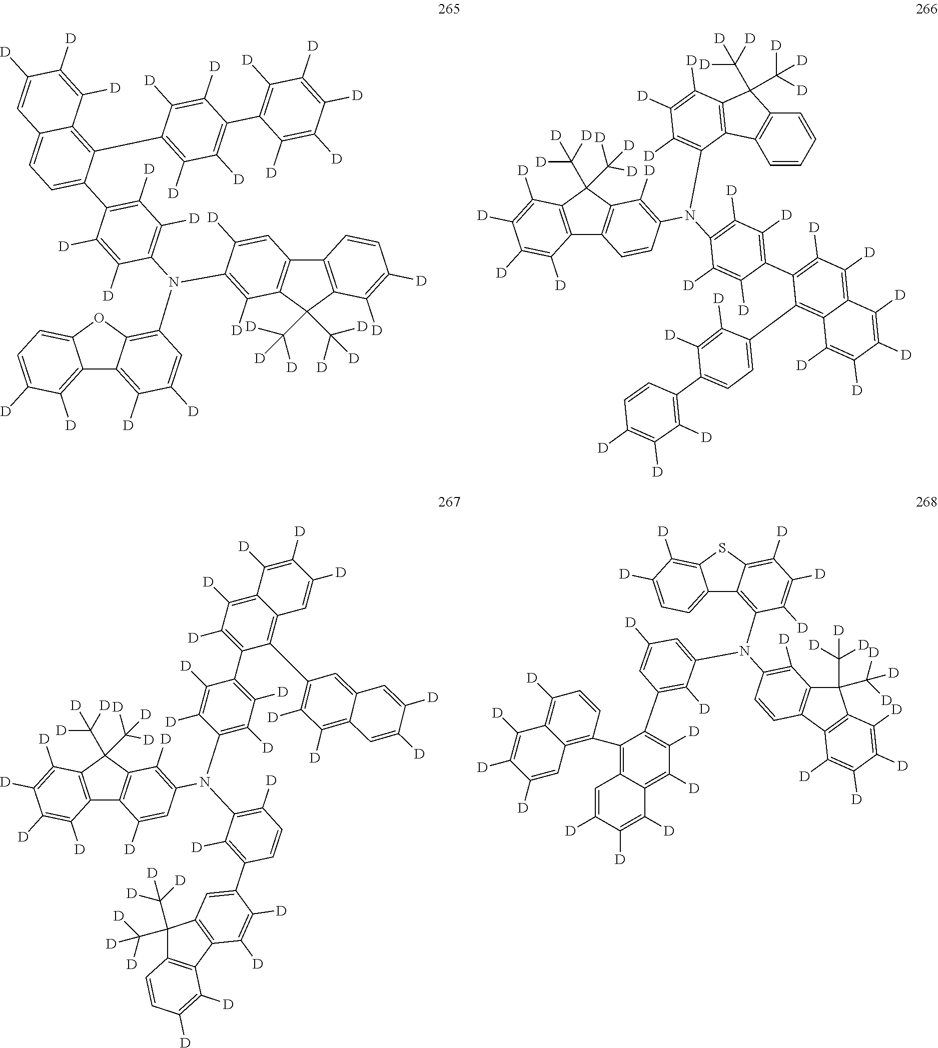

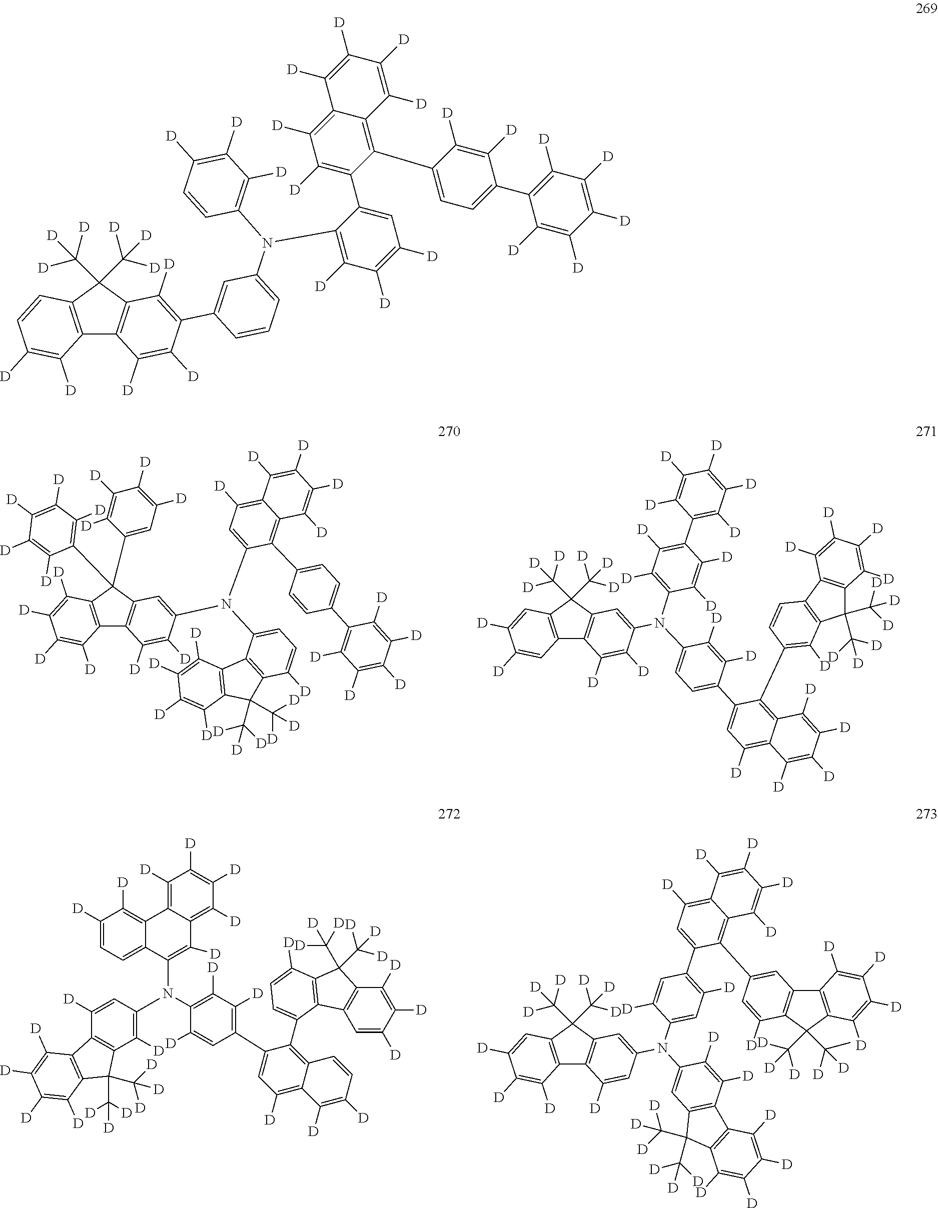

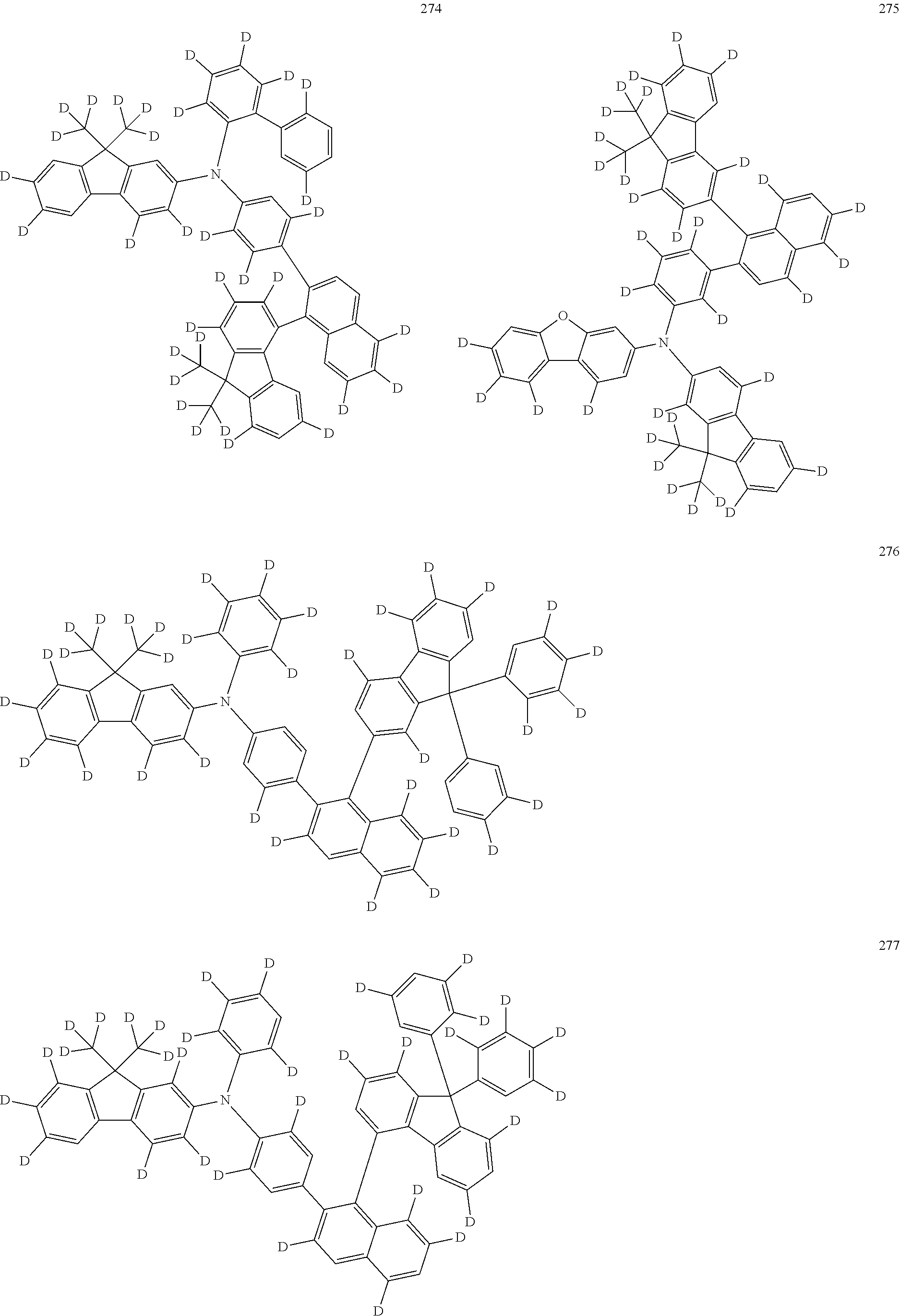

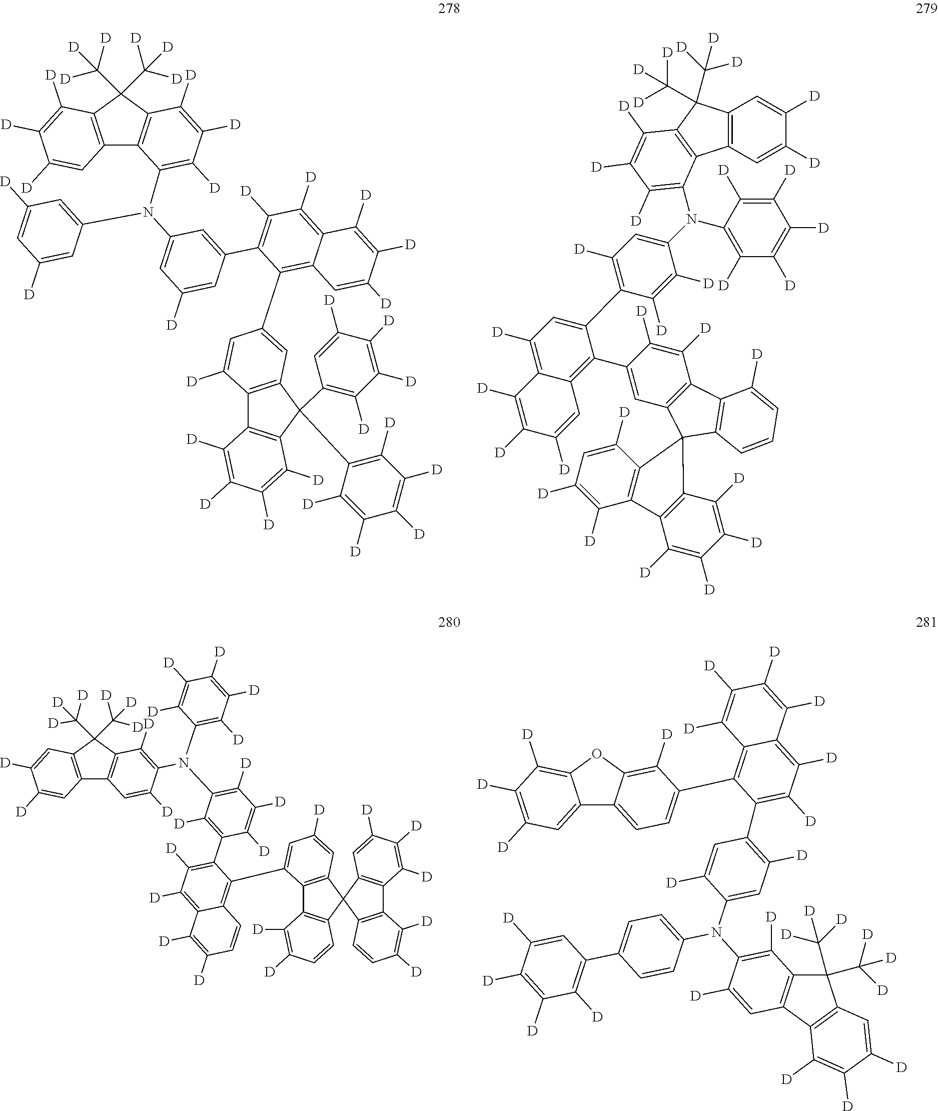

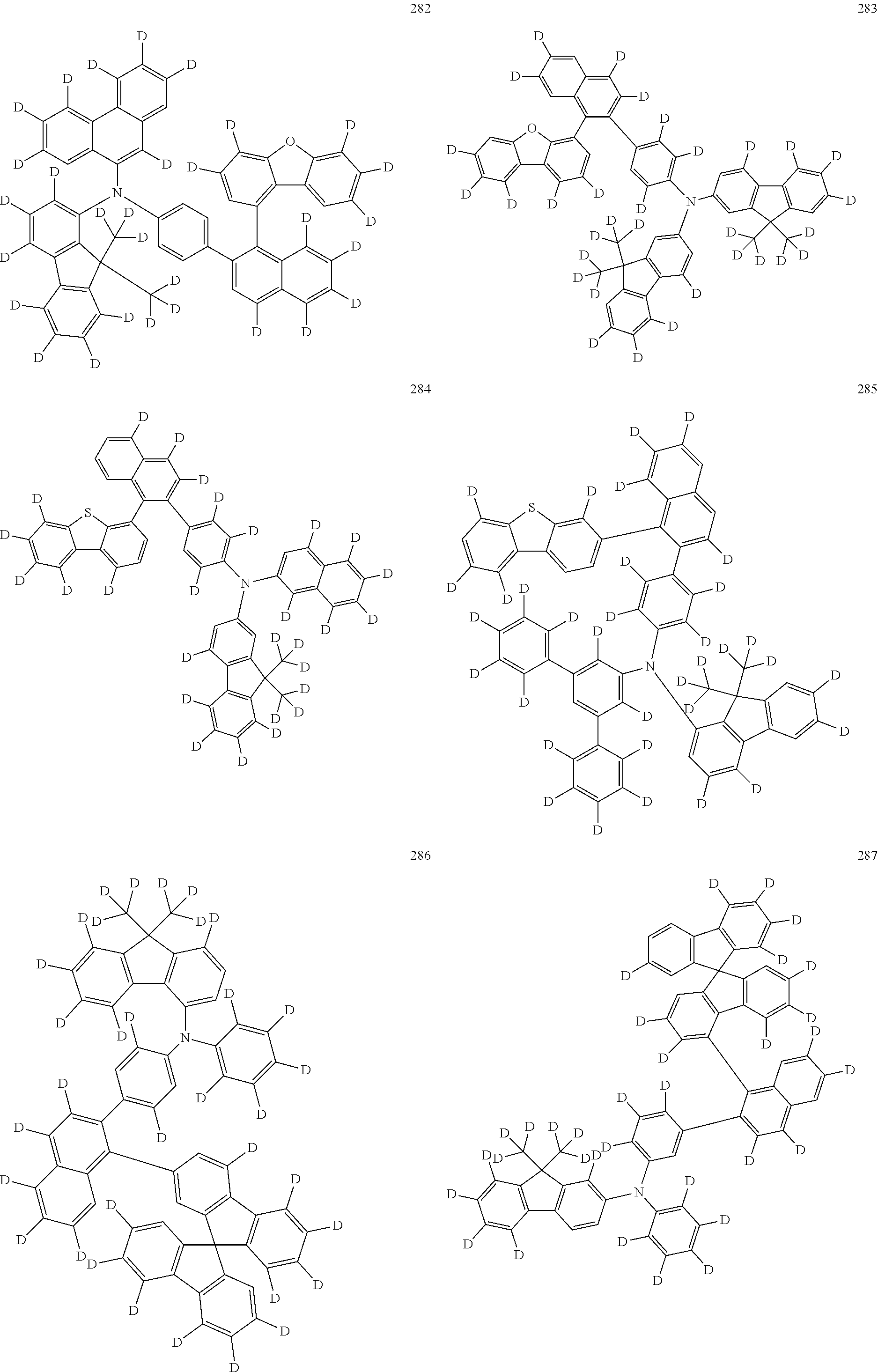

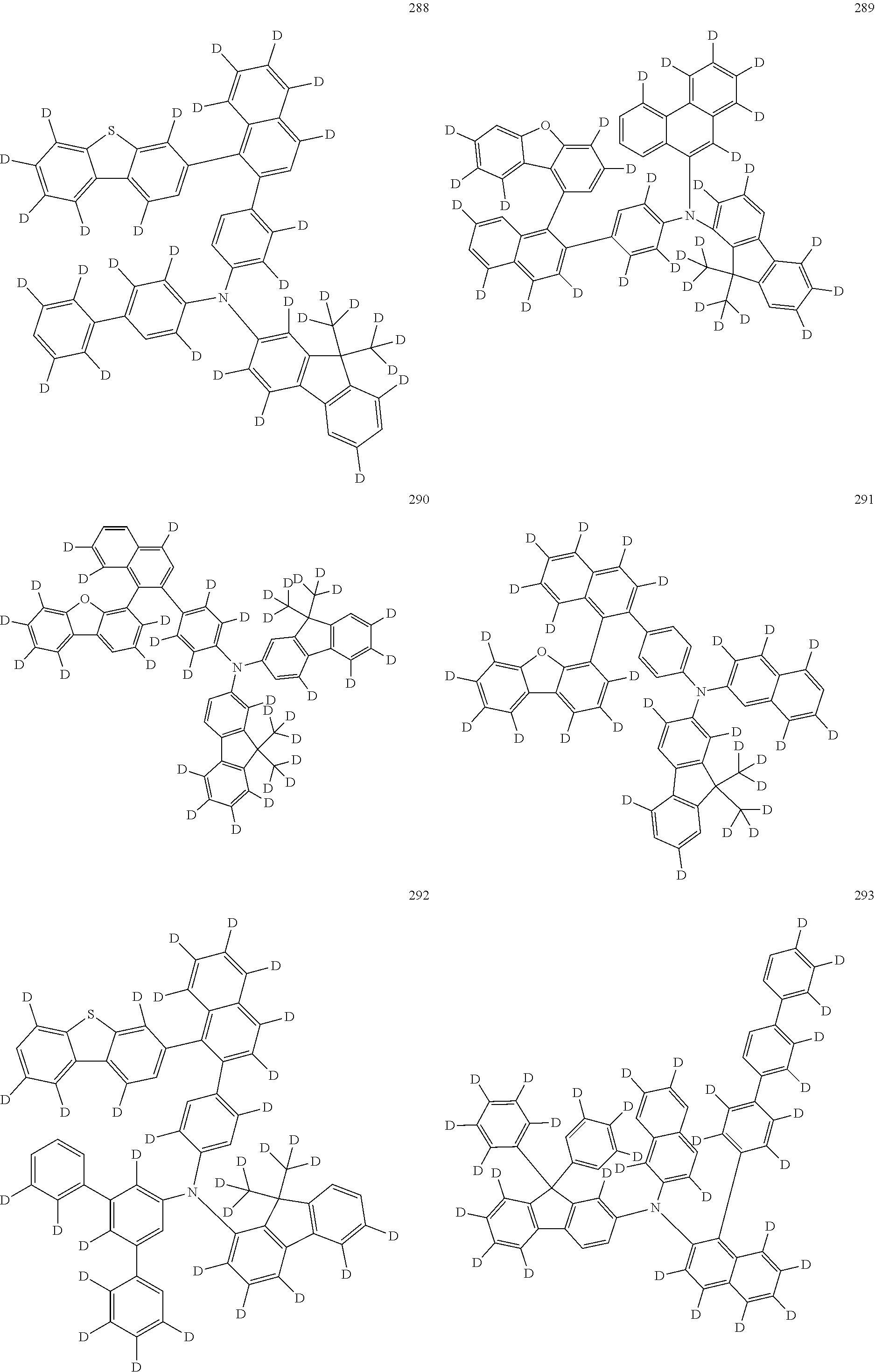

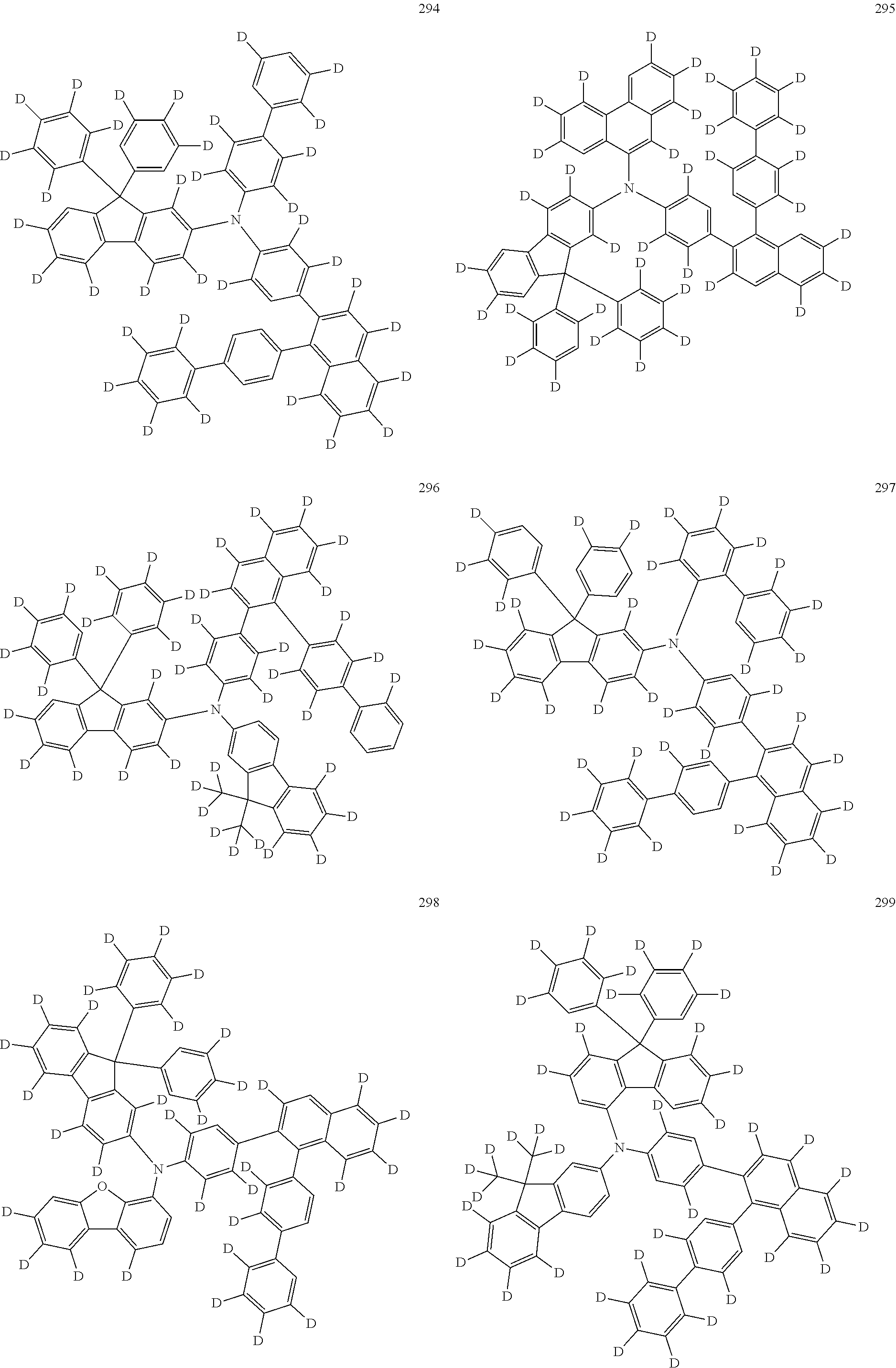

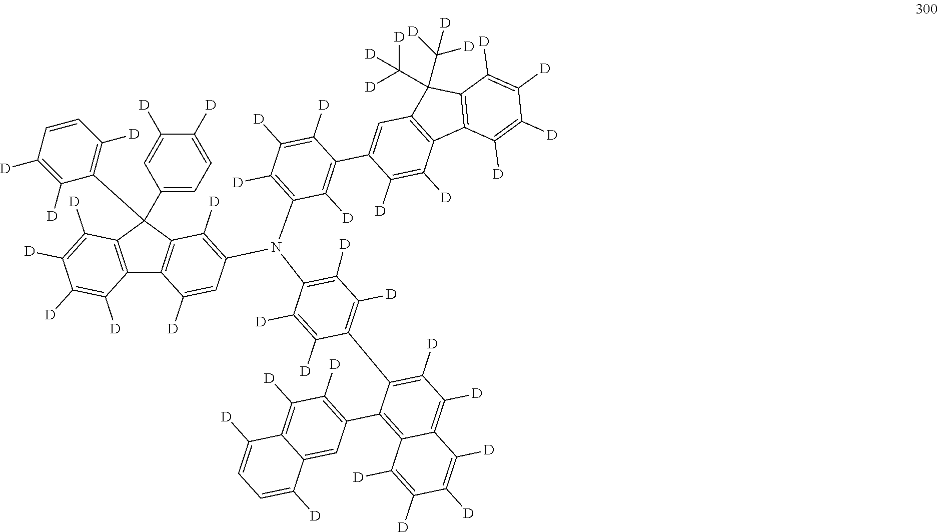 [SMILES: [2H]c1ccc(C2(c3c([2H])ccc([2H])c3[2H])c3c([2H])c(N(c4c([2H])c([2H])c(-c5c([2H])c([2H])c6c([2H])c([2H])c([2H])c([2H])c6c5-c5cc6c([2H])ccc([2H])c6c([2H])c5[2H])c([2H])c4[2H])c4c([2H])c([2H])c([2H])c(-c5cc6c(c([2H])c5[2H])-c5c([2H])c([2H])c([2H])c([2H])c5C6(C([2H])([2H])[2H])C([2H])([2H])[2H])c4[2H])cc([2H])c3-c3c([2H])c([2H])c([2H])c([2H])c32)cc1[2H]]

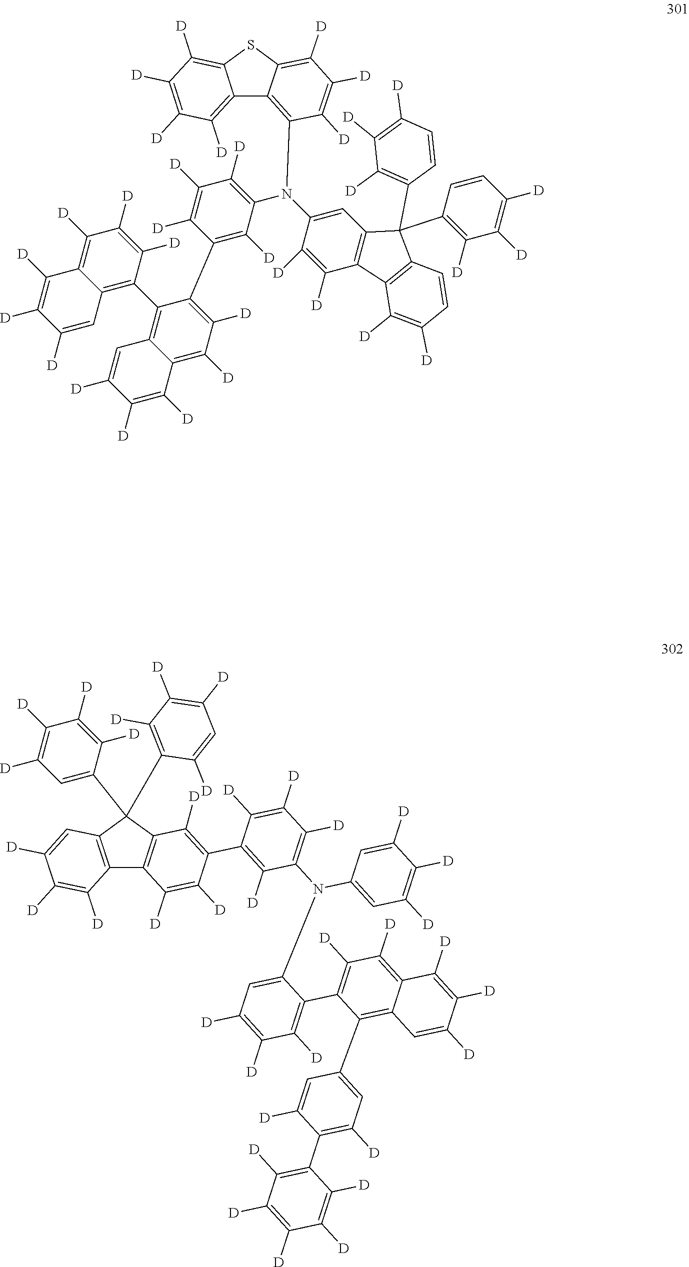

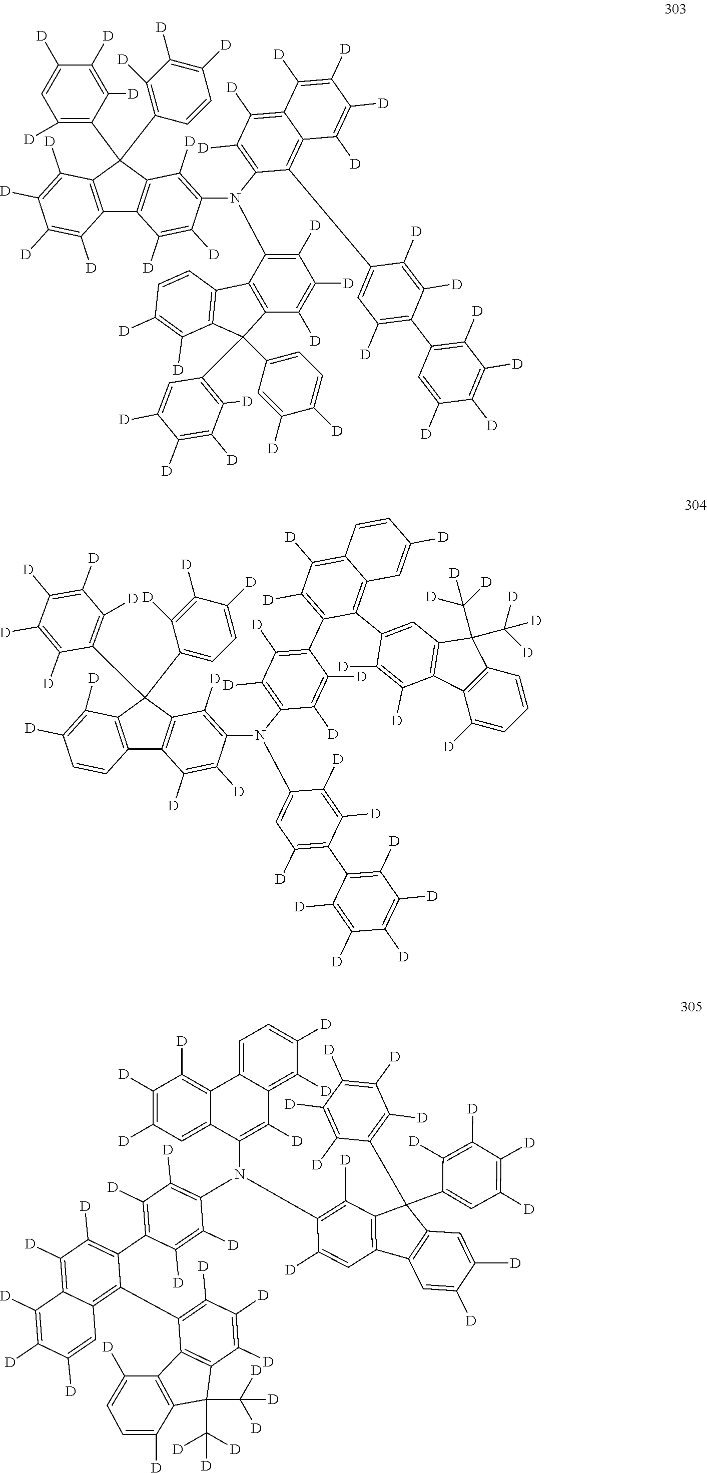

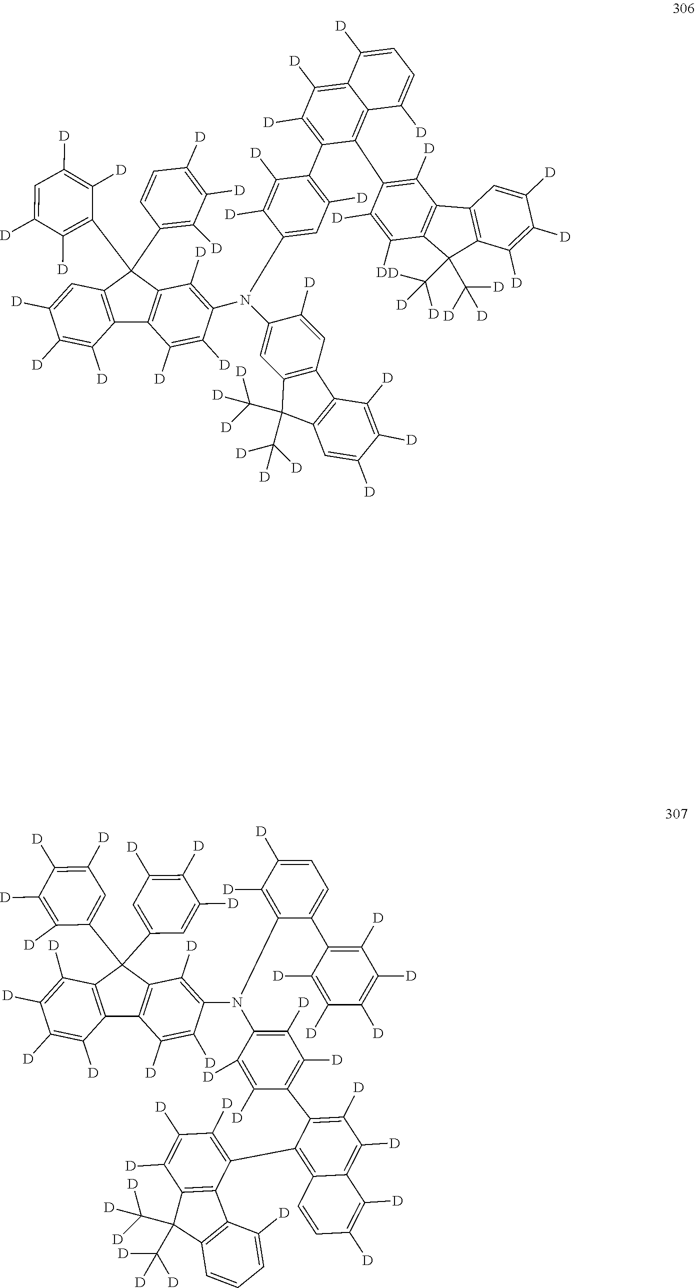

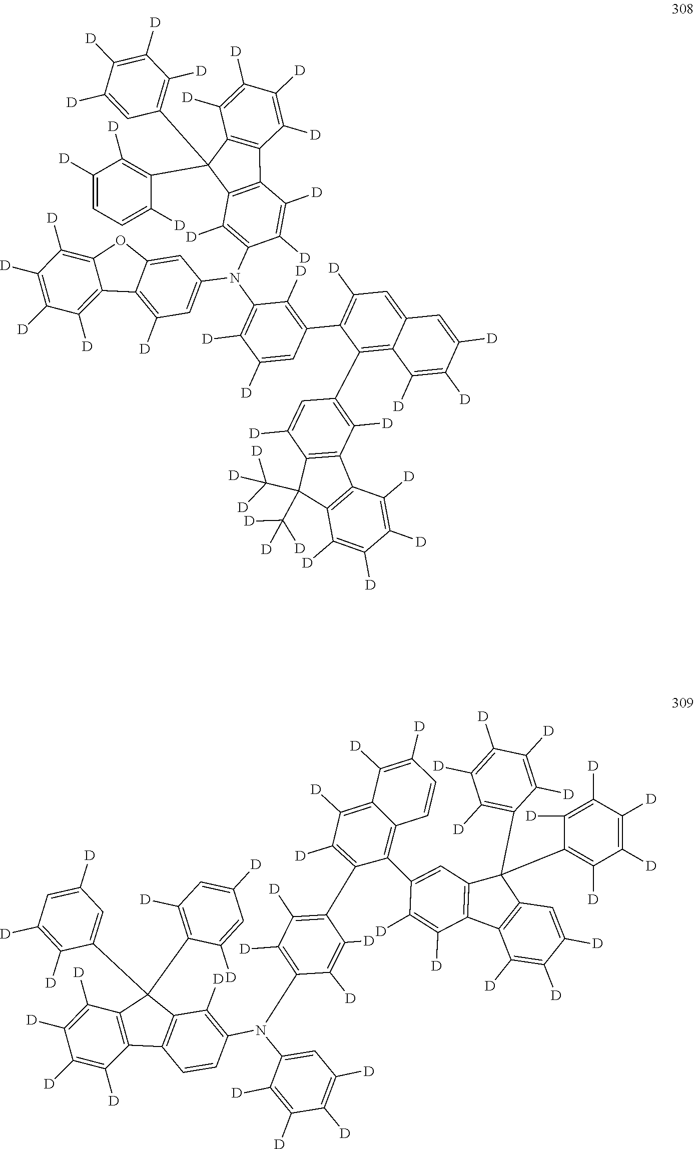

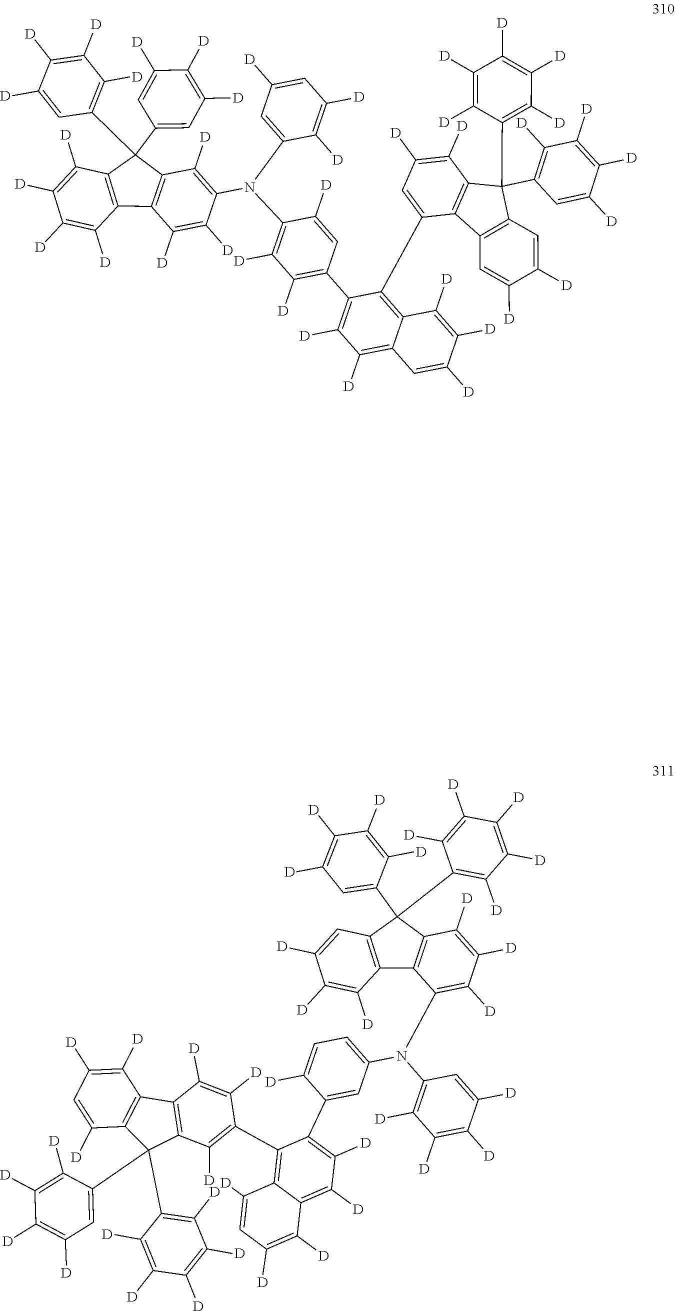

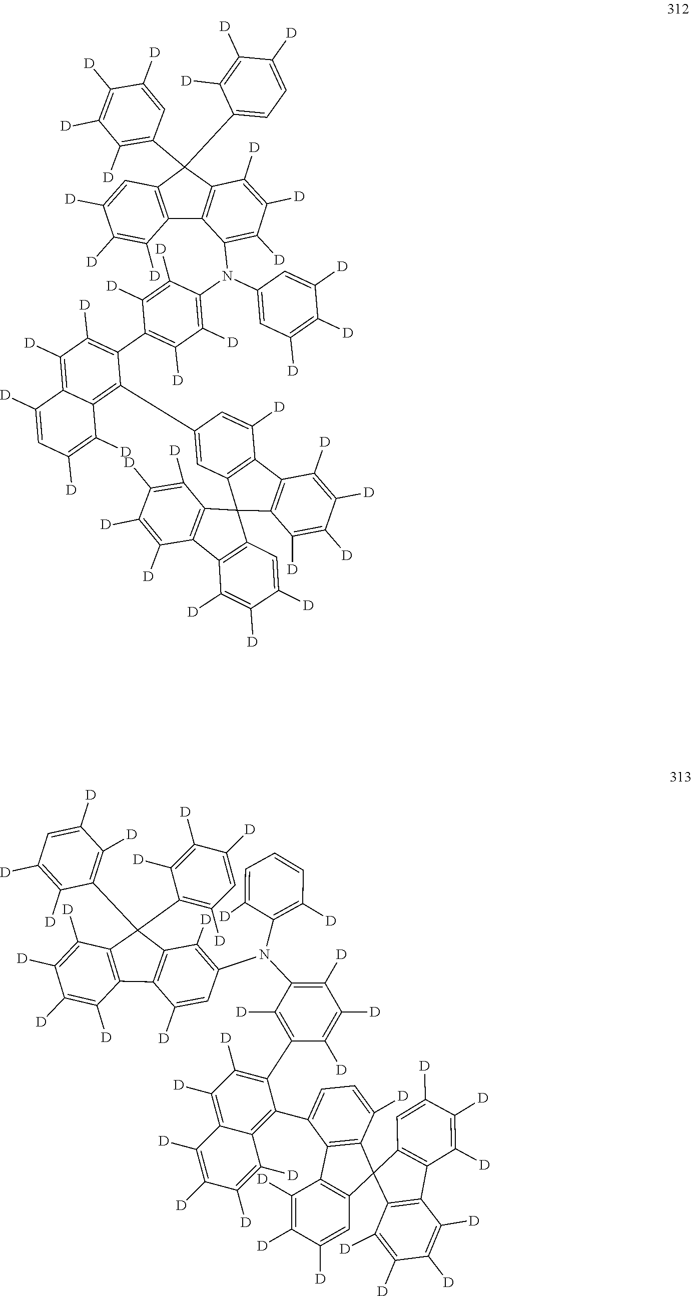

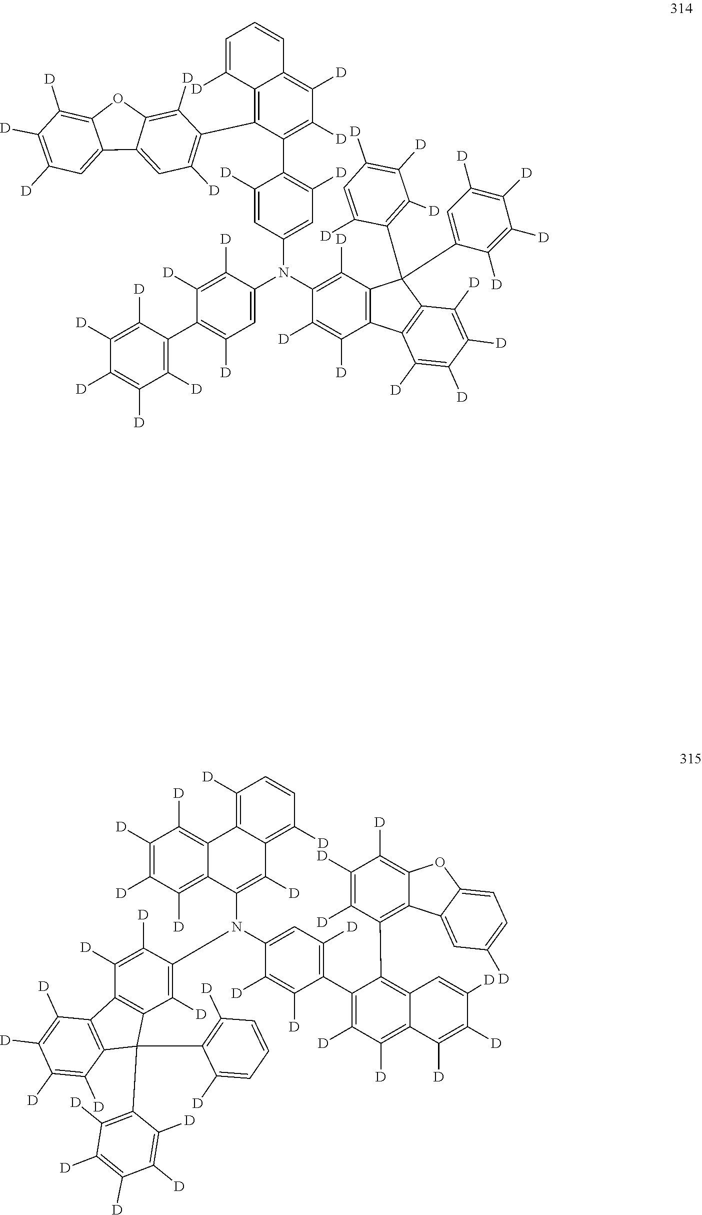

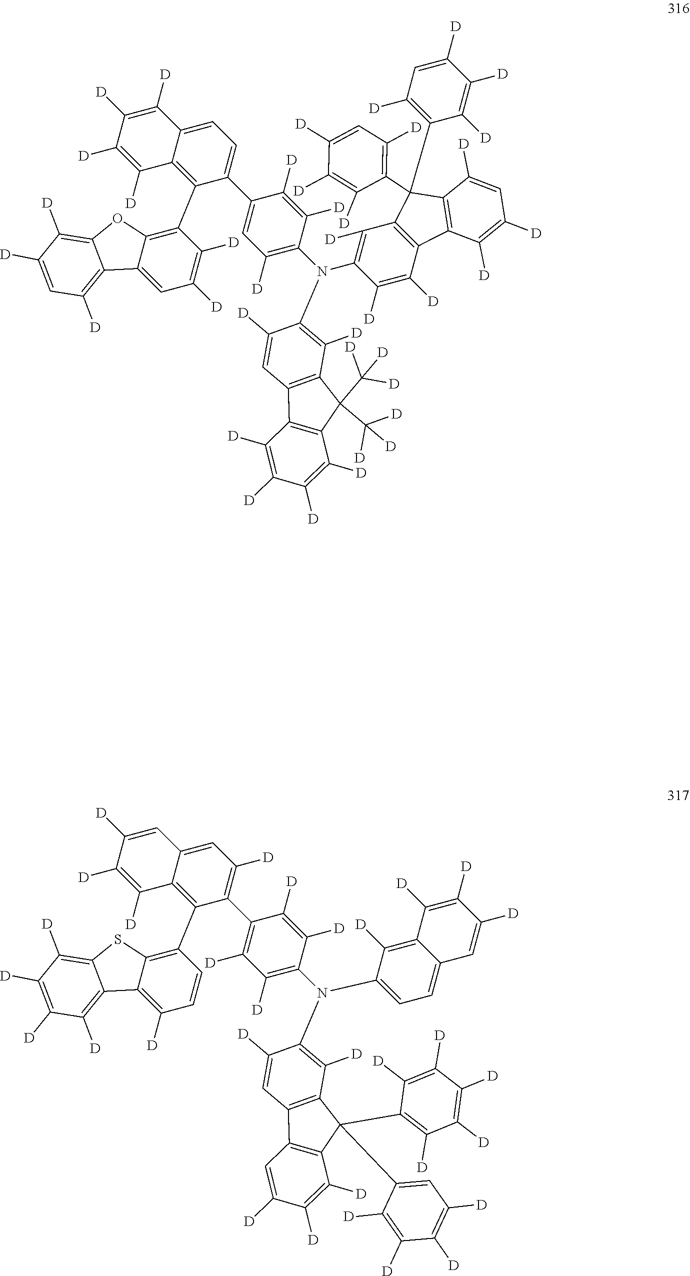

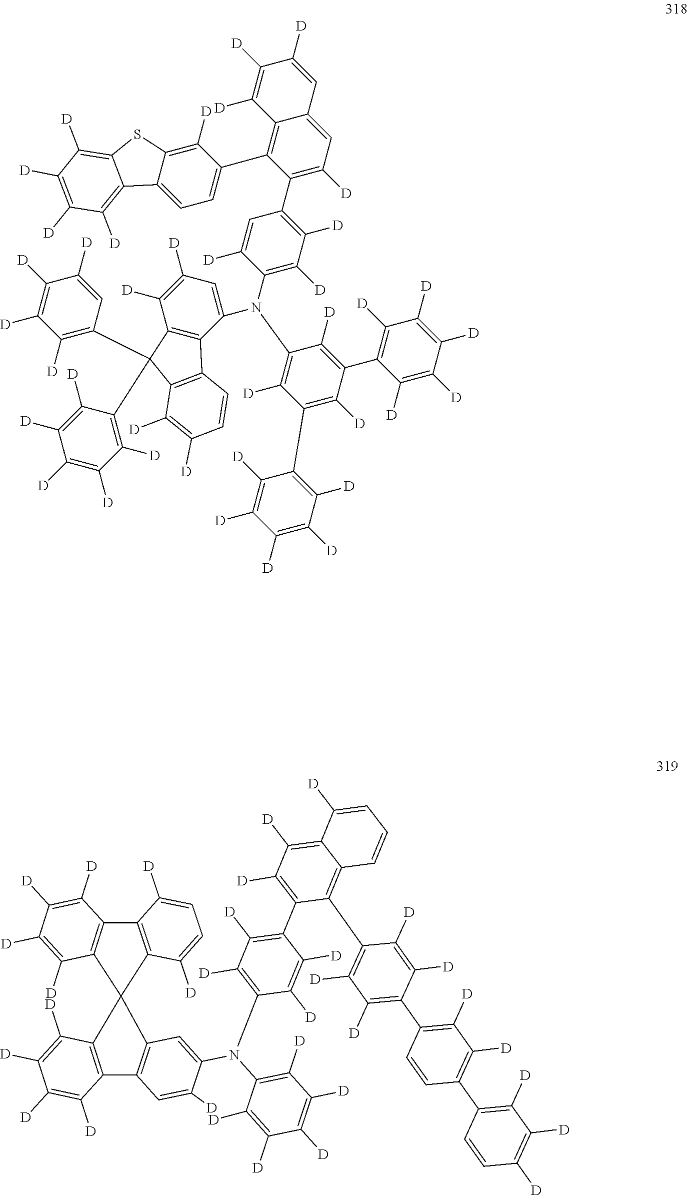

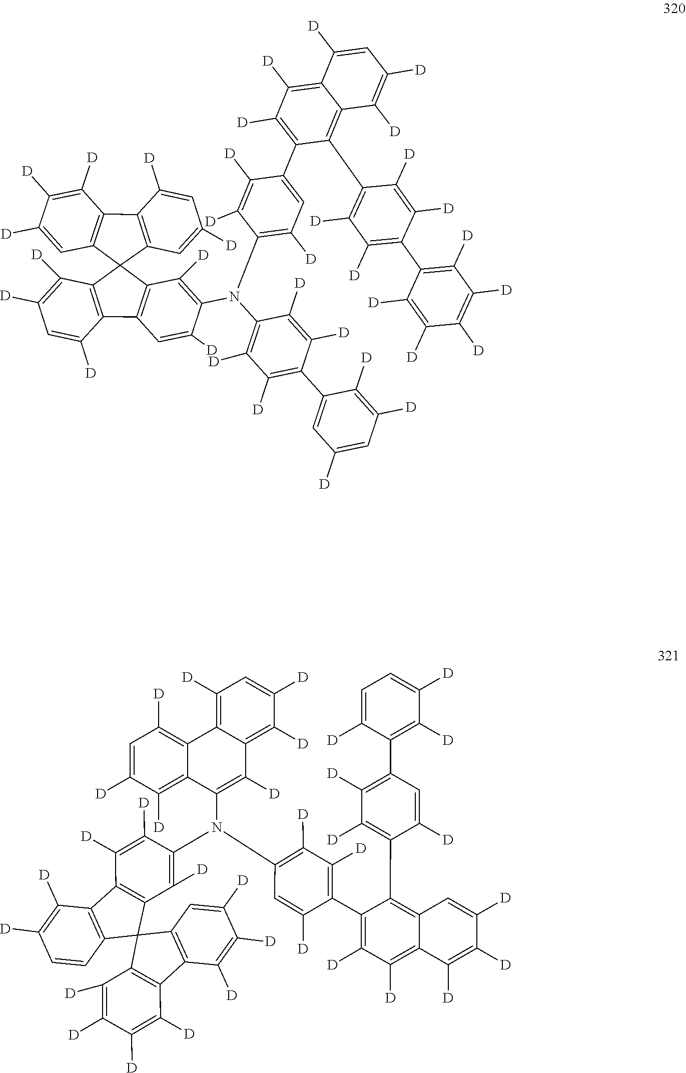

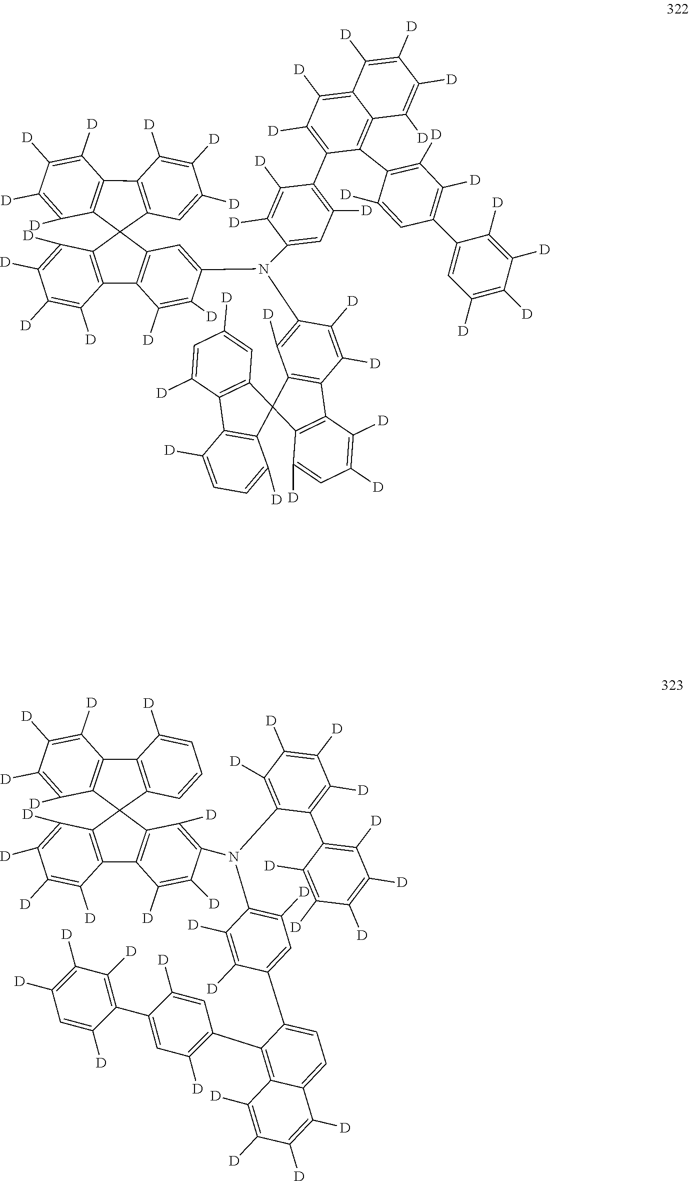

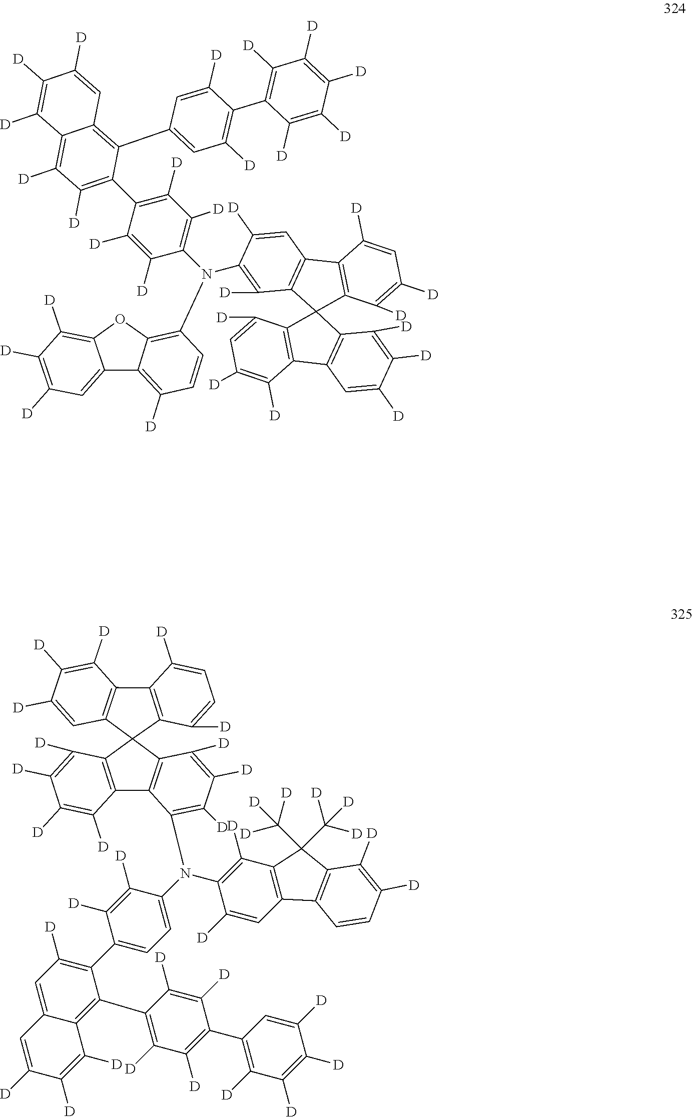

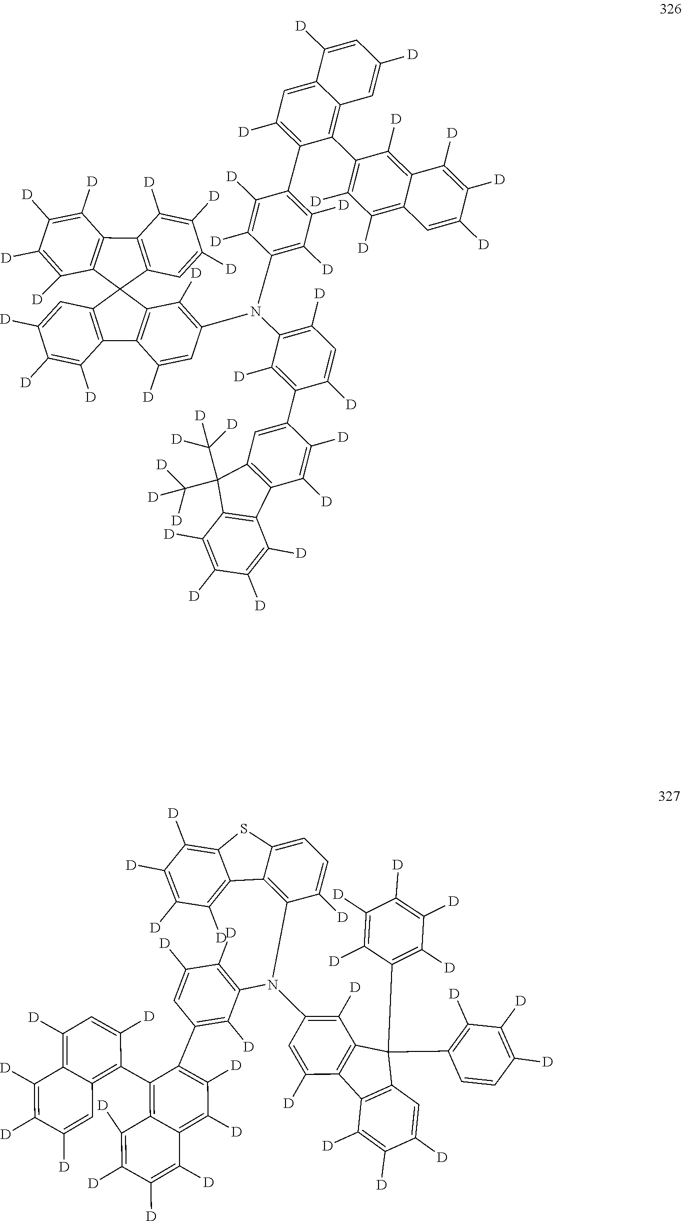

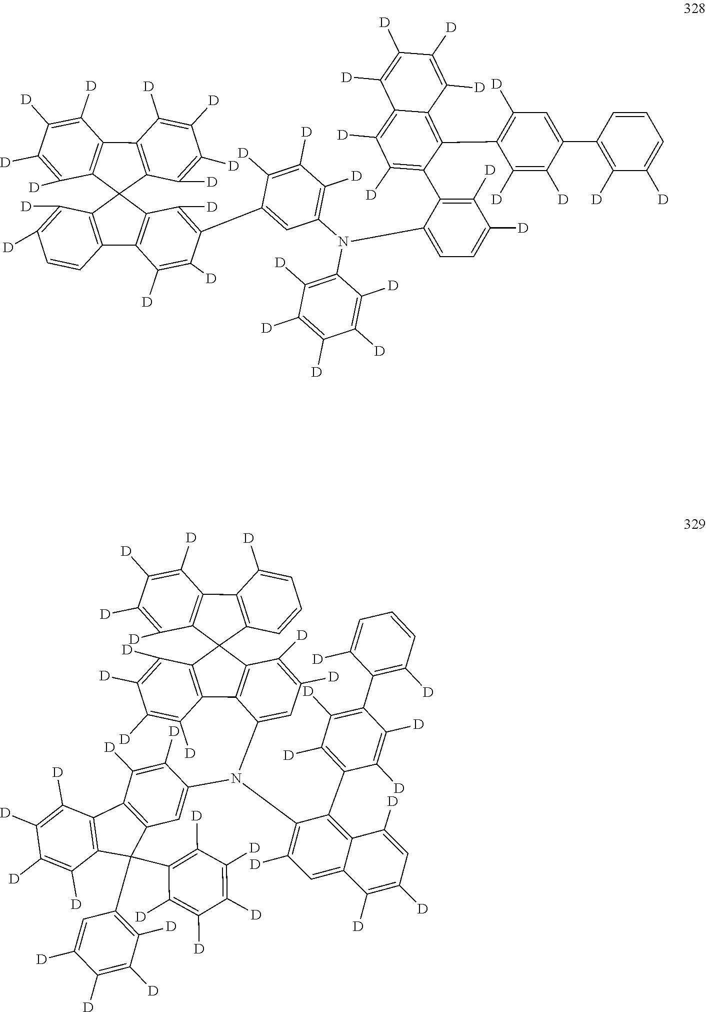

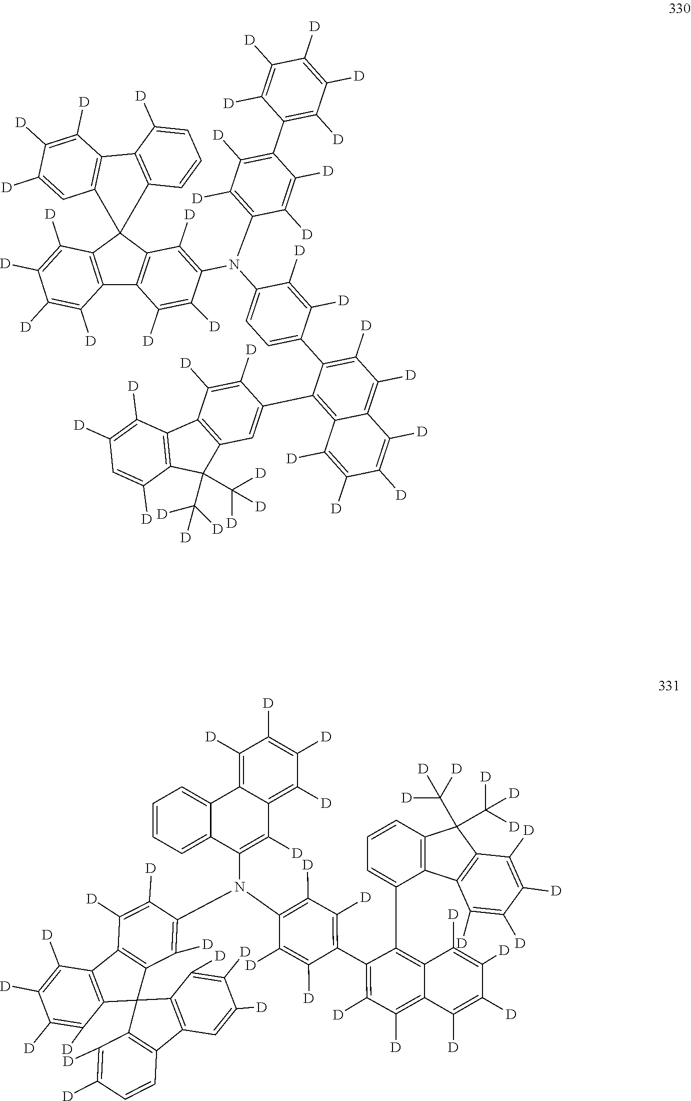

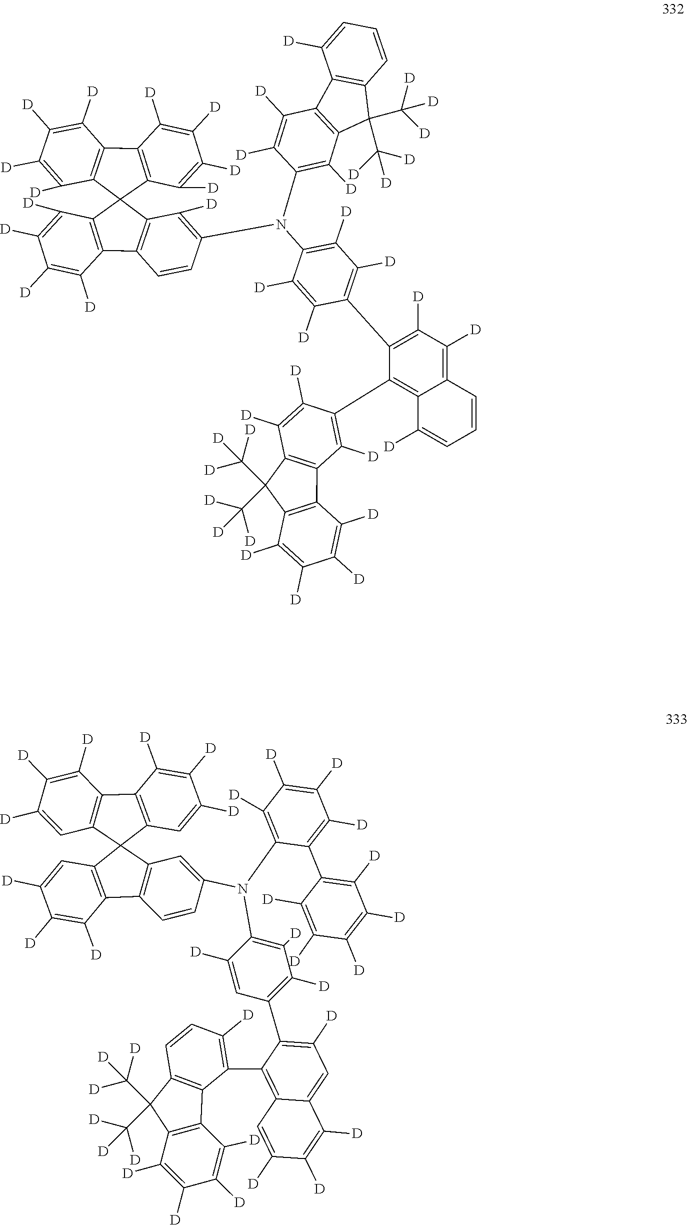

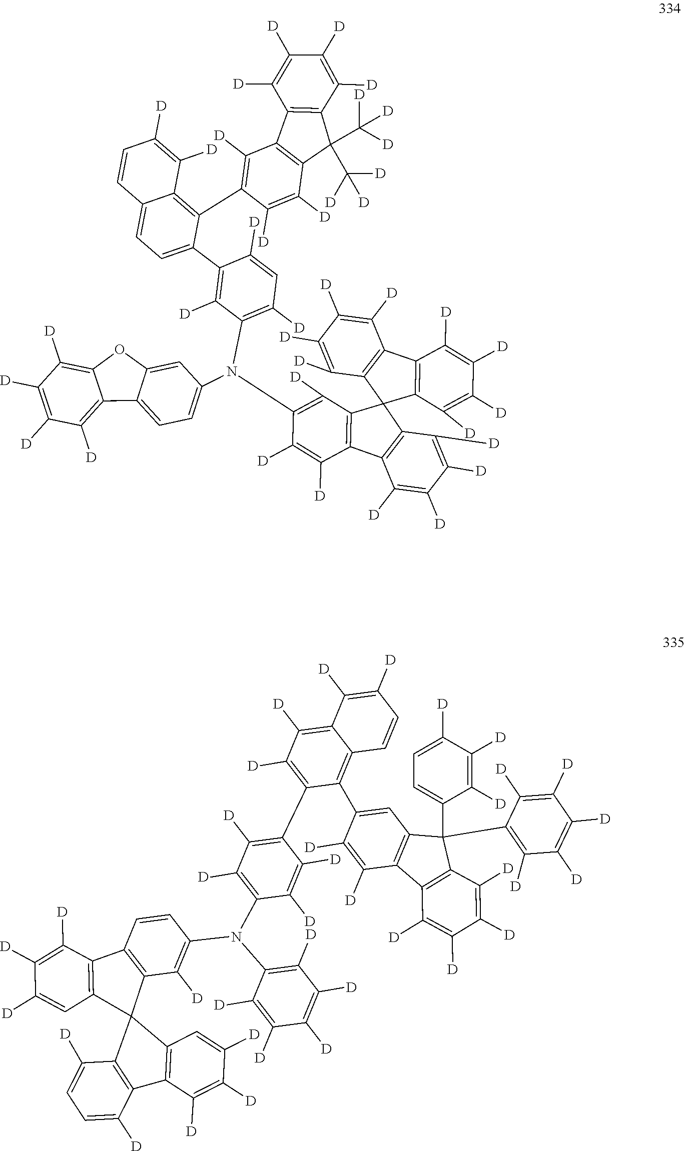

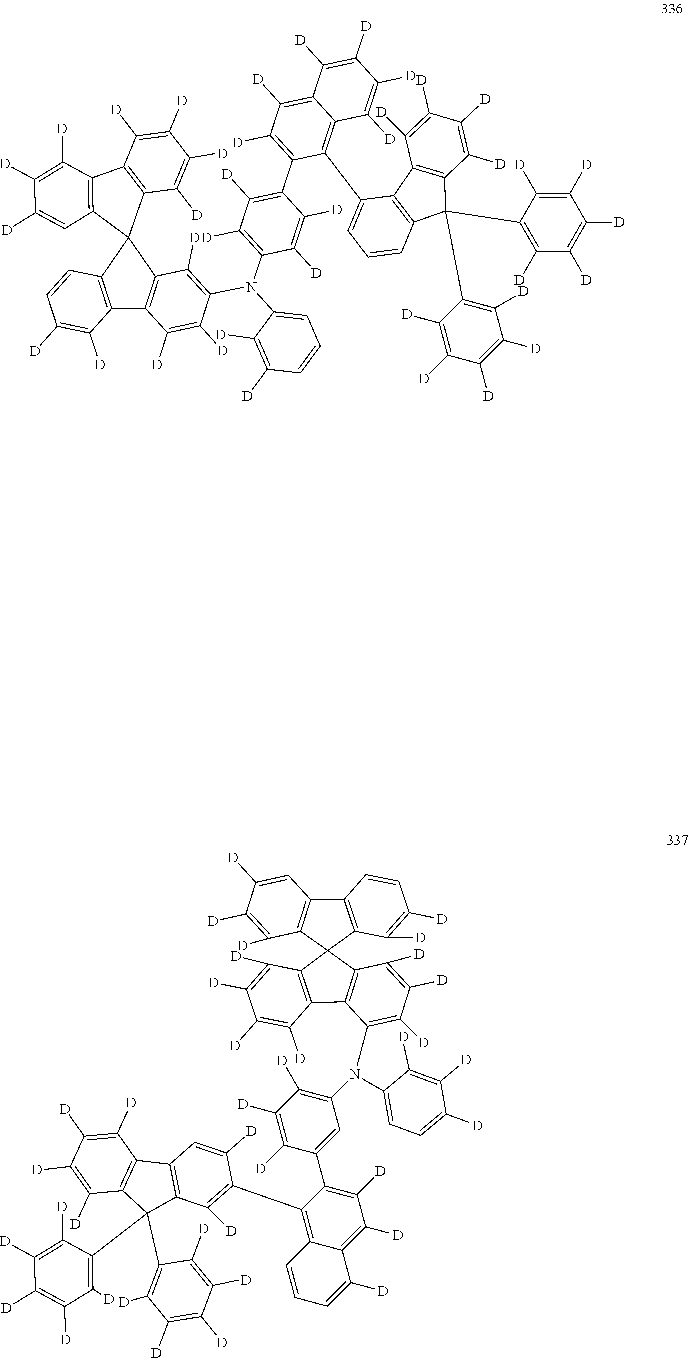

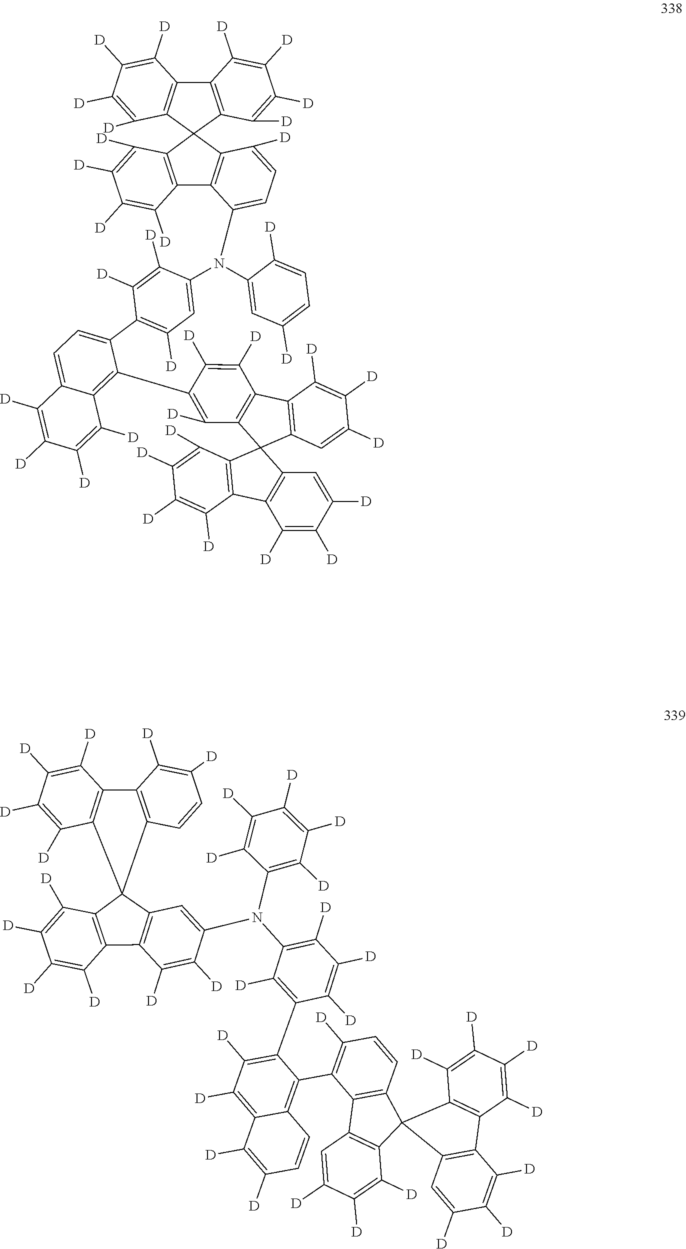

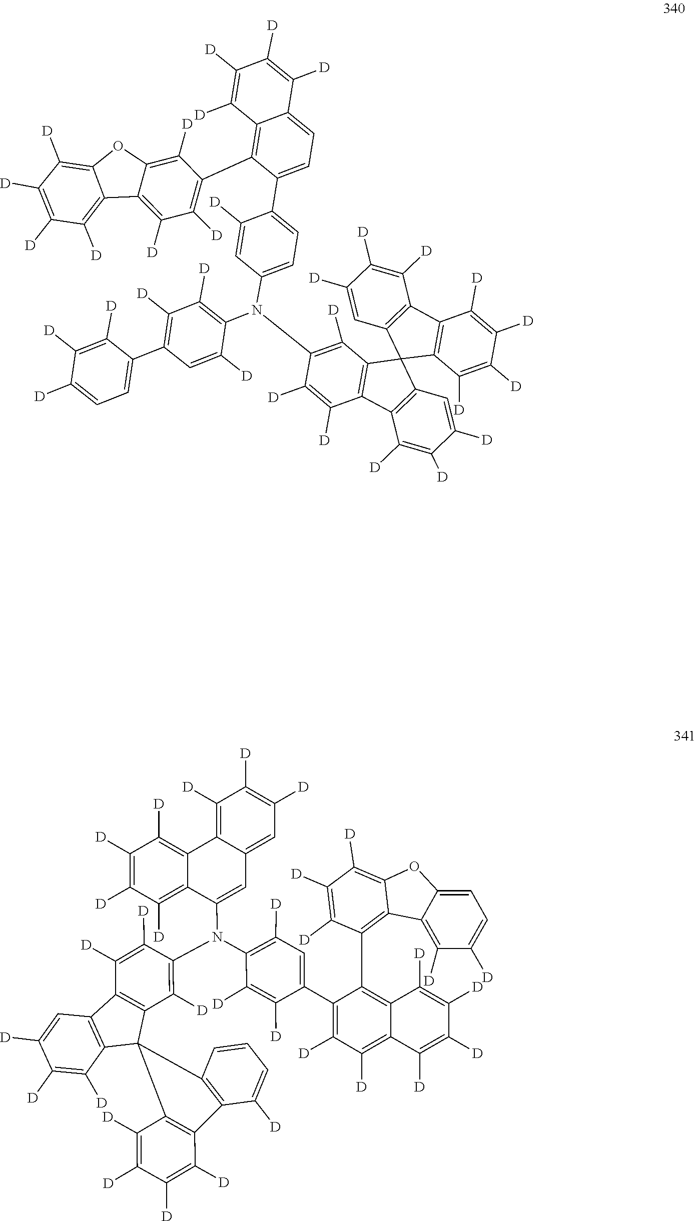

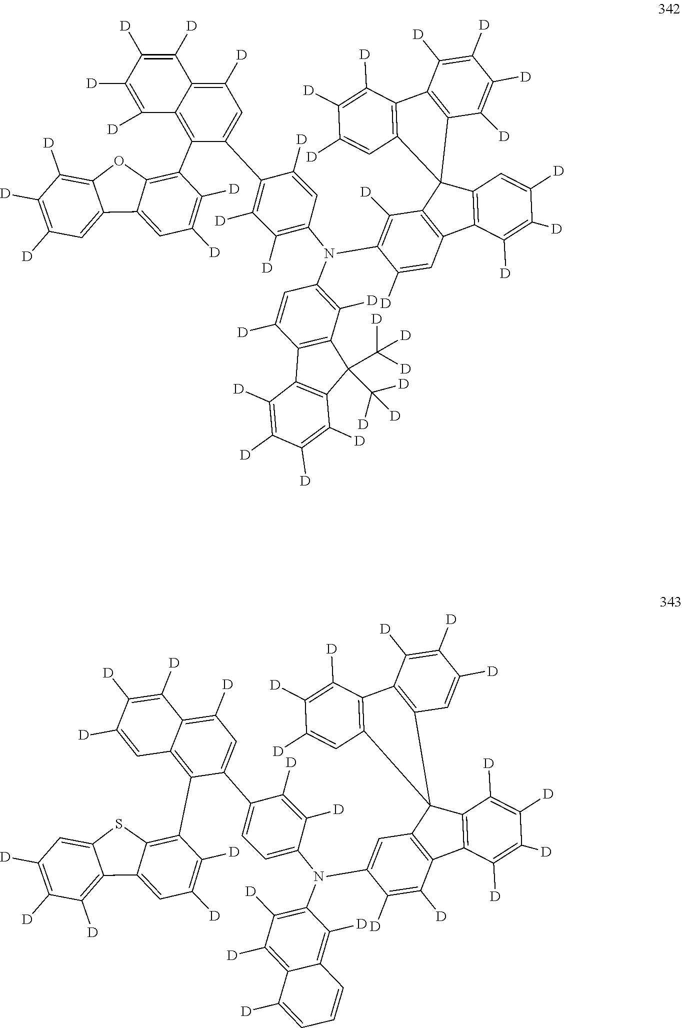

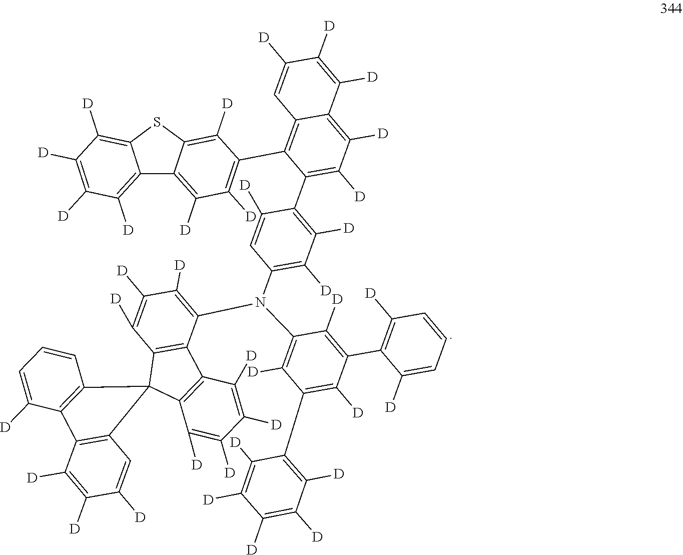 [SMILES: [2H]c1cc2c(c([2H])c1[2H])-c1c([2H])cccc1C21c2c([2H])c([2H])c([2H])c([2H])c2-c2c(N(c3cc([2H])c(-c4c([2H])c([2H])c5c([2H])c([2H])c([2H])cc5c4-c4c([2H])c([2H])c5c(sc6c([2H])c([2H])c([2H])c([2H])c65)c4[2H])c([2H])c3[2H])c3c([2H])c(-c4c([2H])cccc4[2H])c([2H])c(-c4c([2H])c([2H])c([2H])c([2H])c4[2H])c3[2H])c([2H])c([2H])c([2H])c21]

7. An organic light emitting device comprising:
a first electrode;
a second electrode; and
an organic material layer having the one or more layers provided between the first electrode and the second electrode,
wherein one or more layers of the organic material layer comprise one or more amine compounds of claim 1.

8. The organic light emitting device of claim 7, wherein the organic material layer comprises a hole transport layer, and the hole transport layer comprises the amine compound.

9. The organic light emitting device of claim 7, wherein the organic material layer comprises a hole auxiliary layer, and the hole auxiliary layer comprises the amine compound.

10. The organic light emitting device of claim 7, wherein the organic material layer comprises a light emitting layer, and the light emitting layer comprises the amine compound as a host material of a light emitting material.

11. The organic light emitting device of claim 7, further comprising one or more layers selected from the group consisting of a light emitting layer, a hole injection layer, a hole transport layer, an electron injection layer, an electron transport layer, an electron blocking layer and a hole blocking layer.
